(19) United States
(12) Patent Application Publication
(10) Pub. No.: US 2020/0405824 A1
(43) Pub. Date: Dec. 31, 2020

(54) USE OF RIBONUCLEOTIDE REDUCTASE ALONE OR IN COMBINATION WITH MICRO-DYSTROPHIN TO TREAT DUCHENNE MUSCULAR DYSTROPHY STRIATED MUSCLE DISEASE

(71) Applicant: UNIVERSITY OF WASHINGTON, Seattle, WA (US)

(72) Inventors: Guy L. ODOM, Seattle, WA (US); Michael REGNIER, Seattle, WA (US); Jeffrey S. CHAMBERLAIN, Seattle, WA (US); Stephen D. HAUSCHKA, Seattle, WA (US)

(73) Assignee: UNIVERSITY OF WASHINGTON, Seattle, WA (US)

(21) Appl. No.: 16/913,735

(22) Filed: Jun. 26, 2020

Related U.S. Application Data
(60) Provisional application No. 62/866,986, filed on Jun. 26, 2019.

(51) Int. Cl. A61K 38/44; A61K 38/17; A61K 47/69

(52) U.S. Cl. CPC A61K 38/446; A61K 47/6901; A61K 38/177

(57) ABSTRACT

The present disclosure relates generally to methods of treating a subject having muscular dystrophy or DMD. The present disclosure also relates generally to methods of prophylactically treating a subject at risk of developing muscular dystrophy or DMD. In some embodiments, the methods may include administering a pharmaceutical composition including an RRM1 gene, an RRM2 gene, and a delivery vehicle to a subject. In another embodiment, the methods may include administering a pharmaceutical composition including an RRM1 gene and an RRM2 gene coupled to a regulatory cassette to a subject. In yet another embodiment, the methods may include administering a pharmaceutical composition including an RRM 1 gene, an RRM2 gene, a regulatory cassette, and a delivery vehicle to a subject.

### CROSS-REFERENCE TO RELATED APPLICATIONS

This application claims benefit under 35 U.S.C. § 119(e) of U.S. Provisional Application No. 62/866,986 filed Jun. 26, 2019, the contents of which are incorporated herein by reference in its entirety.

### GOVERNMENT SUPPORT

This invention was made with government support under Grant No. W81XWH-18-1-0624, awarded by the Department of Defense and Grant Nos. R01 HL122332 and R01 HL128368 and R56 AG055594, awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 26, 2020, is named 034186-095790USPT_SL.txt and is 3,439,088 bytes in size.

### TECHNICAL FIELD

The technology described herein relates to methods of treating muscular dystrophy and related pathology.

### BACKGROUND OF THE INVENTION

Duchenne muscular dystrophy (DMD) is caused by mutations in the gene encoding dystrophin, a protein that links the cytoplasmic contractile components of muscle cells to the extracellular matrix. The clinical manifestations of disease progression include severe peripheral muscle weakness, respiratory insufficiency, and cardiomyopathy that advances to heart failure in many patients.

A need exists to rescue muscle function in subjects with muscular dystrophy, such as DMD.

### SUMMARY OF THE INVENTION

The present disclosure relates to a cardiac function-enhancing gene therapy approach that targets myosin in contractile filaments by overexpressing the enzyme ribonucleotide reductase (RNR). RNR converts ADP to deoxy-ADP (dADP), which is rapidly converted to dATP in cells. In humans, RNRs may be encoded by the RRM1 and RRM2 genes. Duchenne muscular dystrophy (DMD) is caused by mutations in the gene encoding dystrophin, a protein that links the cytoplasmic contractile components of muscle cells to the extracellular matrix. When dystrophin is absent or aberrant, the compromised linkage function may cause membrane damage during muscle contraction, which may lead to progressive structural and functional deterioration in cardiomyocytes and skeletal muscle cells. The clinical manifestations of disease progression may include severe peripheral muscle weakness, respiratory insufficiency, and cardiomyopathy that may advance to heart failure in patients. Gene replacement approaches for DMD in animal models and patients can partially ameliorate muscle functional deficits, though given the progressive nature of the disease, it is unclear whether these approaches can adequately address the associated cardiomyopathy. In the present study, the relative cardiac responses in an advanced-age DMD cardiomyopathy mouse model following intravenously administered recombinant adeno-associated viral (rAAV) vectors carrying muscle-specific micro-dystrophin (μDys) or ribonucleotide reductase (RNR) were compared. The results in the working examples demonstrate that both μDys and RNR treatments of DMD hearts can rescue baseline cardiac dysfunction and high workload contractile performance in isolated heart preparations. Systolic function is significantly improved by striated muscle-specific expression of μDys, but only cardiac muscle-specific expression of RNR improved both systolic and diastolic function. It was unexpected that CK8, which is actually stronger in cardiac muscle cells than cTNT, did not work as well for driving RNR expression to improve diastole—that is strength of expression alone is not sufficient to provide the best improvement in diastole. Therefore, cardiac-specific RNR expression can provide a beneficial contractile augmentation therapy for muscular dystrophy. Combination of striated muscle-specific expression of μDys with cardiac muscle-specific expression of RNR can provide further therapeutic benefits.

In one aspect, described herein is a method of treating a subject having muscular dystrophy or DMD. In another aspect, described herein is a method of prophylactically treating a subject at risk of developing muscular dystrophy or DMD. In another aspect, described herein is a method of treating a subject diagnosed with muscular dystrophy or DMD that is at risk of developing cardiomyopathy.

In one embodiment of any of the aspects, the methods comprise administering a pharmaceutical composition including an RRM1 gene, an RRM2 gene, and a delivery vehicle to a subject. In another embodiment of any of the aspects, the methods comprise administering a pharmaceutical composition including an RRM1 gene and an RRM2 gene coupled to a regulatory cassette to a subject. In another embodiment of any of the aspects, the methods include administering a pharmaceutical composition including an RRM 1 gene, an RRM2 gene, a regulatory cassette, and a delivery vehicle to a subject.

In another embodiment of any of the aspects, the methods comprise administering a first pharmaceutical composition including an RRM1 gene in a first delivery vehicle and a second pharmaceutical composition including an RRM2 gene in a second delivery vehicle, such that the first delivery vehicle and the second delivery vehicle are not the same vehicle. In another embodiment of any of the aspects, the methods comprise administering a first pharmaceutical composition including an RRM1 gene coupled to a first regulatory cassette in a first delivery vehicle and a second pharmaceutical composition including an RRM2 gene coupled to a second regulatory cassette in a second delivery vehicle, such that the first delivery vehicle and the second delivery vehicle are not the same vehicle.

In another embodiment of any of the aspects, the methods comprise administering a pharmaceutical composition including (i) an RRM1 gene and/or an RRM2 gene, operably coupled to a first regulatory cassette; (ii) a micro-dystrophin gene encoding a protein, operably coupled to a second regulatory cassette; and (iii) one or more delivery vehicles. In some embodiments, the methods comprise administering a pharmaceutical composition including (i) an RRM 1 gene, operably coupled to a first regulatory cassette; (ii) an RRM2 gene, operably coupled to a second regulatory cassette; (iii) a micro-dystrophin gene encoding a protein, operably coupled to a third regulatory cassette; and (iv) one or more delivery vehicles.

In another embodiment of any of the aspects, the methods comprise administering (i) a first pharmaceutical composition including an RRM1 gene, an RRM2 gene, a first regulatory cassette, and a first delivery vehicle, and (ii) a second pharmaceutical composition including a micro-dystrophin gene, a second regulatory cassette, and a second delivery vehicle, such that the first delivery vehicle and the second delivery vehicle are separate delivery vehicles. In another embodiment of any of the aspects, the methods comprises administering (i) a first pharmaceutical composition including an RRM1 gene, a first regulatory cassette, and a first delivery vehicle, (ii) a second pharmaceutical composition including an RRM2 gene, a second regulatory cassette, and a second delivery vehicle; and (iii) a third pharmaceutical composition including a micro-dystrophin gene, a third regulatory cassette, and a third delivery vehicle, such that the first delivery vehicle, the second delivery vehicle, and the third delivery vehicles are separate delivery vehicles.

In another embodiment of any of the aspects, the regulatory cassettes are selected from the group consisting of: a cardiac troponin T (cTNT) regulatory cassette; a creatine kinase regulatory cassette; a muscle creatine kinase (MCK) regulatory cassette; a CK8 regulatory cassette; a MHCK7 regulatory cassette; CK7 regulatory cassette; and any fragment or combinations thereof.

In another embodiment of any of the aspects, the methods comprise administering (i) a first pharmaceutical composition including an RRM1 gene, an RRM2 gene, a cTnT regulatory cassette, and a first delivery vehicle, and (ii) a second pharmaceutical composition including a micro-dystrophin gene, a CK8 regulatory cassette, and a second delivery vehicle.

In another embodiment of any of the aspects, the methods comprise administering (i) a first pharmaceutical composition including an RRM1 gene, a cTnT regulatory cassette, and a first delivery vehicle, (ii) a second pharmaceutical composition including an RRM2 gene, a cTnT regulatory cassette, and a second delivery vehicle; and (iii) a third pharmaceutical composition including a micro-dystrophin gene, a CK8 regulatory cassette, and a third delivery vehicle.

DNA can be introduced into a subject's cells in several ways. There are transfection methods, including chemical methods such as calcium phosphate precipitation and liposome-mediated transfection, and physical methods such as electroporation. There are also methods that use recombinant viruses. Current viral-vector mediated gene delivery methods include, but are not limited to, retrovirus, lentivirus, adenovirus, herpes virus, pox virus, and adeno-associated virus (AAV) vectors.

In another embodiment of any of the aspects, the delivery vehicle includes an adeno-associated virus (AAV) vector or a recombinant adeno-associated virus vector (rAAV).

In another embodiment of any of the aspects, the pharmaceutical compositions is configured to reduce a pathological effect or symptom of a muscular dystrophy. In another embodiment of any of the aspects, the muscular dystrophy is selected from the group consisting of: myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, and/or another suitable muscular dystrophy.

In another aspect, described herein is a method of improving cardiac diastole in a subject in need thereof, the method comprising administering to the subject a therapeutically effective amount of a first pharmaceutical composition comprising an RRM1 gene and an RRM2 gene operably coupled to a first regulatory cassette, whereby cardiac diastole is improved in the subject.

In one embodiment of this or any aspect described herein, cardiac systole is also improved in the subject by said administering.

In another embodiment of this or any aspect described herein, the first regulatory cassette comprises a cardiac muscle-specific regulatory cassette.

In another embodiment of this or any aspect described herein, the cardiac muscle-specific regulatory cassette comprises a cTnT regulatory cassette.

In another embodiment of this or any aspect described herein, the method further comprises administering an effective amount of a second pharmaceutical composition comprising a μDys polypeptide operably coupled to a second regulatory cassette, wherein the second regulatory cassette is different from the first regulatory cassette.

In another embodiment of this or any aspect described herein, the first regulatory cassette comprises a cardiac muscle-specific regulatory cassette, and the second regulatory cassette comprises a striated muscle-specific regulatory cassette.

In another embodiment of this or any aspect described herein, the cardiac muscle-specific regulatory cassette comprises a cTNT regulatory cassette and the striated muscle-specific regulatory cassette comprises a CK8 regulatory cassette.

In another embodiment of this or any aspect described herein, the subject has muscular dystrophy.

In another embodiment of this or any aspect described herein, the subject's muscular dystrophy is a dystrophin-related muscular dystrophy.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its definition provided herein, the definition provided within the specification shall prevail.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, the term “muscular dystrophy” refers to a class of inherited diseases involving progressive weakness and loss of muscle mass. Muscular dystrophies include various forms involving mutation or dysregulation of the expression of the dystrophin gene or its protein product; dystrophin-related muscular dystrophies include Duchenne muscular dystrophy (DMD) and Becker muscular dystrophy (BMD), as well as DMD-associated dilated cardiomyopathy. Other non-limiting forms of muscular dystrophies include: myotonic muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy.

As used herein, the term “cardiac muscle-specific regulatory cassette” refers to a gene expression regulatory cassette that drives expression of an operatively linked gene sequence in cardiac muscle cells, but substantially not in other muscle cells (including skeletal muscle cells) or other non-muscle cells. By “substantially not” in this regard is meant that the expression of an operatively linked gene sequence is at least 20-fold lower in non-cardiac muscle cells, preferably at least 30-fold lower, at least 40-fold lower, at least 50-fold lower, at least 75-fold lower or at least 100-fold lower in non-cardiac muscle cells. Thus, a cardiac muscle-specific regulatory cassette will drive expression at least 20-fold, at least 30-fold, at least 40-fold, at least 50-fold, at least 75-fold or at least 100-fold more strongly than in non-cardiac muscle cells.

As used herein, the term “striated muscle-specific regulatory cassette” refers to a gene expression regulatory cassette that drives expression of an operatively linked gene sequence in striated muscle cells, but substantially not in non-striated muscle cells or other non-muscle tissues. By “substantially not” in this regard is meant that the expression of an operatively linked gene sequence is at least 20-fold lower in non-striated muscle cells, preferably at least 30-fold lower, at least 40-fold lower, at least 50-fold lower, at least 75-fold lower or at least 100-fold lower in non-striated muscle cells. Thus, a striated muscle-specific regulatory cassette will drive expression at least 20-fold, at least 30-fold, at least 40-fold, at least 50-fold, at least 75-fold or at least 100-fold more strongly than in non-striated muscle cells. It should be understood that cardiac muscle is a type of striated muscle—as such, a striated muscle-specific regulatory cassette will drive gene expression in cardiac, as well as in other striated muscle cells, e.g., skeletal muscle cells.

As used herein, the terms “treat,” “treatment,” “treating,” or “amelioration” refer to therapies, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder associated with a muscular dystrophy. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation or at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

As used herein “preventing” or “prevention” refers to any methodology where the disease state does not occur due to the actions of the methodology (such as, but not limited to, administration of a pharmaceutical composition or other therapeutic described herein). In one aspect, it is understood that prevention can also mean that the disease is not established to the extent that occurs in untreated controls. Accordingly, prevention of a disease encompasses a reduction in the likelihood that a subject can develop the disease, relative to an untreated subject (e.g. a subject who is not treated with the methods or compositions described herein) likely to develop the disease.

The terms “increased” or “increase” are used herein to mean an increase by a statically significant amount. In some embodiments, the terms “increased” or “increase” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level (e.g., the absence of an isolated nucleic acid molecule, polypeptide, vector, composition, or pharmaceutical composition described herein), or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of an isolated nucleic acid molecule, polypeptide, vector, composition, or pharmaceutical composition described herein) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., Rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that provide animal models of disease e.g., cardiac disease or disorder, such as myocardial infarction or myocardial ischemia. A subject can be male or female.

A subject can be one who has been previously diagnosed with or identified as suffering from or having a condition in need of treatment (e.g., muscular dystrophy) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition (e.g., muscular dystrophy or a complication thereof), diagnosed as having that condition, or at risk of developing that condition. As non-limiting examples, a subject diagnosed with or suffering from a given condition, a subject determined to have a mutation predisposing to a given condition, and a subject whose parent or sibling is known to carry a mutation predisposing to a given condition are each subjects in need of treatment.

In some embodiments, a polypeptide described herein (or a nucleic acid encoding such a polypeptide) can be a functional fragment of one of the polypeptides described herein, e.g., a functional fragment of a dystrophin (including a μDys), RRM1 or RRM2 polypeptide.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise.

Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

### BRIEF DESCRIPTION OF THE DRAWINGS

The patent or application file contains at least one drawing executed in color. Copies of this patent application publication with color drawings will be provided by the Office upon request and payment of the necessary fee.

FIG. 1A shows a graphic representation of rAAV6 vectors utilized in the present disclosure. The ribonucleotide reductase vector contains the human cDNA for the RRM1 and RRM2 subunits whose expression is driven by the cardiac specific (cTnT455) regulatory cassette (RC). The human micro-dystrophin (ΔR2-R15/ΔR18-R22/ΔCT) vector has expression driven by the CK8 muscle specific RC. The control vector utilized in the present study carries the firefly luciferase transgene whose CMV early promoter/enhancer RC has been deleted. FIG. 1B shows an outline of animal enrollment, vector administration, and experimental protocols implemented following a treatment period of 5 months.

FIG. 2 shows Kaplan Meier analysis was performed on mdx^{4cv} mice treated with control vector (mdx^{4cv}, n=6), ribonucleotide reductase (mdx^{4cv}+RNR, n=6), or micro-dystrophin (mdx^{4cv}+μDys, n=5). All mice were followed for 20 weeks post injection.

FIG. 3A-3D shows hearts isolated from mdx^{4cv} mice treated with control vector (mdx^{4cv}, n=6), ribonucleotide reductase (mdx^{4cv}+RNR, n=6), or micro-dystrophin (mdx^{4cv}+μDys, n=5). Age-matched, non-diseased, non-treated wild-type mice were used as controls (WT, n=8). All hearts were perfused with a glucose-pyruvate buffer. Functional assessment was performed at spontaneous heart rates. FIG. 3A shows left ventricular developed pressure (LVDevP, the difference between systolic and diastolic pressures). FIG. 3B shows rate pressure product (RPP, the product of LVDevP and HR). FIG. 3C shows positive rate of pressure change calculated by the first derivative of the ascending LV pressure wave (+dP/dt), used as an index of ventricular contractility. FIG. 3D shows negative rate of pressure change calculated by the first derivative of the descending LV pressure wave (−dP/dt), used as an index of ventricular relaxation. *P<0.05 mdx^{4cv} vs. WT; #P<0.05 mdx^{4cv}+RNR vs. mdx^{4cv}.

FIG. 4A-4E shows hearts isolated from mdx^{4cv} mice treated with control vector (mdx^{4cv}, n=6), ribonucleotide reductase (mdx^{4cv}+RNR, n=6), or micro-dystrophin (mdx^{4cv}+μDys, n=5). Age-matched, non-diseased, non-treated wild-type mice were used as controls (WT, n=8). The pressure-volume relationship (i.e., Frank-Starling mechanism) was evaluated by gradually increasing the volume of the LV balloon. Hearts were paced at 450 bpm throughout the protocol. FIG. 4A shows left ventricular systolic pressure (LVSP). FIG. 4B shows LV end-diastolic pressure (LVEDP). FIG. 4C shows left ventricular developed pressure (LVDevP, the difference between systolic and diastolic pressures). FIG. 4D shows negative rate of pressure change calculated by the first derivative of the descending LV pressure wave (−dP/dt), and is used as an index of ventricular relaxation. FIG. 4E shows positive rate of pressure change was calculated by the first derivative of the ascending LV pressure wave (+dP/dt), and is used as an index of ventricular contractility. *P<0.05 mdx^{4cv} vs. WT; #P<0.05 mdx^{4cv}+RNR vs. mdx^{4cv}; $ P<0.05 mdx^{4cv}+μDys vs. mdx^{4cv}.

FIG. 5A-5D shows hearts isolated from mdx^{4cv} mice treated with control vector (mdx^{4cv}, n=6), ribonucleotide reductase (mdx^{4cv}+RNR, n=6), or micro-dystrophin (mdx^{4cv}+μDys, n=5). Age-matched, non-diseased, non-treated wild-type mice were used as controls (WT, n=4). All hearts were perfused with a glucose-pyruvate buffer containing high calcium (4.0 mmol/L) to simulate a high workload challenge for 20 min. Hearts were paced at 450 bpm throughout the protocol. FIG. 5A shows left ventricular developed pressure (LVDevP, the difference between systolic and diastolic pressures). FIG. 5B shows rate pressure product (RPP, the product of LVDevP and HR). FIG. 5C shows positive rate of pressure change calculated by the first derivative of the ascending LV pressure wave (+dP/dt), is used as an index of ventricular contractility. FIG. 5D shows negative rate of pressure change calculated by the first derivative of the descending LV pressure wave (−dP/dt), is used as an index of ventricular relaxation. *P<0.05 mdx^{4cv} vs. WT; #P<0.05 mdx^{4cv}+RNR vs. mdx^{4cv}; $ P<0.05 mdx^{4cv}+μDys vs. mdx^{4cv}.

FIG. 6 demonstrates that 5-months following vector administration, cryosections were prepared and immunostained with antisera against dystrophin or ribonucleotide reductase. A considerable level of protein is detected for each ribonucleotide reductase subunit-1 (human specific) as indicated by immunofluorescent staining (Red) localized primarily within the cytoplasm of cardiomyocytes with occasional perinuclear accumulation (upper panel). Noteworthy on the lower panel, is the robust level of expression for dystrophin in WT and in aged mdx^{4cv} mice treated with AAV6-CK8-micro-dystrophin (laminin staining, inset image).

FIG. 7 shows a representative full-view photomicrographs of Masson trichrome staining of the hearts from mdx^{4cv} mice displaying control vector (4CMV), and rAAV6-treated with either RNR or μDys from mdx^{4cv} mice. Similarly, 20× enlarged view of the corresponding images (*) is shown.

FIG. 8A shows body weights and FIG. 8B shows heart weights were obtained from mdx^{4cv} mice treated with control vector (mdx^{4cv}, n=6), ribonucleotide reductase (mdx^{4cv}+RNR, n=6), or micro-dystrophin (mdx^{4cv}+μDys, n=5). FIG. 8C shows heart weights (HW) normalized to body weights (BW) to obtain HW/BW ratio. No statistical differences were noted among the variables.

FIG. 9A shows RNR and μDys protein expression detection as revealed by immunoblotting of cardiac whole tissue lysates using either RRM1, RRM2 or anti-dystrophin antibody. FIG. 9B shows HPLC-MS/MS intracellular [dATP] quantification from methanol extracted cardiac tissue. FIG. 9C shows qPCR analysis of vector genomes from cardiac tissue revealed similar vector genomes being represented for all treated cohorts.

FIG. 10 shows The presence of empty capsids aided the transduction efficiency of AAV6 and AAV9 in mature human myotube cultures, but appear to hinder that of AAV9 in MM14 cultures. The transduction efficiency of AAV8 was the lowest compared to AAV6 and 9 in mouse and human mature myotube cultures, but was similar to AAV6 in canine myotube cultures. In contrast, AAV9 transduced poorly in canine myotube cultures.

FIG. 11A-11B shows Intravenous dose response of rAAV6-CMV-hPLAP transduction in striated muscle. Mice were injected via tail vein with increasing doses of vector, and tissues harvested 2 weeks post-injection. FIG. 11A shows chemiluminescent assay of alkaline phosphatase activity in muscle lysates. RLU, relative light units. The data represent mean values±SEM (n=3 for all cohorts except 0 vg, n=7; 1.3×10¹² vg, n=7; 2.5×10¹², n=4). FIG. 11B shows representative sections of muscles stained for alkaline phosphatase activity from mice receiving increasing doses of vector. Hrt, heart; Dia, diaphragm; TA, tibialis anterior. Scale bar=100 μm.

FIG. 12A-12C shows effect of empty capsids on intravenous administration of rAAV6-CMV-hPLAP. Mice were injected with 1.3×10¹² vector genomes of “full capsids”±empty capsids of various serotypes. FIG. 12A shows representative sections of muscles stained for alkaline phosphatase activity. Hrt, heart; Dia, diaphragm; Sol, soleus; Liv, liver. Scale bar=100 um. FIG. 12B shows chemiluminescent assay of alkaline phosphatase activity in tissue lysates. FIG. 12C shows vector genomes normalized to diploid mouse genomes, quantified by qPCR to either the vector sequence or sequence of the murine LDL receptor. The data in (FIG. 12B-12C) represent mean values±SEM. (Fulls alone, +AAV1 empties, +AAV6 empties: n=5; +AAV2 empties, +AAV8 empties: n=4)<0.05, ** P<0.01 vs. “rAAV6 Fulls” by one-way ANOVA with Dunnett's post-test. For (FIG. 12B-12C), note the different scales on the ordinate for each tissue.

FIG. 13A-13F shows that various engineered RNRs can increase dATP activity in both young and old mdx^{4cv} mouse hearts. Vector comparisons are saline (control=no AAV), a promotor-less construct (A-RNR), cTnT promotor (cTnT-RNR), CK8e promotor (CK8-RNR), CK8e with double mutation in RRM2 to resist ubiquitination (CK8-RNR-DM, CK8e-R1.R2dm), CK8m with RNR double mutation (CK8mR1.R2dm) and CK8e with a different (gene) R2 subunit (CK8e-R1.R2b) that is naturally degradation resistant. Data are presented as quantity in pmol/mg tissue (top graphs) and as a % of the ATP pool (bottom graphs). FIG. 13A-13C shows the dATP content in ventricular tissues (pmol/mg). FIG. 13A shows dATP content in old mice that received a promotor-less (A-RNR), a cTnT promotor (cTnT-RNR), and a CK8e promotor (CK8-RNR). FIG. 13B shows dATP content in young mice that received a promotor-less (A-RNR), a cTnT promotor (cTnT-RNR), a CK8e promotor (CK8-RNR), and a CK8e with double mutation in RRM2 (CK8-RNR-DM). FIG. 13C shows dATP content in young mice that received saline (control), rAAV-6-CK8e-R1R2, rAAV-CK8e-R1.R2dm, and rAAV6-CK8e-R1R2b, compared with un-injected mice. FIG. 13D-13F shows dATP as % of total ATP pool as in FIG. 13A-13C.

### DETAILED DESCRIPTION

Duchenne Muscular Dystrophy (DMD) and its milder and allelic form, Becker muscular dystrophy (BMD), are the most frequent muscular dystrophies, occurring once in ˜5000 male births, and are due to mutations in the dystrophin gene (1). DMD patients typically die due to cardiac and respiratory muscle failure; thus, maintenance of adequate function in both cardiac and skeletal muscle is important for optimal DMD therapy. The primary function of dystrophin is to provide a structural role by mechanically linking the subsarcolemmal cytoskeleton to the extracellular matrix (ECM) through the dystrophin-glycoprotein complex (DGC) (2). This linkage transmits the forces of contraction to the extracellular matrix (ECM) and protects muscles from contraction-induced injury (3-7). In addition to a structural or mechanical role, the DGC also serves as a scaffold for cytoplasmic and membrane-associated signaling proteins and ion channels (8-11). The complete absence of dystrophin results in drastic reductions of all DGC components (12-14). Together, an absence of dystrophin and reduction in the DGC components causes membrane destabilization and permeability defects that lead to myofiber degeneration, repeated cycles of degeneration/regeneration, and the gradual replacement of muscle fibers with fibrotic, connective, and adipose tissue.

In contrast, some in-frame deletions, truncations, and missense mutations lead to reduced dystrophin expression associated with milder phenotypes. These pathologies are largely curtailed in mouse (mdx) and canine (cxmd) models of DMD following the vector mediated delivery of muscle-specific expression of highly functional miniaturized versions of dystrophin, micro-dystrophin (μDys) (15-24). In mdx mice, muscle pathology may be milder than in humans; however, the dystrophic phenotype may worsen with increasing age including the development of cardiac dysfunction (25-32). Administration of rAAV-mediated μDys therapy in mdx mice preceding the onset of cardiomyopathy may be highly cardioprotective (33-35). However, when mdx mice are treated with μDys at a late stage of cardiomyopathy, such as would be the case for a number of DMD patients, a full rescue of the dysfunctional cardiac phenotype is not achieved (30,35-37).

The present disclosure relates to a cardiac function-enhancing gene therapy approach that targets myosin in contractile filaments and overexpresses the enzyme ribonucleotide reductase (RNR). RNR converts ADP to deoxy-ADP (dADP), which can be rapidly converted to dATP in cells. In numerous in vitro studies, it has been shown that dATP can increase cross bridge binding and cycling, which results in stronger, faster contraction and faster relaxation (38-46). Furthermore, dATP can improve the contractile properties of the myocardium from end-stage human heart failure (HF) in vitro (43) and in dog models with end-stage idiopathic dilated cardiomyopathy (47). In normal rodent muscle, increases in cardiomyocyte and cardiac function can occur with as little as ˜1% of the ATP pool in the dATP form (40,48). Similarly, rAAV-mediated delivery of RNR under cardiac specific regulatory control can result in enzyme overexpression exclusively in cardiomyocytes and significantly improved left ventricular function without adverse cardiac remodeling in normal and infarcted rodent hearts (49). Thus, dATP can rescue the pre-load responsiveness of failing hearts, restoring the pressure and volume to normal.

In the working examples, the relative therapeutic capacity of muscle-specific microdystrophin (μDys) or ribonucleotide reductase (RNR), via intravenously administered recombinant adeno-associated viral (rAAV) vectors in an advanced age, DMD cardiomyopathy mouse model, were compared. A restoration of myocardial workload was demonstrated as indicated by rate pressure product (RPP), for baseline function in mdx^{4cv} mice treated with RNR. This outcome was primarily attributed to the normalization of left ventricular developed pressure (LVDevP). Although mdx^{4cv} mice treated with μDys appeared to normalize LVDevP, this did not result in a significant increase in RPP. Upon further evaluation of cardiac function, the pressure-volume relationship revealed that systolic pressure response with increased preload was significantly improved with the treatment of either RNR or μDys. However, only RNR treatment resulted in significant improvements in diastolic functional parameters, returning them to values that were similar to wild-type control hearts. As a further assessment of cardiac function, hearts were tested using a high workload challenge protocol. Both RNR and μDys treatments improved systolic function in mdx^{4cv} hearts without compromising cardiac reserve. The results in the examples described herein demonstrate that targeted expression of RNR within the myocardium significantly improves contractile performance in an advanced age model of DMD cardiomyopathy and can be a valuable therapeutic for the prevention and treatment of muscular dystrophy and DMD patients. Surprisingly, cardiac-specific expression of RNR improved systolic and diastolic function of the heart to a greater extent than striated muscle-specific expression of RNR, despite the actual level of RNR driven in cardiac cells by a cardiac-specific regulatory cassette being lower than expression from the striated muscle-specific cassette.

### Muscular Dystrophy

Compositions and methods are provided herein for treating muscular dystrophy by delivering one or more constructs encoding ribonucleotide reductase (RNR) activity to muscle in a subject in need thereof. In some embodiments, the construct is delivered alone—i.e., no other therapeutic constructs are delivered, and the RNR improves muscle function, including but not limited to cardiac muscle function, in a manner effective to treat the muscular dystrophy. In other embodiments, the construct is delivered in combination with one or more additional constructs encoding one or more additional therapeutic polypeptides. In such embodiments, the additional therapeutic polypeptide can encode, for example, a microdystrophin. The combination of RNR and microdystrophin can together attack both structural (dystrophin-related) and functional (dATP supply) deficits that contribute to the pathology, thereby more significantly improving muscular function. Where the RNR is driven by a cardiac muscle specific regulatory element or cassette, the benefit in countering cardiomyopathy stemming from muscular dystrophy can be pronounced.

There are several types of muscular dystrophy, including but not limited to: (1) myotonic dystrophies, generally characterized by an inability to relax muscles following contractions; (2) facioscapulohumeral (FSHD) dystrophies, characterized by muscle weakness typically beginning in the face, hip and shoulders, onset of FSHD usually occurs in the teenage years but can begin in childhood or as late as age 50; (3) congenital muscular dystrophy, that affects boys and girls and is apparent at birth or before age 2; and (4) limb-girdle muscular dystrophies, generally characterized by hip and shoulder muscle weakness, difficulty lifting the foot, and frequent tripping. Complications of muscular dystrophy include for example, trouble walking, difficulty using arms or legs, shortening of muscles or tendons, breathing problems, scoliosis, cardiovascular failure and arrhythmias, and swallowing problems.

Duchenne muscular dystrophy (DMD) is a recessively-inherited muscular dystrophy that affects approximately 1 in 3500 males. DMD patients carry a mutation in the dystrophin gene that causes aberrant expression or loss of expression of the dystrophin protein. DMD patients experience progressive wasting of skeletal muscles and cardiac dysfunction, which leads to loss of ambulation and premature death, primarily due to cardiac or respiratory failure.

An absence of dystrophin and reduction in the dystrophoin-glycoprotein complex (DGC) components causes membrane destabilization and permeability defects that lead to myofiber degeneration, repeated cycles of degeneration/regeneration, and the gradual replacement of muscle fibers with fibrotic, connective, and adipose tissue. This effect can lead to decreased systolic and diastolic performance in DMD hearts.

Current available treatments for DMD are generally only able to slow the pathology of DMD (see Emery, A. E. H. and Muntoni, F., Duchenne Muscular Dystrophy, Third Edition (Oxford University Press, 2003)). Gene therapy approaches for DMD have been demonstrated in dystrophic animal models by either directly targeting a class of mutations, as with exon skipping, or replacing the mutated gene with viral-vector mediated delivery (see Koo, T. and Wood, M. J. Human Gene Therapy 24, (2013); Benedetti, S., et al., The FEBS Journal 280, 4263-4280, (2013); and Seto, J. T., et al., Current Gene Therapy 12, 139-151 (2012)). Recombinant adeno-associated virus (rAAV) vectors are a potential vehicle for gene therapy, being already tested in clinical trials for both DMD and limb-girdle muscular dystrophies (see Mendell, J. R., et al., The New England Journal of Medicine 363, 1429-1437, (2010); Mendell, J. R., et al., Annals of Neurology 68, 629-638 (2010); and Herson, S., et al., Brain: A Journal of Neurology 135, 483-492, (2012)). Several serotypes of adeno-associated virus (AAV) demonstrate a high degree of tropism for striated muscles (see Seto, J. T., et al., Current Gene Therapy 12, 139-151 (2012)).

Pre-clinical studies designing and testing newer generations of therapeutic constructs for DMD can be confined by the approximately 4.9 kb size of a single-stranded rAAV vector genome (see Dong, B., et al., Molecular Therapy: The Journal of the American Society of Gene Therapy 18, 87-92, (2010) and Wu, Z., et al., Molecular Therapy: The Journal of the American Society of Gene Therapy 18, 80-86, (2010)). Packaging the entire approximately 13.9 kb cDNA of the muscle-specific isoform of dystrophin into a single rAAV capsid cannot be achieved, accordingly, miniaturized, synthetic versions of the muscle-specific isoform of dystrophin cDNA may be used.

Although in vivo recombination of two and three rAAV vector genomes has been demonstrated to deliver a mini- or full-length dystrophin coding sequence (see, Odom, G. L., et al., Molecular Therapy: The Journal of the American Society of Gene Therapy 19, 36-45, (2011); Lostal, W., et al., Human Gene Therapy, (2014); and Koo, T., et al., Human Gene Therapy 25, 98-108, (2014)), the efficiency of delivering multiple vectors for reconstituting full-length dystrophin may be suboptimal and can increase the overall dose of viral capsid proteins needed for delivering vectors. However, beneficial rAAV-mediated gene therapy has been achieved using rationally-designed miniature versions of the dystrophin cDNA based in part on mRNA expressed in mild Becker muscular dystrophy patients carrying in-frame deletions within the gene (see Beggs, A. H., et al., American Journal of Human Genetics 49, 54-67 (1991); Koenig, M., et al., American Journal of Human Genetics 45, 498-506 (1989); Goldberg, L. R., et al., Annals of Neurology 44, 971-976, (1998); and England, S. B., et al., Nature 343, 180-182 (1990)). Studies in transgenic and vector treated dystrophic mice expressing various dystrophin truncations have identified several elements of the dystrophin gene that need to be present in a functional micro-dystrophin (μDys) (see Harper, S. Q., et al., Nature Medicine 8, 253-261, (2002)). See additional below re: microdystrophins.

The methods provided herein provide a cardiac function-enhancing approach to therapeutically treat muscular dystrophy by targeting myosin in contractile filaments via overexpression of ribonucleotide reductase (RNR) without adverse cardiac remodeling (see, e.g., Kolwicz et al. JACC Vol 4, No 7, 2019, which is incorporated herein by reference in its entirety).

The nucleic acid constructs provided herein affect the cardiac pressure-volume relationship by significantly improving systolic preload response. Accordingly, administration of RNR alone can improve diastolic (at rest) functional parameters of the dystrophic heart in animal models of DMD, a surprisingly beneficial effect of the compositions described herein. This is because current therapeutics targeting cardiovascular complications of DMD only improve structural and/or systolic (contraction) function of the heart and do not necessarily improve diastolic function or cardiovascular energetics. Where most therapies for muscle-related cardiac pathologies focus on improving contraction, a therapeutic approach that improves diastolic function or relaxation can improve the efficiency of the heart because improved relaxation permits a greater volume of blood to enter the chamber before contraction drives it out.

Methods of measuring cardiac function and energetics (e.g., pressure and volume) in a subject include, but are not limited to, echocardiography, magnetocardiogram, and a Langendorff perfusion in a test animal. See also, e.g., Kolwicz S C, Jr. and Tian R. Assessment of cardiac function and energetics in isolated mouse hearts using 31P NMR spectroscopy. J Vis Exp. 2010; 42: e2069.

Given that the RNR increases dATP in the heart, the nucleic acid constructs described herein can be used prophylactically to support cardiac function in subjects with muscular dystrophy and prevent or decrease the severity of cardiovascular complications. As shown in the working examples, RNR overexpression results in elevated dATP, which can be used by cardiac myosin (in place of ATP), and increases cross-bridge binding and cycling, resulting in stronger, faster contraction and faster relaxation in mouse models of DMD.

### Dystrophins

The full-length striated muscle isoform of dystrophin plays a role in transmitting contractile force through the sarcolemma and out to the extracellular matrix. In addition to maintaining the mechanical link between the intracellular cytoskeleton and the membrane bound dystrophin glycoprotein complex (DGC), dystrophin can also be a scaffold for signaling proteins (see e.g., Ozawa, E. in Myology (ed. Franzini-Armstrong C Engel A) 455-470 (McGraw-Hill, 2004); Winder, S. J. Journal of Muscle Research and Cell Motility 18, 617-629 (1997); and Campbell, K. P. and Kahl, S. D. Nature 338, 259-262, (1989)), which are incorporated herein by reference in their entireties. The amino-terminal domain of dystrophin can bind to F-actin filaments of the intracellular cytoskeleton (see e.g., Way, M., et al., FEBS Letters 301, 243-245 (1992); Hemmings, L., et al., The Journal of Cell Biology 116, 1369-1380 (1992); Fabbrizio, E., et al., Biochemistry 32, 10457-10463 (1993); and Pavalko, F. M. and Otey, C. A. Proceedings of the Society for Experimental Biology and Medicine 205, 282-293 (1994), which are incorporated herein by reference in their entireties). The human dystrophin gene, mRNA and polypeptide sequences is known in the art, see, e.g., SEQ ID NO: 31-33, or a variant thereof.

The middle, rod domain is the largest and is composed of 24 spectrin-like repeats (SRs) that are flanked and interspersed with at least four hinge sub-domains. The rod domain can give dystrophin elasticity and flexibility for maintaining the integrity of the sarcolemma during muscle contractility (see Winder, S. J. Journal of Muscle Research and Cell Motility 18, 617-629 (1997)). Various SRs provide unique regions that can serve as additional binding sites for the intracellular cytoskeleton, the sarcolemma, as well as members of the DGC (see Rybakova, I. N., et al., The Journal of Cell Biology 135, 661-672 (1996); Warner, L. E., et al., Human Molecular Genetics 11, 1095-1105 (2002); Metzinger, L., et al., Human Molecular Genetics 6, 1185-1191 (1997); Lai, Y., et al., The Journal of Clinical Investigation 119, 624-635, (2009)). In particular, the cysteine-rich domain and the adjacent Hinge 4 region form the (3-dystroglycan binding domain (Dg BD) (see Blake, D. J., et al., Physiological Reviews 82, 291-329, (2002); Ishikawa-Sakurai, M., et al., Human Molecular Genetics 13, 693-702, (2004)), while the carboxy-terminal domain is a scaffold for additional DGC components (see Abmayr S, in Molecular Mechanisms of Muscular Dystrophies (ed. Winder, S. J.) 14-34 (Landes Biosciences, 2006)).

Partially functional micro-dystrophins can improve the dystrophic pathology in striated muscle by protecting the sarcolemma from contraction-induced injury and increasing the capacity to generate force. These parameters can be achieved by binding to F-actin filaments and β-dystroglycan through the amino-terminal domain and the Dg BD (see Harper, S. Q., et al., Nature Medicine 8, 253-261, (2002); Warner, L. E., et al., Human Molecular Genetics 11, 1095-1105 (2002); Cox, G. A., et al., Nature Genetics 8, 333-339, (1994); Greenberg, D. S., et al., Nature Genetics 8, 340-344, (1994); Gardner, K. L., et al., Gene Therapy 13, 744-751, (2006); Corrado, K., et al., The Journal of Cell Biology 134, 873-884 (1996); and Rafael, J. A., et al., The Journal of Cell Biology 134, 93-102 (1996)). Without being bound by any one particular theory, prior studies indicate these two domains must be connected by at least four SRs from the central rod domain, but there are numerous ways in which miniaturized dystrophins containing at least four SRs can be constructed. While some combinations of SRs have been shown to improve the dystrophic pathophysiology, other combinations have not yielded proteins with significant functional capacity (see Harper, S. Q., et al., Nature Medicine 8, 253-261, (2002) and Abmayr S, in Molecular Mechanisms of Muscular Dystrophies (ed. Winder, S. J.) 14-34 (Landes Biosciences, 2006)). Selection of specific SRs in μDys design can restore additional DGC components to the sarcolemma. Neuronal nitric oxide synthase (nNOS) is a signaling protein that can be involved in vasodilation in response to muscle contractile activity (see Stamler, J. S. and Meissner, G. Physiological Reviews 81, 209-237 (2001); Brenman, J. E., et al., Cell 82, 743-752 (1995); Kobayashi, Y. M., et al., Nature 456, 511-515, (2008); and Torelli, S., et al., Neuropathology and Applied Neurobiology 30, 540-545, (2004)), and the presence of SRs 16 and 17 can be involved in proper association of nNOS with the DGC (see 28 Lai, Y. et al., The Journal of Clinical Investigation 119, 624-635, (2009) and Lai, Y., et al., Proceedings of the National Academy of Sciences of the United States of America 110, 525-530, (2013)).

Sequences within spectrin-like repeats 20-24 as well as Hinge 4 can play a role in proper association of dystrophin with microtubules, which can be important for maintaining the intracellular architecture and torque production in skeletal muscle (see Prins, K. W. et al., The Journal of Cell Biology 186, 363-369, (2009) and Belanto, J. J., et al., Proceedings of the National Academy of Sciences of the United States of America 111, 5723-5728, (2014)). Nonetheless, the carboxy-terminal domain and most of the SR domains have been found dispensable without severely compromising the health of striated muscles (see McCabe, E. R., et al., The Journal of Clinical Investigation 83, 95-99, (1989); Crawford, G. E., et al., The Journal of Cell Biology 150, 1399-1410 (2000); and Dunckley, M. G., et al., FEBS Letters 296, 128-134 (1992)).

Any micro-dystrophin (referred to herein as μDys or mDys) known in the art can be administered in combination with the RNR constructs described herein. By way of example only, the RNR constructs described herein can be administered in combination with any of the micro-dystrophins described in Ramos et al. “Development of novel micro-dystrophins with enhanced functionality.” Mol Ther 2019; 27:623-635; (2019) and/or the micro-dystrophins described in U.S. Pat. No. 10,479,821 B2, the contents of each of which is incorporated herein by reference in their entirety. In some embodiments, the micro-dystrophin comprises amino sequence SEQ ID NO: 34, a nucleic acid encoding SEQ ID NO: 34, a fragment, or a variant thereof.

### Ribonucleotide Reductase (RNR)

Ribonucleotide reductase (RNR), also known as ribonucleotide diphosphate reductase (rNDP), is an enzyme that catalyzes the reaction of ribonucleotides to deoxyribonucleotides, which are essential components in the synthesis of DNA. RNR is conserved in all living organisms. The RNR enzyme catalyzes the de novo synthesis of dNDPs. Catalysis of ribonucleoside 5′-diphosphates (NDPs) involves a reduction at the 2′-carbon of ribose 5-phosphate to form the 2′-deoxy derivative-reduced 2′-deoxyribonucleoside 5′-diphosphates (dNDPs). This reduction is initiated with the generation of a free radical. Following a single reduction, RNR requires electrons donated from the dithiol groups of the protein thioredoxin, which is regenerated via NADPH mediated reduction of disulfide groups of thioredoxin.

Three classes of RNR have similar mechanisms for the reduction of NDPs. All classes use free-radical chemistry. Class I reductases use an iron center with ferrous to ferric conversion to generate a tyrosyl free radical. Reduction of NDP substrates occurs under aerobic conditions. Class I reductases are divided into IA and IB due to differences in regulation. Class IA reductases are distributed in eukaryotes, eubacteria, bacteriophages, and viruses. Class IB reductases are found in eubacteria. Class IB reductases can also use a radical generated with the stabilization of a binuclear manganese center. Class II reductases generate the free radical 5′-deoxyadenosyl radical from cobalamin (coenzyme B12) and have a simpler structure than class I and class III reductases. Reduction of NDPs or ribonucleotide 5′-triphosphates (NTPs) occurs under either aerobic or anaerobic conditions. Class II reductases are distributed in archaebacteria, eubacteria, and bacteriophages. Class III reductases use a glycine radical generated with the help of an S-adenosyl methionine and an iron sulphur center. Reduction of NTPs is limited to anaerobic conditions. Class III reductases are distributed in archaebacteria, eubacteria, and bacteriophages. Organisms are not limited to having one class of enzymes. For example, E. coli have both class I and class III RNR. The RNR complex consists of two subunits—RRM1 and RRM2. The larger RRM1 subunit contains the catalytic site and 2 allosteric sites that can bind dATP, whereas the smaller RRM2 subunit contains the free radical generator. The RNR complex is tightly allosterically regulated, with ≤5% of the ATP pool present as dATP. Each RNR1 monomer consists of three domains: (1) one mainly helical domain comprising the 220 N-terminal residues; (2) a second large ten-stranded α/β structure comprising 480 residues; and (3) a third small five-stranded α/β structure comprising 70 residues.

As used herein, “RRM1” or “ribonucleotide reductase catalytic subunit M1” or “an RRM1 construct” refers to the large, catalytic site containing, subunit of the RNR complex. Sequences for RRM1 are known for a number of species, e.g., human RRM1 (NCBI Gene ID: 6240) mRNA (NCBI Ref Seq: NM_001033.5) and polypeptide (NCBI Ref Seq: NP_001024.1). In some embodiments of any of the aspects, the RRM1 nucleic acid or polypeptide can be an isoform, ortholog, variant, and/or allele of SEQ ID NO: 1-SEQ ID NO: 12, respectively.

As used herein, “RRM2” or “ribonucleotide reductase catalytic subunit M2” or an “RRM2 construct” refers to the small subunit of the RNR complex. Sequences for RRM2 are known for a number of species, e.g., human RRM2 (NCBI Gene ID: 6241) mRNA (NCBI Ref Seq: NM_001034.4) and polypeptide (NCBI Ref Seq: NP_001025.1). In some embodiments of any of the aspects, the RRM2 nucleic acid or polypeptide can be an isoform, ortholog, variant, and/or allele of SEQ ID NO: 13-SEQ ID NO: 24, respectively. RRM1 and RRM2 proteins as described herein need to be capable of forming an active RNR complex. Brignole et al., eLife 2018; 7:e31502, which is incorporated herein by reference, describes a 3.3A resolution cryo-EM structure of human ribonucleotide reductase complexed with substrate and allosteric regulators (ATP and dATP)—this near-atomic resolution structure illustrates amino acids and structural domains in the two subunits that interact with each other and illustrates domains necessary for allosteric regulation.

One aspect described employs expression of an RNR complex comprising, consisting of, or consisting essentially of, wild type RRM1 and RRM2 proteins. As used herein, “RNR complex” refers to an RRM1 polypeptide and an RRM2 polypeptide in physical association with each other in the form that provides RNR activity. In this context, the RRM1 and/or RRM2 polypeptide can be a variant that differs in one or more amino acids from the wild-type yet retains the ability to complex with the respective RRM subunit and to catalyze the generation of dATP. One skilled in the art can assess whether the RNR complex is formed, for example, by sucrose gradient analysis or co-immunoprecipitation under non-denaturing conditions. In certain embodiments, it is contemplated that a variant of either or both of RRM1 and/or RRM2 is delivered in one or more therapeutic constructs. Variants include, for example, versions of either or both polypeptides that are rendered more stable, e.g., by modification of a cleavage substrate site for one or more degrading enzymes. Examples are described, for example in U.S. Ser. No. 16/457,441, which is incorporated herein by reference. The increased stability of, e.g., the RRM2 subunit can provide increased activity of the RNR complex.

Where it is important to maintain the function of a variant polypeptide, i.e., complex formation of a mutant RRM2 with RRM1 and/or ribonucleotide reductase activity in complex with RRM1, it can be beneficial to modify a site or sites via conservative amino acid substitution(s). In a conservative substitution, a given amino acid can be replaced by a residue having similar physicochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g., complex formation with Rrm1 and/or ribonucleotide reductase activity for the Rrm1/Rrm2 mutant polypeptide complex is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of ordinary skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure. Indeed, it can be helpful in determining whether a given region of a polypeptide is likely to tolerate mutation, whether conservative or not, by alignment of the polypeptide's sequence from one species, e.g., human, with the sequence of one or more homologous polypeptides from other species, e.g., the sequences of the homologous polypeptide from one or more of rat, mouse, chicken, bovine, porcine or other species in order to determine which regions of the polypeptide molecule are more highly conserved than others throughout evolution. Indeed, it can also help, for a polypeptide connected to a process as centrally important as dATP production, to consider alignments with Rrm2 sequences from more distantly-related eukaryotes, such as fish, reptiles or others. Those regions more highly conserved are more likely to be important for function, meaning that if a ubiquitination site occurs in such region, care should be taken when choosing mutations to introduce so as not to overly interfere with enzymatic function. In such instances, it can be helpful to try several different conservative substitutions at a chosen site—if the change is not marked enough to interfere sufficiently with ubiquitination, no benefit would be expected for such mutant, but a more dramatic change is more likely to interfere with other function(s) of the polypeptide. On the other hand, if a ubiquitination site or ubiquitin-binding degron occurs in a less conserved region of the polypeptide, the polypeptide may well tolerate substitution with one or more non-conservative amino acids to interfere with ubiquitination, as well as tolerating conservative substitution(s).

In some embodiments, a polypeptide described herein (or a nucleic acid encoding such a polypeptide) can be a functional fragment of one of the polypeptides described herein, e.g., a functional fragment of an RRM2 polypeptide. As used herein, a “functional fragment” is a fragment or segment of a peptide which retains at least 50% of the wildtype reference polypeptide's activity according to an assay known in the art or described below herein. For example, a functional fragment described herein would retain at least 50% of the RRM2 function, e.g., can form a complex with Rrm1 and together catalyze the reaction(s) catalyzed by RNR. One skilled in the art can assess the function of an RRM2 enzyme using standard techniques, for example those described herein below. A functional fragment can comprise conservative substitutions of the sequences disclosed herein.

Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan.

A variant amino acid or DNA sequence can be at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant or other reference (e.g., homologue, variant, etc.) sequence can be determined, for example, by comparing the two sequences using freely available computer programs commonly employed for this purpose on the world wide web (e.g. BLASTp or BLASTn with default settings).

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

The compositions and methods described herein comprise a first pharmaceutical composition comprising an RRM1 gene operably linked to a regulatory cassette. In another aspect, the compositions and methods described herein comprise a first pharmaceutical composition comprising an RRM1-encoding gene sequence and an RRM2-encoding gene sequence operably coupled to a first regulatory cassette. It is preferred, but not absolutely necessary, that the gene sequences encoding RRM1 and RRM2 are encoded on a single construct—this arrangement provides for closer management of the stoichiometry of the two subunits of the active enzyme complex. However, in another aspect, the methods and compositions can comprise a first pharmaceutical composition comprising an RRM1 gene operably coupled to a first regulatory cassette in a first delivery vehicle, and a second pharmaceutical composition comprising an RRM2 gene operably coupled to a second regulatory cassette in a second delivery vehicle. It is also contemplated that delivery of just the catalytic subunit of RNR can be overexpressed as a way to increase cellular dATP overall; in this approach, the overexpression of RRM2 can balance the natural degradation of naturally-encoded RRM2, thereby leading to a higher level of RNR activity overall.

In one embodiment of any of the aspects described herein, variant RRM1 and/or RRM2 polypeptides and/or RNR complex provided herein comprise the same enzymatic function of a wild-type RRM1 and/or RNR complex, for example, catalyzing the formation of deoxyribonucleotides from ribonucleotides. Assays for assessing the enzymatic function of a complex provided herein include, but are not limited to nucleotide binding assays, for example, as described in Chimploy, K., and Mathews, C K. J of Biol Chem, 2001; Hendricks, S P, and Mathews C K. J of Biol Chem, 1997; and Hendricks, S P, and Mathews C K. J of Biol Chem, 1998; see also the ribonucleotide reductase assay described by Jong et al., J. Biomed. Sci. 5: 62-68 (1998), the content of each of which are incorporated herein by reference in their entireties.

In another embodiment of any of the aspects, the RRM2 and RRM1-encoding nucleic acids are encoded on the same vector, delivery vehicle, and/or under the control of the same promoter.

In some embodiments of any of the aspects, the RRM1 or RRM2 comprises a mutation that prevents ubiquitination. Mutations found within the ubiquitin binding domain (i.e., the site of ubiquitin addition or ubiquitination) of RRM2 are shown, e.g., in U.S. Ser. No. 16/457,441 to decrease ubiquitination of RRM2, increase RRM2 stability (e.g., half-life of RRM2), and result in increased dATP in the cell. Accordingly, provided herein is an isolated nucleic acid molecule encoding an RRM2 polypeptide that, together with RRM1 polypeptide comprises ribonucleotide reductase activity, the encoded RRM2 polypeptide comprising a mutation that increases the intracellular level of the polypeptide as compared to wild-type RRM2 polypeptide. In one embodiment, the mutation is in a ubiquitin binding degron of RRM2. In another embodiment, the ubiquitin binding degrons of RRM2 are found at nucleotides 88-96 (which encode amino acids that can associate with the APC/FZR1 proteasome) and nucleotides 97-99 and 145-153 (which can associate with the SCF/CyclinF proteasome) of wild-type RRM2 (SEQ ID NO: 13). In another embodiment, the ubiquitin binding degrons of RRM2 are found at amino acids 30-32 (which can associate with the APC/FZR1 proteasome) and amino acids 33 and 49-51 (which can associate with the SCF/CyclinF proteasome) of wild-type RRM2 (SEQ ID NO: 13).

A mutation described herein can be an amino acid substitution, deletion, or insertion. It is contemplated herein that a mutation can be any amino acid change within the ubiquitin binding domain that results in at least decreased ubiquitination of RRM2, increased stability of RRM2, and/or increased dATP levels in the cell. Considerations for mutating a ubiquitination site while maintaining RRM2 activity in terms of complex formation and ribonucleotide reductase activity with RRM1 are discussed herein above. In some embodiments, the mutation is found near a ubiquitin binding degron, e.g., within 1-10 nucleotides of a ubiquitin binding degron, i.e., nucleotides not encoding a ubiquitin binding degron. In some embodiments, the mutation is found near a ubiquitin binding degron, e.g., within 1-10 amino acids of a ubiquitin binding degron, i.e., amino acids not encoding a ubiquitin binding degron.

Alterations of the native amino acid sequence (e.g., of RRM1 or RRM2) can be accomplished by any of a number of techniques known in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Assays for detecting the stability and/or degradation of a protein are known in the art, and include, treating a cell lysate or an in vitro system having the protein of interest and components of the ubiquitin mediated degradation system with cyclohexamide to halt protein translation and measuring the level of the protein of interest over time (e.g., in a time course) via Western blotting. Alternatively, protein stability can be measured using a standard pulse-chase experiment.

### Linkers

The RNR described herein are expressed as a fusion protein in which the RRM1 and RRM2 polypeptides are joined by a linker peptide. The constructs described herein can thus further comprise a linker. Linkers can be configured according to a specific need, e.g., to have a sufficient length and flexibility such that it can allow for a cleavage at a target site. Methods of synthesizing fusion proteins and linkers are known in the art.

In some embodiments of any of the aspects, the RRM2-encoding nucleic acid is linked to the RRM1-encoding nucleic acid, e.g., through a type 2A peptide-encoding sequence, such as P2A. P2A is a non-limiting example of a 2A self-cleaving peptide, which can induce the cleavage of the recombinant protein when expressed in a cell. See, e.g., Kolwicz et al., Molecular Therapy 24: 240-250 (2016), which is incorporated herein by reference in its entirety. Non-limiting examples of 2A self-cleaving peptides include T2A, P2A, E2A, and F2A. Any self-cleaving peptide sequence known in the art can be used to link RRM1 to RRM2.

SEQ ID NO: 25 is an exemplary nucleic acid
sequence comprising a Kozak sequence, RRM1, P2A,
and RRM2.
GCTAGCGAATTCGCCACCATGCACGTCATCAAGAGAGACGGGAGGCAGGA
AAGAGTCATGTTCGATAAAATCACTTCAAGAATCCAGAAACTGTGTTACG
GGCTGAACATGGACTTCGTCGATCCTGCCCAGATTACCATGAAAGTGATC
CAGGGACTGTACTCTGGCGTCACCACAGTGGAGCTGGACACACTGGCCGC
TGAAACCGCAGCCACACTGACTACCAAACACCCAGATTATGCAATTCTGG
CTGCACGGATCGCCGTGAGTAATCTGCATAAGGAGACAAAGAAAGTCTTC
TCAGACGTGATGGAGGACCTGTACAATTATATCAACCCTCACAATGGGAA
ACATTCACCAATGGTCGCTAAGAGCACTCTGGACATTGTGCTGGCCAACA
AAGATCGGCTGAACAGCGCTATCATCTACGACCGGGATTTCAGTTACAAC
TACTTCGGCTTTAAGACACTGGAGAGATCATATCTGCTGAAAATCAATGG
GAAGGTGGCCGAACGGCCTCAGCACATGCTGATGAGAGTCAGCGTGGGCA
TTCATAAGGAGGACATTGATGCCGCTATCGAAACTTACAACCTGCTGAGC
GAGCGCTGGTTCACCCACGCTTCCCCTACACTGTTTAACGCAGGAACCAA
TCGACCACAGCTGAGCAGCTGCTTCCTGCTGAGCATGAAGGACGATTCCA
TCGAGGGCATCTACGACACCCTGAAACAGTGCGCACTGATTTCTAAGAGT
GCCGGCGGGATCGGAGTCGCTGTGAGTTGTATTCGGGCAACCGGCTCATA
TATCGCCGGCACAAACGGCAACAGCAACGGGCTGGTCCCCATGCTGAGGG
TGTACAACAATACAGCCCGCTATGTGGATCAGGGAGGCAACAAGAGACCA
GGAGCATTTGCCATCTACCTGGAACCCTGGCACCTGGACATTTTCGAGTT
TCTGGATCTGAAGAAAAATACTGGCAAAGAGGAACAGAGGGCTCGCGACC
TGTTCTTTGCACTGTGGATTCCCGACCTGTTCATGAAGAGGGTGGAGACC
AACCAGGACTGGAGCCTGATGTGCCCCAATGAGTGTCCTGGGCTGGATGA
AGTGTGGGGAGAGGAATTTGAAAAACTGTACGCCAGTTATGAGAAGCAGG
GCCGAGTGCGGAAAGTGGTCAAGGCCCAGCAGCTGTGGTACGCTATCATT
GAGAGCCAGACAGAAACTGGCACCCCCTACATGCTGTATAAAGACTCTTG
CAACCGCAAGAGTAACCAGCAGAATCTGGGGACCATCAAATGCAGCAATC
TGTGTACAGAGATTGTGGAATATACTTCCAAGGATGAGGTCGCCGTGTGT
AACCTGGCATCACTGGCCCTGAATATGTACGTCACAAGCGAGCACACTTA
TGACTTCAAGAAACTGGCTGAAGTGACCAAAGTGGTCGTGAGGAATCTGA
ACAAGATCATTGACATCAACTACTATCCCGTGCCTGAGGCCTGCCTGAGC
AATAAGAGACATAGGCCCATCGGGATTGGAGTGCAGGGCCTGGCTGACGC
ATTCATCCTGATGCGCTACCCTTTTGAGTCCGCCGAAGCTCAGCTGCTGA
ACAAGCAGATTTTTGAAACAATCTACTACGGGGCTCTGGAGGCATCTTGT
GACCTGGCCAAAGAACAGGGACCCTACGAGACTTATGAAGGCTCCCCTGT
GTCTAAGGGCATCCTGCAGTACGATATGTGGAACGTCACACCAACTGACC
TGTGGGATTGGAAAGTGCTGAAGGAGAAAATTGCAAAGTATGGCATCCGG
AACAGCCTGCTGATCGCCCCAATGCCCACTGCCTCTACCGCTCAGATTCT
GGGCAACAATGAGTCCATCGAACCATACACTTCTAACATCTACACCCGGA
GAGTCCTGAGCGGGGAGTTCCAGATCGTGAATCCCCACCTGCTGAAAGAC
CTGACCGAACGGGGACTGTGGCATGAGGAAATGAAGAACCAGATCATTGC
CTGCAATGGCAGTATCCAGTCAATTCCTGAGATCCCAGACGATCTGAAAC
AGCTGTACAAGACAGTCTGGGAGATCAGCCAGAAAACTGTGCTGAAGATG
GCAGCCGAAAGAGGGGCTTTCATTGATCAGTCACAGAGCCTGAACATCCA
CATTGCCGAGCCCAATTACGGAAAGCTGACCTCCATGCATTTTTATGGGT
GGAAACAGGGACTGAAGACTGGCATGTACTATCTGCGCACCCGACCAGCT
GCAAACCCCATCCAGTTTACCCTGAATAAGGAGAAACTGAAGGACAAAGA
AAAGGTGTCCAAAGAGGAAGAGGAAAAGGAGAGAAACACAGCCGCTATGG
TGTGTTCTCTGGAGAATAGGGATGAATGCCTGATGTGTGGCAGTGGAAGC
GGAGCTACTAACTTCAGCCTGCTGAAGCAGGCTGGAGACGTGGAGGAGAA
CCCTGGACCTCTGAGTCTGAGGGTCCCACTGGCACCTATCACCGATCCAC
AGCAGCTGCAGCTGAGCCCACTGAAAGGCCTGAGTCTGGTCGATAAAGAG
AACACACCACCTGCACTGAGTGGCACTCGGGTGCTGGCATCAAAGACCGC
CCGGAGAATTTTCCAGGAGCCAACCGAACCCAAAACAAAGGCCGCTGCAC
CTGGGGTCGAGGACGAACCACTGCTGAGAGAGAATCCCAGGCGCTTCGTG
ATTTTTCCTATCGAATACCACGATATTTGGCAGATGTATAAGAAAGCTGA
GGCAAGTTTCTGGACAGCTGAGGAAGTGGACCTGAGCAAAGACATCCAGC
ACTGGGAATCCCTGAAGCCAGAGGAAAGGTACTTCATTTCTCATGTGCTG
GCATTCTTTGCCGCTAGTGACGGGATCGTGAACGAGAATCTGGTCGAACG
CTTTAGCCAGGAGGTGCAGATCACTGAAGCCCGATGCTTCTATGGATTTC
AGATTGCTATGGAGAACATCCATTCAGAAATGTACAGCCTGCTGATTGAC
ACCTATATCAAAGATCCTAAGGAGCGCGAGTTCCTGTTTAATGCCATTGA
GACAATGCCATGTGTGAAGAAAAAGGCAGACTGGGCTCTGCGATGGATCG
GCGATAAGGAGGCTACTTACGGGGAAAGAGTGGTCGCATTCGCAGCCGTG
GAGGGAATTTTCTTTTCTGGCAGTTTCGCTTCCATCTTTTGGCTGAAAAA
GCGAGGCCTGATGCCTGGGCTGACCTTTTCCAACGAGCTGATTTCTCGCG
ACGAAGGCCTGCACTGCGATTTCGCCTGTCTGATGTTTAAACACCTGGTG
CATAAGCCCTCTGAGGAACGAGTCCGGGAGATCATTATCAACGCAGTGAG
GATCGAGCAGGAGTTCCTGACAGAAGCCCTGCCTGTCAAACTGATTGGCA
TGAATTGCACTCTGATGAAGCAGTACATCGAGTTTGTGGCCGACAGGCTG
ATGCTGGAACTGGGATTCTCAAAGGTGTTTCGCGTCGAGAACCCATTCGA
TTTTATGGAGAATATCAGCCTGGAAGGCAAAACAAACTTCTTTGAGAAGA
GAGTCGGGGAATATCAGAGGATGGGCGTGATGAGCAGCCCCACTGAGAAT
AGCTTCACCCTGGACGCCGATTTTTGAGCTAGC
SEQ ID NO: 26 is an exemplary Kozak sequence (as
found in SEQ ID NO: 25).
GCCACC
SEQ ID NO: 27 is an exemplary RRM1 sequence (as
found in SEQ ID NO: 25).
ATGCACGTCATCAAGAGAGACGGGAGGCAGGAAAGAGTCATGTTCGATAA
AATCACTTCAAGAATCCAGAAACTGTGTTACGGGCTGAACATGGACTTCG
TCGATCCTGCCCAGATTACCATGAAAGTGATCCAGGGACTGTACTCTGGC
GTCACCACAGTGGAGCTGGACACACTGGCCGCTGAAACCGCAGCCACACT
GACTACCAAACACCCAGATTATGCAATTCTGGCTGCACGGATCGCCGTGA
GTAATCTGCATAAGGAGACAAAGAAAGTCTTCTCAGACGTGATGGAGGAC
CTGTACAATTATATCAACCCTCACAATGGGAAACATTCACCAATGGTCGC
TAAGAGCACTCTGGACATTGTGCTGGCCAACAAAGATCGGCTGAACAGCG
CTATCATCTACGACCGGGATTTCAGTTACAACTACTTCGGCTTTAAGACA
CTGGAGAGATCATATCTGCTGAAAATCAATGGGAAGGTGGCCGAACGGCC
TCAGCACATGCTGATGAGAGTCAGCGTGGGCATTCATAAGGAGGACATTG
ATGCCGCTATCGAAACTTACAACCTGCTGAGCGAGCGCTGGTTCACCCAC
GCTTCCCCTACACTGTTTAACGCAGGAACCAATCGACCACAGCTGAGCAG
CTGCTTCCTGCTGAGCATGAAGGACGATTCCATCGAGGGCATCTACGACA
CCCTGAAACAGTGCGCACTGATTTCTAAGAGTGCCGGCGGGATCGGAGTC
GCTGTGAGTTGTATTCGGGCAACCGGCTCATATATCGCCGGCACAAACGG
CAACAGCAACGGGCTGGTCCCCATGCTGAGGGTGTACAACAATACAGCCC
GCTATGTGGATCAGGGAGGCAACAAGAGACCAGGAGCATTTGCCATCTAC
CTGGAACCCTGGCACCTGGACATTTTCGAGTTTCTGGATCTGAAGAAAAA
TACTGGCAAAGAGGAACAGAGGGCTCGCGACCTGTTCTTTGCACTGTGGA
TTCCCGACCTGTTCATGAAGAGGGTGGAGACCAACCAGGACTGGAGCCTG
ATGTGCCCCAATGAGTGTCCTGGGCTGGATGAAGTGTGGGGAGAGGAATT
TGAAAAACTGTACGCCAGTTATGAGAAGCAGGGCCGAGTGCGGAAAGTGG
TCAAGGCCCAGCAGCTGTGGTACGCTATCATTGAGAGCCAGACAGAAACT
GGCACCCCCTACATGCTGTATAAAGACTCTTGCAACCGCAAGAGTAACCA
GCAGAATCTGGGGACCATCAAATGCAGCAATCTGTGTACAGAGATTGTGG
AATATACTTCCAAGGATGAGGTCGCCGTGTGTAACCTGGCATCACTGGCC
CTGAATATGTACGTCACAAGCGAGCACACTTATGACTTCAAGAAACTGGC
TGAAGTGACCAAAGTGGTCGTGAGGAATCTGAACAAGATCATTGACATCA
ACTACTATCCCGTGCCTGAGGCCTGCCTGAGCAATAAGAGACATAGGCCC
ATCGGGATTGGAGTGCAGGGCCTGGCTGACGCATTCATCCTGATGCGCTA
CCCTTTTGAGTCCGCCGAAGCTCAGCTGCTGAACAAGCAGATTTTTGAAA
CAATCTACTACGGGGCTCTGGAGGCATCTTGTGACCTGGCCAAAGAACAG
GGACCCTACGAGACTTATGAAGGCTCCCCTGTGTCTAAGGGCATCCTGCA
GTACGATATGTGGAACGTCACACCAACTGACCTGTGGGATTGGAAAGTGC
TGAAGGAGAAAATTGCAAAGTATGGCATCCGGAACAGCCTGCTGATCGCC
CCAATGCCCACTGCCTCTACCGCTCAGATTCTGGGCAACAATGAGTCCAT
CGAACCATACACTTCTAACATCTACACCCGGAGAGTCCTGAGCGGGGAGT
TCCAGATCGTGAATCCCCACCTGCTGAAAGACCTGACCGAACGGGGACTG
TGGCATGAGGAAATGAAGAACCAGATCATTGCCTGCAATGGCAGTATCCA
GTCAATTCCTGAGATCCCAGACGATCTGAAACAGCTGTACAAGACAGTCT
GGGAGATCAGCCAGAAAACTGTGCTGAAGATGGCAGCCGAAAGAGGGGCT
TTCATTGATCAGTCACAGAGCCTGAACATCCACATTGCCGAGCCCAATTA
CGGAAAGCTGACCTCCATGCATTTTTATGGGTGGAAACAGGGACTGAAGA
CTGGCATGTACTATCTGCGCACCCGACCAGCTGCAAACCCCATCCAGTTT
ACCCTGAATAAGGAGAAACTGAAGGACAAAGAAAAGGTGTCCAAAGAGGA
AGAGGAAAAGGAGAGAAACACAGCCGCTATGGTGTGTTCTCTGGAGAATA
GGGATGAATGCCTGATGTGTGGCAGT
SEQ ID NO: 28 is an exemplary P2A sequence (as
found in SEQ ID NO: 25).
GCTACTAACTTCAGCCTGCTGAAGCAGGCTGGAGACGTGGAGGAGAACCC
TGGACCT

### Regulatory Cassettes

The RRM1, RRM2 and/or micro-dystrophin-coding sequences for the constructs described herein can be operably coupled to a regulatory cassette.

A regulatory cassette directs the expression of a gene (e.g., RRM1, RRM2, μDys). A regulatory cassette generally comprises a promoter element and other sequences necessary to direct the assembly of an active transcriptase complex in a desired cell type. A regulatory cassette can also include, for example, a 3′ untranslated sequence including a polyadenylation signal downstream of the region where an open reading frame encoding the desired polypeptide is or can be inserted. Exemplary promoters that can be used include, but are not limited to, constitutive promoters, repressible promoters, and/or inducible promoters, some non-limiting examples of which include viral promoters (e.g., CMV, SV40), tissue specific promoters (e.g., striated muscle CK8), cardiac muscle (e.g., cTnT), eye (e.g., MSK) and synthetic promoters (SP1 elements) and the chicken beta actin promoter (CB or CBA).

In some embodiments, the regulatory cassette can be positioned at the 5′ end of the RRM1, RRM2, or the micro-dystrophin described herein. In others, the cassette flanks the sequence to be encoded.

In some embodiments of any of the aspects, the regulatory cassette is a muscle-specific regulatory cassette. Exemplary muscle-specific regulatory cassettes include, but are not limited to, a cardiac troponin T (cTNT) regulatory cassette; a creatine kinase regulatory cassette; a muscle creatine kinase (MCK) regulatory cassette; a CK8 regulatory cassette; a MHCK7 regulatory cassette; CK7 regulatory cassette; and any fragment or combinations thereof. The nucleic acid constructs described herein can be prepared by synthetic and/or cloning methods known in the art.

In some embodiments of any of the aspects, the pharmaceutical compositions described herein includes a CK8 regulatory cassette. In some embodiments, the CK8 regulatory cassette has at least 80% sequence identity to the nucleic acid sequence of SEQ ID NO: 29.

| CK8 promoter (SEQ ID NO: 29): | |
|---|---|
| ctagactagc atgctgccca tgtaaggagg caaggcctgg ggacacccga gatgcctggt | 60 |
| tataattaac ccagacatgt ggctgccccc ccccccccaa cacctgctgc ctctaaaaat | 120 |
| aaccctgcat gccatgttcc cggcgaaggg ccagctgtcc cccgccagct agactcagca | 180 |
| cttagtttag gaaccagtga gcaagtcagc ccttggggca gcccatacaa ggccatgggg | 240 |
| ctgggcaagc tgcacgcctg ggtccggggt gggcacggtg cccgggcaac gagctgaaag | 300 |
| ctcatctgct ctcaggggcc cctccctggg gacagcccct cctggctagt cacaccctgt | 360 |
| aggctcctct atataaccca ggggcacagg ggctgccctc attctaccac cacctccaca | 420 |
| gcacagacag acactcagga gccagccagc | 450 |

The CK8 regulatory cassette can display strong, muscle-restricted expression. The CK8 regulatory cassette is less than 500 bps in size (see, e.g., Goncalves, M. A., et al., Molecular Therapy: The Journal of the American Society of Gene Therapy 19, 1331-1341, (2011) and Martari, M., et al., Human Gene Therapy 20, 759-766, (2009), which are incorporated herein by reference in its entirety.

In some embodiments of any of the aspects, the pharmaceutical compositions described herein includes a cTNT regulator cassette. In some embodiments, the cTNT regulatory cassette has at least 80% sequence identity to the nucleic acid sequence of SEQ ID NO: 30.

| hum-cTnT455 (SEQ ID NO: 30): | |
|---|---|
| ctgctcccag ctggccctcc caggcctggg ttgctggcct ctgctttatc aggattctca | 60 |
| agagggacag ctggtttatg ttgcatgact gttccctgca tatctgctct ggttttaaat | 120 |
| agcttatctg ctagcctgct cccagctggc cctcccaggc ctgggttgct ggcctctgct | 180 |
| ttatcaggat tctcaagagg gacagctggt ttatgttgca tgactgttcc ctgcatatct | 240 |
| gctctggttt taaatagctt atctgagcag ctggaggacc acatgggctt atatggggca | 300 |
| cctgccaaaa tagcagccaa cacccccccc tgtcgcacat tcctccctgg ctcaccaggc | 360 |
| cccagcccac atgcctgctt aaagccctct ccatcctctg cctcacccag tccccgctga | 420 |
| gactgagcag acgcctccag gatctgtcgg cagct | 455 |

The human cTnT455 regulatory cassette (SEQ ID NO: 30) targets the transient expression of the pharmaceutical composition in wounded and/or regenerating cardiac muscle. cTnT455 can lead to high expression in the heart but little to no expression in other tissue. In some embodiments, expression of the pharmaceutical compositions disclosed herein prevents the loss of cardiac muscle and/or of cardiomyocytes. In some embodiments, expression of the pharmaceutical compositions disclosed herein regenerate skeletal muscle. In some embodiments, expression of the pharmaceutical compositions disclosed herein prevent muscle cell necrosis and/or wasting of skeletal muscle.

### Delivery Vehicles

The methods and compositions described herein involve the introduction of sequences encoding therapeutic polypeptides to muscle cells in vivo, including, for example, cardiac muscle cells, among others. These methods permit practitioners to introduce DNA coding for a therapeutic polypeptide directly into a patient or subject (in vivo gene therapy) or into cells isolated from a patient, a subject, or a donor (ex vivo gene therapy). The introduced DNA then directs the patient's or subject's own cells or grafted cells to produce the desired protein product. Gene therapy can also permit practitioners to select specific organs or cellular targets (e.g., muscle, liver, blood cells, brain cells, etc.) for therapy. Sequences to be introduced to cells in vivo (or ex vivo, for that matter) can be cloned into an appropriate vector. Examples of mammalian expression vectors include pCDM8 (Seed, 1987. Nature 329: 840) and pMT2PC (Kaufman, et al., 1987. EMBO J. 6: 187-195), and are further described in, e.g., U.S. Pat. Nos. 8,187,836; 8,455,219; 8,980,626; 7,384,776; and 6,451,539; the contents of which are incorporated herein by reference in their entireties. When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989; and Muller, D., et al. (2006) Microbial Cell Factories.

In some embodiments, the recombinant mammalian expression vector is capable of directing expression of the synthetic nucleic acid preferentially in a particular cell type (e.g., tissue-specific regulatory elements are used to express the nucleic acid in, for example, a cardiomyocyte). Tissue-specific regulatory elements are known in the art. Non-limiting examples of suitable cardiac tissue-specific promoters include the cTnT promoter, the NCX1 promoter (e.g., as described in Nicholas S B., et al. Am J Physiol. 1998), the MLC-2v (e.g., as described Griscelli, F., et al. C R Acad Sci III. 1997 February; 320(2):103-12); and the cardiac troponin-I proximal promoter (TNNI3) (e.g., as described in Gallo, P., et al. Gene Therapy. 15, pages 161-170 (2008). All citations provided herein are incorporated herein by reference in their entireties. The CK8 promoter described elsewhere herein is an example of a striated muscle-specific promoter.

The RRM1 and RRM2 constructs described herein can be administered to a subject in need in one vector, or in two vectors or delivery vehicles. In some embodiments of any of the aspects, a first delivery vehicle and a second delivery vehicle are separate delivery vehicles. In some embodiments of any of the aspects, the delivery vehicle is a viral vector.

Current viral-mediated gene delivery methods include, but are not limited to, retrovirus, adenovirus, herpes virus, pox virus, and adeno-associated virus (AAV) vectors.

### AAV Vectors

AAV is a parvovirus which belongs to the genus Dependoparvovirus. AAV has several attractive features not found in other viruses. First, AAV can infect a wide range of host cells, including non-dividing cells. Second, AAV can infect cells from different species. Third, AAV has not been associated with any human or animal disease and does not appear to alter the biological properties of the host cell upon integration. Indeed, it is estimated that 80-85% of the human population has been exposed to the virus. Finally, AAV is stable at a wide range of physical and chemical conditions which lends itself to production, storage, and transportation requirements.

The AAV genome is a linear, single-stranded DNA molecule containing 4681 nucleotides. The AAV genome generally comprises an internal non-repeating genome flanked on each end by inverted terminal repeats (ITRs). The ITRs are approximately 145 base pairs (bp) in length. The ITRs have multiple functions, including as origins of DNA replication and as packaging signals for the viral genome.

The internal non-repeated portion of the genome includes two large open reading frames, known as the AAV replication (rep) and capsid (cap) genes. The rep and cap genes code for viral proteins that allow the virus to replicate and package the viral genome into a virion. In particular, a family of at least four viral proteins are expressed from the AAV rep region, Rep78, Rep68, Rep52, and Rep40, named according to their apparent molecular weight. The AAV cap region encodes at least three proteins, VP1, VP2, and VP3.

AAV is a helper-dependent virus; that is, it requires co-infection with a helper virus (e.g., adenovirus, herpesvirus, or vaccinia) in order to form AAV virions. In the absence of co-infection with a helper virus, AAV establishes a latent state in which the viral genome inserts into a host cell chromosome, but infectious virions are not produced. Subsequent infection by a helper virus “rescues” the integrated genome, allowing it to replicate and package its genome into infectious AAV virions. While AAV can infect cells from different species, the helper virus must be of the same species as the host cell. Thus, for example, human AAV will replicate in canine cells co-infected with a canine adenovirus.

An “AAV vector” comprises a vector derived from an adeno-associated virus serotype, including without limitation, AAV1, AAV2, AAV3, AAV4, AAV5, AAV6, AAV7, AAV8, and AAV9. AAV vectors can have one or more of the AAV wild-type genes deleted in whole or part, e.g., the rep and/or cap genes, but retain functional flanking ITR sequences. Functional ITR sequences are necessary for the rescue, replication, and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

A “recombinant AAV vector” or “rAAV vector” comprises an infectious, replication-defective virus composed of an AAV protein shell encapsulating a heterologous nucleotide sequence of interest that is flanked on both sides by AAV ITRs. An rAAV vector is produced in a suitable host cell comprising an AAV vector, AAV helper functions, and accessory functions. In this manner, the host cell is rendered capable of encoding AAV polypeptides that are required for packaging the AAV vector (containing a recombinant nucleotide sequence of interest) into infectious recombinant virion particles for subsequent gene delivery.

In various embodiments, the delivery vehicle may comprise an adeno-associated virus (AAV) vector or a recombinant adeno-associated virus (rAAV) vector. The AAV vector may be a serotype 6 AAV (AAV6). Likewise, the rAAV vector may be a serotype 6 rAAV (rAAV6). The AAV vector may be a serotype 8 AAV (AAV8). Likewise, the rAAV vector may be a serotype 8 rAAV (rAAV8). The AAV vector may be a serotype 9 AAV (AAV9). Likewise, the rAAV vector may be a serotype 9 rAAV (rAAV9). The rAAV vector may be comprised of AAV2 genomic inverted terminal repeat (ITR) sequences pseudotyped with capsid proteins derived from AAV serotype 6 (rAAV2/6). Other suitable serotypes of the AAV or rAAV known in the art can be used. AAV6 is particularly attractive due to efficient infection and transduction of muscle cells, including cardiac muscle cells.

### Pharmaceutical Compositions

One aspect provided herein is a pharmaceutical composition comprising, consisting of, or consisting essentially of any of the isolated nucleic acids, vectors, polypeptides, or RNR complexes described herein. As used herein, the term “pharmaceutical composition” refers to the active agent in combination with a pharmaceutically acceptable carrier e.g., a carrier commonly used in the pharmaceutical industry.

For clinical use of the methods and compositions described herein, administration of the RRM1, RRM2, and/or micro-dystrophin constructs described herein can include formulation into pharmaceutical compositions or pharmaceutical formulations for parenteral administration, e.g., intravenous; muscular e.g., intramuscular or intracardiac delivery; or other mode of administration. In some embodiments, the nucleic acid compositions described herein can be administered along with any pharmaceutically acceptable carrier compound, material, or composition which results in an effective treatment in the subject. Thus, a pharmaceutical formulation for use in the methods described herein can contain the RRM1 and/or RRM2 genes in combination with one or more pharmaceutically acceptable ingredients. The phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent, media, encapsulating material, or solvent encapsulating material, involved in maintaining the stability, solubility, or activity of, a nucleic acid or viral vector construct as described herein. Each carrier must be “acceptable” in the sense of being compatible with the other ingredients of the formulation and not injurious to the patient. The terms “excipient,” “carrier,” “pharmaceutically acceptable carrier” or the like are used interchangeably herein.

Suitable excipients are, for example, water, saline, dextrose, glycerol, ethanol or the like and combinations thereof. In addition, if desired, the composition can contain minor amounts of auxiliary substances such as pH buffering agents and the like which enhance the effectiveness of the active ingredient. The therapeutic composition of the present technology can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used with the methods described herein that will be effective in the treatment of a particular disorder or condition will depend on the nature of the disorder or condition, and can be determined by standard clinical techniques.

Therapeutic pharmaceutical compositions described herein can also contain more than one active compound as necessary for the particular indication being treated, preferably those with complementary activities that do not adversely affect each other.

### Administration, Dosage, and Efficacy

The RNR constructs and pharmaceutical compositions described herein can be formulated, dosed, and administered in a fashion consistent with good medical practice. Factors for consideration in this context include the particular muscular dystrophy or complication being treated, the particular subject being treated, the clinical condition of the individual subject, the cause of the disorder, the site of delivery of the pharmaceutical composition, the method of administration, the scheduling of administration, and other factors known to medical practitioners.

The therapeutic formulations to be used for in vivo administration, such as parenteral administration, in the methods described herein can be sterile, which is readily accomplished by filtration through sterile filtration membranes, or other methods known to those of skill in the art.

The RNR construct described herein and pharmaceutical compositions thereof can be administered to a subject in need thereof by any appropriate route which results in an effective treatment in the subject. As used herein, the terms “administering,” and “introducing” are used interchangeably and refer to the placement of a pharmaceutical composition, RRM1, RRM2, RNR, and/or micro-dystrophin construct, into a subject by a method or route which results in at least partial localization of such pharmaceutical compositions at a desired site, such that a desired effect(s) is produced. A pharmaceutical composition can be administered to a subject by any mode of administration that delivers the nucleic acid constructs systemically or to a desired surface or target, and can include, but is not limited to, injection, infusion, instillation, and inhalation administration. To the extent that RRM1, RRM2, and/or micro-dystrophin constructs described herein can be protected from inactivation in the gut, oral administration forms are also contemplated. “Injection” includes, without limitation, intravenous, intramuscular, intra-arterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The phrases “parenteral administration” and “administered parenterally” as used herein, refer to modes of administration other than enteral and topical administration, usually by injection. The phrases “systemic administration,” “administered systemically”, “peripheral administration” and “administered peripherally” as used herein refer to the administration of a therapeutic agent other than directly into a target site, tissue, or organ, such as a site of cardiac dysfunction, such that it enters the subject's circulatory system and, thus, is subject to metabolism and other like processes. In other embodiments, the pharmaceutical composition is administered locally, e.g., by direct injections, and the injections can be repeated periodically.

In some embodiments, the compositions described herein are administered by intravenous injection, orally, intracardiac delivery, or intramuscular injection.

The term “effective amount” as used herein refers to the amount of a pharmaceutical composition needed to alleviate or prevent at least one or more symptoms of a muscular dystrophy, disease or disorder, and relates to a sufficient amount of pharmacological composition to provide the desired effect, e.g., increase cardiac output, reduce cardiomyopathy, reduce pathology, or any symptom associated with or caused by the loss of dystrophin. The term “therapeutically effective amount” therefore refers to an amount of a pharmaceutical composition described herein using the methods as disclosed herein, that is sufficient to effect a particular effect when administered to a typical subject. An effective amount as used herein would also include an amount sufficient to delay the development of a symptom of the disease, alter the course of a symptom disease (for example, but not limited to, slow the progression of a symptom of the disease), or reverse a symptom of the disease. Thus, it is not possible to specify the exact “effective amount.” However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the RRM1, RRM2, or a combination thereof), which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay. The dosage can be determined by a physician and adjusted, as necessary, to suit observed effects of the treatment.

The pharmaceutical compositions described herein can be formulated, in some embodiments, with one or more additional therapeutic agents currently used to prevent or treat muscular dystrophy, for example. The effective amount of such other agents depends on the amount of the nucleic acid constructs in the formulation, the type of disorder or treatment, and other factors discussed above. These are generally used in the same dosages and with administration routes as used herein before or about from 1 to 99% of the heretofore employed dosages.

The dosage ranges for the pharmaceutical compositions described herein depend upon the potency, and encompass amounts large enough to produce the desired effect. The dosage should not be so large as to cause unacceptable adverse side effects. Generally, the dosage will vary with the age, condition, and sex of the patient and can be determined by one of skill in the art. The dosage can also be adjusted by the individual physician in the event of any complication. In some embodiments, the dosage ranges from 0.001 mg/kg body weight to 100 mg/kg body weight. In some embodiments, the dose range is from 5 μg/kg body weight to 100 μg/kg body weight. Alternatively, the dose range can be titrated to maintain serum levels between 1 μg/mL and 1000 μg/mL. For systemic administration, subjects can be administered a therapeutic amount, such as, e.g., 0.1 mg/kg, 0.5 mg/kg, 1.0 mg/kg, 2.0 mg/kg, 2.5 mg/kg, 5 mg/kg, 7.5 mg/kg, 10 mg/kg, 15 mg/kg, 20 mg/kg, 25 mg/kg, 30 mg/kg, 40 mg/kg, 50 mg/kg, or more. Dosages of viral vectors can also be expressed as numbers of viral genomes (vg) per kilogram. These doses can be administered by one or more separate administrations, or by continuous infusion. For repeated administrations over several days or longer, depending on the condition, the treatment is sustained until, for example, the muscular dystrophy is treated, as measured by the methods described above or known in the art. However, other dosage regimens can be useful.

While a goal of gene therapy is generally to introduce a therapeutic construct or sequence once or a limited number of times to effect a durable treatment, the duration of a therapy using the methods described herein can continue for as long as medically indicated or until a desired therapeutic effect (e.g., those described herein) is achieved. As will be appreciated by one of skill in the art, appropriate dosing regimens for a given composition can comprise a single administration or multiple ones. In certain embodiments, the administration of a pharmaceutical composition as described herein can be repeated, e.g., monthly, quarterly, biannually, yearly or over a more distantly separated period, depending upon duration of therapeutic effect.

The precise dose to be employed in a formulation will also depend on the route of administration and should be decided according to the judgment of the practitioner and each patient's circumstances. Ultimately, the practitioner or physician will decide the amount of the RNR, RRM1, RRM2, or mDys constructs or vectors to administer and how often to administer them based on desired effect and measured efficacies.

In some embodiments of these methods and all such methods described herein, the pharmaceutical compositions described herein are administered in an amount effective to provide cardioprotection, improve cardiac function, treat or prevent muscular dystrophy or complications thereof, and/or alleviate at least one symptom of a muscular dystrophy.

“Alleviating a symptom of a muscular dystrophy” is ameliorating any condition or symptom associated with the muscular dystrophy, e.g., cardiac dysfunction. Alternatively, alleviating a symptom of a muscular dystrophy can involve increasing contractile function, increasing systolic function, and/or increasing diastolic function in the subject relative to an untreated control. As compared with an equivalent untreated control, such reduction or degree of prevention is at least 5%, 10%, 20%, 40%, 50%, 60%, 80%, 90%, 95%, or 100% as measured by any standard technique.

The effects of the RNR pharmaceutical compositions described herein can be determined, for example, by detecting and measuring cardiac function in a subject, a test animal, or cell.

Methods for detecting, measuring, and determining cardiac function are known in the art. Non-limiting examples of clinical tests that can be used to assess cardiac functional parameters include echocardiography (with or without Doppler flow imaging), electrocardiogram (EKG), exercise stress test, Holter monitoring, or measurement of natriuretic peptide (e.g., atrial natriuretic peptide).

Where necessary or desired, animal models of muscular dystrophy can be used to gauge the effectiveness of a particular composition as described herein. For example, an mdx mouse model, or DMD canines can be used. Animal models of cardiac function are useful for monitoring infarct zones, coronary perfusion, electrical conduction, left ventricular end diastolic pressure, left ventricular ejection fraction, heart rate, blood pressure, degree of hypertrophy, diastolic relaxation function, cardiac output, heart rate variability, and ventricular wall thickness, etc.

In other embodiments, the nucleic acid constructs described herein may be used to treat a muscular dystrophy or a complication thereof, or improve survival, e.g., to reduce the onset, incidence of severity of a cardiovascular event. The efficacy of a therapeutic treatment can be assessed by the presence or absence of a symptom of a disease by functional output (e.g., measuring cardiac output or renal function), markers, levels or expression (e.g., serum levels of cardiac enzymes, markers of ischemia, renal function or insufficiency), and/or echocardiographic and electrographic means (e.g., an electrocardiogram or an echocardiogram). Further, as will be appreciated by a skilled physician, the ability to modify the nucleic acid constructs described herein can permit them to customize a treatment based on a subject's particular set of symptoms and/or severity of disease and further to minimize side effects or toxicity.

A patient who is being treated for a muscular dystrophy can be one whom a medical practitioner has diagnosed as having such a condition. Diagnosis can be by any suitable means. Diagnosis and monitoring can involve, for example, detecting the level of dystrophin in a biological sample (for example, a tissue biopsy, blood test, or urine test), detecting the level of creatine kinase (CK) in a biological sample, detecting symptoms associated with muscular dystrophy, or detecting the electrical activity of a muscle via electromyography (EMG) or an electrocardiogram (EKG). Genetic sequencing can also provide an indication of a mutation in one or more sequences involved in or linked to a congenital muscular dystrophy, including but not limited to a mutation that affects the structure or expression level of dystrophin. A patient in whom the development of a muscular dystrophy is being prevented may or may not have received a diagnosis of a muscular dystrophy. One of ordinary skill in the art will understand that these patients may have been subjected to the same standard tests as described above or may have been identified, without examination, as one at high risk due to the presence of one or more risk factors (such as family history of a muscular dystrophy).

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

The following examples are illustrative of disclosed methods and compositions. In light of this disclosure, those of ordinary skill in the art will recognize that variations of these examples and other examples of the disclosed methods and compositions would be possible without undue experimentation.

### Example 1: Methods

### 1. Animal Experiments

Male wild-type C57Bl/6J (The Jackson Laboratory, Bar Harbor, Me.) and mdx^{4cv} (generated in house) mice were utilized for these studies (17). All animals were experimentally manipulated in accordance with the Institutional Animal Care and Use Committee (IACUC) of the University of Washington. Experimental mice were administered vector at 22-24 months of age via the retro-orbital sinus with a 200-μl bolus injection in Hanks Balanced Saline Solution (HBSS) at a dose of 2×10¹⁴ vg/kg. All mice were housed in a specific-pathogen free animal care facility using a 12-hr light/12-hr dark cycle with access to food and water ad libitum.

### 2. Vector Production

Recombinant AAV genomes containing the CK8 regulatory cassette (expressed exclusively in skeletal and cardiac muscle) and the human codon optimized (GenScript) μDys (ΔR2-15/ΔR18-22/ΔCT) (24), followed by the rabbit beta-globin poly-adenylation (pA) signal, were generated using standard cloning techniques. The rAAV genomes containing the cardiac-muscle specific cTnT455 regulatory cassette, the codon optimized human RNR transgene flanked by 100-bp UTR's, and the rabbit beta-globin pA were generated as previously described (49). The ‘dead’ rAAV genomes or promoter-less firefly luciferase followed by the human growth hormone (hGH) pA (kindly provided by JSC, University of Washington, Seattle Wash.) were used to generate the control rAAV genomes. The resulting constructs were co-transfected with the pDG6 packaging plasmid into HEK293 cells to generate rAAV vectors carrying serotype 6 capsids, that were harvested, enriched, and quantitated as previously described (50).

### 3. Vector Genome Quantification

Total DNA was extracted from flash-frozen tissue samples with Tri-Reagent (MRC Inc.), according to manufacturer's instructions. All real-time PCR reactions were performed on a QuantStudio 3 Real Time PCR System (Applied Biosystems, Foster City, Calif.) in a total volume of 15 consisting of 5 μl sample DNA, 10.0 μl TaqMan Universal PCR Master Mix (Applied Biosystems, Foster City, Calif.), 0.2 μM of each primer, and 0.1 μM TaqMan custom probe (Applied Biosystems, Foster City, Calif.). Reaction conditions were 50° C. for 2 minutes, 95° C. for 10 minutes, and 40 cycles of 95° C. for 15 seconds followed by 60° C. for 1 minute. Each sample was analyzed in triplicate for concentration of total murine genomes and of total vector genomes. For vector genome detection by qPCR, the primers used to amplify either the rAAV6-cTnT455-RNR or rAAV6-CK8-μDys or rAAV6-ACMV-Luc (control vector) were unique to each vector. For the RNR vector the amplicon spanned from the distal region of the cTnT regulatory cassette, continuing into the proximal RNR1 subunit. For the μDys vector the amplicon was contained within the CK8 regulatory cassette, while the amplicon for the control vector resided within the human growth hormone (hGH) poly-adenylation. hGH Primers: 5′-CACAATCTTGGCTCACTGCAA-3′, 5′-GGAGGCTGAGGCAGGAGAA-3′, TaqMan Probe: 5′-6FAM-CTCCGCCTCCTGGGTTCAAGCG-MBGNQ-3′; CK8 RC Primers: 5′-CCCGAGATGCCTGGTTATAATT-3′, 5′-CGGGAACATGGCATGCA-3′, TaqMan Probe: 5′-6FAM-CCCCCCAACACCTGCTGCCTCT-MBGNQ-3′; cTnT455-RNR1 Primers: 5′-CCCAGTCCCCGCTGAGA-3′, 5′-AGGTTCCAGGCGCTGCT-3′, TaqMan Probe: 5′-6FAM-ACTCATCAATGTATCTTATCATG-MBGNQ-3′. Results were presented relative to DNA content in each 5 μl DNA tissue sample to determine vector genomes per ng DNA.

### 4. Tissue Processing and Imaging Analysis

Tissues were collected and analyzed 5 months post-administration of vectors and compared with age-matched male control vector (rAAV6-ACMV-Luc) injected mdx^{4cv} and wild-type (WT) mice. Hearts were either snap frozen in liquid nitrogen or were embedded in Optimal Cutting Temperature (O.C.T.) compound (VWR International) and flash frozen in liquid nitrogen cooled isopentane for histochemical or immunofluorescence analysis. The snap frozen samples were further processed by grinding to a powder under liquid nitrogen in a mortar kept on dry ice for subsequent extraction of nucleic acid and protein.

Heart cross-sections (10 μm) were co-stained with antibodies raised against alpha 2-laminin (Sigma, rat monoclonal, 1:200), the hinge-1 domain of dystrophin (alexa488 conjugated MANEX1011b, Developmental Studies Hybridoma Bank, University of Iowa, mouse monoclonal, 1:200), the human RRM1 (Abcam, rabbit monoclonal, 1:200), and the human RRM2 (Abcam, rabbit monoclonal, 1:200). Conjugated secondary antibodies (Jackson Immuno, Goat anti-Rabbit) were used at a 1:500 dilution. Slides were mounted using ProLong Gold with DAPI (Thermo Fisher Scientific) and imaged via a Leica SPV confocal microscope. Confocal micrographs covering a majority of the heart left ventricular muscle sections were acquired and montaged via the Fiji toolset (ImageJ) and InDesign (Adobe). For histology, Masson's trichrome staining was used to examine heart cross sections. Briefly, 10-μm muscle cryosections were sequentially stained in Wiegerts' iron hematoxylin (10 min), 1% Ponceau-acetic acid (5 min), and 1% aniline blue (5 s).

### 5. Western Blotting

Radioimmunoprecipitation analysis buffer (RIPA) supplemented with 5 mM EDTA and 3% protease inhibitor cocktail (Sigma, Cat #P8340), was used to extract muscle proteins for 0.5 hour on ice with gentle agitation every 10 min. Total protein concentration was determined using Pierce BCA assay kit (ThermoFisher). Muscle lysates from WT, control mdx^{4cv} and treated mdx^{4cv} (30 μg) mice were denatured at 99 degrees Celsius for 10 min, quenched on ice, and separated via gel electrophoresis after loading onto Criterion 4-12% Bis-Tris polyacrylamide gels (BioRad). Overnight protein transfer to 0.45 mm PVDF membranes was performed at constant 43 volts at 4-degrees Celsius in Towbin's buffer containing 20% methanol. Blots were blocked for 1 hour at room temperature in 5% non-fat dry milk for 1 hour before overnight incubation with antibodies raised against the hinge-1 region of dystrophin (Developmental Studies Hybridoma Bank, University of Iowa, 1:300), anti-RRM1 (Abcam, rabbit monoclonal, 1:1,000), anti-RRM2 (Abcam, rabbit monoclonal 1:1,000), and anti-GAPDH (Sigma, Rabbit polyclonal, 1:50,000). Horseradish-peroxidase conjugated secondary antibody staining (1:50,000) was performed for 1 h at room temperature before signal development using Clarity Western ECL substrate (BioRad) and visualization using a Chemidoc MP imaging system (BioRad).

6. Quantification of Cardiac [dATP]

Approximately 25 pg of flash frozen, freshly ground ventricle cardiac tissue was used for direct quantification of intracellular dATP using the HPLC-MS/MS method previously described (51). Briefly, samples were extracted 1-3 days before measurement using a 50% methanol solution. The supernatant was stored at −20° C. until ready for injection into the HPLC-MS/MS system. A Water's Xevo-TQ-S mass spectrometer coupled with a Water's Acquity I-Class HPLC was used for the analysis (Milford, Mass., USA). Monitoring in negative mode via an electrospray ionization (ESI) was used to acquire MS-MS ions. dATP concentrations were quantified with standards and normalized to tissue weight.

### 7. Langendorff Isolated Perfused Heart Experiments

Ex-vivo cardiac function was assessed in Langendorff isolated heart preparations as previously described (48,49,52). Hearts were perfused at a constant pressure of 80 mmHg with a modified Krebs-Henseleit (KH) buffer supplemented with glucose and pyruvate. The perfusate contained (in mmol/L): 118 NaCl, 25 NaHCO₃, 5.3 KCl, 2.0 CaCl2, 1.2 MgSO4, 0.5 EDTA, 10.0 glucose, and 0.5 pyruvate, equilibrated with 95% 02 and 5% CO2 (pH 7.4). Temperature was maintained at 37.5° C. throughout the protocol. Left ventricular (LV) function was monitored via a water-filled balloon inserted into the LV and connected to a pressure transducer. LV systolic pressure (LVSP), end diastolic pressure (EDP), heart rate (HR), and minimum and maximum rate of pressure change in the ventricle (±dP/dt) were obtained from the attached data acquisition system (PowerLab, ADInstruments, Colorado Springs, Colo.). After 5 minutes of stabilization, hearts were equilibrated for 10 minutes at spontaneous heart rates and then fixed at a heart rate of ˜450 bpm with an electrical stimulator (Grass Technologies, Warwick, R.I.). Pressure-volume relationships (i.e., Frank-Starling curves) were assessed by gradually increasing the volume of the LV balloon. After a 5-minute recovery period, the perfusate was changed to an identical buffer as above except for the addition of 4.0 mmol/L CaCl2 to simulate a high workload (HWL) challenge for 20 minutes.

### 8. Statistical Analysis

All values are reported as means±standard error of the mean (SEM). Starling curves and HWL function were analyzed by two-way repeated measures analysis of variance (ANOVA) followed by Tukey's post hoc analysis. End-point data was analyzed via one-way ANOVA or t-tests as appropriate. Mantel-Cox tests were used to analyze survival curves. Significance was tested at the P<0.05 level.

### Example 2: Results

i. Improvements in Baseline Cardiac Function in Vector-Treated mdx^{4cv} Hearts

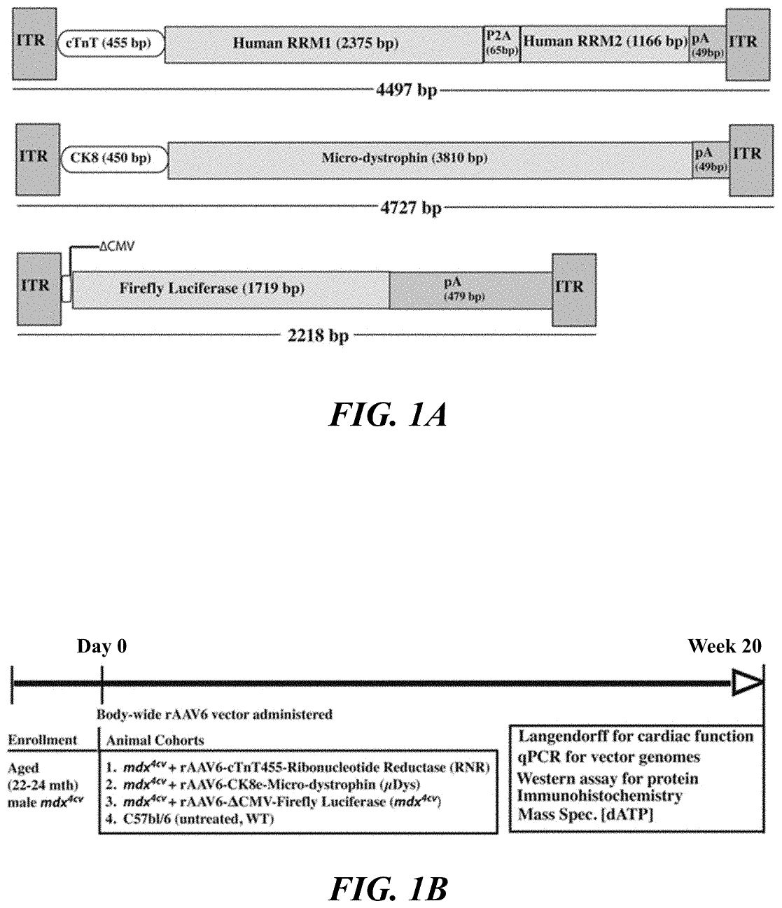

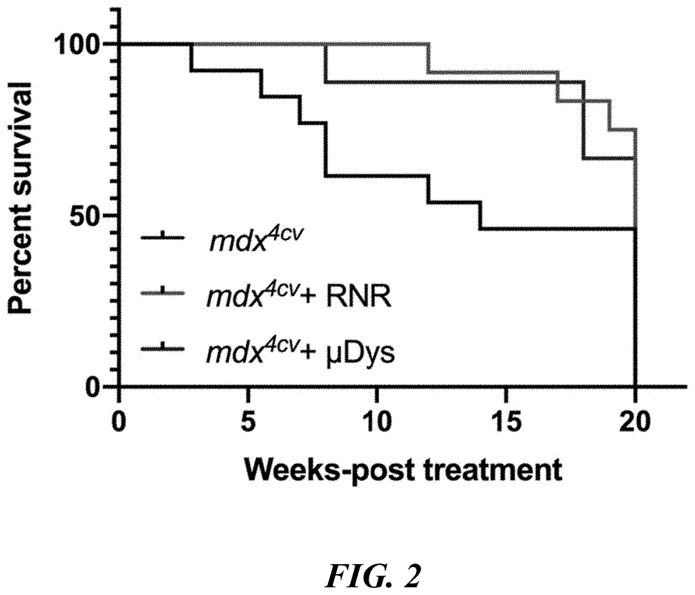

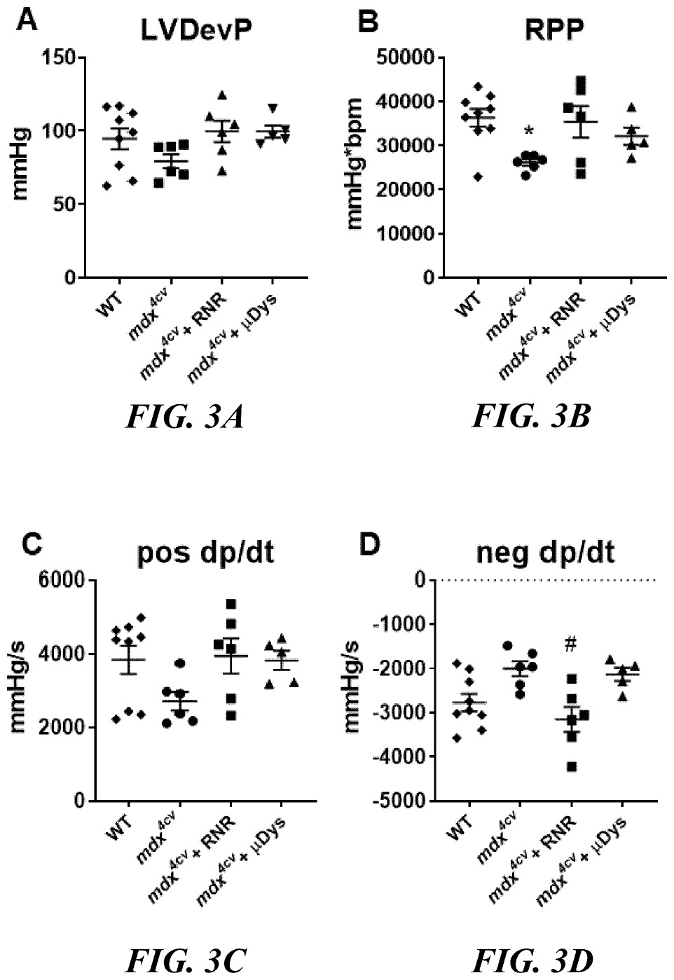

As depicted in FIG. 1, 22-24 month-old mdx^{4cv} mice were administered one of three treatments: rAAV6-cTnT455-ribonucleotide reductase (RNR; referred to as mdx^{4cv}+RNR); rAAV6-CK8-micro-dystrophin (μDys; referred to as mdx^{4cv}+μDys), or rAAV6-ACMV-Firefly Luciferase control vector (referred to as mdx^{4cv}) at a dose of 2×10″ vg/kg. By the end of the 20-week treatment period, both mdx^{4cv}+RNR and mdx^{4cv}+μDys mice showed improvements in survival rates compared to mdx^{4cv} mice, although this did not reach statistical significance (FIG. 2). At the end of 5 months, an extensive evaluation of ex-vivo cardiac function using the Langendorff isolated heart preparation was performed. The isolated heart technique allows for the direct assessment of inherent myocardial function without the confounding effects of neuro-humoral or other systemic variables. An additional cohort of age-matched, untreated C57BL6 mice (WT) were used as comparison controls. At baseline, RPP was significantly decreased in mdx^{4cv} hearts due to an approximate 20% decrease in LVDevP (FIGS. 3A and 3B). RNR treated mdx^{4cv} mice exhibited a restoration of RPP (P=0.0564) primarily due to a normalization of LVDevP (FIGS. 3A and 3B). Although μDys treated mdx^{4cv} hearts appeared to normalize LVDevP, this did not lead to a significant improvement in RPP (FIGS. 3A and 3B). Both +dP/dt and −dP/dt, an index of ventricular contractility and relaxation, respectively, were decreased 30% in mdx^{4cv} hearts (P=0.06). The +dP/dt was similar to control in both RNR treated mdx^{4cv} and μDys treated mdx^{4cv} hearts. However, only RNR treated mdx^{4cv} hearts demonstrated −dP/dt values similar to control levels (FIGS. 3C and 3D).

2. Positive Changes in Frank-Starling Mechanics in Vector-Treated mdx^{4cv} Hearts

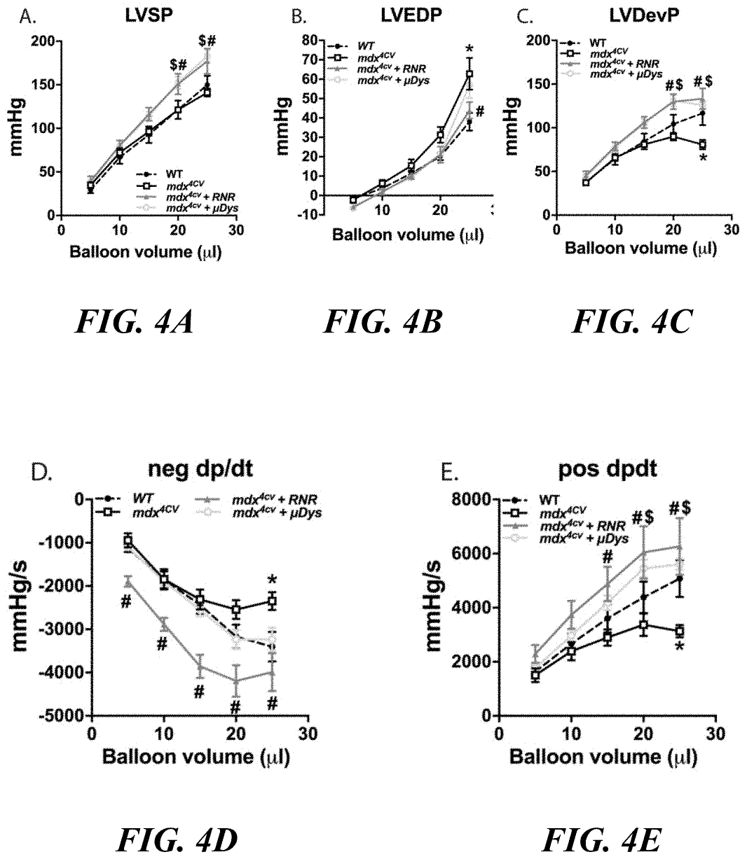

To evaluate further systolic and diastolic function in vector treated-mdx^{4cv} hearts, the pressure-volume relationship (i.e., Frank-Starling mechanism) in the isolated perfused heart preparation were examined. The left ventricular systolic pressure (LVSP) response to increased preload was significantly improved in both in mdx^{4cv}+RNR and mdx^{4cv}+μDys hearts compared to mdx^{4cv} (FIG. 4A). However, only RNR treatment improved the diastolic response in mdx^{4cv} hearts, to levels similar to WT (FIG. 4B). Both contractility and relaxation (i.e., +dP/dt and −dP/dt, respectively) were impaired in mdx^{4cv} compared to age-matched controls (FIGS. 4D and 4E). Both mdx^{4cv}+RNR and mdx^{4cv}+μDys hearts had significantly elevated +dP/dt values above mdx^{4cv} (FIG. 4E). Interestingly, treatment of mdx^{4cv} hearts with RNR also significantly improved −dP/dt values (FIG. 4D). These data suggest that both RNR and μDys treatment can improve systolic function in mdx^{4cv} hearts. However, these data showed that only the RNR treatment corrected diastolic dysfunction in mdx^{4cv} hearts.

3. Augmented Response to Increased Cardiac Workload in Treated mdx^{4cv} Hearts

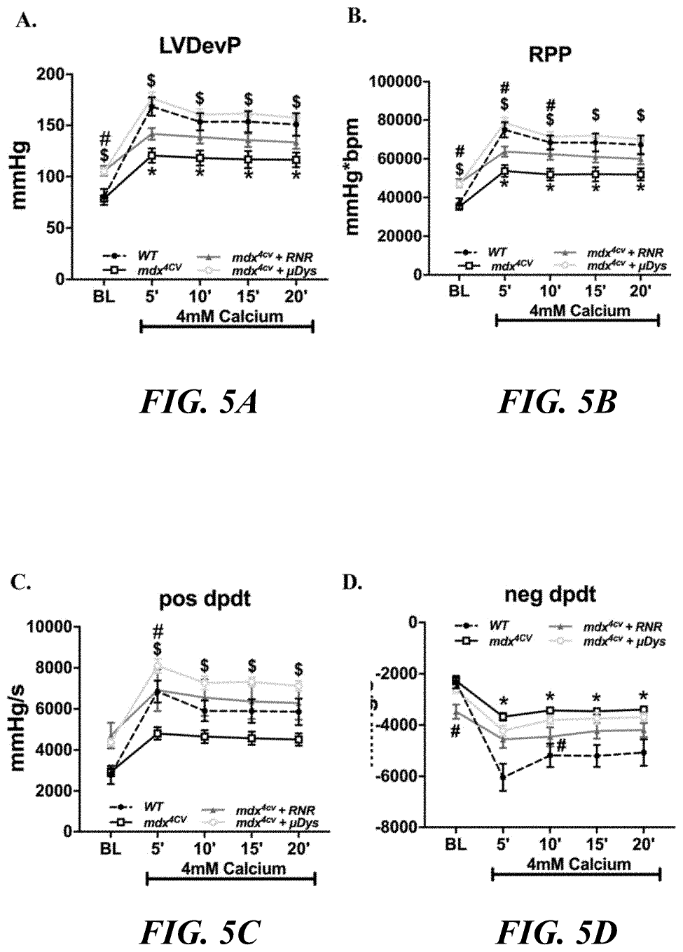

It was previously reported that RNR overexpression in transgenic or vector-treated mouse hearts elevated baseline function but did not impair the response to an acute physiological increase in cardiac work (48,49). To verify that the improved systolic and diastolic function in RNR treated mdx^{4cv} hearts at baseline was not associated with an inability to respond to an increased energetic demand, hearts were stressed with a combination of high calcium and elevated heart rates, via pacing stimulation. As shown in FIGS. 5A and 5B, mdx^{4cv} hearts had a blunted response to the increased workload as both LVDevP and RPP were ˜25-30% lower than wild-type hearts. In addition both +dP/dt and −dP/dt were impaired in mdx^{4cv} relative to wild-type hearts (FIGS. 5C and 5D). Systolic parameters in mdx^{4cv}+μDys hearts were effectively improved and similar to age-matched wild-type hearts for the entire duration of the workload challenge (FIGS. 5A to 5C). Measures of systolic function significantly increased in mdx^{4cv}+RNR hearts during the initial half of the high workload protocol and remained ˜15% higher than mdx^{4cv} (FIGS. 5A to 5C). Interestingly, −dP/dt values tended to be elevated only in mdx^{4cv}+RNR hearts during the physiological challenge (FIG. 5D). These data show that both RNR and μDys treatments improve systolic function in mdx^{4cv} hearts without compromising cardiac reserve. Combined with the baseline and pressure-volume relationship assessments, these data demonstrate that, in addition to the systolic enhancements, RNR has an added benefit of improving diastolic function.

### 4. RNR and μDys Transduction, Expression, and Cardiomyocyte Localization

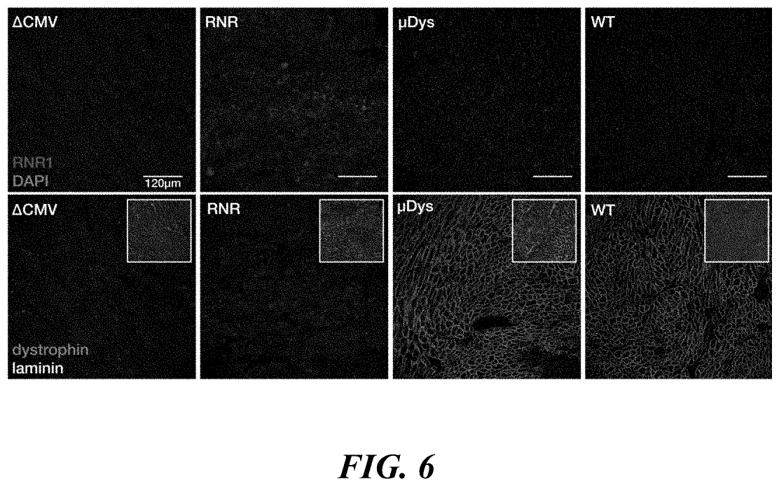

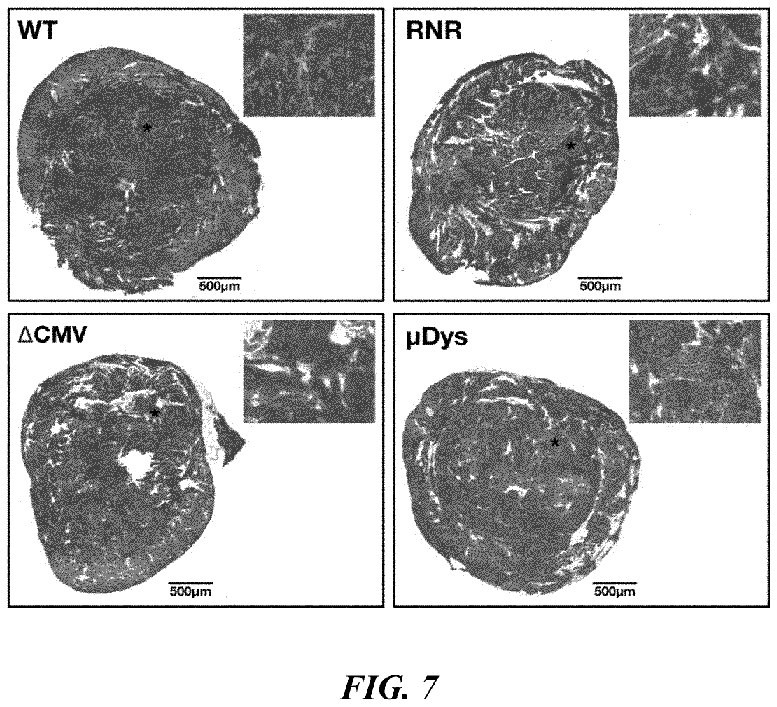

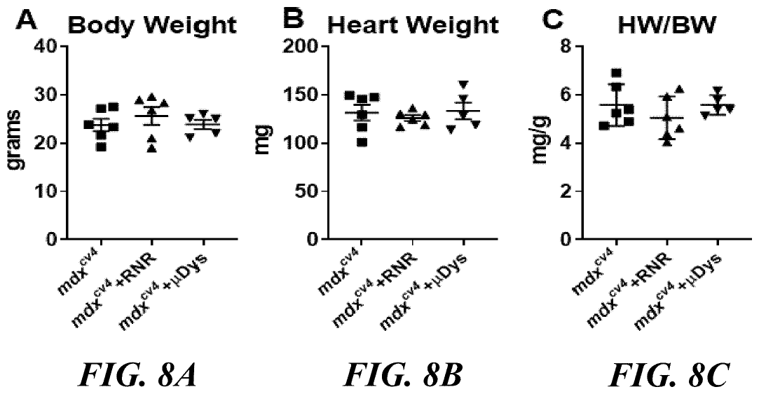

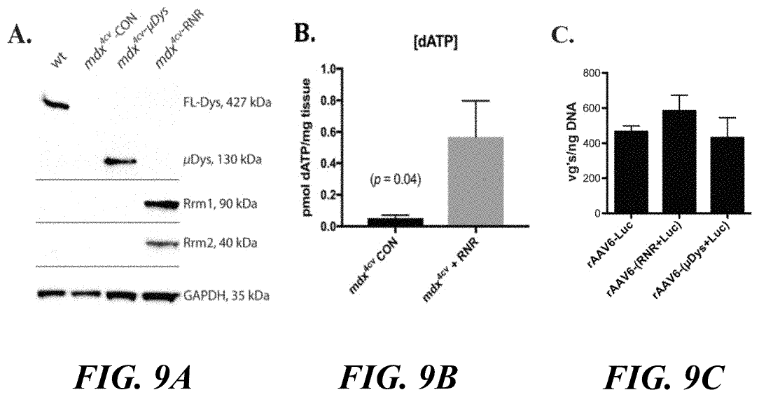

To evaluate the localization of RNR and micro-dystrophin protein within the hearts of mice, immunofluorescence imaging was performed. As shown in FIG. 6, the RNR subunit (RRM1) was robustly expressed in ventricles of RNR treated mice. The expression of μDys appeared to be saturated relative to full-length dystrophin levels, with both being properly localized to the sarcolemma of cardiomyocytes. Evaluations of general muscle histopathology and potential differences in myocardial fibrosis by Masson trichrome staining were also performed, and no discernable difference between treated or untreated mdx^{4cv} mice were observed (FIG. 7). In addition, neither RNR nor μDys treatment significantly altered body weight (BW), heart weight (HW), or the HW to BW ratio (FIG. 8). Western blotting was performed to determine the extent of rAAV6-mediated RNR and μDys protein expression profiles in ventricular tissue (FIG. 9). μDys protein expression in ventricular tissue that approached levels similar to wild-type mice were observed, while both human RNR subunits (RRM1 and RRM2) were found to be elevated to comparable levels within ventricular tissue (FIG. 9A). To evaluate the relative proportions of dATP concentrations within ventricular tissue, HPLC-MS/MS analysis was performed on ground ventricular tissue from mdx^{4cv} and mdx^{4cv}+RNR mice. The concentration of dATP within the ventricular tissue obtained from mdx^{4cv} mice treated with RNR (0.568±0.22 pmol dATP/mg) was approximately 10-fold higher relative to mdx^{4cv} controls (0.051±0.02 pmol dATP/mg) (FIG. 9B). For adult wildtype, an average dATP value of 0.021 pmol/mg tissue with a standard deviation of 0.007 was previously reported (51). Additionally, cardiac vector genome data was comparable relative to the vector dose administered (FIG. 9C).

### Example 3: Animal Models

Provided herein are animal models used in pre-clinical research for DMD therapeutic development.

i. mdx

Mouse models have been used extensively to elucidate the pathogenic mechanisms of DMD, and have been indispensable in the development of therapeutic approaches. The mdx mouse is the most commonly used animal model for the analysis dystrophin expression and function. The mdx mouse contains a premature stop codon in exon 23 that leads to loss of full-length dystrophin, although smaller isoforms are still expressed.^{1,2} The mdx skeletal muscle shows moderate signs of dystrophy, young mice exhibit modest weakness and live ˜80% as long as controls, significantly more than that of DMD patients.³

Histological examination of mdx muscle during various stages of development reveals that muscle fiber necrosis and cellular infiltration begin at approximately 3 weeks of age. This is followed by a crisis period that peaks at approximately 4-6 weeks of age and is characterized by the presence of extensive necrosis, regenerating muscle fibers with centrally located nuclei, and elevated levels of serum creatine kinase (CK).^{1,4} After 12 weeks, the cycles of necrosis and regeneration begin to slow, although necrotic myofibers are present for the remainder of their lifespan. The fibrosis and infiltration of inflammatory cells in skeletal and cardiac muscle of the mdx are much milder than that observed in DMD patients.^{5,6} Similarly cardiomyopathy does not typically manifest until advanced age and often requires sensitive assays for functional deficit detection.^{7,8} In contrast, the mdx mouse diaphragm exhibits severe pathological changes and functional deficits comparable to that of DMD limb muscle.^{9,10} Four additional strains of mdx mice, mdx2cv-5cv, have been generated with N-ethylnitrosourea chemical mutagenesis.¹¹ All these strains have point mutations that lead to loss of full-length dystrophin isoforms. The relative location of these mutations results in a series of mdx mouse mutants that vary in their expression of different dystrophin isoforms.¹² Regardless of their differences, all five mdx strains display essentially identical muscle pathology as mdx mice, although additional phenotypes have been observed.^{11,13,14}

2. mdx4cv

The mdx4cv strain displays a low background of reverent dystrophin containing fibers, making it a particularly useful strain in gene transfer studies exploring the feasibility of DMD therapy.¹⁴⁻¹⁶ Genetically, the mdx4cv mouse, has a point mutation that creates a stop codon in exon 53, and like other mdx strains displays a late-onset cardiomyopathy.¹⁷ Nonetheless, the mdx4cv was chosen as the model to demonstrate the robust benefits of AAV-mediated RNR & micro-dystrophin expression toward improvement of cardiac function.¹⁸

3. mdx:utrn−/− and mdx:utrn−/+

In efforts to make the mdx muscle phenotype more similar to that of patients, several additional mutations have been crossed onto the mdx background to generate double knockouts (DKOs). The most widely used is a dystrophin:utrophin DKO (mdx:utrn−/−).^{19,20} DKO mice display a severe phenotype including advanced cardiomyopathy, mild skeletal muscle fibrosis and an average lifespan of only ˜3 months. The severity of the phenotype supports the concept that utrophin upregulation in dystrophic muscles partially compensates for the absence of dystrophin. Further, the DKO mice have proved useful in gene therapy studies, where the phenotype can be largely eliminated by muscle-specific expression of utrophin, mini-utrophin, or mini- or micro-dystrophin.^{15,16,19-22} Additionally, the mdx:utrn+/−(het) mice have been quite useful, which display a normal (“mdx”) lifespan (˜2 yr) with severe skeletal muscle fibrosis and cardiomyopathy progression more similar to DMD patients making them an attractive model, particularly for cardiac studies.^{23,24}

### 4. Dmdmdx Rat

Generated by TALENs targeting exon 23, two lines of Dmdmdx rats both demonstrate undetectable levels of dystrophin.²⁵ At 3-months of age the Dmdmdx hearts are notably dilated showing increased left ventricular (LV) diameter with LV wall thinning²⁶ At 7 months, limb and respiratory muscles also showed severe fibrosis and some adipose tissue infiltration. Concomitment with the histopathology results, Dmdmdx rats also showed significant reduction in muscle strength and a decrease in locomotion.²⁶ Demonstrating a more clinically relevant disease progression, particularly as it relates to cardiac function and histopathology, the Dmdmdx rat has gained momentum for the evaluation of gene therapies.

### 5. Canine Model of Duchenne Muscular Dystrophy

As a large animal model for DMD, spontaneous mutations causing dystrophinopathy have been identified in several breeds of dog.27,28 This led to the generation of multiple colonies of the golden retriever muscular dystrophy dog (GRMD) being created, and is the most extensively studied breed for this model.29,30 Due to prior use in research and its smaller size, the GRMD mutation has been bred onto the beagle background.31,32 Severe symptoms commonly appear at 6 months of age in the GRMD dog, but unlike the mdx mouse, the degree of severity and time of progression are quite variable. However, use of the GRMD model for potential therapies has gained much emphasis with its more clinically similar pathology than the mdx mouse model.

### Example 4: Delivery Vehicles

### AAV Serotypes for Neuromuscular Disease

Recombinant adeno-associated viral vectors (rAAV) have received considerable attention as prospective gene delivery vectors for the treatment of genetic diseases¹⁻⁶. In the case of severe neuromuscular conditions such as Duchenne muscular dystrophy, using rAAV for gene therapy for intervention would require the transduction of at least 40-50% of muscle fibers in the body⁷⁻¹⁰.

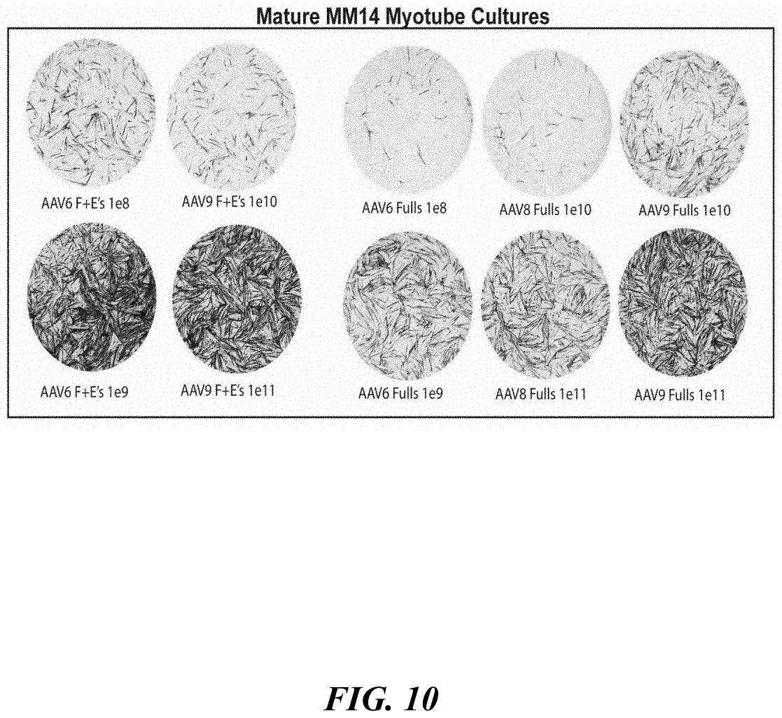

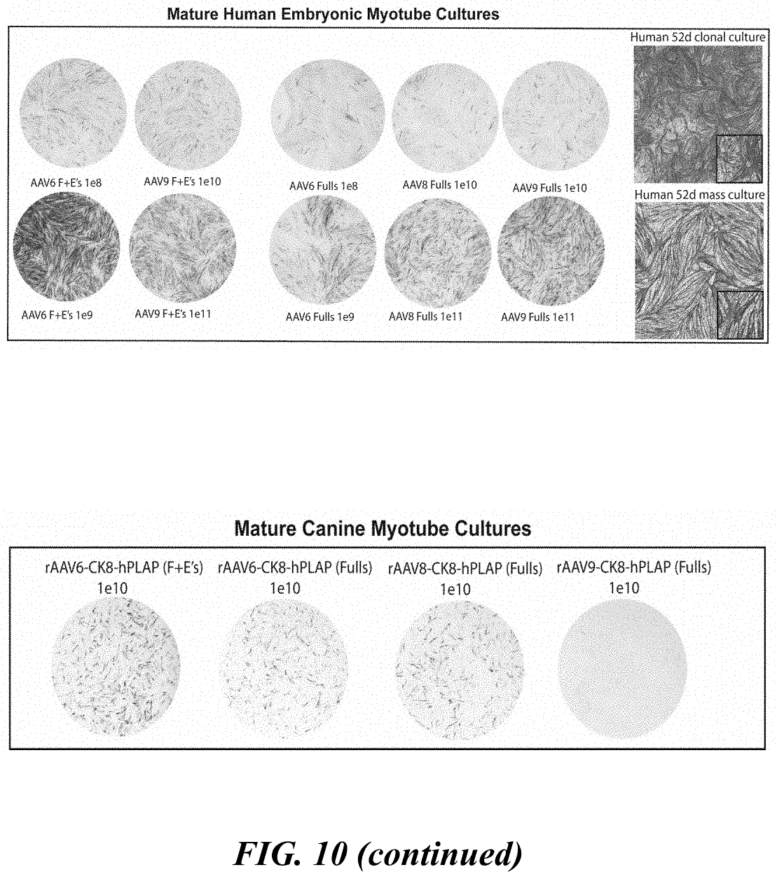

A number of recombinant AAV serotypes, in particular serotypes 1, 6, 8, and 9, have been shown to transduce striated muscle with high efficiency^{11-14.} Indeed, our group has investigated numerous serotype comparisons over the past decade or so. As an example, in vitro myotube cultures (mouse (MM14), canine, & human) were grown, inoculated & compared for indicated reporter gene expression utilizing AAV6, AAV8, & AAV9 serotypes where each species demonstrates a preferential expression pattern with AAV6 transduction (FIG. 10). All vector preparations included the muscle specific regulatory cassette CK8e driving expression of human placental alkaline phosphatase (hPLAP) as previously described.¹⁵ In this study, the presence of empty capsids aided the transduction efficiency of AAV6 and AAV9 in mature human myotube cultures, but appear to hinder that of AAV9 in MM14 cultures. The transduction efficiency of AAV8 was the lowest compared to AAV6 and 9 in mouse and human mature myotube cultures, but was similar to AAV6 in canine myotube cultures. In contrast, AAV9 transduced poorly in canine myotube cultures.

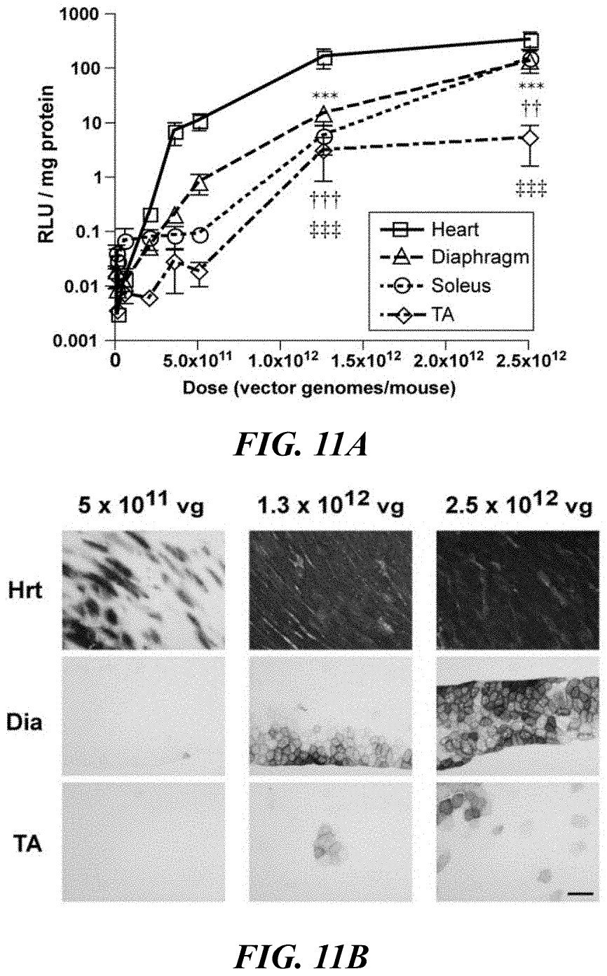

While previous reports have studied the dose response effects of rAAV in intramuscular injections, or in systemic injections of a vector encoding a secreted protein¹⁶⁻¹⁸ we sought to examine the relative expression levels of a non-secreted protein in various striated muscles following systemic rAAV6 administration at increasing doses. We observed an apparent dose-response threshold common to all striated muscles, as well as an individual muscle-specific transduction profile (FIG. 11).

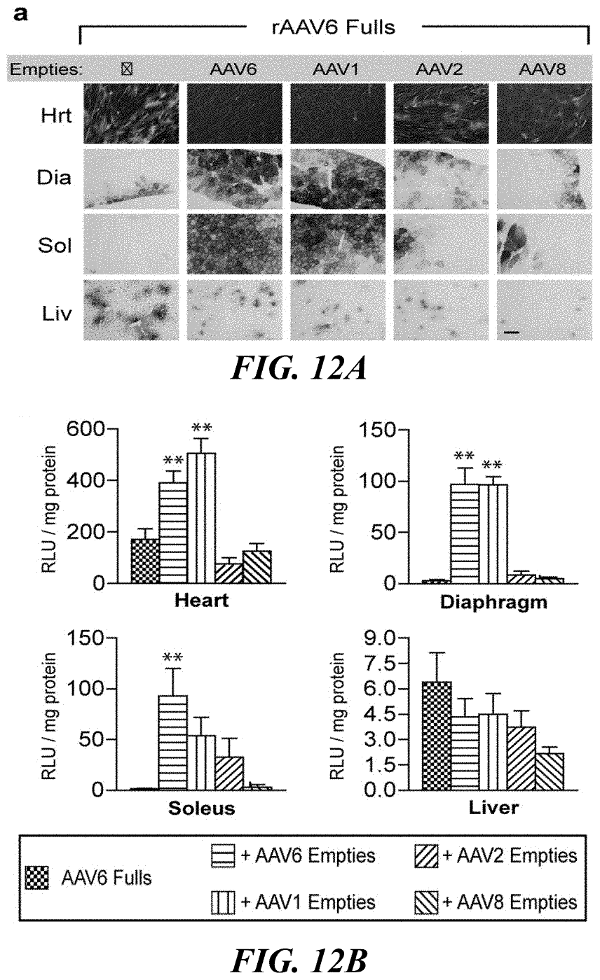

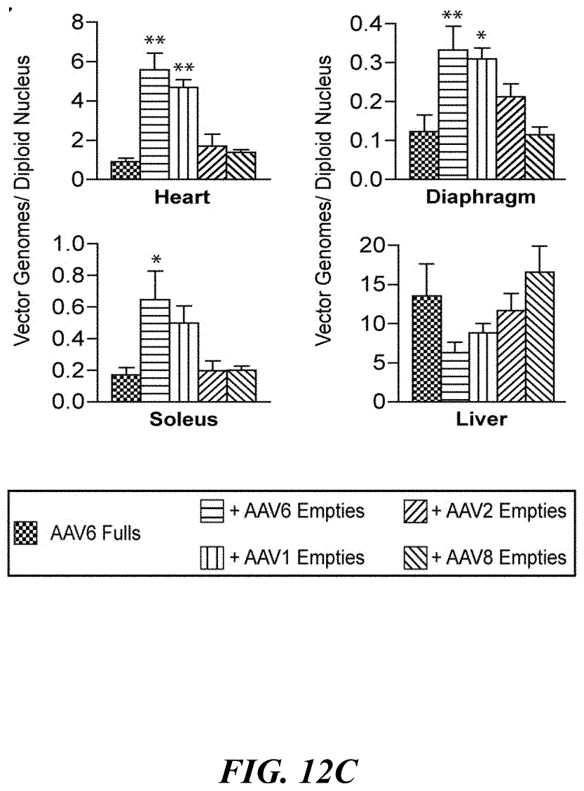

Finally, common methods of rAAV production typically generate a yield comprising 80-90% genome-devoid (or so-called “empty”) capsids that may be included or removed from the vector preparation prior to use, depending on purification/enrichment methods^{18,19} As empty capsids have been reported to decrease transduction in intramuscular injections¹⁸, we sought to test the hypothesis that a supplementary dose of empty capsids may affect transduction by “full” vectors when administered via systemic co-delivery. We found that empty capsids enhance transduction in striated muscles via intravenous administration, in a serotype-specific manner (FIG. 12). Collectively, our results suggest a capsid-specific protein load dependent mechanism of whole body transduction with rAAV6. Improving upon striated muscle transduction continues to be an interest of our combined group moving forward.

### Example 3: Additional Examples of Engineered RNR Constructs

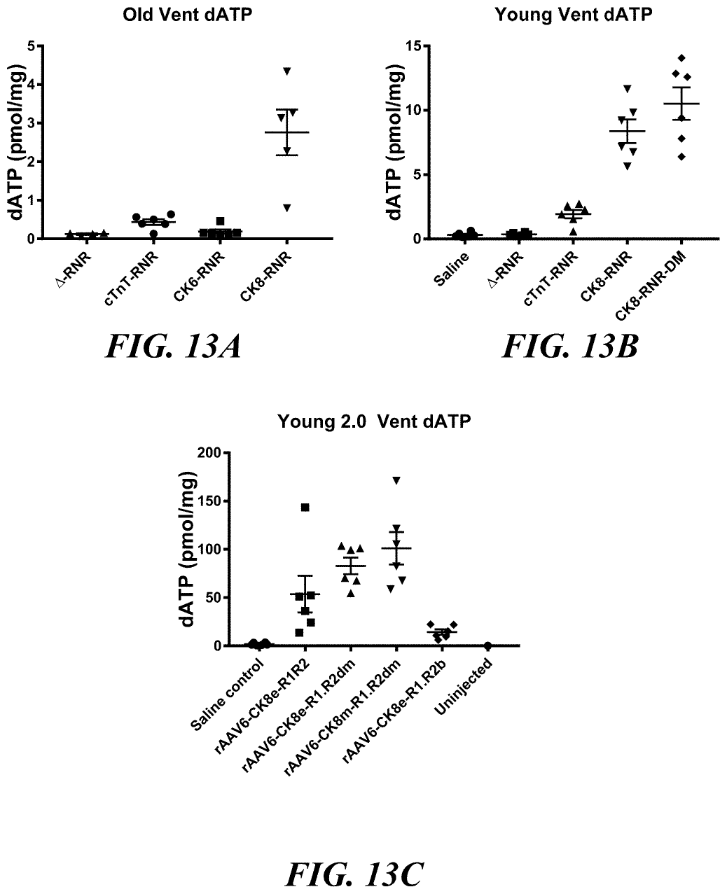

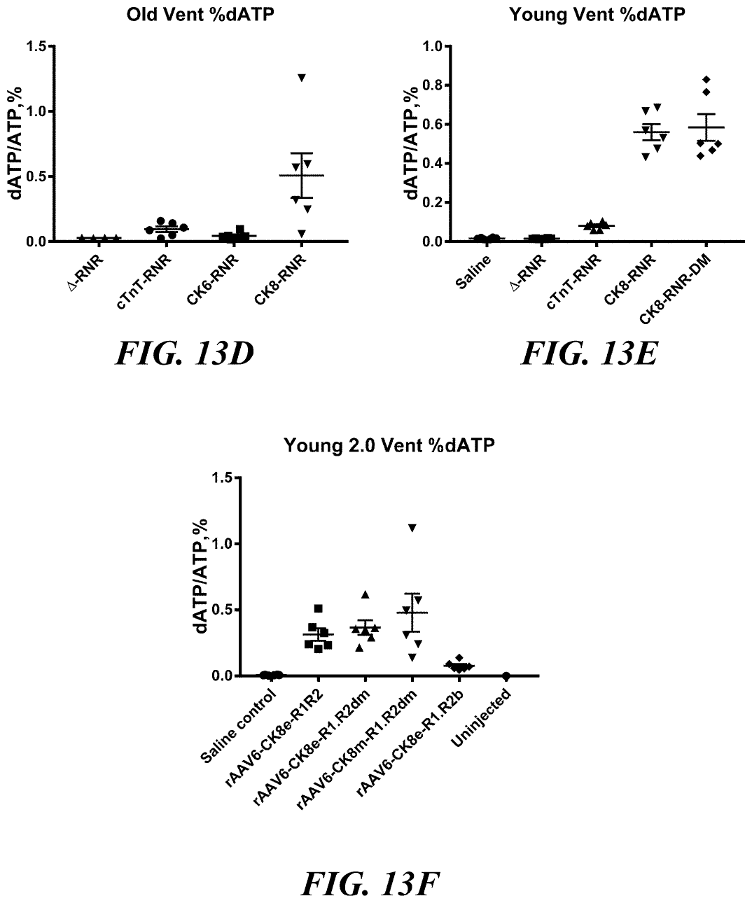

Various combinations of different promotors, an engineered version of the RNR enzyme that resists degradation, and an RNR construct that contains a different gene for the RRM2b subunit that also resists degradation were compared. Three separate studies were conducted in young and old mice. Mice received systemic injections of the AAV vectors and hearts were harvested one month later for analysis of dATP content (FIG. 13A-13F).

It will be readily understood that the embodiments, as generally described herein, are exemplary. The following more detailed description of various embodiments is not intended to limit the scope of the present disclosure, but is merely representative of various embodiments. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The applicants expect skilled artisans to employ such variations as appropriate, and the applicants intend for the various embodiments of the disclosure to be practiced otherwise than specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

Furthermore, numerous references have been made to patents and printed publications throughout this application. Each of the references and printed publications recited in this application are individually incorporated herein by reference in their entirety.

It is to be understood that the embodiments of the present disclosure are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure.

Definitions and explanations used in the present disclosure are meant and intended to be controlling in any future construction unless clearly and unambiguously modified in the examples or when application of the meaning renders any construction meaningless or essentially meaningless in cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Ed. Anthony Smith, Oxford University Press, Oxford, 2004).

It will be apparent to those having skill in the art that many changes may be made to the details of the above-described embodiments without departing from the underlying principles of the invention.

### APPENDIX

### References

i. Emery A E and Muntoni F. Duchenne Muscular Dystrophy. 3rd ed. Oxford: Oxford University Press. 2003: 270.
ii. Ervasti J M. Structure and function of the dystrophin-glycoprotein complex. in Molecular Mechanisms of Muscular Dystrophies. S. J. Winder, Editor. Landes Biosciences: Georgetown. 2006: 1-13.
iii. Cox G A, Phelps S F, Chapman V M, and Chamberlain J S. New mdx mutation disrupts expression of muscle and nonmuscle isoforms of dystrophin. Nature Genet. 1993; 4: 87-93.
iv. Brooks S V and Faulkner J A. Contractile properties of skeletal muscles from young, adult and aged mice. Journal of Physiology (London). 1988; 404: 71-82.
v. Campbell K P. Three muscular dystrophies: loss of cytoskeleton-extracellular matrix linkage. Cell. 1995; 80: 675-679.
vi. Petrof B J, Shrager J B, Stedman H H, Kelly A M, and Sweeney H L. Dystrophin protects the sarcolemma from stresses developed during muscle contraction. Proc. Natl. Acad. Sci. USA. 1993; 90: 3710-3714.
vii. Ramaswamy K S, Palmer M L, van der Meulen J H, et al. Lateral transmission of force is impaired in skeletal muscles of dystrophic mice and very old rats. J Physiol. 2011; 589(Pt 5): 1195-208.
viii. Chao D S, Gorospe J R, Brenman J E, et al. Selective loss of sarcolemmal nitric oxide synthase in Becker muscular dystrophy. J Exp Med. 1996; 184(2): 609-18.
ix. Froehner S C. Just say N O to muscle degeneration? Trends Mol Med. 2002; 8(2): 51-3.
x. Grady R M, Grange R W, Lau K S, et al. Role for alpha-dystrobrevin in the pathogenesis of dystrophin-dependent muscular dystrophies. Nat Cell Biol. 1999; 1(4): 215-20.
xi. Lapidos K A, Kakkar R, and McNally E M. The dystrophin glycoprotein complex: signaling strength and integrity for the sarcolemma. Circ Res. 2004; 94(8): 1023-31.
xii. Ervasti J M and Campbell K P. A role for the dystrophin-glycoprotein complex as a transmembrane linker between laminin and actin. J Cell Biol. 1993; 122(4): 809-23.
xiii. Ibraghimov-Beskrovnaya O, Ervasti J M, Leveille C J, et al. Primary structure of dystrophin-associated glycoproteins linking dystrophin to the extracellular matrix. Nature. 1992; 355(6362): 696-702.
xiv. Yoshida M and Ozawa E. Glycoprotein complex anchoring dystrophin to sarcolemma. J Biochem (Tokyo).

1990; 108(5): 748-52.
xv. England S B, Nicholson L V, Johnson M A, et al. Very mild muscular dystrophy associated with the deletion of 46% of dystrophin. Nature. 1990; 343: 180-182.
xvi. Valentine B A, Winand N J, Pradhan D, et al. Canine X-linked muscular dystrophy as an animal model of Duchenne muscular dystrophy: a review. Am. J. Med. Genet. 1992; 42: 352-356.
xvii. Im W B, Phelps S F, Copen E H, et al. Differential expression of dystrophin isoforms in strains of mdx mice with different mutations. Hum Mol Genet. 1996; 5(8): 1149-53.
xviii. Phelps S F, Hauser M A, Cole N M, et al. Expression of full-length and truncated dystrophin mini-genes in transgenic mdx mice. Hum. Mol. Genet. 1995; 4: 1251-1258.
xix. Crawford G E, Faulkner J A, Crosbie R H, et al. Assembly of the dystrophin-associated protein complex does not require the dystrophin COOH-terminal domain. Journal of Cell Biology. 2000; 150(6): 1399-410.
xx. Harper S Q, Hauser M A, DelloRusso C, et al. Modular flexibility of dystrophin: Implications for gene therapy of Duchenne muscular dystrophy. Nature Medicine. 2002; 8(3): 253-61.
xxi. Wang B, Li J, and Xiao X. Adeno-associated virus vector carrying human minidystrophin genes effectively ameliorates muscular dystrophy in mdx mouse model. Proceedings of the National Academy of Sciences of the United States of America. 2000; 97(25): 13714-9.
xxii. Sakamoto M, Yuasa K, Yoshimura M, et al. Micro-dystrophin cDNA ameliorates dystrophic phenotypes when introduced into mdx mice as a transgene. Biochem Biophys Res Commun. 2002; 293(4): 1265-72.
xxiii. Chamberlain J S. Gene therapy of muscular dystrophy. Hum Mol Genet. 2002; 11(20): 2355-62.
xxiv. Hakim C H, Wasala N B, Pan X, et al. A Five-Repeat Micro-Dystrophin Gene Ameliorated Dystrophic Phenotype in the Severe DBA/2J-mdx Model of Duchenne Muscular Dystrophy. Mol Ther Methods Clin Dev. 2017; 6: 216-230.
xxv. Cox G A, Cole N M, Matsumura K, et al. Overexpression of dystrophin in transgenic mdx mice eliminates dystrophic symptoms without toxicity [see comments]. Nature. 1993; 364(6439): 725-9.
xxvi. Stedman H H, Sweeney H L, Shrager J B, et al. The mdx mouse diaphragm reproduces the degenerative changes of Duchenne muscular dystrophy. Nature. 1991; 352: 536-539.
xxvii. Brooks S V and Faulkner J A. Skeletal muscle weakness in old age: underlying mechanisms. Medical

Science and Sports Exercise. 1994; 26(4): 432-9.
xxviii. DelloRusso C, Crawford R, Chamberlain J, and Brooks S. Tibialis anterior muscles of mdx mice are highly susceptible to contraction-induced injury. Journal of Muscle Research and Cell Motility. 2001; 22: 467-475.
xxix. Lynch G S, Hinkle R T, Chamberlain J S, Brooks S V, and Faulkner J A. Force and power output of fast and slow skeletal muscles from mdx mice 6-28 months old. J Physiol. 2001; 535(Pt 2): 591-600.
xxx. Bostick B, Shin J H, Yue Y, et al. AAV micro-dystrophin gene therapy alleviates stress-induced cardiac death but not myocardial fibrosis in >21-m-old mdx mice, an end-stage model of Duchenne muscular dystrophy cardiomyopathy. J Mol Cell Cardiol. 2012; 53(2): 217-22.
xxxi. Gregorevic P, Blankinship M J, Allen J M, and Chamberlain J S. Systemic microdystrophin gene delivery improves skeletal muscle structure and function in old dystrophic mdx mice. Mol Ther. 2008; 16(4): 657-64.
xxxii. Quinlan J G, Hahn H S, Wong B L, et al. Evolution of the mdx mouse cardiomyopathy: physiological and morphological findings Neuromuscul Disord. 2004; 14(8-9): 491-6.
xxxiii. Gregorevic P, Allen J M, Minami E, et al. rAAV6-microdystrophin preserves muscle function and extends lifespan in severely dystrophic mice. Nat Med. 2006; 12(7): 787-9.
xxxiv. Shin J H, Nitahara-Kasahara Y, Hayashita-Kinoh H, et al. Improvement of cardiac fibrosis in dystrophic mice by rAAV9-mediated microdystrophin transduction. Gene Ther. 2011; 18(9): 910-9.
xxxv. Townsend D, Blankinship M J, Allen J M, et al. Systemic administration of micro-dystrophin restores cardiac geometry and prevents dobutamine-induced cardiac pump failure. Mol Ther. 2007; 15(6): 1086-92.
xxxvi. Bostick B, Shin J H, Yue Y, and Duan D. AAV-microdystrophin therapy improves cardiac performance in aged female mdx mice. Mol Ther. 2011; 19(10): 1826-32.
xxxvii. Townsend D, Yasuda S, Chamberlain J, and Metzger J M. Cardiac consequences to skeletal muscle-centric therapeutics for Duchenne muscular dystrophy. Trends Cardiovasc Med. 2009; 19(2): 50-55.
xxxviii. Adhikari B B, Regnier M, Rivera A J, Kreutziger K L, and Martyn D A. Cardiac length dependence of force and force development kinetics with altered cross-bridge cycling. Biophysical Journal. 2004; 87: 1784-1794.
xxxix. Clemmens E W, Entezari M, Martyn D A, and Regnier M. Different effects of cardiac versus skeletal muscle regulatory proteins on in vitro measures of actin filament speed and force. J Physiol. 2005; 566(Pt 3): 737-46.
xl. Korte F S, Dai J, Buckley K, et al. Upregulation of cardiomyocyte ribonucleotide reductase increases intracellular 2 deoxy-ATP, contractility, and relaxation. J Mol Cell Cardiol. 2011; 51(6): 894-901.
xli. Kreutziger K L, Piroddi N, Belus A, Poggesi C, and Regnier M. Ca2+-binding kinetics of troponin C influence force generation kinetics in cardiac muscle. Biophysical Journal. 2007; 92(Suppl): 477a.
xlii. Kreutziger K L, Piroddi N, J. T. M, et al. Cooperative activation and tension kinetics in cardiac muscle are strongly modulated by calcium binding kinetics of troponin C. Journal of Molecular and Cellular Cardiology. 2011; 50(1): 165-174.
xliii. Moussavi-Harami F, Razumova M V, Racca A W, et al. 2-Deoxy adenosine triphosphate improves contraction in human end-stage heart failure. J Mol Cell Cardiol. 2015; 79: 256-63.
xliv. Regnier M, Lee D M, and Homsher E. ATP analogs and muscle contraction: mechanics and kinetics of nucleoside triphosphate binding and hydrolysis. Biophys J. 1998; 74(6): 3044-58.
xlv. Regnier M, Martin H, Barsotti R J, et al. Cross-bridge versus thin filament contributions to the level and rate of force development in cardiac muscle. Biophys J. 2004; 87(3): 1815-24.
xlvi. Regnier M, Rivera A J, Chen Y, and Chase P B. 2-deoxy-ATP enhances contractility of rat cardiac muscle. Circ Res. 2000; 86(12): 1211-7.
xlvii. Cheng Y, Hogarth K A, O'Sullivan M L, Regnier M, and Pyle W G. 2-Deoxyadenosine triphosphate restores the contractile function of cardiac myofibril from adult dogs with naturally occurring dilated cardiomyopathy. Am J Physiol Heart Circ Physiol. 2016; 310(1): H80-91.
xlviii. Nowakowski S G, Kolwicz S C, Korte F S, et al. Transgenic overexpression of ribonucleotide reductase improves cardiac performance. Proc Natl Acad Sci USA. 2013; 110(15): 6187-92.
xlix. Kolwicz S C, Jr., Odom G L, Nowakowski S G, et al. AAV6-mediated Cardiac-specific Overexpression of Ribonucleotide Reductase Enhances Myocardial Contractility. Mol Ther. 2016; 24(2): 240-50.
l. Halbert C L, Allen J M, and Chamberlain J S. AAV6 Vector Production and Purification for Muscle Gene Therapy. Methods Mol Biol. 2018; 1687: 257-266.
li. Olafsson S, Whittington D, Murray J, Regnier M, and Moussavi-Harami F. Fast and sensitive HPLC-MS/MS method for direct quantification of intracellular deoxyribonucleoside triphosphates from tissue and cells. J Chromatogr B Analyt Technol Biomed Life Sci. 2017; 1068-1069: 90-97.
lii. Kolwicz S C, Jr. and Tian R. Assessment of cardiac function and energetics in isolated mouse hearts using 31P NMR spectroscopy. J Vis Exp. 2010; 42: e2069.
liii. Kadota S, Carey J, Reinecke H, et al. Ribonucleotide reductase-mediated increase in dATP improves cardiac performance via myosin activation in a large animal model of heart failure. Eur J Heart Fail. 2015; 17(8): 772-81.
liv. Lundy S D, Murphy S A, Dupras S K, et al. Cell-based delivery of dATP via gap junctions enhances cardiac contractility. J Mol Cell Cardiol. 2014; 72: 350-9.
lv. Khairallah M, Khairallah R, Young M E, et al. Metabolic and signaling alterations in dystrophin-deficient hearts precede overt cardiomyopathy. J Mol Cell Cardiol. 2007; 43(2): 119-29.
lvi. Zhang W, ten Hove M, Schneider J E, et al. Abnormal cardiac morphology, function and energy metabolism in the dystrophic mdx mouse: an MRI and MRS study. J Mol Cell Cardiol. 2008; 45(6): 754-60.
lvii. Stuckey D J, Carr C A, Camelliti P, et al. In vivo MRI characterization of progressive cardiac dysfunction in the mdx mouse model of muscular dystrophy. PLoS One. 2012; 7(1): e28569.
lviii. Bostick B, Yue Y, and Duan D. Gender influences cardiac function in the mdx model of Duchenne cardiomyopathy. Muscle Nerve. 2010; 42(4): 600-3.
lix. Bell R M, Mocanu M M, and Yellon D M. Retrograde heart perfusion: the Langendorff technique of isolated heart perfusion. J Mol Cell Cardiol. 2011; 50(6): 940-50.
lx. Liao R, Podesser B K, and Lim C C. The continuing evolution of the Langendorff and ejecting murine heart: new advances in cardiac phenotyping. Am J Physiol Heart Circ Physiol. 2012; 303(2): H156-67.
lxi. Kaspar R W, Allen H D, Ray W C, et al. Analysis of dystrophin deletion mutations predicts age of cardiomyopathy onset in becker muscular dystrophy. Circ Cardiovasc Genet. 2009; 2(6): 544-51.
lxii. Feng J, Yan J, Buzin C H, Towbin J A, and Sommer S S. Mutations in the dystrophin gene are associated with sporadic dilated cardiomyopathy. Mol Genet Metab. 2002; 77(1-2): 119-26.
lxiii. Ortiz-Lopez R, Li H, Su J, Goytia V, and Towbin J A. Evidence for a dystrophin missense mutation as a cause of X-linked dilated cardiomyopathy. Circulation. 1997; 95(10): 2434-40.
lxiv. Singh S M, Bandi S, Shah D D, Armstrong G, and Mallela K M. Missense mutation Lys18Asn in dystrophin that triggers X-linked dilated cardiomyopathy decreases protein stability, increases protein unfolding, and perturbs protein structure, but does not affect protein function. PLoS One. 2014; 9(10): e110439.
lxv. Lee T H, Eun L Y, Choi J Y, et al. Myocardial atrophy in children with mitochondrial disease and Duchenne muscular dystrophy. Korean J Pediatr. 2014; 57(5): 232-9.
lxvi. Matsuoka S, Ii K, Akita H, et al. Clinical features and cardiopulmonary function of patients with atrophic heart in Duchenne muscular dystrophy. Jpn Heart J. 1987; 28(5): 687-94.
lxvii. Konstam M A, Kramer D G, Patel A R, Maron M S, and Udelson J E. Left ventricular remodeling in heart failure: current concepts in clinical significance and assessment. JACC Cardiovasc Imaging. 2011; 4(1): 98-108.
lxviii. Silva M C, Meira Z M, Gurgel Giannetti J, et al. Myocardial delayed enhancement by magnetic resonance imaging in patients with muscular dystrophy. J Am Coll Cardiol. 2007; 49(18): 1874-9.
lxix. Shirokova N and Niggli E. Cardiac phenotype of Duchenne Muscular Dystrophy: insights from cellular studies. J Mol Cell Cardiol. 2013; 58: 217-24.
lxx. Nigro G, Comi L I, Politano L, and Bain R J. The incidence and evolution of cardiomyopathy in Duchenne muscular dystrophy. Int J Cardiol. 1990; 26(3): 271-7.
lxxi. Li Y, Zhang S, Zhang X, et al. Blunted cardiac beta-adrenergic response as an early indication of cardiac dysfunction in Duchenne muscular dystrophy. Cardiovasc Res. 2014; 103(1): 60-71.
lxxii. Barbin I C, Pereira J A, Bersan Rovere M, et al. Diaphragm degeneration and cardiac structure in mdx mouse: potential clinical implications for Duchenne muscular dystrophy. J Anat. 2016; 228(5): 784-91.
lxxiii. Costas J M, Nye D J, Henley J B, and Plochocki J H. Voluntary exercise induces structural remodeling in the hearts of dystrophin-deficient mice. Muscle Nerve. 2010; 42(6): 881-5.
lxxiv. Nakamura A, Yoshida K, Takeda S, Dohi N, and Ikeda S. Progression of dystrophic features and activation of mitogen-activated protein kinases and calcineurin by physical exercise, in hearts of mdx mice. FEBS Lett. 2002; 520(1-3): 18-24.

SEQUENCES

SEQ ID NO: 1 is the nucleotide sequence encoding human RRM1, isoform 1.
GeneID: 6240
NCBI Reference Sequence: NG_027992.2
SEQ ID NO: 2 is the amino acid sequence for human RRM1, isoform 1.
MHVIKRDGRQERVMFDKITSRIQKLCYGLNMDFVDPAQITMKVI
QGLYSGVTTVELDTLAAETAATLTTKHPDYAILAARIAVSNLHKETKKVFSDVMEDLY
NYINPHNGKHSPMVAKSTLDIVLANKDRLNSAITYDRDFSYNYFGFKTLERSYLLKIN
GKVAERPQHMLMRVSVGIHKEDIDAAIETYNLLSERWFTHASPTLFNAGTNRPQLSSC
FLLSMKDDSIEGIYDTLKQCALISKSAGGIGVAVSCIRATGSYIAGTNGNSNGLVPML
RVYNNTARYVDQGGNKRPGAFAIYLEPWHLDIFEFLDLKKNTGKEEQRARDLFFALWI
PDLFMKRVETNQDWSLMCPNECPGLDEVWGEEFEKLYASYEKQGRVRKVVKAQQLWYA
IIESQTETGTPYMLYKDSCNRKSNQQNLGTIKCSNLCTEIVEYTSKDEVAVCNLASLA
LNMYVTSEHTYDFKKLAEVTKVVVRNLNKIIDINYYPVPEACLSNKRHRPIGIGVQGL
ADAFILMRYPFESAEAQLLNKQIFETIYYGALEASCDLAKEQGPYETYEGSPVSKGIL
QYDMWNVTPTDLWDWKVLKEKIAKYGIRNSLLIAPMPTASTAQILGNNESIEPYTSNI
YTRRVLSGEFQIVNPHLLKDLTERGLWHEEMKNQIIACNGSIQSIPEIPDDLKQLYKT
VWEISQKTVLKMAAERGAFIDQSQSLNIHIAEPNYGKLTSMHFYGWKQGLKTGMYYLR
TRPAANPIQFTLNKEKLKDKEKVSKEEEEKERNTAAMVCSLENRDECLMCGS
SEQ ID NO: 3 is the mRNA sequence for human RRM1, isoform 1.
>NM_001033.5 Homo sapiens ribonucleotide reductase catalytic subunit M1
(RRM1), transcript variant 1, mRNA
CCCTTTGTGCGTCACGGGTGGCGGGCGCGGGAAGGGGATTTGGATTGTTGCGCCTCTGCTCTGAAGAAAG
TGCTGTCTGGCTCCAACTCCAGTTCTTTCCCCTGAGCAGCGCCTGGAACCTAACCCTTCCCACTCTGTCA
CCTTCTCGATCCCGCCGGCGCTTTAGAGCCGCAGTCCAGTCTTGGATCCTTCAGAGCCTCAGCCACTAGC
TGCGATGCATGTGATCAAGCGAGATGGCCGCCAAGAACGAGTCATGTTTGACAAAATTACATCTCGAATC
CAGAAGCTTTGTTATGGACTCAATATGGATTTTGTTGATCCTGCTCAGATCACCATGAAAGTAATCCAAG
GCTTGTACAGTGGGGTCACCACAGTGGAACTAGATACTTTGGCTGCTGAAACAGCTGCAACCTTGACTAC
TAAGCACCCTGACTATGCTATCCTGGCAGCCAGGATCGCTGTCTCTAACTTGCACAAAGAAACAAAGAAA
GTGTTCAGTGATGTGATGGAAGACCTCTATAACTACATAAATCCACATAATGGCAAACACTCTCCCATGG
TGGCCAAGTCAACATTGGATATTGTTCTGGCCAATAAAGATCGCCTGAATTCTGCTATTATCTATGACCG
AGATTTCTCTTACAATTACTTCGGCTTTAAGACGCTAGAGCGGTCTTATTTGTTGAAGATCAATGGAAAA
GTGGCTGAAAGACCACAACATATGTTGATGAGAGTATCTGTTGGGATCCACAAAGAAGACATTGATGCAG
CAATTGAAACATATAATCTTCTTTCTGAGAGGTGGTTTACTCATGCTTCGCCCACTCTCTTCAATGCTGG
TACCAACCGCCCACAACTTTCTAGCTGTTTTCTTCTGAGTATGAAAGATGACAGCATTGAAGGCATTTAT
GACACTCTAAAGCAATGTGCATTGATTTCTAAGTCTGCTGGAGGAATTGGTGTTGCTGTGAGTTGTATTC
GGGCTACTGGCAGCTACATTGCTGGGACTAATGGCAATTCCAATGGCCTTGTACCGATGCTGAGAGTATA
TAACAACACAGCTCGATATGTGGATCAAGGTGGGAACAAGCGTCCTGGGGCATTTGCTATTTACCTGGAG
CCTTGGCATTTAGACATCTTTGAATTCCTTGATTTAAAGAAGAACACAGGAAAGGAAGAGCAGCGTGCCA
GAGATCTTTTCTTTGCTCTTTGGATTCCGGATCTCTTCATGAAACGAGTGGAGACTAATCAGGACTGGTC
TTTGATGTGTCCAAATGAGTGTCCTGGTCTGGATGAGGTTTGGGGAGAGGAATTTGAGAAACTATATGCA
AGTTATGAGAAACAAGGTCGTGTCCGCAAAGTTGTAAAAGCTCAGCAGCTTTGGTATGCCATCATTGAGT
CTCAGACGGAAACAGGCACCCCGTATATGCTCTACAAAGATTCCTGTAATCGAAAGAGCAACCAGCAGAA
CCTGGGAACCATCAAATGCAGCAACCTGTGCACAGAAATAGTGGAGTACACCAGCAAAGATGAGGTTGCT
GTTTGTAATTTGGCTTCCCTGGCCCTGAATATGTATGTCACATCAGAACACACATACGACTTTAAGAAGT
TGGCTGAAGTCACTAAAGTCGTTGTCCGAAACTTGAATAAAATTATTGATATAAACTACTATCCTGTACC
AGAGGCATGCCTATCAAATAAACGCCATCGCCCCATTGGAATTGGGGTACAAGGTCTGGCAGATGCTTTT
ATCCTGATGAGATACCCTTTTGAGAGTGCAGAAGCCCAGTTACTGAATAAGCAGATCTTTGAAACTATTT
ATTATGGTGCTCTGGAAGCCAGCTGTGACCTTGCCAAGGAGCAGGGCCCATACGAAACCTATGAGGGCTC
TCCAGTTAGCAAAGGAATTCTTCAGTATGATATGTGGAATGTTACTCCTACAGACCTATGGGACTGGAAG
GTTCTCAAGGAGAAGATTGCAAAGTATGGTATAAGAAACAGTTTACTTATTGCCCCGATGCCTACAGCTT
CCACTGCTCAGATCCTGGGGAATAATGAGTCCATTGAACCTTACACCAGCAACATCTATACTCGCAGAGT
CTTGTCAGGAGAATTTCAGATTGTAAATCCTCACTTATTGAAAGATCTTACCGAGCGGGGCCTATGGCAT
GAAGAGATGAAAAACCAGATTATTGCATGCAATGGCTCTATTCAGAGCATACCAGAAATTCCTGATGACC
TGAAGCAACTTTATAAAACTGTGTGGGAAATCTCTCAGAAAACTGTTCTCAAGATGGCAGCTGAGAGAGG
TGCTTTCATTGATCAAAGCCAATCTTTGAACATCCACATTGCTGAGCCTAACTATGGCAAACTCACTAGT
ATGCACTTCTACGGCTGGAAGCAGGGTTTGAAGACTGGGATGTATTATTTAAGGACAAGACCAGCGGCTA
ATCCAATCCAGTTCACTCTAAATAAGGAGAAGCTAAAAGATAAAGAAAAGGTATCAAAAGAGGAAGAAGA
GAAGGAGAGGAACACAGCAGCCATGGTGTGCTCTTTGGAGAATAGAGATGAATGTCTGATGTGTGGATCC
TGAGGAAAGACTTGGAAGAGACCAGCATGTCTTCAGTAGCCAAACTACTTCTTGAGCATAGATAGGTATA
GTGGGTTTGCTTGAGGTGGTAAGGCTTTGCTGGACCCTGTTGCAGGCAAAAGGAGTAATTGATTTAAAGT
ACTGTTAATGATGATAATGATTTTTTTTTTAAACTCATATATTGGGATTTTCACCAAAATAATGCTTTTG
AAAAAAAGAAAAAAAAAACGGATATATTGAGAATCAAAGTAGAAGTTTTAGGAATGCAAAATAAGTCATC
TTGCATACAGGGAGTGGTTAAGTAAGGTTTCATCACCCCTTTAGCACTGCTTTTCTGAAGACTTCAGTTT
TGTTAAGGAGATTTAGTTTTACTGCTTTGACTGGTGGGTCTCTAGAAGCAAAACTGAGTGATAACTCATG
AGAAGTACTGATAGGACCTTTATCTGGATATGGTCCTATAGGTTATTCTGAAATAAAGATAAACATTTCT
AAGTGATTGTATGAGATTAATTTTGTCATTTACTTTCATATAAAAGTCAAATTTGAAAAACA
SEQ ID NO: 4 is the nucleotide sequence encoding mouse RRM1, isoform 1.
NCBI-GeneID: 20133
SEQ ID NO: 5 is the amino acid sequence for mouse RRM1, isoform 1.
>NP_033129.2 ribonucleoside-diphosphate reductase large subunit
[Mus musculus]
MHVIKRDGRQERVMFDKITSRIQKLCYGLNMDFVDPAQITMKVIQGLYSGVTTVELDTLAAETAATLTTK
HPDYAILAARIAVSNLHKETKKVFSDVMEDLYNYINPHNGRHSPMVASSTLDIVMANKDRLNSAITYDRD
FSYNYFGFKTLERSYLLKINGKVAERPQHMLMRVSVGIHKEDIDAAIETYNLLSEKWFTHASPTLFNAGT
NRPQLSSCFLLSMKDDSIEGIYDTLKQCALISKSAGGIGVAVSCIRATGSYIAGTNGNSNGLVPMLRVYN
NTARYVDQGGNKRPGAFAIYLEPWHLDIFEFLDLKKNTGKEEQRARDLFFALWIPDLFMKRVETNQDWSL
MCPNECPGLDEVWGEEFEKLYESYEKQGRVRKVVKAQQLWYAIIESQTETGTPYMLYKDSCNRKSNQQNL
GTIKCSNLCTEIVEYTSKDEVAVCNLASLALNMYVTPEHTYDFEKLAEVTKVIVRNLNKIIDINYYPIPE
AHLSNKRHRPIGIGVQGLADAFILMRYPFESPEAQLLNKQIFETIYYGALEASCELAKEYGPYETYEGSP
VSKGILQYDMWNVAPTDLWDWKPLKEKIAKYGIRNSLLIAPMPTASTAQILGNNESIEPYTSNIYTRRVL
SGEFQIVNPHLLKDLTERGLWNEEMKNQIIACNGSIQSIPEIPDDLKQLYKTVWEISQKTVLKMAAERGA
FIDQSQSLNIHIAEPNYGKLTSMHFYGWKQGLKTGMYYLRTRPAANPIQFTLNKEKLKDKEKALKEEEEK
ERNTAAMVCSLENREECLMCGS
SEQ ID NO: 6 is the mRNA sequence for mouse RRM1, isoform 1.
>NM_009103.3 Mus musculus ribonucleotide reductase M1 (Rrm1), mRNA
TCAATATGGCGGCCAAGGGACTCGTGTGCTGTCTGTCTACTGCTCAGTTTCCGCCCATTCAACTCCCGGC
GTTGAAACGTCAAGAACGTCATTCGAATTCCGTCCGTCGCGTTGCTCTGCACGTCACGGGTGGCGGGAGC
GGGAAGGAGTTCGTAATTCGGTTAGTCTGCTCTGGTGAGGAAAGTGCTGTCTATCGCGCAGCTTCCATCC
CTCCGTCCGAGCAGCCTCTCGGAGTCCAACCCTTCACATCTGACAGTCGTCTCTGTCCCTTCTTCGCCTC
GGAGCTGCTAACTGGTCTCGAACCTCTCAGCACTTCAGCTTCTAGCGGCGATGCATGTGATCAAGCGAGA
TGGCCGCCAAGAGCGAGTTATGTTTGACAAAATTACATCACGAATCCAGAAACTCTGTTATGGACTCAAC
ATGGACTTTGTTGATCCTGCTCAGATCACCATGAAAGTAATCCAAGGCCTATATAGTGGGGTCACCACAG
TGGAACTGGACACCCTGGCTGCTGAGACAGCCGCGACCTTGACCACGAAGCACCCTGACTATGCCATCCT
GGCAGCAAGGATAGCCGTCTCTAACTTGCACAAAGAAACAAAGAAAGTGTTCAGTGATGTGATGGAGGAT
CTCTACAACTACATAAATCCGCACAACGGCAGACACTCTCCCATGGTGGCCAGCTCAACACTCGACATTG
TTATGGCCAATAAGGATCGCCTGAATTCTGCCATTATCTATGACCGAGATTTCTCTTATAACTACTTTGG
CTTTAAGACACTGGAACGGTCATATTTGTTGAAGATCAATGGTAAAGTGGCTGAAAGACCACAGCATATG
TTGATGAGGGTTTCTGTGGGGATTCACAAAGAAGATATTGATGCTGCAATTGAAACCTACAACCTACTTT
CTGAGAAGTGGTTCACTCATGCCTCTCCTACTCTCTTCAATGCTGGGACCAACCGCCCACAGCTGTCTAG
CTGTTTCCTCTTGAGTATGAAAGATGACAGCATTGAAGGAATTTATGATACTCTGAAGCAGTGTGCCTTG
ATTTCTAAGTCCGCTGGGGGAATTGGTGTTGCTGTGAGTTGTATTCGGGCCACTGGTAGCTACATCGCTG
GGACTAATGGCAATTCTAATGGCCTTGTGCCAATGCTGAGAGTATATAACAACACAGCTCGCTATGTGGA
TCAAGGTGGAAACAAGCGCCCAGGCGCGTTTGCTATTTACCTGGAGCCTTGGCACTTAGACATCTTTGAG
TTCCTTGACTTGAAGAAGAACACAGGCAAGGAAGAACAGCGAGCACGCGATCTCTTCTTTGCACTTTGGA
TCCCAGATCTCTTCATGAAGCGAGTGGAGACTAACCAGGACTGGTCATTGATGTGTCCCAATGAGTGTCC
TGGTCTGGACGAGGTCTGGGGAGAGGAGTTTGAGAAGTTATATGAAAGTTACGAGAAGCAGGGTCGTGTC
CGAAAAGTTGTAAAAGCTCAGCAGCTTTGGTATGCCATCATTGAGTCCCAGACGGAGACCGGTACCCCAT
ACATGCTCTACAAAGATTCCTGTAACCGGAAGAGCAACCAGCAGAACCTGGGAACCATCAAATGCAGCAA
CCTGTGTACAGAAATAGTAGAGTACACCAGTAAAGATGAGGTTGCAGTTTGTAACTTGGCTTCTCTGGCT
CTGAATATGTATGTCACACCGGAACATACGTATGACTTTGAGAAACTGGCAGAAGTCACTAAAGTCATTG
TCCGAAATCTGAATAAAATAATTGATATAAACTACTACCCTATTCCAGAGGCACACTTATCAAATAAACG
CCATCGGCCCATTGGAATTGGGGTACAAGGTTTAGCAGATGCTTTCATCCTGATGAGATACCCCTTTGAG
AGCCCAGAAGCCCAGTTATTAAATAAGCAGATCTTTGAAACCATTTACTATGGAGCCCTGGAAGCCAGCT
GTGAACTAGCCAAGGAGTATGGCCCCTATGAAACGTATGAGGGATCTCCAGTCAGCAAGGGTATTCTTCA
GTATGACATGTGGAATGTTGCTCCTACAGACCTGTGGGACTGGAAGCCTCTCAAGGAGAAGATTGCAAAG
TATGGTATAAGGAACAGTTTACTTATTGCCCCAATGCCTACTGCTTCAACTGCCCAGATTCTGGGGAATA
ATGAGTCCATTGAGCCTTATACCAGTAACATCTACACTCGAAGAGTCTTGTCAGGGGAATTTCAGATTGT
GAATCCTCACTTACTGAAAGATCTTACTGAGCGGGGCTTGTGGAATGAAGAGATGAAAAATCAGATTATT
GCATGCAATGGCTCCATTCAGAGCATACCAGAAATTCCTGATGACCTGAAGCAACTCTATAAGACCGTGT
GGGAAATCTCTCAGAAGACTGTTCTCAAGATGGCAGCCGAGAGAGGTGCTTTCATCGATCAGAGCCAGTC
TTTAAACATCCATATTGCTGAGCCCAACTACGGCAAACTCACTAGTATGCACTTCTACGGTTGGAAGCAG
GGTTTAAAGACTGGAATGTATTACTTAAGGACGAGGCCTGCCGCTAATCCAATCCAGTTCACTCTGAACA
AGGAAAAACTGAAAGATAAGGAAAAGGCACTGAAGGAGGAGGAGGAGAAGGAGAGGAACACAGCAGCCAT
GGTGTGCTCTTTGGAGAACAGAGAGGAGTGCCTGATGTGTGGATCCTGAGAAAATCAGGGCCTGGGAGAC
GCAGCGGGCTCTCCTGCCCGCCGAGGCAGACGATTTGAGCATAGATAGGATAGTGGGTTTGCTTGGTTAT
CAGCAGCTCTGCTTGGACGTGCCTGCCAGGACAGGGAGCCACGACTTACAGTACTGTTTCTACACAGTGT
AAATATCATTTTTAACAAACAGAAAACCAAAGCCAGCTTTGATATTAGGAATCAAAGTAGAGGCTTTGGG
AATACTAAAGAGCCTTCCTGCAAATTAGTGAGGAGACTTAGGAAGTCTCGTCTCTCCAGCTTTCCCTGCC
TGGCCATTCTCAGTTTGGGCAAAGAGATTTAGTTTGATTTGACTGATTGCCTAGAAGTAAAATCAAGCAA
TTACTCATCAGCTAAAGACCTTTGTCTAGACAAACTTCTATAGGTCATTTTGAAATAAACATTTCTAAGT
GATTGTGTGGTACTAAACTTGTCATCTATTATCATACAAGACAGTTTAGGGGAAAAAACCCAAAAACCCA
ACATTTTCTGTTGAGTTCAGAGAGACAAACTTTAAAGACATTTAGATTGTATAGATATCTAGTGTTAACA
TATGCCCTTTCCTGCCCCAGGATGAAATCTTGTTAACATAAAATTGACAGTTTCTTTCATTTATAATTTG
ATTCTGTGGCATTTAGTTCATTCACACTGTTGTATAAACTGTCATCCACACCATTTCCAAAACATTCCAT
CATTCCAAATAGAGACTCTACTCATAACCACACTTCTTACACCTTTTTGAATATGTATTCCTCATACACA
TAATGCAGTATTTGCACTTGTATGGCTTGCATGATATCTAGATACATCACAGTGTGATACGCTGTCCCTC
CATATGTGCACACCATCTTGTATCCATCCTGTCATCTGTTCATGAAACTTGGTTGTTTCCTCCCTTTAGA
TAGGGAGAATAATGGCTGCCATGAACATTGGTCTACAAATATCTATTGGATTCCTGCTTTTAGGTTTGGT
GGTTCTATACAGCAAAGAATTGCTGGACTATATAATTCTGTTTGACTTTGAGGAGCTATATTTGCAGCAC
CATTATATTTCTATAAAAGTACTAAAAGGCCTTATTCTGTCTTCATACCTTATAACACTCGATTTTCATA
TTTTTGATAAAGCCCTTCTATGAGTGGGGAATTTCCTGGTCTTGTAGATTGACTTGTTTCGTTAAACCCG
AGTTTTGGGGCATTTTCTCCCTTTAGTCATCACATCCTTTTTTTCCCTTATGAAACTCATAATAAATCTG
CTTTACG
SEQ ID NO: 7 is the nucleotide sequence encoding rat RRM1, isoform 1.
NCBI GeneID: 685579
SEQ ID NO: 8 is the amino acid sequence for rat RRM1, isoform 1.
>NP_001013254.1 ribonucleoside-diphosphate reductase large subunit
[Rattus norvegicus]
MHVIKRDGRQERVMFDKITSRIQKLCYGLNMDFVDPAQITMKVIQGLYSGVTTVELDTLAAETAATLTTK
HPDYAILAARIAVSNLHKETKKVFSDVMEDLYNYINPHNGRHSPMVASSTLEIVMAHKDRLNSAITYDRD
FSYNYFGFKTLERSYLLKINGKVAERPQHMLMRVSVGIHKEDIDAAIETYNLLSEKWFTHASPTLFNAGT
NRPQLSSCFLLSMKDDSIEGIYDTLKQCALISKSAGGIGVAVSCIRATGSYIAGTNGNSNGLVPMLRVYN
NTARYVDQGGNKRPGAFAIYLEPWHLDIFEFLDLKKNTGKEEQRARDLFFALWIPDLFMKRVETNQDWSL
MCPNECPGLDEVWGEEFEKLYESYEKQGRVRKVVKAQQLWYAIIESQTETGTPYMLYKDSCNRKSNQQNL
GTIKCSNLCTEIVEYTSKDEVAVCNLASLALNMYVTPEHTYDFEKLAEVTKVIVRNLNKIIDINYYPIPE
AHLSNKRHRPIGIGVQGLADAFILMRYPFESPEAQLLNKQIFETIYYGALEASCDLAKEYGPYETYEGSP
VSKGILQYDMWNVTPTDLWDWKLLKEKIAKYGIRNSLLIAPMPTASTAQILGNNESIEPYTSNIYTRRVL
SGEFQIVNPHLLKDLTERGLWNEEMKNQIIACNGSIQSIPEIPEDLKQLYKTVWEISQKTVLKMAAERGA
FIDQSQSLNIHIAEPNYGKLTSMHFYGWKQGLKTGMYYLRTRPAANPIQFTLNKEKLKDKEKALKEEEEK
ERNTAAMVCSLENREECLMCGS
SEQ ID NO: 9 is the mRNA sequence for rat RRM1, isoform 1.
>NM_001013236.1 Rattus norvegicus ribonucleotide reductase catalytic
subunit M1 (Rrm1), mRNA
CGGGTGGCGGGAGCGGGAAGGAGTTCGTAATTTGGTTCGTCCCTTCTGGAGGAGAAAGTGCTGTCTGTCC
GGCAGTTTCAACCTCTCGGTCTGAGCGGCCCCTAAGGAGTCCAACCCTTCACATCTGACAGTCGTCTCTA
TCCTATCTTCGCCTCGGAGCTGCTAACTGGTCTCGAACCCCTCAGCACTTCAGCTTCTAGCGGCGATGCA
TGTGATCAAGCGAGATGGCCGCCAAGAGCGAGTTATGTTTGACAAAATTACATCCCGAATCCAGAAACTC
TGTTATGGACTCAATATGGACTTTGTGGATCCTGCTCAGATCACCATGAAAGTAATCCAAGGCCTATACA
GTGGGGTCACCACAGTGGAACTGGACACCCTGGCTGCTGAGACAGCTGCCACCTTGACTACGAAGCACCC
TGACTATGCCATCCTGGCAGCAAGGATCGCTGTCTCTAACTTGCACAAGGAAACAAAGAAAGTGTTCAGT
GACGTGATGGAGGATCTCTACAACTACATAAATCCACACAACGGCAGACATTCTCCCATGGTGGCCAGCT
CAACACTCGAGATTGTTATGGCCCATAAGGATCGCCTGAATTCTGCCATTATCTATGACCGGGATTTCTC
TTACAACTACTTTGGTTTTAAGACACTGGAACGGTCATATTTGTTGAAGATCAATGGAAAAGTGGCTGAA
AGACCACAGCACATGTTGATGAGGGTATCTGTGGGGATTCACAAAGAAGATATTGATGCTGCAATTGAAA
CATACAATCTACTTTCTGAGAAGTGGTTTACTCACGCCTCTCCGACTCTCTTCAATGCTGGGACCAACCG
CCCACAGTTGTCCAGCTGTTTCCTCTTGAGTATGAAAGATGACAGCATTGAGGGGATTTATGATACTCTG
AAGCAGTGTGCCTTGATTTCTAAGTCTGCTGGAGGAATTGGTGTTGCCGTGAGTTGTATTCGGGCCACTG
GCAGCTACATTGCTGGGACTAATGGCAATTCTAATGGCCTTGTGCCAATGCTGAGAGTCTATAACAACAC
AGCTCGTTATGTGGATCAAGGTGGAAACAAGCGCCCAGGGGCATTTGCTATTTACCTGGAGCCTTGGCAC
CTGGACATCTTTGAGTTTCTTGACTTGAAGAAGAACACAGGCAAGGAAGAACAGCGCGCGCGGGATCTCT
TCTTTGCACTGTGGATCCCAGATCTCTTCATGAAGCGAGTGGAGACCAACCAGGACTGGTCACTGATGTG
TCCCAATGAGTGTCCTGGTCTGGACGAGGTCTGGGGAGAGGAGTTTGAGAAGTTATATGAAAGTTACGAG
AAGCAGGGCCGTGTCCGAAAAGTTGTGAAGGCTCAGCAGCTTTGGTACGCCATCATTGAGTCTCAGACGG
AGACGGGCACCCCATACATGCTCTACAAAGACTCCTGTAACCGGAAGAGCAACCAGCAGAACCTGGGAAC
CATCAAGTGCAGCAACCTGTGCACAGAGATAGTAGAGTACACCAGTAAAGATGAGGTTGCGGTTTGTAAC
TTGGCTTCTCTGGCTCTGAACATGTATGTCACACCAGAACACACGTATGACTTTGAGAAACTGGCAGAAG
TCACTAAAGTCATTGTCCGAAATCTGAATAAAATAATTGATATAAACTACTATCCTATTCCAGAGGCACA
CTTATCAAATAAACGCCATCGGCCCATTGGAATTGGGGTACAAGGTCTAGCAGATGCTTTCATCCTGATG
AGGTATCCCTTTGAGAGCCCAGAAGCCCAGCTACTAAATAAGCAAATCTTTGAAACCATCTATTATGGAG
CCCTGGAAGCCAGCTGTGACCTAGCCAAGGAGTATGGCCCCTACGAAACGTATGAGGGATCTCCAGTCAG
CAAGGGTATTCTTCAGTATGATATGTGGAATGTTACTCCTACAGACCTGTGGGACTGGAAGCTTCTCAAG
GAGAAGATTGCAAAGTACGGTATAAGAAACAGTTTACTTATTGCCCCAATGCCTACTGCTTCAACTGCTC
AGATTCTGGGGAATAATGAGTCCATTGAGCCTTACACCAGTAACATCTACACTCGCAGAGTCTTGTCAGG
AGAATTTCAGATTGTGAATCCTCACTTACTGAAAGATCTTACTGAGCGGGGCTTGTGGAATGAAGAGATG
AAAAATCAGATTATTGCCTGCAATGGCTCCATTCAGAGCATACCAGAAATTCCTGAGGACCTGAAGCAGC
TCTATAAGACCGTGTGGGAAATCTCTCAGAAGACTGTTCTCAAGATGGCAGCCGAGAGAGGTGCTTTCAT
CGATCAAAGCCAGTCTTTAAACATCCATATCGCTGAGCCCAACTATGGCAAACTCACTAGTATGCACTTC
TACGGTTGGAAGCAGGGTTTAAAGACTGGGATGTATTATTTAAGGACAAGACCTGCCGCTAATCCAATCC
AGTTCACTCTGAACAAGGAAAAGCTGAAAGATAAGGAAAAGGCACTGAAGGAGGAAGAAGAGAAGGAGAG
GAACACAGCAGCCATGGTGTGCTCTTTGGAGAACAGAGAGGAGTGTCTGATGTGTGGATCCTGAGACAAG
GCCTAGAAGAGCCAGCGTCTTTCCGCCATAGCAGACCATGTGACATAGATAGGCATAGTGGGTTTGCTTG
ATTAAGGGAAAGCTTTGCCGGACATTTCTGCCAGGAGAAGAATCCTTGATTTGCAGTACTGTTTCTCTAT
AGTGTAAAGGTCATTTTAAACAAAACAAAAAACCAAAGCCAGCTTTGATATTAGGAATCAAAGTACAGGT
TTTGGGAATGCAGAAGAGCCTTCCTGGAAATAGTGATGTTGTTTAGGAAGTCTCTTCTCCCTCCAGCTTT
CCCTGTCTGACTGTCTCAGTTTGGGCAAAGAGCTTTAGTTCGCTTTGACCGATGGCCTAGAAGTAAAATC
AAGCAATAAGTCACCAGCTGGAGATCTAGACAAACTTCCATAGTTGTTTTGAAATAAAAATTTCTAAGTG
AAAAAAAAAAAAAAAAAAAAAAAAAAAA
SEQ ID NO: 10 is the nucleotide sequence encoding canine RRM1, isoform 1.
NCBI Gene ID: 476823
SEQ ID NO: 11 is the amino acid sequence for canine RRM1, isoform 1.
>XP_534027.2 ribonucleoside-diphosphate reductase large subunit [Canis
lupus familiaris]
MHVIKRDGRQERVMFDKITSRIQKLCYGLNMDFVDPAQITMKVIQGLYSGVTTVELDTLAAETAATLTTK
HPDYAILAARIAVSNLHKETKKVFSDVMEDLYNYINPHNGRHSPMVAKSTLDIVLANKDRLNSAITYDRD
FSYNYFGFKTLERSYLLKINGKVAERPQHMLMRVSVGIHEEDIDAAIETYNLLSEKWFTHASPTLFNAGT
NRPQLSSCFLLSMKDDSIEGIYDTLKQCALISKSAGGIGVAVSCIRATGSYIAGTNGNSNGLVPMLRVYN
NTARYVDQGGNKRPGAFAIYLEPWHLDIFEFLDLKKNTGKEEQRARDLFFALWIPDLFMKRVETNQDWSL
MCPNECPGLDEVWGEEFEKLYESYEKQGRVRKVVKAQQLWYAIIESQTETGTPYMLYKDSCNRKSNQQNL
GTIKCSNLCTEIVEYTSKDEVAVCNLASLALNMYVTSEHTYDFKKLAEVTKVIVRNLNKIIDINYYPVPE
ACLSNKRHRPIGIGVQGLADAFILMRYPFESPEAQLLNKQIFETIYYGALEASCDLAKEHGPYETYEGSP
VSRGILQYDMWNVTPTELWDWKLLKEKIAKYGVRNSLLIAPMPTASTAQILGNNESIEPYTSNIYTRRVL
SGEFQIVNPHLLKDLTERGLWNEEMKNQIIACNGSIQSIPEIPDDLKQLYKTVWEISQKIVLKMAAERGA
FIDQSQSLNIHIAEPNYGKLTSMHFYGWKQGLKTGMYYLRTRPAANPIQFTLNKEKLKDKEKATKEEEEK
ERNTAAMVCSLENREECLMCGS
SEQ ID NO: 12 is the mRNA sequence for canine RRM1, isoform 1
>XM_534027.6 PREDICTED: Canis lupus familiaris ribonucleotide reductase
catalytic subunit M1 (RRM1), mRNA
CGAGGCGCTGGCGGCGTCGGGTAACGTCATTCGAGCTCCGTCGCGCCGCTTTTGCGCGGCTTTTGCGTCT
CGGGTGGCGGGAGCGGGAAGGGGATTCGGATTGTCGCGCCTCCGCTCGGTGGAGGAAAGTGCCCTCTGGC
CCCCAAATCAGTCCTTGCACCTGAGCACCCCCGGGAACCGGACCCTTCGCACTCTACCTACCACCTTCTC
GATCCCGCCGGCGCCTCGGAATTGCAGCCCCGTCTCGATTCCCTCAGCGCCTCAGCCACTAGCCGCGATG
CACGTGATCAAGCGAGATGGCCGCCAAGAGCGGGTTATGTTTGACAAAATTACATCTCGAATCCAGAAGC
TATGTTATGGACTTAATATGGATTTTGTTGATCCTGCTCAGATCACCATGAAAGTAATCCAAGGCTTATA
CAGTGGGGTCACTACTGTGGAACTGGATACTTTGGCTGCTGAGACAGCTGCAACCTTGACGACTAAGCAT
CCTGACTATGCCATCCTGGCAGCAAGGATTGCCGTCTCCAACTTGCACAAAGAAACAAAGAAAGTGTTCA
GTGATGTGATGGAAGATCTCTACAATTACATAAATCCACATAATGGCAGACATTCTCCCATGGTGGCCAA
GTCAACACTGGATATTGTTTTGGCCAATAAAGATCGCCTGAACTCTGCCATTATCTATGACCGAGATTTC
TCTTACAATTACTTTGGATTTAAGACACTGGAGCGGTCCTATTTGTTGAAGATCAATGGAAAAGTGGCAG
AAAGACCACAACATATGTTGATGAGAGTGTCTGTGGGGATTCACGAAGAAGATATTGATGCTGCTATTGA
AACATACAACCTTCTTTCTGAGAAGTGGTTTACCCATGCCTCTCCCACTCTGTTTAATGCTGGTACCAAC
CGCCCACAGCTTTCTAGCTGTTTCCTTCTGAGTATGAAAGATGATAGTATTGAAGGCATTTATGACACTC
TAAAGCAGTGTGCATTGATTTCCAAGTCTGCTGGAGGAATTGGTGTTGCTGTGAGTTGCATTCGAGCTAC
TGGCAGCTACATTGCTGGGACTAACGGCAACTCCAATGGCCTTGTACCAATGCTGAGAGTATATAACAAC
ACAGCTCGATATGTGGATCAAGGTGGAAACAAGCGACCTGGGGCATTTGCTATTTACCTGGAACCTTGGC
ATTTAGACATTTTTGAGTTTCTTGATTTAAAGAAGAACACGGGAAAGGAAGAACAGCGTGCCAGGGACCT
TTTCTTTGCTCTTTGGATTCCAGATCTGTTCATGAAACGAGTGGAGACTAATCAGGACTGGTCTTTGATG
TGTCCAAATGAATGTCCTGGATTGGATGAGGTTTGGGGAGAGGAATTTGAAAAGCTATATGAAAGTTATG
AGAAACAGGGTCGTGTCCGCAAAGTTGTAAAAGCTCAACAGCTTTGGTATGCCATCATTGAGTCTCAGAC
AGAGACAGGTACCCCGTACATGCTCTACAAAGATTCCTGTAATCGGAAAAGCAACCAGCAGAACTTGGGA
ACGATCAAATGCAGCAACCTGTGCACAGAAATAGTAGAGTATACCAGCAAAGATGAGGTTGCAGTCTGTA
ACTTGGCTTCCCTGGCCCTGAATATGTATGTTACATCAGAACACACATACGATTTTAAGAAGCTGGCTGA
AGTCACCAAAGTCATTGTCCGAAACTTGAATAAAATTATTGATATTAACTATTACCCTGTCCCAGAGGCA
TGCTTATCAAATAAACGCCATCGCCCCATTGGAATTGGGGTACAAGGTCTGGCAGATGCTTTTATTCTGA
TGAGGTATCCTTTTGAGAGTCCAGAAGCCCAGCTACTGAATAAGCAAATCTTTGAAACCATTTATTATGG
AGCCTTGGAGGCCAGCTGTGACCTGGCCAAGGAGCATGGGCCATATGAAACCTATGAAGGTTCTCCAGTC
AGCAGAGGAATCCTTCAGTATGATATGTGGAATGTTACTCCCACAGAACTATGGGACTGGAAACTTCTCA
AGGAAAAGATTGCAAAGTATGGTGTAAGAAACAGTTTACTTATTGCCCCAATGCCTACTGCTTCAACTGC
TCAGATTCTGGGAAATAATGAGTCCATTGAACCTTATACCAGCAACATCTATACTCGAAGAGTCTTATCA
GGAGAATTTCAGATTGTGAATCCTCACTTACTAAAGGATCTTACTGAGCGGGGCTTGTGGAATGAAGAGA
TGAAAAATCAGATTATTGCATGCAATGGTTCTATCCAGAGCATTCCAGAAATCCCTGATGACCTGAAGCA
ACTTTATAAGACTGTGTGGGAAATTTCCCAGAAAATCGTTCTTAAGATGGCAGCTGAAAGAGGCGCTTTC
ATTGATCAAAGCCAGTCTTTGAACATCCACATTGCTGAGCCTAACTATGGCAAACTCACCAGTATGCACT
TCTATGGCTGGAAGCAGGGTTTGAAGACTGGGATGTATTACTTAAGGACACGACCAGCAGCAAATCCAAT
CCAGTTCACTCTAAATAAGGAGAAGCTGAAAGATAAGGAGAAGGCAACAAAAGAAGAAGAAGAGAAGGAA
AGGAACACAGCAGCCATGGTGTGCTCTTTGGAGAATAGAGAGGAGTGTCTGATGTGTGGGTCCTGAGGAA
AGGCTTAGAAGAGACCAGCACTTCTTCACAGACAAACTACTTCTTGAGCATAGATAGGCATTGTAGGTTT
GTTTGAAGTGCTAAGGCTTTGCTGGATCTCATTGCAGCAAAAGGATCAGTCAATTTAAGGATCAGTCAAT
TTAAAGTACTGTTTCTATATAGTGTGAAAGTATTGATTTTAAAAATTGGTATTTTGGGAATCAAAGTAGA
AGTTTTAGGAGTGCAAAACAAGTCACCTTGCAAATAAGGAATGATTGAGTAGGGTTTCATTGCCCACCTG
GCACCCCTTTTCTGGTGACCTCAGTTTTCATAAGGAGACATGGTTTTGCTGCTTTGACTGGTGAGTCCAT
AGACGCAAAACTGAGTCCTAACCTGTGAGAAGTGCTGATAGGACCTTTCTCTGGATAAGGTCCTATAGGT
CATTCTGAAATAAACATTTCTAAGTGATTGTGTGAGA
SEQ ID NO: 13 is the nucleic acid sequence for human RRM2, isoform 2.
>NC_000002.12:10122568-10211010 Homo sapiens chromosome 2, GRCh38.p13
Primary Assembly
AAAATCGCGCGCGGCCCCGCGGCCAGCCTGGGTAGGGGCAAGGCGCAGCCAATGGGAAGGGTCGGAGGCA
TGGCACAGCCAATGGGAAGGGCCGGGGCACCAAAGCCAATGGGAAGGGCCGGGAGCGCGCGGCGCGGGAG
ATTTAAAGGCTGCTGGAGTGAGGGGTCGCCCGTGCACCCTGTCCCAGCCGTCCTGTCCTGGCTGCTCGCT
CTGCTTCGCTGCGCCTCCACTATGCTCTCCCTCCGTGTCCCGCTCGCGCCCATCACGGACCCGCAGCAGC
TGCAGCTCTCGCCGCTGAAGGGGCTCAGCTTGGTCGACAAGGAGAACACGGTGAGCCCGCGGGGAGGGCG
CTGCGGGCAGGGGAGGGAGGCAGGGAAAGCGAAGCCGCTCCTCACTCACACGCGTCTCCCCGCAGCCGCC
GGCCCTGAGCGGGACCCGCGTCCTGGCCAGCAAGACCGCGAGGAGGATCTTCCAGGAGCCCACGGAGCCG
GTGAGTGGCGGGCGTGGGGCAGAGGGGCCAGGGACGGCCTTGGGCGTCTTGGCGCCAAAGCCGCATTGTT
TCCTCAGCTGTTCACACTCCCGCCCCGGCTCCTTTCCCGCCTAGGCGGCCCCTCCCCAGGGCTGCCTCCC
GCGCCCCTCGGCCCATTTCCCGGTTCGGGCGTGCGCTCCTCTGCTGCGACCCACGGAGTGCGACGGGACA
GCCACGTTTTCACATCGGGCCCCGTGAAATTGCCGCCAATGGAAAGGACTTGGTCCAGAAAAACGTTAGT
TTCATATGGTTCGCCCGGTACTTAAATGTTTTATTTTCTCCCCCAACAGAAAACTAAAGCAGCTGCCCCC
GGCGTGGAGGATGAGCCGCTGCTGAGAGAAAACCCCCGCCGCTTTGTCATCTTCCCCATCGAGTACCATG
ATATCTGGCAGATGTATAAGAAGGCAGAGGCTTCCTTTTGGACCGCCGAGGAGGTAATCGGAGGACCCCA
GAAGACCCCTGCAGGGGTGACCGTCACGCCTCAGACATAAATGCACTTGGAGGTTCCCGTTGGCAAGGGG
GGCTAACTGTGGGGCATAGTAAGTGGTGCCAGCATACTTAAAGTTTGAGTGCTCAGTGTGAGTCCTGTAG
GCTTTACTCTCTTCCTTTTATGCTAAAATTGTGACTTCCGAACCTCAGGTGGACCTCTCCAAGGACATTC
AGCACTGGGAATCCCTGAAACCCGAGGAGAGATATTTTATATCCCATGTTCTGGCTTTCTTTGCAGCAAG
CGATGGCATAGTAAATGAAAACTTGGTGAGTTTCCAAAACATCTTTCATTCATTTGACGTTGACGATCTG
AGGTCGAACTAGTTCGCTTTCCTCGTCTTGTATGTTTTTCCATGCTGAGTGCATCTGTGTGTGTAAGCTG
GGTTTTATATTACATGGCATTTCCTGTTTTGTAACACTTTGCAGTTCTTTCTTATGGTATTTTCCCGACT
CTAGAGAAGCTGAGACAATATTAAGTGGTAGCAATGTGATGACTCTTTGTGGCCACCACATCTGCCCCCT
CTTTTTTTTTTTTTTTTTGAGACAGAGTCTCACTCTGGCCCAGGCTGGAGTGCAGTGGTGTGATCTTGGC
TCACTGCAACCTCCGCCTCCTGGGTTCAAGCGATTCCCCAACCTCAGCCTCATGAGTACCTGGGATTACA
GACGTGCGCCACCATGCCTAGCTAATATCTGTATTTTTAGTAGAGACAGGGTTTTACCATGTTGGCCAGG
CTGGTCTCGAACTGCTGACCTCAGGTGATCCACCCACCTTGGCCTCCCAAAGTGTTGGGATTACAGGCGT
GAGCCACCACGCCCGGCTCTGCTCCCTCCTTTTTGTGGCTTTGCTGTTTTAATAATAATTTGGTTGTATC
TCTTATTGCGAATGGATCTTTCTTGACATAAATTAATTAGGAAATCGAGCGCTCACAAATCCTATTTTAT
ATGTATCTATTTCCTGATATGTAAGTTGAGCATATGACATAAAATATCAAAGAATTGTGACAAATTGGAT
GAAATATATATAGAAATAAACCTTATAATGGTACAAAGAGTGCGATGCTGCCAGTATCCGTTGACAGTTG
CTGCTGTTGGTTTTTTCTCAAGCTTAACTTTGATGTGTTTTGCCACTAGGTGGAGCGATTTAGCCAAGAA
GTTCAGATTACAGAAGCCCGCTGTTTCTATGGCTTCCAAATTGCCATGGAAAACATACATTCTGAAATGT
ATAGTCTTCTTATTGACACTTACATAAAAGATCCCAAAGAAAGGTGAGTATTCAAGTGGTATGCCAAGAT
TTTTAGGACTCACTAATTGTTGATTTATTACACATTTTTAGTTCACCTAGGGATAAAAATGACTCCAGAA
TGACTAAGACAGTCATAGGCATTCCCAGCACCCGTGGTCATGTCTGCTCTTAGCAAGGGGCCTAAATGCA
CTTTATTATTCACTTAGAGTTGTGAAGGTACTCCTTTTAAAGTTGGATGTCTACCAATGTAAAACCTTCT
TTTGAAAAAATTCCTAGATGTTGGGTAAGACAAACTAAAACCTATGTCTGACCATCTTTGCTCATTTGGT
AAAGTTGTTGAGAAGCTAGAATGTGGGGCTGCAGTGGGATGGACGGGGAGGACTTGCCTCCTAAGAAGCC
TGCAGTATAGTATAGGCAAATAAGACTTAGTAGGAGTTACATAAGGCAGAGGCAGCAGTGAACCCTGAGA
CTGATTTAGGCATGCAGGAGTTTGGCTGAATAAAGGTAGCTTAAGGTCTGTTTTGTTTTGGAGATTGGAG
GTGGGGGGATTAGAAATGGGCTGCTGGAGTAGTCTAGATACAAAGGTCAGCTTTAGGGTGGCGCGCGGTG
GTTCTCGCCTGTAATCCCAGCACTTTGGGAGGCTGAAGCGGGCGGACAATGAGGTCAGGAGATCGAGACC
ATCCTGGCTAACACGGTGAAACCCCGTCTCTACTAAAAATAAAAAAAGTGGTGGCGGGCGCCTGTAGTCC
CAGCTGCTTGGGAGGCTGAGACAGGAGAATGGCGTGAACCCGGGAGGCGGAGCTTGCGCTCCAGCCTGGG
TGACAGAGCAAGACTCCGTCTCAAAAAGCAAAACCAAGCAAAAAAAACAAAGGTCAGCTTTGGGGACCAG
AACCTTGTATGGAGTGGAAGTGGTGAAGCTGCAACCTAAAGTAGCCGTTGTAGACTTTGAAGTACATGAA
GAGGAAAAGTGGTAACTTGAAAGGACTGAGGAAACATTGGGAGTAAAGAGATTTGAACATGTTTATAGGT
GGAAATTGAGAAAAGAAGGCAAAGATTAGGGGTACGATCGGGGGCAAATGCCCAGAAGGGGAACAGGAAG
GTCTGCTGGGGAAGCCTCAAAAACAAGGGAGAGGCAGACCCAGGTCTCAGAGAGAGGGACAGTGAGATGG
AAAGAATGAACGACAGCTGGGCATGGTAGTCTGAGCTAGTAGTCCCAGCTACTTGGCAGGCTGAGGCAGA
AGGATGGCTTGAGCCCTGGAGTTTGGTTTTACCGTGAGCTGTGATCATCTCGCTGCACTCTAGCCTGGGC
AACAGAGTGAGACCCTCATCTCTTTAAAAAAAAAAAAAAAAAAAAAAAAAAAAAAGCTGCCCAATGTCTG
GTGCCCTTGGCTTCAGAACACAAAGTCATCTGGGTAGGAACAGTCTGGGAAATGAGTAGCCTCTCAAGGT
GGGCACCAGAATAAAGGGAGGCAGAGGAGGGTGGTAAGGGAGATCCAGTTAACTGTAGTACCCATGGATT
TGCTTTCCTGACCTGGGATCGACAGTGTAGCACAGAGTCCTAGTAGGAAGCAATCTTAGTTTATTGGTTT
AATTATTTTATGATATAGGTGTGGCAACTGAGGCCAAATAATGCACCTAATCATAGTCTGATAATAGCAC
AGCAGTTAGGATTTTATGGTTCTTCAAATTTAAATTCTATGATTCTTCAAATTGAACAATGATCTGGACT
TGAAATAATTTTAAAGGCAACAAATGTCCCTGCTGTACTGGACTATGTTTTACTGTCTGTAGACCCTGAA
GCTCAATATGAACTACAGAATACCCAAACTTGTATTAATGTAAATCAAGTGTTGAGGTTTTTAAAAGAAC
ACTGGAGGGAAAAACTGACCAGTAAAAATAAAACATTTCGGTGTGAGTTCTTCCTTTAGGAAGAGGATTG
GCAAATACTTGAATTTGGCCTTTGTCCCAGAGCTCTTATCTAGCAGTTGGTAATCGGAGGTCTTTTACTG
TAATGCTTCAATTGCTGATACCGTATGTGCCTACTAGGGAATTTCTCTTCAATGCCATTGAAACGATGCC
TTGTGTCAAGAAGAAGGCAGACTGGGCCTTGCGCTGGATTGGGGACAAAGAGGCTACCTATGGTAAGGAG
ACCCTTGCCCCTACTTAAACCTGAGCTTCATTTTCCAAGTAATGTTACTGGATTTTTGGCCCTTGAATAC
CAACTCACTAGAATCATGTTGGTGTTAACTCCTAAATAGGTGAACGTGTTGTAGCCTTTGCTGCAGTGGA
AGGCATTTTCTTTTCCGGTTCTTTTGCGTCGATATTCTGGCTCAAGAAACGAGGACTGATGCCTGGCCTC
ACATTTTCTAATGAACTTATTAGCAGAGATGAGGTGAGTCTAAGTCAAATAATAGGGTGACCTAAACCCC
AAACACAACTCGGGCATGCTCTTGTGTTCACTGACGGGGACCTGAGATGCTAGATGGCATATATCCACAT
TTAATGTGTGAGTTCAACCATACACATACTTGACAAAAGAAGGAAATACTTTCATTTACTGAAACTGTTT
TACTTGCATTCTCAATATATTGTAATACATTTGTACATATGTATTCCCCTATAGGCTTTGAATGCATAAA
ACTACAAGTTCTTTGTTTTTTGAGGTGACGGAATCTTGCTCTGTCGTTCCAGGCTGGAGTGCAATGGCGT
GATCTTGGCTCACTGCAACCTCTGCCTCCTAGGTTCAAGTGATTCTCCCGCCTCAGCCTCCCAAGTAGCT
GGGATTGTAGGTGCCTGCCACCATGCCCAGCTAATTTTCGTCTTTTTATACAGACGGGGTTTCACCATGT
TTGCCAGACTGGGGTTGAACTCCTGACCTCAGGTGATCAGGTGATCCACCCGCCTCGGCCTCCCAGAGTG
CTGGGATTATAGGCATGAGCCACCATGCCCAGCCAAAACTACAAGTTATTGATGGGATTGGGATTTTAAG
GGATGTTTTATTATTTTTGCCTGGTATTAATATGTTATCCCTTTTTCCGTAAAAATGTTCATAGTAGAGC
CAGGAGCGGTGGCTCATGCCTGTAATCCCAGCACTTTGGTAGGCCGAGGCGGGTGGATCATGAGGTCAGG
AGATTGAGACCATCCTGGCTAACACGGTGAAACCCCATCTCTACTAAAAATACAAAAAATTAGCCGGGTA
TGGTGGACGTGCCTATTGTCCCAGCTACTTGAGAGGCTGAGGTAGGAGAATCGCCTGAACCTGGGAGGTG
GAGGTTGCAGTGAGTCAAGATCACACCACTGCACTCCAGCCTGGGTGACAGTCCCCCCGAAAAAGAATGT
TCATAGTAGCCATTATGTTTCTCCTGTTTGATCTAGAAATTGCCCCTCTACTTCAATATTAATAAGCATT
TCAATGAAATGAGTATACATTTTGGTCTAGTGTATGTCTTTGATTAAGTCACATTTGAAAAGCCAGGAGC
ATGAACTCCATCTTACTTGGAGCCCAGTGGGCAAATCAAATATGGTTACCTTGTAGGAGGGCCTTCCTTA
CTGGATTGGGAGATAAGCTGTGAAGCTTGATGTTTAATGCAGTAACTTGCAAACTTGATTTACTTGAAAT
TGCATACAAATTTCCTGAGCATCTAAAAACTAGCCTTATTACTGAGCTTTGCCTTTCCTGCTGGGAGTAG
TGGCAAAATTAGCACTCATGGCTGTAGAAAGATCACTGAGTGAAGCTCTGACTCCTCCTTTGCCAACACA
CAGCAGAGCAAGAAATACACCTTGCCTGTCTTCATCTAGGTGGCAACTTTGAGGGTCTTGAATGGGACTG
AGCTTGCCTTGGTAGTGACATCAGCAGAGAAGTCAGTAGTTGAAGTCATCTTCCCTTTGAGAGTTCAAGT
GCTCTCAGTATGGCTGAGCATGTTGGATAAGGAGAATGCAGAAAAGGACAAAGTAATTTCATATTCCATG
TTAATGACAGAAGTCTTCTGGCTTTAGTGATCTTGAACTTTTTTTTCTAGGGTTTACACTGTGATTTTGC
TTGCCTGATGTTCAAACACCTGGTACACAAACCATCGGAGGAGAGAGTAAGAGAAATAATTATCAATGCT
GTTCGGATAGAACAGGTAAAGTGGGTGATGAAATGGGTCACTCAAGCTTGCTAGAAAATGCCTGTGCTTT
AGTTGTATTCAGAAGCTGTATTTTGGTTCCTAGGAGTTCCTCACTGAGGCCTTGCCTGTGAAGCTCATTG
GGATGAATTGCACTCTAATGAAGCAATACATTGAGTTTGTGGCAGACAGACTTATGCTGGAACTGGGTTT
TAGCAAGGTAAAGTATTGTTTACATAGCCTTTTGCTTGTTTTGAAGCTGGTGCTCTGTATTTATATCTTG
ATGTGAACCTTTTCAGGTTTTCAGAGTAGAGAACCCATTTGACTTTATGGAGAATATTTCACTGGAAGGA
AAGACTAACTTCTTTGAGAAGAGAGTAGGCGAGTATCAGAGGATGGGAGTGATGTCAAGTCCAACAGAGA
ATTCTTTTACCTTGGATGCTGACTTCTAAATGAACTGAAGATGTGCCCTTACTTGGCTGATTTTTTTTTT
CCATCTCATAAGAAAAATCAGCTGAAGTGTTACCAACTAGCCACACCATGAATTGTCCGTAATGTTCATT
AACAGCATCTTTAAAACTGTGTAGCTACCTCACAACCAGTCCTGTCTGTTTATAGTGCTGGTAGTATCAC
CTTTTGCCAGAAGGCCTGGCTGGCTGTGACTTACCATAGCAGTGACAATGGCAGTCTTGGCTTTAAAGTG
AGGGGTGACCCTTTAGTGAGCTTAGCACAGCGGGATTAAACAGTCCTTTAACCAGCACAGCCAGTTAAAA
GATGCAGCCTCACTGCTTCAACGCAGATTTTAATGTTTACTTAAATATAAACCTGGCACTTTACAAACAA
ATAAACATTGTTTGTACTCACAAGGCGATAATAGCTTGATTTATTTGGTTTCTACACCAAATACATTCTC
CTGACCACTAATGGGAGCCAATTCACAATTCACTAAGTGACTAAAGTAAGTTAAACTTGTGTAGACTAAG
CATGTAATTTTTAAGTTTTATTTTAATGAATTAAAATATTTGTTAACCAACTTTAAAGTCAGTCCTGTGT
ATACCTAGATATTAGTCAGTTGGTGCCAGATAGAAGACAGGTTGTGTTTTTATCCTGTGGCTTGTGTAGT
GTCCTGGGATTCTCTGCCCCCTCTGAGTAGAGTGTTGTGGGATAAAGGAATCTCTCAGGGCAAGGAGCTT
CTTAAGTTAAATCACTAGAAATTTAGGGGTGATCTGGGCCTTCATATGTGTGAGAAGCCGTTTCATTTTA
TTTCTCACTGTATTTTCCTCAACGTCTGGTTGATGAGAAAAAATTCTTGAAGAGTTTTCATATGTGGGAG
CTAAGGTAGTATTGTAAAATTTCAAGTCATCCTTAAACAAAATGATCCACCTAAGATCTTGCCCCTGTTA
AGTGGTGAAATCAACTAGAGGTGGTTCCTACAAGTTGTTCATTCTAGTTTTGTTTGGTGTAAGTAGGTTG
TGTGAGTTAATTCATTTATATTTACTATGTCTGTTAAATCAGAAATTTTTTATTATCTATGTTCTTCTAG
ATTTTACCTGTAGTTCATACTTCAGTCACCCAGTGTCTTATTCTGGCATTGTCTAAATCTGAGCATTGTC
TAGGGGGATCTTAAACTTTAGTAGGAAACCATGAGCTGTTAATACAGTTTCCATTCAAATATTAATTTCA
GAATGAAACATAATTTTTTTTTTTTTTTTTGAGATGGAGTCTCGCTCTGTTGCCCAGGCTGGAGTGCAGT
GGCGCGATTTTGGCTCACTGTAACCTCCATCTCCTGGGTTCAAGCAATTCTCCTGTCTCAGCCTCCCTAG
TAGCTGGGACTGCAGGTATGTGCTACCACACCTGGCTAATTTTTGTATTTTTAGTAGAGATGGAGTTTCA
CCATATTGGTCAGGCTGGTCTTGAACTCCTGACCTCAGGTGATCCACCCACCTCGGCCTCCCAAAGTGCT
GGGATTGCAGGCGTGATAAACAAATATTCTTAATAGGGCTACTTTGAATTAATCTGCCTTTATGTTTGGG
AGAAGAAAGCTGAGACATTGCATGAAAGATGATGAGAGATAAATGTTGATCTTTTGGCCCCATTTGTTAA
TTGTATTCAGTATTTGAACGTCGTCCTGTTTATTGTTAGTTTTCTTCATCATTTATTGTATAGACAATTT
TTAAATCTCTGTAATATGATACATTTTCCTATCTTTTAAGTTATTGTTACCTAAAGTTAATCCAGATTAT
ATGGTCCTTATATGTGTACAACATTAAAATGAAAGGCTTTGTCTTGCATTGTGAGGTACAGGCGGAAGTT
GGAATCAGGTTTTAGGATTCTGTCTCTCATTAGCTGAATAATGTGAGGATTAACTTCTGCCAGCTCAGAC
CATTTCCTAATCAGTTGAAAGGGAAACAAGTATTTCAGTCTCAAAATTGAATAATGCACAAGTCTTAAGT
GATTAAAATAAAACTGTTCTTATGTCAGTTTCTTGATTGGTAAAATTTGCATTTTAATTCAGGAAGAGAA
ATATTTTTTGGCCAGGCATGGCTGTAATCCCAGCACTTTGGGAGACCAAGGTAGGCAGATCACCTGAGCT
CAGGAGTTCGAGATCAGCCTGGCCAACATGGTGACACCCCATCTCTACTAAAAATACAAAAATTAGCCTG
GCATGGTGGCACACGCCTGTAATCCCAGCTACTCGTGAGGCTGAGGCAGGAGAATCACTTGAACCCGGGA
GGCAGAGGTTGCAGTGAGCTGAGATTGCACCGCTGCACTCCAGCCTGGGCAGCAGAGTGAGACTGTCTGA
AAAAAAAAGGTGTTTTTTGTAAAGGCTAACGAATTCATTTGCTTTCCACTGGTTCTGGGCAAGAGACTTG
CCTTGTGCCTATTGGCACAAGGTGTATAGGAGACAGGTACACCCGAAAGGTGGTGCCCAAAAATACTAAC
TGCCATACTGCACGTGGGGTTTGTGAAGCCGGGGCTGAGTTAACTTCTCAACCGTGGGGGAGCCACTCCT
GGGGCTCTTTTCCCGTTTGCAAAACAGGTGGGGCTAGAGGTCTTCCCAGCTGGAGTTTTGCTCTGCTGTC
CCACATCTGACCTGTGTGGACTCCAGCACAGGTTTGGATTGGTCCCTGTGTCTATAAAGGCCCTTTCCTG
ACTCGGAATCCCACCCACTTTGATAAAGCCTTTTAGAATTCATGACACCCATCCCCAAACGAACCAATCA
TTCCTTGTACCTCACGCCACTGCCATTCAGTTCATTGAACAAACAGCAGCTCTCTTGTCAGGTGACGTTT
TCTACCTGCATTTTAATTCAGGAAGATGGGGCGCTAAGCCAGAGGGGAGGCCCCTCCCTCTGGAGCTTGG
GTTTACTCCTAGAGAGGAAAACTGATAGATGAGTAGATCTGAATTGTTGGGCAGTGGTGGGGGCAGCAGA
GAATTCTAAGGTGGATGGGAGTGATTGGGAGAGGTCTCATGGGGGGAGATGATGTTTCCTTAGTGGAGGG
GACTGGGACTAGTGTCTGAGGTTGTGCTGCTTCCGACTCCTGTGTTCTCATGAAGATTTCTTCCCGTGCT
GCTCTGTAAGAGAAAGTTGTCTTTGAGGGCACAGATTTTTATCTGTCTTTATATTTATAAGCATAAGGCC
TCCAAGGATCAGGTATTTAAGTGACTGGTACATGGGGAAATAAACTAATTGGAACTGAAGTATTTTGGGG
TGGATGGTATCTTGGGTAAAAGTGTGATCTGTGTCCCAGAGGAACCTAGTAGAGAGCTTTGCCTTTACAC
CTAAAAGTGTTCAGTTAAGGTCATTTGATTTGTAATGTCAGGTTGGCGCTGGGCCTATTGCACAAGTTCG
GGGCAGCCAGGCGTCAAGAAGATGACCAACTACTAGGACAGCCTCCCTGTGGGTGGCCTGCAGTCTGTTC
TGCTCCCCCCGGCCCCATGCCAGCTGCCATGCTCTATAGAACATGTCTCCCATGCTGCCCGAGGAGGGCC
TGCAGAGAGTTGAGTGGTCAGGCTGCTGAGTCAATTGCCCTGGGTACTCATTAGATACATCCTCCCCGGC
CTCACCCCCAGACCTACTGAATCAGTCTGGGGGTAAACCAGGGACCCTGTAATCTTAATGGGAGATCTCA
GACACTTGAGATCGGGTGGGATAGACTCCTGACATAAAGTTCAAACCAGTGGACGTCAGTCCTGGGTGTG
AATTATAATCACCTGGGGGCTTTTAAAAGCTACTAAAGTCTGGATCTCACTTGGGGAAAAGGCAGCCCTG
CTGAGCTTCAGCATGTTCCAGATGTGTTTTCTGGTGTGTTCTAGACATGCTGTGTAAGAGTTACACTTCA
TTGTGTGTGCACATTCGGGGCCCTGCCCAGCTGCAGTGGCCAGGCCTGGCTGCTAAAAGCAGACCTACCA
AAACCTCCCTTCACCTGGTACTGCTGTGGCCTCTGACCTGAGGACTTTGTCATGCAAAGGAGGAACCAGA
TGGGTGTTCTGTCACCTGGCCAGGGAGCTAACTGGCTGTATTTTGAGGATCAGTGGCCCTGCCAGTGTCG
GTCTGGAGATCCTGATAATGGTTTAACTCCTCTTAGCAAGACAGGCACAGGCCCAGCCCCTCATCCGTGA
GTGGCTGCAGTTGGACTGCGTGGCCTGGCCTCTTCCAGCAGTCCCTGAGTATAGAGGGGCTACCCTCCTG
GTGTCTGTCTGGACACAGAAGGGAACACATCAGTGGTGTCTCCCTGCCATTCCCTGGAGGGAATATGACA
TCAGGATTTTTTTTTTTTTTTTTTTTAATGATGAGAATAGCTGAACCCATTGCTGCTTAAGGTTCAGTAG
TCATGTTCACCTTAGCCTTGGCTCTAGAGACTGGAGTGGCTCCAGCAAGGTGGTGGACTAAGGAACCGAA
CTCCTCTTGCTTCAAACACCTGCCCATGATAAATAGCACAGCAAAAAGTTAAATAGGTGGGCCGGGCATG
GTGGCTCGCGCCTGTAATCCCAGCACTTTGGGAGGCTGAGGTGGGCGGATCACCTGAGGTCAGGAGTTTG
AGACCAGCCTGGCCAACATGGTGAGACCCCATCTCTACTAAAAATACAAAATTTAGCCGGGTGTGGTGGC
CCATGTCTGTAATCCCATTACTCCGGAGGCTGAGGCAGGAGAATCACTTGAACCTGGGAGGCAGAGGTTG
TAGTGAGCTGAGATTGCACTCCAGCCTGGGCGACAAGAGTGAAACTCCATCTCAAAAAAAAAAAAAAAAA
AAAAAGTTAAATAGATGATCACCTCCTGGGAAAGGAGAATGACCTCTTTTGGGAACAAAGAAAGTGACCA
TGGAGGCCCCGGAACTTGTAGCAGCTCTCCGAGTGGTTGTTAGTCCCTTAGAGAAATTAAATGGGCAGGG
GCTGACGTGCAATTGGATTTGGAGGGAGAGGTTAATCTAAACCAGGGGAGGAGTTGGGACCAAAACCGTG
GCATAAAGCTAGGGGCCCCAGTGACACGGGAACTAGACAAAAATCCCATTGGCACAGTGGGGAAGATAAG
GGTTTTAATCCTTCTGGATTCTTAAAAACCTTCACATAAATTTGTGGTCTAGGAACTACTCAAAGATAAA
TTAATCCCCAGTATTGATCCCTAGCCTGGTTGCACAGGATTCCCACAGGTGGGGTTCACACTTCTTAATT
AGAAACTCAGACAACACAACAGATAAAAGGAAAAAACACGGTATGGTGAAGAAATAGATTTTCGGAATCA
GGTAGAATGGGATACAGCATAACTACTAGAGTTCAGGCTCTTAGAGCTATTTCAACACAGCTTAGTAAGA
TTAGACATAGCTGAAGAGTGGACTTGTGAGTTGGAAGGGATGTATCTAAGGGAATTATTCTAAAGAAGAT
GAAAGAAAGCATGGAGAGAGTGGACAGGCAGTGTTTGAGGAGGTAAAGACTGGTGACTTTCCAGAATGGA
CTCATCCTTGAGATCAGGAAGTCCCCAAGGAATCACACATGAGAAGCAAACATTCATTCCTAGTCTCATT
TGAATGAAGCTGCAGAACATGAACAAAAAGAACAATCATTAAACGAATGTTTCAACGTAAGCCAAAAGAT
TATGAAATCATATCTTCAATGCTGAGGGAATCATTGGACAAAATTATTATTCAGAAAGAAGATACATTTT
CATGCCAAGGTTTTACCATTCACATCCCCAAGCCAAAATAATTCCTAGCGAATTGAGCCAAAAGAGGATG
GTGATGAGCAGAGAAATTGGTTGGTCCACAGTGAATCAATAGAATCAAAAGGAAGCTGGGCATCATGGTG
CATGCCTGTGATCCCAGCTACTCACGAGGCTGAGGTGGGAGGATCACTTGAGTCCAGCCTGAGCAACAGT
GAGACCGAACTCAAACAACAAATGGAACTAGAATAATGGACGCTGCAGAGGGATAAAGAATACCAAGGTC
TTTATACTATTAGGCCTTGGAGGTATATGGTTTAAAGTTTAAGGATGACCACTGGAGGCATAGCACCAGA
AGATTTAACTTCAACTTCCAAAGTAGGGGCCCTGGTGGGGGAGGGGAGGGGCATGCGTGCGGGTGTGGGA
TGTGTTGCAGAGGAGTTAAAGCTTGACGGGCTTAGAAGGTGGAAAGGGTCGAGATGCAGCAGAGAACAAG
TCAATGGAAAGCACAAAATAGGATGGTAGAAATAAAAGCACAGTAGATGTAAATAAACTTAAGAGTTAAA
ACTCTTAAGGCTCAAATTCTAGCTATAAATGATTGCCAAACTAAACACTTAAGAGTGTAAACAATTACTA
TAGGAAAAAGACTTTAAAGCAAAAAGCCCATTAGAGATAGAAGTTATTGGCCTAAAGAATGATTGTTCCT
CTGGGAAAACACGATGGTGATAACTGCACCAAGCAACATGGTCTCTGCAGTGTTTGGGTGTGACTTCATC
CCTGCCTCCCCAGGGACTGATGAGTCCTGGGCTCAGGCTCAGAGTGATGAAGCGGGTTATTGTCTTGGTG
CTGCCATATTCTCTTAGTCATGTGTTTTTTTTTTATTCATTGTCCTCTGGCCCTACACACAGTCCCTAGG
TTTGCTTTTATGCCAATTGCAGAGAATCTCCATGGCTCTACTGGCAATTGCTTGAGAGCTCACATTCCTA
CCGGATGTGAGAAGATGTTCCATCTTCCTGTGTCCGGGCAGCACCACCAAAGTGCGGGGCTGGGGCAAGC
CTGGATGGGGTAGGGTGTGGTCAAATGACTGCTCACAAGTGAATGGTTTGGATTATGCAACGTGGCCACA
CTGGAATCTCAATAAAGGGCAGAGGGGTTGAGGCCCAGATGATACCAGGTGAAAAGACAAGACAGGTTTG
CAGCATGTGCTGCGATGGAGATGCTGGGGCCTCCTAGTCTGGGGCAGGCTGAGCCTTCCTGGGTGTGGGT
GGGCCTCCACTGTGCAGGGTGCTGTCCTTCCAAGCTTTGGCAAGATGGAATCTGTGTTGGGTGTGGGTGT
GGCCTTGAAAAGGCACGCTGTCATCTAGAGGCCACTGCTTGACCCCCTGTTGCCCTTTATCAGAGGTGCG
GGTGGCTAGTGCCTGCAATGTCAACACTGGGAGGCCAAGGGGGGAATCCCTGGCTTGAGGCCAGGATTTC
AAGACCAGCCTAGGCAATATAAGGAGACCCTATCTCTATAGAAAAATTTTTAAAAATTAGCCATGTGTGG
TGGCATGCATCTGTAGTCCCGGCTACTCAAGAGGCTGAGGCAGAAGGATCACTGGAACCCAGGAGGTTGA
GGCTGCAGTGAGCCATGCTTGCACCACTGCACTCTAGCCTGGGCAACAGAGCAAGACCCACTCTTAAAAA
CAAATACCCTGGCCATAGCCCCTGGGAGTCTAGTCTCCATGCAACCAGGACCCCATGAGCCACACTTTAA
GGCTTTTACTTTGGGGGTGAGGGGTGGGTAGAAGGGAGGCCAAAGGTTGGGGCTGTTGAACCCATGAGGG
TAGACCTGTCCAAGAGTGAGGCCCTGACTGAGGAAAGGAGAGCTGATCTACAGAGAGGCTGAAGCCTCAT
CATATTCATTTTCTGAGTCCAGTGAATTCTGAAGTCCACATCCCAGATGTTTCACAGAAGTAAATCAATA
CATTCCTGATAGGATAAGGTTGATTTTCCTGCCACGTGCAGTCAGTTCCACCCCAACAGAATTCACTTGA
TCTGCACAGCCACCGTGGGGCAGAGATGATTGCCCCCATTTTACAGATATGGCCGGGAGGCTCAGGGAGG
CCATGTGCCTCACAGAGGACTGGTCTGCACAAAGACCAGTGGGTGACCAGAACTTGCACCTGTCTACATT
CGTCCCCTCACTGCATGTGCCCCTCTTGATGGGAGAGCAGGCACCTGCCAGCCCCATGCTGGTCCGCCCA
AGGAATGGCTCCTAAAACCACCAGCTGGCCAGTGAGTCATATGGAAGGGTTTGGTCATGCCTACACTCGT
CCTGCCCTTCCTCCCTGGTCATTACACTGTGGTAGTGGGGGAGGGTGACCTCTGCTTTGCCCCTTGTCAC
CTGGGGCAAAGGGTGGGATCCTGTCCATATAGGACCAGGGGCTCTCTTACTGTCTGCACTTCACTAGTGG
GGGCCACTTTCAGTTTGCTTCTACCTGGGACGCTCTGGGTACTATGCTAGAAAGGTAGCTTATGGCCACA
AAGTCTAGGGACAGGTACTCACATGCCCAAATGGAGCCTCACGCTTAGGAATGGGGTGAGTGGGAAGCAG
TAGCTTCCCTTGGAGGTGGAAAACGGGGAGATTTCAGTTTCCAGAAAGCCCTGGGGCAGCAGCTGCCTGG
GTGAGGTCTTCCCTGAGCCTCTGAGTCTTCCCTCTCCTCTACTTCAGGGGGGTCTAGCCTCTCCTCCAGA
AGGGGTGCCAGGACGAGATGGGAAGGAATTTGCTTTGGAAGGCTGGGAACAGGTCCTGCAGCTGCTCTGG
CCTTCCTCTGGACTGATGTGGGTCCCAATGTTACTGAAGGGCACTTCCAGGTCCTGTTAGTGCAGGGGCC
GCTGCAGCAAAGGGCTGACTTTAGGCTCCCTCTTTGTTTTGTTTTGTTTTTGAGATGGAGTCTTGCTGCG
ACCCCCAGGCTGGAGTGCAGTGACGTGATCTCGGCTCACTGCAACCTCTGCCTCCCAGGTTCAAGTGATT
TTCATGCCTCAGCCTCCTGAGCAGCTGGAATTACAGGCTCCTGCCATCATGCCCGGCTAATTTTTTTTTT
TTTTTTTGAGATGGAGTTTCTCCCTTGTTGCCCAGGTTGGAGTGCAATGGCGCAATCTCAGCTCACCACA
ATCTCTGCCTCCCGGGTTCAAGTGATTCTCCTGCCTCAGCCTCCTGAGTAGCTGGGATTACGGGCACATG
CCACCATGCCCTGTTAATTTTGTATTTTTAGTAGAGACGGGGTTTCTGCATGTTGGTCAGGCTGGTCTCG
AACTCCTGACCTCAGGTGATCCGCCTGCCTTGGCCTCCCAAAGTGCTGGGATTACAACCATTAGCCACTG
TGCCTGGCCAATTTTTGTATTTTTTAGTAGAGACTGGGTTTCACCATGTTGGTTGGGCTGGTCTCGAACT
CCTGACCTCAAGTGATCTGCCCATATTGGCCTCCCCGTGTTGGGATTACAGGTGTGAGCCACTGCACCCG
GCCTTTCACCTCCCTTTTGAAAAACTGACTGCTCTTGGAGCCAGTTCTCACTGCTTATGAAACGGTTGGG
GACATCACATCACTGCTCTCAGCCTCAGTTTCCACATCGTGAACTAGGATTTGTAATCCAGGCCCAACAG
GCTTGTGAGAATTTAGATGAGTGAAGTGCTTTCATAAGCAACTTTTTCACGTGCCATTCAGTTTGCCCAT
CTAAAGGGTATGGCTCTTGGCCAGGCGTGGTGGCTCACGCCTGTAATCCCAACACTTTGGGAGGCCAAGG
TGGGCGGATCATTTGAGGTCAGGAGTTTGAGACCAGCCTGGCCAACATGGTGAAACCCCATCTCTACTAA
GAATACAAAAATTAGCCGGACGTGGTGATGGGTGCCTGTAATCCCAGCTACTTGGGAGACTGAGGCAGGA
GAATTGCTTGAGCCCGGGAGGTGGCGGTTGTAGTGAGTAGAGGTTGCGCCATTGCACTCCAGCCTGGGTG
ACAGAGTGAGACTGACTCAAAAAAAATAAAGGGTGTGGTTCAGTGGTCTAGTATATCCACAGGTATGGGC
AGTCATCTCCACAGTTGAACATCTTGCCCACCCCAAAAAGAAAGCTCGTGTCCCTTAGTCACTCCCTGTC
TCCCGTCTGCCGCCTCAGCCCTGGGTAGCCACTGAGCCACTTTCTCTGGTTTGATCTGGCACTTCTAAAG
TAGAAGTTATTATTGGCAAAGGCACTGGCTCATATTTCTAAGAGATCATTTAGAATGCCAGGGGCAGCAG
AGGATGGGTCTTCCTTGCAGAGAGAAGTTTGATTTACATTTCAGAAGGATCTTTCCTGGCATTTTAGGCC
ACTTGGCATTAAAAAGAGACTTGTGGGGCATTGTCTGGAAGGGGACTTTTGCCACTGTGAGCTGGTGACA
AGCTGACTAGAAGTGGGGGTGTCAAAGGCTGGAATCACCACATTGCTGGGGGCGGATGAGGGGACAGTTA
GTGGATTTGGGGTCTGTTCTTTCTCATCTCTCTTGGTGACCCTTCCCCTCCATCAGGAAATGAGGTGCTG
AGCCATCTCTATGGTCCCAGCCTTCGGTTTTGCTCTGTTTGGGGCGTTTCAGAGAGCTGGACTGTCCCAG
GCCCCTTGGGAATTGGAAGCAAGTCCTAGAACTTTCTGGAACACAGAGAGTGGTCTGTTGGGCCTTCCTT
TGCTTCCTCAGTTCCCACCCTCCTCCTCCACTCCTTTCTAGGACTTTCCTTCCTCCACCCCTGGATCCTC
CAATGTTAACGGAGCAGGGGTCAGGAAATAGCCCGTGGGTGGGATCTGGTTCACCGCCTGGTTTTATACA
TACCAGGAGCTAGGAATATGTTTTAAAAATCAAAAGAATGGCTGGGCGTGGTGTCTCATGCCTGTAATCC
CAGCACTTTGGGAGGCTGAGGCGGGTGGATCAAGAGGTCAGGAGATCGAGACCATCCTGGCTAACACAGA
GAAAGCCCCTCTCTACTAAAAATACAGAAATTAGCTGGGCGTGGTGGCGGGCACCTGTAGTCCCAGCTAC
TTGGGAGGCTGAGGCAGGAAAGTGGCGTGAATCCGGGAGGCGGAGGTTGCAGTGAGCCGAGATTGCACCA
CTGCACTCCAGCCTGGGCGACAGAGCAAGACTCTGTCTCTAAAAAAGAAAAGAAAAAAAAATCAAAAGAA
CATTTCGTGTTGCATGAAGATTCTATGAAATTCAAATTTCAGGGCCCATAAATAGCTTCGTTGGGGCGCA
GCCACACTTGTTTGTTTACGTATGGTCTGTGGCTGCCTTCACCATGGATAAGAGACTGGAATAGCTGCTG
TGGTCTGTATTTTCTATCTTTACAGAAAATGCTTGATGAGCTCTGAGTAAGGCCTGACCATGTTCCTGAG
AGCTCCGTAGTGGACCTGATGGGAAAGTAGTGGATGGCATTGGTGGTGGCTGAGCCAGCAGGGTGCCTGC
TCAGAGAGCCGGTTATTAATGGAGTGCTAACCAGTCACTCTAGATGAGAAATACACAGATCACATTTAAA
CACAGGGAAAGCTGTTTACAGGAAAATGATAAAATAGCAGAACGTAAAGTTATGGGTATACTGTGCTTAC
GTTGTCATAAAAATGTGTACATATGGATAGAGAGGAAGGTGTCCCACGTGGAGAAATGAAAAGGTGCCTT
AGGGGGCAGGTTGGGAGTAAGAGCAGTTTTTCTTTCTATTCTATTTTTCTGTTTAAATTTTCCTATAATG
TTCTTATAATAAGTGTTGATGAGCACTCGCAGCCACCAGGGGAAGCAGCTACCCAGGCAGAAGGCTATTC
TGGGTCTCAGGTGGGCTTTAGGGAGAGGGACCCTGAGCTCTGGACCAGGGTTAGGAGGAGGTGCCCCGGG
CATGTGGAGGCTGGCAGCCCTCGCTCCTGTGAACTGGCGGCTGGGGCTGCATCTCGCCCACGTCGCTGTC
ATGTGCGGGGCCCACGTTGTGAGTTGTGTGTCTGCTCACTATTGTGCTGTAGCCTCTGGAAGTTGCTTAG
GAGCTTGGGATTTCGTGGAGAGGTGGGAGTTAGTTGCTTCTGTCCAAAGAGGTCGGCCAATGGGGGCCAT
TCTGAGTTCAGAAACCGCTGGCTTGGAGCCCGATGGACCCAGCCAGGCCCAGCTCTGCTGTTGACCAGTT
ATGTGCTCTGGGACTCAGGTTAAGTCTGTGGGTGGCAAAACTGGCACCTCCCATCCCATCTTCCCTCCAT
CCCCTCCCCCCACCCCCATGTCTCACTCTGAGCCTTAGGGAGTTGGTGCCACCTAGCTACGCTCACTGAC
ACGCATCTCATGCTGTGCTAGGATGTTGTCCCCATGTTAGTGTGCCCTCTCCAGGGAGACTGCCAGCCCT
CGTGGGTGTGCATCTTCTGCTTCTGCATCCTTGGTCACTGCCAGCAGAGGTCTGGGCACATGGTGGTGGA
CAGTGGCTATTAACTGTGGTTGTGCTGGAATGGAGGAGGCCTGGTGTCTGGTCTTGGAGTCAAGAATGCA
TGTGCTTAAGGATTCCAAGATCACACGGAGGAGGGTATGATTAGCTCTGGGAAGCGGGGAGGCAGGAAGG
AGGGCAAGAGACCTCGACCTGTTCTTGGGAAGGAAAGAGCAGGTGAGGGTGGGGTACTGTTCCAAGCACG
GGAAACAGAGCCCCAGGAGGTGGCCATGGCCTGGGGCTGGGGCTGGGGCTGGGAGGCGGTTGCTCCAGCA
TGCAGTCACTGAGCCCTCCTGTATGCACTGCTGCAGGTGCTGGGAGACCGTGAAGTGCAAAGCAGATGGA
GTCCAGGCCCTCGGGGAGACAGCACGCCAGTGAGGGAGATGGAGACCAATCACCCACCCAGTGTGCGGTA
AGTCAGACGGTGACAAGCTCTGAAGACAAGGAAAGCAGGGAGGAAGGGGCGGGCTATGCCCCTTGCTCTT
TCATGTGGGGAGCCAGAGGGAGCCAGAGGATGTGGGGATCGACCACGTGGTTAGGGGAGGAAAGCATGTT
TCAGGCGGAGGGTGGGCAAGCGCAAAGGCCCTGTGGCAGGCGCGTCTGTGAGTGGTCAGAGCAGAGTGAG
GGGTGGGAGATGGGGCTGGGTCAGTGGAGTGGGTGTGTACATGCAGGTCTGCAGACCCAGGTAAAGAGTC
TGGATTTCATTTCTAAGGGGATGAGAAGCTCGGGAAGGTCTGACCAGCCTTACGTCTTGAAAGGGAATCC
CCTGCTTCTGCGTGTCAGGTGTTACTCGGGGTGCGCCAGTGGAAGCGGGAGGCAGTTGCAGCTTTCAGGT
GAGAAATGATGGCAACTCGCACGGTGGGGGAAGAGGGGCCACTGTGGAGGTGGCTTGCGGGGCCTGTCAT
CTGCTGTTTCCTAGCTCAGTGTACAGGGCCCCCACGCACCCCTGCCAGGTCGGAAATCCAGGTACTGCCA
GCCTCTGCCGTTCTACGTGTGGCCAGGCGGGGGAGCTTCGCCCTCCCATGTCCCTCACCCCATGCCCCAG
ACAGCACGGTCCTGCGATCCCCCTCCCATTCCAGTGCTCAGTATAGCCGTATCCCTGCAGGCTCAGGCCC
CCACCATGGCCAGATCCCCCACCCCACCTTCACCCTCCGCCAGTCCATTGCACACATAGCATCTGGAATG
GTCTCGGGCAGCACCTCAGACGATGTGGCTGTGTTGCATGAGCCCCTCCCCTCACCGCAGCTCTCGGCCC
TGCCTGAGCCCAGGTGCCCGCGGGCTCCCCGCTCACCTCTGCCTTGAACCTTCCTGAGTCTGAGCTTCTT
TTTTGTTTTGTTTTGTTTGGTTTTGAGACAGAGTCTTGCTCTGTCACCCAGGCTGCAGTGCAGTGGCGCG
ATCTCGACTCACTACAAGCTCTGCCTCCTGGGTTCATGCCGTTCTCCGGCCTCAGCCTCCCGTGTAGCTG
GGACTACAGGCGCCCACCACCTCGCCTGGCTAATTTTTTGTATTTTTAGTAGAGACAGAGTTTCACCGTG
TTAGCCAGGATGGTCTTGATCTCCTGACCTCGTGATCTGCCTGCCTTGGCCTCCCAAAGGGCCGGGATTA
CAGGCGTGAGCCACCATGCCTGGCCTGAGCCTGAGCTTCTTAAACACCACATCCTCTAGCCTCCTGCCTT
CACACAGGCTGTTCCCTTAGCCTGGACTTTGTCCCCACCCACCCGCTCCTGATCTTTCAGGTCTCAACTT
ACAGGTCACTTTCCCTGGGAACCTCCTCAGACCCAGTGAGGCCAGATTAGGGGCCTCCTCAGAGCCCTCA
TGGCCCCAAGTGTTTACCACTTCGCTTTTTCATACTATCCAAAGTGCACAGAATCGTACACTTTTCTGAT
GCCTTGTTCCAGCTGAGTGCCCTGAGGGCAGGGATGGGGTCCGTGTCCTCCGGTGTGCTCAGCCAGCACC
TGGCCAGGCACTCTCAGTGTTGTTGAATGAATGAGCGAGTGAGTGACCCGCAGGGGAAGCTGCTGTGTCT
GACACGGTAACGTGCAGGTTGCACTGGGGGGGCCTGGTTGAGATCATGACTGGAGTCCCTGGCCCCCAGA
GTGGGAGTCATCAGTGGGGCTCGCTACCTCTGCACAATTTTTTTTTTTCTTTGAGACGGAGTCTTGCTCT
GTCGCCAGGTGGTACAGTGGTACGATCTTGGCTCACTGCAACCTCCGCCTCCTGGGTTCAAGCAATTCTC
CTGCCTCAGCCTCTCGAGTAGCTGGGACTACAGGTGTGCACCACCGTGTCCAGCTAATTTTTGTATTTGT
AGTAGAGACGGGGTTTCACCATGTTGGCCAGGATGGTCTCGATCCCTTGATCTTGTGATCCGCCCGCCTT
GGCCTCCCAAAGTGCTGGGATTACAGGCGTGAGCCACCACACCCGGCCCCTCTGCCCACTTTCTTCCCCT
TCCTCCTCTCCCGTCTCCACCAAGGCTGTCTCTGGAGTGGCTGTGGATCTGACAGAATGCATGTCCGCTC
TGCCATCACTATTCACGCACAGCGCCTGTTGGGGACTCTCCTGTAACCCAAGGGGGTAGCGGAAGGATGG
AGCTTCAGGCCTTGAGGGCCAGGAAATGAATGTGGAAGACTGAATCCTCAGGGGACCCCAACTCTGGGCC
GGGTCTGGGGCTCTGTTTTGAGGACGGGGAGTGGGAGGAGGTGGCAGCAGGAGCCGGCGTCTGAGGCATC
TCCTCACACTCTTCCAAGGTCAGAGACGAGGTCTCAGATACGCTTGCTAATGCACGATTTATTTCTTTTT
CAATTTCCTTTGGAACCTGTGCTGTGCAGATGTTCACATGATGTGGTGGGGAAGTGAGTTTTATAAAACT
CTTGGCAGAGTACCATTCGGCATCCCTGTGGTTCTAGAGATTTCTGGGGCCTCGGGGCTGTAGCACCAGC
TGCAGACAGCTGCCTGCCTTCTTACAGTGCTCAGCTTTTGGGTCCAGCAGCTTTCTCAGTCCCAAACACA
TGTGACCTCACGATTCTGGCCCTTCCATGATCCGCAACAAACTGTTCTGGTGTGTAGGTGGAATTCCTAT
TTTTAATAGCCAAGTTGTTGAGAAATTGTAGGGCTTCTACTTCCTTTTGAACGGAAGGGCTCCGGAGATA
TTTATATCTTTGCTTCTATAGAATCTAGGAGGTTGTCTTTACCCAGGGAGGGTCAGCATGTGGAGAGGGT
CACCTGGGAGAGGCCCCGGTGTCAGCCTGCACTTGCCGTCAGTAAATGGTACGACATTGAGCACCAAGGA
GCTCAGCCAGCCCCTCGAGGGAAGTCCTAGCTGCCCACTGGGGGAAGGGCGGGGAGGCATGAAAACAATG
ACCCCACTGGCTGGTGGGGGTGACCGTGTTATGCAAAGTGTACACCATGTAGCATGAGCACCGGGCCGGG
AGCCCCCAAGGCTGCTGGGGAATCAGGGAGACACCAAAGAGGAGGTGGCCTTTCCCTTGAAGGGTAAGGG
ATCTGTAGGAGTCTGTGGGAGAGTGGAAGCTGCGGGAGGGAAAGGAGGGAGGGAAGGAGGCCTGGAGAGA
GGAGGGAGGCTCTTGCATTCCAGGCAGGTATGGGAGCTCCTGGCAGAGTTTGCAGTACCCAGTGGCTCAG
TGTGGTTGGAGTTGGCTGTGGGCCAGGTGGGTGGGTAGACAGGGGCCAGGTCTGGAGGGGTTTTTAGTGC
TGTGCTGAGGGGTCTGAAGTCCATCCCGAAAGTTGTAACTGGGGAGTGGCACGGTGGTGAGTTAGAGCCA
CGTCATTCAAACTTTCTCAAATTCCCATACATAGAGACCTAGTGTGCACACTCGCAGCCCAGGCTTGTGA
GCTGCAGTCGCCCTTGCCATGTGCAGCTCGTTCTGGCGTGTTCTGTTCTGCTTCTATTCTGGCTCATTTG
TTCTATGAACAATGCTGTTTGCAATCCTCTACATTGGCTTCCTCTCTCCCTGAATATTTTTGATCTGCAG
TTAGAACAACACTGGATTGGCAAGAGGGAGACTGAGGCGGGAACCCAGGCAGAGGCTGCTGTGAGTCCAG
CTAGGATGAGACGTGGGTGCCAGACAGTGGGCCTGGGGCAGACCCCAGGGACTCCGCAGTGAAGACCCCA
CCTCAGTGGTCCTGGGCCCCAGGCCTCAAGAGACTTGGGGCAGCTCCCTAAGCAGCAAGAGGGGTCCTGT
CATCGTGAGGCGTGTTGAGGATCGAGAAGCTGTGGTTTCATAACTCCGTCACTGTGTGGTGTCTGAAAGC
CATTTCATTAAATGTCACATGGAATCAAAATAGAAATTACATCTCTGGGACAAAGCCCATTGAGCCAGGA
GCCAGGGATTCTGAATTGGAACCAGAATAGCCTCCGAAGCTGGGACTGTGGATCCAAGGCTGCCCCCACC
CCCTGGGTCTCGCTCACATCTGCACTCCCCAGAGGGGGCAGGTGGTCCTGGAGCCTAGCCTACCTGCCGA
GAGTCAGAGGTGGCTGCAGGGGAACCATGGCAGCCCTCCTTCTCACACTCATCCTGGGCACCCTGCACCA
GCAGAAGGGTTTACATGTACAATCACCCATCCCTAGCCCCTTCTGGGAGGGAAGCATATCTTACGGATGG
CGACCTTGAGGCTCAGGGAGGTTAAGGTGCCAGCCTGAGATCACACAGCCAGTGAGAGGCAGAGACAGGG
CTTAAACTCCAAACGATGGCTCCAGAGCCCCCTCTCTTTTCCATGCCCTGGGCTGCCTCTTTCCCCAGTG
CACCTTGCTTTTTGGAACCAGATGACCAATGTGGAAAGACACGAACTGATTCAATCAGAGTGTATGGAGA
AGGGACTTAGAGACCCTGGTATTTTTAAAGCTCCCTGGTAATTCTCATGTGCAGCTAGGGTGGGGCGCCT
CTGCTCTGCGGAATAGGGAAGGGGTGTAAGTGGCCCTGTGTCTCCCCTCTGTCCCCACCTGCCACCTGCT
GGTCCATCCATTCAGCCGTTGCACGGACAGGTTGCCTTCTGGGGGCTGCTAGCCCCCTTGCACTGGGCAA
CGCTGTGTCCCCTCTGTCCCTCCCCCCAGCGCATGCTCCTCGCTCTGCCCCTGCTGCAGGTGGGCCATGC
TTGGAGGGCGCAGGAGAGCTGAGATGGGGTGGGGATGGATCAGGCCTTGAGAGGGTGCCTGGTAAGCCCC
GGGAAGGGTAGGGAGGAGGAGGAGGGAGAGGAGAGGGCAGTGCAAGGGCAGCGACCCCCAGCCTTGCCCC
CGTTTTGAGCACGGGGAAAGTGTACACAGGTAGTGAGGAAATGCCTGCGTTTGGGTGCGTGTTCATTTCT
AATCCATTTTCACTTTTTGTGTATTCTTTCTATCTACTTTTTAAAGGTTTGTTTCTTTTCTAACTTCCTG
TTTTAGATGTATACTTTAGTTTCTTTATTTATTTTTATTTATTTTTTTGAGATGGAGTCTCTGTTGACAG
GCTGGAGCGCAGTGGCGTGATCTTGGCTCACTGCAACCTCTGCCTCCCGGGTTCAAGCGATTCTCCTGCC
TCAGCCTCCTGAGTAACTGGGATTACAGGCGCGCACTACCACGCCCAGCTAATTTTTGCATTTTTAGTAG
ACATGGGGTTTCACCTTGTTGGCCAGGATGGACTTGATCTCTCGACCGCGTGATCTGCCCACCTCGGCCT
CCCAAAGTGCTGGGATTACACGTGTGAGCCACTGGGCCCGGCTTAGTTTCTTTATTTAAAGATAAGGGTT
TTTTTTTGTTTTGTTTTGTTTTTGAGACAGAATCTCACTCTGTTGCCCAGGCAGGAGTGCAGTGGCACAG
TTGTAGCTCACTGCAACCTCAACCTCCTGGGCTCAAGTGATCCTCCCACCTCATCTGAGTATCAGGGACT
ACAGGCATGCACTACCACACCCAGCTAATTTTTGTATTTTTTTTGTAGAGACAGGGTTTCGCCCTGTTGC
CCAGGCAACAAGCAAATCTGCCCACCTCAGCCTTCCAAAATGCTGGGATTACAGGCGTGAGCCACCACGC
CCGGCTAGAGATAAGTTTCTCAAACTCCTGGGCTTAAGCCATCCACCCACCTTGGCCTTCCAAAATGTTG
AGACTACAGGTGTGAGCCTTTGCATTCGCCTTGAATTCCTTTTTCAATAGTATGTTTCCTACTAAAAACA
CTTATGAAAAGTGTGTATTTTCTCTTACCCCTTCTCCTTTTTTGCCATCTAATTTTAGATTATATTTCTT
AGTGTTTGTCTTTAAAATGTACTTATACCTCTATGCTATCTTTTTCTTATTTTTGCCCCCTCCCCCATAA
GAAAGATAAAGAAATCAGAGACTTAGACCAGGTGTGGTGGTTCCCGCCTGTAATCCCAGCACTTTGGGAG
GCTGAGGCGGGTGGATCACTCGAGGTCAGGAGTTTGAGACCAGCCTGGCCAACATGGTGAAACCCTGTCT
CTAGTAAAAATACAAAGTTAGCCAGGCGTAGTGGCAGGCGCCTGTAATCCCAGCTACTCCAGAGGCTGAG
GCAGGAGAATTGCTTGAACCTGGGAAGTGGAGGTTGCCATGAGCCAGGATCATGCCACTGCACTCCAGCC
TGGGCAACAGAGTGAGACCCTGACTCAAAAAAAAAAAAAAAAAAAAAAAAAAAAAAGAAATCAGATACTA
ACAACTCTCTCCTTCTTTCTTTTCTTCCCAATTTTTGTTTAATGTATCATTTCTAAATTCATGGTTTATA
TTTATATATGTCCTTAATCCTCACTCACATTGCCCTACAGGTAGATTCATTGCTCACTGTCAGTTCTCTT
GCTGAAGTTTTCCTATTTTTCTCTTGATTTGCTGAAATTCCTTCTCCAGTAGTTTAATCAAAAGGGACTA
AATGAAAAAAAAATATTCAGTTATTGCAAGTTCAAAAAGGTTTCTAGTCTTTGTGTTTGATTGACAGCTT
TCCAGAATATAAAATTCTTAGGCCACACTTTCTTTCCTTGAGAACTTCACAGATGTCACTTCTGTCTCTA
GAGTTAAATGCCACTGTGGGAAAGTCTGAGTCTAACTTCTATTTTGTTACCCTTTATGAATTGATGTTTT
CACTTGACTGTCCAAAGTCTTTTTTATTTAGCTGTTTCCCCCTTTCTTTTATATTTTTAGTCTAGTTACT
TTCATAGAAATTACCTTGTTATTGACAGATTTTTGTCATTTTCCCCAAGACATGGTGTGCCCTTTCAGTT
TGTAGATTTATCTTCTTTTACTTCAAGAAAATTTTCTTGAATGATATCTTTAAATATTTATGTTCCCCTA
TTTGAGTTTTCTATTCTGGGATATATGATGGGTCCTTTGTAGATCTTCCAAATCTGTAATTTTCTCTGTA
ATCTCTTTACACCGTTCATTTTCATTTCCTTTTGCTCACTTTCCTCAGTCTTGTTCTCAGTGTCTTGATT
GTGTCTTGAGCAATATTTGATGCTCCTCTGCGCACCTTTCCATTTCATCATGACTTTGAAGATACGATGT
TTTTCCTTCTTTCTCCAGCTCTGTCAGCTCCAGTTTCATGTTCCCCCTGAGCTCTCATATCTGTTTTGTG
TGCTTGCTTTCTGGAGAGGATTGCTGTATTCATTTTTTTTTTTTTTTTCGAGATGGAGTCTTACTCTGTT
GCTCAGGCTGGAGTGCAGTGGTGGGATCTCAGCTTGCTGCAACCTCTGCCTCCTGGGATCAAGCGATTCT
CCTACCTCAGCCTCCCAGGTAGCTGGGATTACAGGCTTGCGCCACCATGCCCGGCTAATTTTGGTATTTT
TAGTAGAGATGGTGTTTTGCCGTGTTGGCTAGGCTGGTCTCGAACTCCTGGCCTCAGGTGATCTGCCCGC
CTCAGCCTCCCAAAGTGCTGGGATTGCAGGTGTGAGTCACTGCGCCCAGCCCTGCTTTATTAATTTTTGT
TGTTGTTTAATTTTCAGCGAAAAGTTTGCTGGCAATTTTCATCTGTTCTATGACAACATTTTTACTAGTG
AGTTTTCACTTGCCACTTGTTTTTCCTGTTCCTTTTCTCGTTTTTATTTTTTAATTCTTGCAGTGTCTTC
CTGTAGATGCTGCGCTGTTTGCTTTTTTATTTCTCATCTTTGGGCAAGGTAAGTTTTTCTTCAACCATCT
ATTTGCCAGAGGTTTGTGTGGGAGAAGAAGCGAGGACTACACCCAGTGCCATGTGCAGTTGTAGGGCTGC
TCATGTGCAGTCTGGTGATTCCTCTTTTTGCCTGCAAGTGTGGCTTGTCTGTGTGATTGTCTGTGTCTGA
TCTGCTGCTTCTGCTTCTTGGTTCCAAGCTTACCTGTATCTCACATGCCACTGTCATGAGATCACAGGCC
CCACTCCTGCTCATGCAGATAAGGGACGTTGGTTGGTGGGCAGTGTGATCCCTTCTCACTGCTTCTTCCC
AAACATCTGTGTGGTATTTCCTGCCTGGGCAACCCTCTGACTCGTTTCTAGTTTGGGTTTCATCTCTCAT
CTGTTTCCATTGGAAATGGAGTGTAGCTGGGCGCAGTGGCTCATGCCTGTAATCCCAGCACTTTTGGAGG
ACGAGGCGGGTGGATTGCTTGAGCCCAGGAGTTCGAGAGCAGTCTGGCCAAGAAGGTGAAACCCCATCTC
TACTAAAAACACAAAATTAGCCGGGCGTGGTGGTGCAAGCCTGTAATCCCAGCACTTTGGGAGGTCGAGG
CGGGTGGATCACGAGGTCAGGAGATCGAGACCATCCTGGCTAACACGGTGAAACCCCATCTCTACTAAAA
ATACAAAAAATTAGTCAGGCACGGTGGCAGGTGCTTATAATCCCAGCTACTGGGGAGGCTGAGGCAGGAG
AATCGCTTGAATCTGGGAGGTGGAAGTTGCAGTGAGCCGAGATCACGCCACTGCACTCCAACCTGGCGAC
AGAGCGAGACTCTGCCTCAAAAAAAAAAAAAAAAAGATGGCGTTTGCATCCTGTTTCTCTTTCTCCTTGT
TACTATGGGATGATTTTTTTTTTTCGACTTTATTGGTGTATAATTGACATACAATAAACTGCGCCTATTG
AATGTGTTATTAGTAAGTTCTGACATATGTATACACCCATGCAGCTGTCACAACCATCAGCCACTAGACA
TCCTGTCACCCTCCACAGCTTCCTCATGCTGTTTCTTACCTCCACTCCCATTTTCAGACAAACTGACTGC
TTTCCGTCGCCAGAGTTTACATTTTCTAGAATTTCATGTAAATAGAATCCTACAGTGTGTTGTGTTTTTT
TTTTTTTTTTTTTGATGTGGTTTCTCTCACTTAGCATAGTTTTTTTTTCTTTTGAGAAAGAGTTTTGCTC
TTGTTGCCTAGGCTAGAGTGCAATGGCGCGATCTCGGCTCACTGCAACCTCCACCTCCTGGGTTCAAGCG
ATTCTCCTGCCTCAGCCTCCCAAGTAGCTGGGATTACAGGTGCCTGCTACCACGCCCGACTAATTTTTTG
TATTTTTAGTAGAGATGGGGTTTCACCATGTTGGCCAGGCTGGTCTCAAACCCCTGACCTCAGGTGATCC
ACCCGCCTTGGCCTACCATTGCTGGAATTACAGGCATGCGCCACCACGCCTGGCCTAGCATAGTTATTTT
GAAGTCCATCAGTTGTATATAACAATGGTTCATTCCTTCTTATTGCTGTGTTGCATTTCATTGTATGGCT
ATGCCATATTTATTCATCCATTCACCTGGTGATGGACATTGGGGCTGTTTCCAGCTTTTGGCTATGATGA
ATCAAGTTGCTGCCAGTCTGCATGTAGATTTTTTTTTTTTTTTTTTTGTAGACAGAGTCTTGCTCTGTTG
CCCAGACTGGAGTGCAGTGGTGCGATCTTGGCTCACTACAGCCTCTGCCTCCTTGGTTCAAGCGATTCTC
CTGCTTCAGCCTCCCAAGTAGCTGGGACTACAGGTGCCCACCAACAGCCCAGCTAATTTTTTTTGTATTT
GTAGTAGAGATGGGGTTTCACTATGTTGGCCAGGCTGGTCTTGAACTCCTGACCTCGTGATCTGCTTGCC
TTGGCCTCCGAAAGTGCTGGGATCACAGGCGTGAGCCACCACACCTGGCCACATTGCTGTTGAGGAGGTG
CATAGGAGCAGAATGACTGGGTCATATAGTAGGTTTCACTTTTTAAGAAGTGACCCAACTGCTCTTCAAA
GTGACCATACCGCTTTACATGCCTCTGAGCACATAAGAGCAACACAAGAGTCCCAGTTGCTTCATACCCT
TGCCAACACTTGGCATGGCCAATCTTTTACATTTTAGCCCTCCGAGTGGGTGTCTAGTGTATATCCTTGT
GGTTTTCATGTACATTTCACCAATAACAACTGGCATGAGCCTTTTTAAATGTACTTATTTTTTATATGTA
CACCATCTTTGGTGAATTGTTCAAATCCTTTGCCCATTTATTTTTTATTTTTTATTTTATTTTTTTTTTG
AGGCAGTGTCTTGCTCTGTCACTCAGGTTGGAGTACTGTGGCCCCATCTCGGCTCACTGCAAGCTCCGCC
TCCCAGGTTCATGCTATTCTCCTGCCTCAGCCTCCCGAGTAGCTGGGACTACAGGCGCCCGCCACCACGC
CCAGCTAATTTTTTGTATTTTTAGTAGAGACAAGCTTTCACCATGTTAGCCAGGATGGTCTCGATCTCCT
GACCTCGTGATCCGCCTGCCTCGGCCTCCTAAAGTGCTGGGATTGCAGGTGTGAGCCACCATGCCCGGCC
CCTTTGCCCATTTAAAAATATCGAGTTGTGTTATATATTAGGTTTAACAGCTCTTTATTCTAGTTACACG
TCTTTTATCAGATATATGACTTGTAAATATTTTCTGTAGTCTGTGCCCTTCTTTTTCATTTTATTCAGTG
TCTTTTGAAAAAGTAAAAGACTTAATTTTGGTGAAGTCCAATTTACTGTTGCTTTTTTTTCTTTTATAGT
TTATGTTTTCTGTGTACTATTTTTTTTTTTTTTTAATTTTTTATTTTTTGAGTCTCGCTGTATCACCCAG
GCCAGAGTGCAGTGGTGTGATCTTGGCTCATTGCAGCTTTGGCCTCCTGGGCTCAAGCAGTTCTCCCACC
TCAGCCTCCCAAGTAGCTGGGATTACAGGCGCCCGCAAGCATGTGTGGTTAATTTTTGCATTTTTAGTAG
ATATGCAGTTTCACCATGTTGGCCAGGCTGGTCTCGAACTCTTCACCTCAGGTGATTTGCCTGCCTCGGC
CTCCCAAAGTGCTGGGATTATAGGCAAGAGCCACCACACCTGGCCCCTGTGTACTATTCTTAAGAAATCT
TTCCCTGTGGTGAAAAGGGAACTCTTGGACATGGTTGGTGGGGGTGTCGATTGGTACAGCCATTATGGAA
AGCAGTATGGAGGTTTCTAAATAAATAAAAAATAGAACTACCATATGACTGACCAAAGGAAATTATATCA
CCACCTTGTAAAGATAGCTGCACTCCTGTGTTAATTGCAGCATTATTCACATTAGCCACGATATGGAAAC
AACCTAGGTGTTGATGAATGAAGGGATCAAAGGGCCGGGTGTGGTGGCTCATGCCTGTAATCCCAGCACT
TTGGGAGGCCAAAGTAGGTGAATCACTTGAGGTCAGGAGTTTGAGACCAGCCTAGCCAACATTGTGAAAT
CCTGTCTCTACCAAAAATACAAAAATCAGCTGAGTGTGTGCTGGCGCGCACCTGTAGTCCCAGCTACTCG
GGAGGCTGAGGAAGGAGAATCACTTGAATCTAGGAGGCGGAGGTTGCAGTGAGCCAAGATCATGCCACTG
CACTCCAGCCTGGGTGACAGAGTGAGGCCCTGTCTCCAAAAAAAGATCTTTTTTGACTCTGCGATACAAA
GATGTTTTCTTCTAGGTACTTCATAGTTTTTACATTTAGGTCTCTGCTGCCTTGTTAGTTAATTTTTGTG
TATGGTATGAGGTTGGAGATTGGGGTTTATTTTTCTGTTCTCTTTGAGCCGGTTTCTGGGAGGAGAGAGA
GGACCCACCAGCTCCTCTGCTGTGTCAAGCTATTATACAAGGCCCCATATAGGCCGAGGTTTCTGTTTTT
TTCTTCTCTAGGAAAAAAAGAGGAAAAGCCATCAGCAATACCAAGGGAAACAGTAAGCTAAGATTGGCAT
AGTTCTCAGCAAGCCTCACAGCGATTCTCTGCTCCCTCCCTCACCCCTTGCCTACCTTTAATCCAAAAAT
GATTTTACCAGAATGTCCAACAAATAAGACAAGACCCAAAAGCACTGAGAACTTTTTCTTGCTGCCAAGT
ATAAAACACAGACCAGTATAGTGGCTTAAAAAAGCAAATTCCCAGGAAAATTTATAGAGATGGAAAATAG
GACAGTAGAATAGCAGGGACTGGGGAGGGGAGAAGGGAGAGGTGCTGTTGAACAGGTAGAGTTTCTGTTT
GGGCTGATGGAAAAGTTCTGGAAAAGAAATTGTTGATGGTTGCACAAGATTGTGAATATTCATAATACCA
TTGAATGGCACAGTTAAAAATGATTAAAATGGTACATTGGGTTACCTATGTTTTACCACAATTAAAGAGT
ATTTTTAAAAAAGCATATTCCCTCTGGGGTTAGTTAGCTGTAGTCAACACCTAAGTTCCCCAGTGATGAA
CTGAAGTATGGACCATCAGCATAAATGTGAATTAAAATTAAATATTGCCAGGCAGGCTGGGCGTGGTGGC
TCACGCCTGTAATCCCAGCACTTTGGGAGGCCTAGGCGGGCAGATCGCTTGAGGTCAGGGGTTCTAGACC
AGCCTGGCCAACATAGCGAGACCTCGCCTCTACTAAAATAAATACAAAAATTAGCTGGGTGTGGTGGCAC
ACACCTGTAGTCCCAGCTACTTGGGAGGCTGAGGCAGGAGAATTGCTTGAACCTGGTGGGTGGTTGGGTG
GGGGGTGGGCGCACAGAGGTTGCAGTGAGTCAAGATGGCATCATTGCACTCCAGCCTGGGTGACAGAGCG
AGACTCCATCTCAAAAAAAAAAAAAAAAAATTATCAGGCAGTATGAGGTTGCATGGTCAATATCCTAACA
GTCACAAAGCAAGCAAGTGATCTTGAGGGGGAAAGGGAGGAGGTGGGAAGAAAGGAGGAGGACCAAGTTC
CGCTGAGTGGCTAATAAATATTATTTTGGTTAAGTCCCTGTCTCCACAGAGTAAGTCCTGATTCTTTTTT
TTTTTTTTTTTTTTTGAGACTGTGTTTCACTCTTGTTGCCCAGGCTGGAGTGCAGTGGCGCGATCTCGGC
TCACTGCAACCTCTGCCTCCCGGGTTCAAGTGATTCTCCTGCCTCAGCCTTCCCAAGTAGCTGGGATTAC
AGGCATGCGCCACCATGCCTGGCTAATTTTGTATTTTTAGTAGAGACGTGGTTTCTCCATGTTGGCCAGG
CTGGTCTTGAACTCCCGACCTCAGGTGATCCGCCTGCCTCGGCCTCCCAAAGTGCTGGGATTACAGGCAT
GAGCCACTGTGCCCGGGCTGTAAGTTCCTGATTCTTAAGCCTAACATAGTTAAATTGCTCTGTAAAAAAA
AAAACAGTGGCTTTATATTCAAGACCATGTTGCTTCCAACTGAATTGGCCACTGACACTGGGATACTGAG
CAGTGAACAGACCACAAGGAAGACATCACTCTCGGATCCTGTTTGAAACCAACAGCTACAGCAAAACACT
AGTCTTGATTCAGGGCCTAATGGGGTTTCTTTAATTAGCAAGTTTACATTCATGGAGTCTTGTGAAAATG
GTTAGATAAATTGCAGAGAAATAAATAAGTATTTTGTACATCTGAGTATTATTGTTAATTTCCACCTGGC
TACATGAGGCTCCCTCCTCTGGGTTTTCTCTCTATGCCCTTTTGCTGTGGACAGAAGGTACCTTGCTCAT
TCATCCATCCAGCCGGTGAGCCAGGGGGCAGCCCGGAGGGAGGAGTGGAGGGGATGTGGGCCTGGAATTA
GACTGAGCTTGGTGTGTCCCCAGCTCAGGCCGCACTGCCAGGCCCTGGTTTCGTTACTATGAAATGAGGG
GTGGGGTGGGTATCTGGTCTCCCACTCCCCATTACTGAGGGGTTCCCGGGCTCTCCTCCACCCTCTGATT
CCTCCCCCTCTTCTGTTCCAGTCCAGAATGCCTCCTTTCTGCCTAAACGCCTCTGATCTTCATTTGATCA
TCAGCAGCTGCAGACCCATGAGCCACTGGGCCTGACCTGGGAGACACTGGCCCTCCACTTCCAGTGCTCT
CCAGCTGGATTTCCTCCAGAACAGTGGCAAAGAGTTCGCCCAGAGCTGCGTCCCCTCCCTGGGAAGCAGC
GCTGAGTCAGGGTTACCTGGCTCCACTGTGCCCCTAGCGAGGTGAAAGTTCGCACAATGACATGGGCTCT
CTGTTGAGGATAAGGAGGCTACAGCCCAAGGAGCTTGTGCACCGCAGGCAGAGCCACTCAGGTCATCTTC
CAGAGCGGGATGTCTGACTCCAGAGCCCGCCAGCCCACACTGCTCTCCTGAGGACTGGGTTTCTCTGGCC
TGAGTCTGCCTGAGTCTACAGGAAGAACCCTGCTGGCACCCCAGTAAGCCCCTCTAGACCTTGGAGCTCT
AACTGCTCTCAGAGCTCACTTTTCAGTTAGCCTGTAAGGCAGGGGTGTCCAATCTTTTGGCTTCCCTTGG
CCACATTGGAAGGAGAATTGGCTTGTGTCACACATAAAATACACTAACACTAACGATAGCTGATGAGCTA
AAAACAATTAAAAATCACAAACTCATAATGTTTTAAGAAAGTTTATAAATTTGTGTTGGGCCACATTCAA
AGCCGTTCTGGGCTACGGGTTGGACAAGCTTGCTATAATGGATCAGAGATATGTACAGTCTTAAAGAAGA
AACAGCTCCCAGGCCTGTAAGACCTATCAGGGCACTCACTGAATGCTGTGAGCAGGGGAAGGGGCAGTCC
TGGGCAGTGGGGTCCTCTCTGGGGGTCCAGCTGTGTCTCCAGTCGTCTGGGGAGCTGGGAACAGCACACC
AGGGTTCTTGCCTCCTGGGAAAGTTCTCACAGAAAGAGAGAACAGAGGTGCAAAAGCTAGGCCATGTTCA
TTATTCTTTCATTCCCTACTTATTGAGCTTTGACAATGTGTGAGGGACTGCTTGATTGATTGACTGATTG
ATTGGGACAGGGTCTCTGTCACCTAGGCTGGAGTGCAGTGGTGCAACCTCAGCTCCCTGTAACTTCCGCC
TCCTGGGCTCAAGCCATCCTCCTACCTCAGCCTCCTGAGTAGCTGGGATTACAGGCCCACGCCACCACAC
CCGGCTACTTTTTGTATTTTTTGGTAGAGACAGGGTTTCGCTATGCTGCCAGGCTGGTCTTGAACAACCA
GGCTCGAGTGATCCTTCTGCCTCCCCTTGGGCTCCCAAAGTGATGGGATTATAGGCTTCAGCCACAGTGC
CCAGCCAGAGGAACGTTTCAGATGCTGGAATCAATGGTAGTGTGTTGGAGGAAGGGAGTTTCTGAGCTCA
GCCCATTTCTTTTTTTTTTTTTTTTTTTTTTTGAGATGGAGTCTCGCTCTGTCGCCCAGGCCGGACTGCG
GACTGCAGTGGCGCAATCTCGGCTCACTGCAAGCTCCGCTTCCCAGGTTCACGCCATTCTCCTGCCTCGG
CCTCCCGAGTAGCTGGGACTACAGGCGCCCGCCACCGCGCCCGGCTAATTTTTTGTATTTTTAGTAGAGA
CGGGGTTTCACCATGTTGGCCAGACTGGTCTTGAACTCCTGACCTCAGGTGATCTGCCTGCCTCTGCTTC
TCAAAGTGCTGGGATTACAGGAGTCAGCCACTGCAACTGGCCCTAATAGACATTTTTTTAAACTAAATTA
TTACACTACAGAAAAATGCACACATCATAAGAGCACAGCTCGATGCCTTTTCTCAAAGTGTATGCACTTG
TGAAGTCAGCCTCCAGATGGAGAAAGAGAACATCATTAGCACCCAGGAAACCCCTCTTGGCTCCTCCCAG
TCCTGACCACACTAAGGCGCCTCCATCTCCACTTCCATCCCCAGAGATGGCCGCTGCTCGGTGTGGTACT
TTATGCAGACAGGATCAAGGATGGTACACTTTTTGGTGCCCTTTTTTGGCTTGACTCAACATTATGCTTG
TGAGACTGTGTTGCAGGTAGCTGTAATCTTTTTTGTTGCTGTAGAGAGTATTCCATTGTTTGACTATACT
ACAGTTTGTTCATTCTACCCCTGATTGGCATTTGTGCATTTCATTTTTAAAATTATGAATAACTCTATTA
GAGGCAACTAATGGCTCAGTTTTGCTCCTTGCTCTGCAAACCAACCCTGCCCATAGGAACTGAACTTGTA
AAGGGGCAGGTGACGTATTTTAGTGAGAACCTTCCATGGCGTAACCTAGTAGAATGCCTAATGTTGGAAC
CTCGGGCATCACAGCTGGCCTATTTATTTTAAATAAATGAGTTCATGAATAAATGAAGGAATAAAGGAAT
GAATGAGAGGAGGGGTAAGTGACTAAATGAATGCATTTCTGGGAAGAGCATTATTGGGTACTCACCTGTG
TCAAACACCTCTGGTCATCTTCATAAATGCTGCCATATGTAATCCTTGCCACAACCCTTTGAGATGGGAT
GGCTACCTTGATTTTACGGATGAAAAAAATAACAAGGCTCAGAGAATCCAGGTCATTTAACTCTTCTGAG
CTCTCCATTTTCCCATGTACGAAATGGGTGTCAGTAGAGCTCCGCAGATTGTTGAGTGGGTGAGGGGGTG
ATACAAAGCGGTCAGCAGTTCTGGCACAGGAAAGCGCTCGCGAACTTCCTGCGCATCATTATTCTGGCTT
GTGCTCATGTAGCCTGTAAGTGGCCTGGCCAGGATTTGAACCTACAGCCTTCCATCATCCCCCTGCACAA
CTCCTGCCACCCCATGCTGCCTGCCTCTGGGAGATTAGGTGAGCAAAGGGGTCTTGGCCATGTCTTTCCA
TCTGAGGCACTCAGTACAGAGGGTGAGGGGTCCCTGAGCCACCTCTGCTGCCTGCCCTAACTCCGGGCTG
TGATGAAGCAGCTCTGTTGGATGAAGCAGCCCTGGCTCCGATGCTCTTCCTCCCCTCACCTTCCTTACCC
AAAGGATGAGGTTGGAATACAATTGCAGTGTCTGTGCTCACTAATTTCATTTTCTGTGTCTGGAGTAATA
GGGGCCCTCAAACTTATTATGCTTATTTGAATTTCAATAATAAGCAGCCAAGCGGGAGACTGGCATGGGG
GCCGGGGCCGTGCTCTGAGGAAGGCCTGGCTGCCCCAGCTGGGGAACAGCTGGGTGCTTCCGAGGAGTGG
CCTTCTCTCCCGGGAGCTCGGGGTTGGTCAGCTGGTCAGCTATGACCAGGCAGAAGCTGACACTGACCTG
ACCCAACCTGGGCAGGCCTAGGAGAGCTCGTCCCCAGACCTTCACCTTGGAGAACCAAGTAGTAGTGAGA
ACAGTGCCGCGGGGCTGGCCCAGATCATCGGTCCTTTGGACGTCTTCTTCCATAAATGAGTCATTTATAT
CACACTCTCTTGTCAAGAGGTGGAAGTCAGTTTCCAATGGGGACCAAATTGGCCCTAATTTTTGTTCACC
TCTGGTACTGAGAGGTAAGGTGGGGGTGGGTGACATTTTCAAAGGGGCTGTATCCCTAAATGAGATAAGA
CCACTCCCTTGTGGGTGGAGACACCCTTAGCCCAAAGGCTTGAGTAGTTGAAGAGCTGTAAACAGTGGCT
ACCAGGTGCCCCAGGTACCCAGACGATGAGCAAAGCCCCAGAGGGATCCGAGGAGGTGACACCAGCATTT
TCCCCTGACTGGTTCTCGCCCTCAGCCTGGGAATGTCGCTGAGTCTTTGGTTCTTCCAGAGACCTGTCTT
TTGGCTGCCAGCCTTTGCATAGTGTCCACTCCTGTATCTAATTCTCAGGAGACTGAGGTCTGGCCTAGGC
GGGTCTCTCCTCCGACTCAGTTCCATGCACTGAATTTGACCCCTGGGCCCAGATCAGACACAGGGTCTTT
CCACGAGGCCCTCACAGAAGGTGGAGCTCTGAGCGGTGTTTTTCAAACAGGATTTTAATGGTGCAGCAAG
AAAAGAAAGACTCCAGGATGACTGAGGTTTAGGCGTCACTGGGTTGAAACGGTTAAAGAGGCTTCCTTCC
TGCAGGGCTGCTCAGAGCCTTTATTGGCCGCTGTATTGTGTGTTCACACACACAGAGATGGAGACTGGAG
CTTTTCCCCAACACTCAGATCTTTGTTTCCCAGAAGCCTCTCCAGGGAGAAGAGCCCAGCGTTTCAGAAA
CGCTGGCTTGAAGGAGCCCTGGGAGCCTGCCCCCCAGTTTCTCTGTAGCCTCACGATTGTGCTTTCTGCT
GGTGAGGGAGGTGGCTGGAGGTGGGAAGCGCCCAGGAGGCCCGGCTCTCCTTGAGGACACAGCTCTGCTC
TTACCCTTTGTGCTTAGTGCATCCCCAGGGCCCTCGGGGGAAAGTCCAGCCCCCTCTCCCTGCCCCTCCA
GGCCCTCTGTGATGAGTTTCTTCCCTGCTCTTGTGTGATTTCCTGTGTGCGCCTACATCCCAGGCAAACC
AGGGGGTTCCTGCTCTTATGGGCTGCCGTCTTCTGCTCATGCTGGGCTCAGCCTAGATACCCTCCCCTAC
GCCTTGCACATAGTTCGGGTTTCACCTGAGCTGCACCCCTTTCCAGGAAGCCTTCCTGATTGTCCTGTGC
TAGCTTTTCATTTCACTGCTCCCCAGCATTTTGTATCAAATCCAAATGCCTTCTGAGCTGACAGCATCTC
ACTCCCTGACAAGATGTGGGCCCCTGGGGAGCGGTGCCAAAGTCTGGTTAATCCCAGGGTCCCTGCTGCC
TGCACGGAGTGGTGCTGAGTGGAGACTCCAGGAAACCCAAGAGCTTCACCTCGGGCTTGCCCATCGGGCC
CTGCAGCTTTGAGCAAATCGACCGAAGGTGCCTACTGTGTGGAACCCAGCAAGGGGAGCCATGCAGACCA
GGGAGAGGAGGCTGGCATGGGCAGGGGCTCCTCCCTAGAGGCTGCTTGGATTTAAGGCGGTTCCCTCATG
CCCAGCTCCTCTCCTTCCTTCCCATATTCACCATAGAGGTTTGGGTCCCCTCTCCCCTCTGCCTCTGCCC
CAGGACCACCCGTGCAGACCACCCCCGCCTGCCAGACCAGTCTCAGGGCATCTGATGGCTTCCCTGCCTG
CTCCGCCCACATCTCAGGCCAAACCCCTGGACGTCAGGACACTGCACCATGCAGCCCACTGTATTTTCAG
TCTTTCCTGGAGCCCTGCCCCCGCCAGGTGAGCTGGCTGCTTTCTTGGGAAGCCAGGACCTTCCTGGCCA
CCTCTGTCTGCCCTGTTGCCTGCGCCCCTGCAGTGCTCCCACCAGGCTTTCTGAAGCTGGCCTGGCCCTG
AGACCCCCAGCCCTGCCTCTCTGTGGAATTGTTTCCTAGCTGGTCGCTCCGTCCTTCCGAGCCTCCCTCT
TTGGGGCTGCAGAAAGCGTGGTCTGAGCTACACAGCACTGAGCTCCACCCTGTCCCAGGCTGGTAGTTCT
TTTTTTGTTTTGTTTTTTGACACAGGGTCTCGCTCTGTGGCCCAGGCTGGAGTGTGCGTGATCTCAGCTC
ACTGCAACCTCCCAGACTCAAGCAATCCTCCCACCTCAGCCTCCCAAGTAGCTGGGACCACAGACATGCA
CCACCATGCCTGACTAAATGTTTGCATTTTTGGTAGAGACAGGGTTTCCTCGTGTTGCCCAGGCTGGTCT
TGAACTCCTGAGCTCAAGTGATCCACTTGTCTCGACCTCTCAAAGTGCTGCAATTACAGGCATGAGCCAC
TGCGCCTGGCCTGGCGGTTCTTTATTAGCACAGGTCCAAGACTTTGACTTCCTTATTGCAGGGCAGATAT
GGATGTGTGTGTCATGACCTCCACCACCCGCGTGCACGAGGGGCACAGCCTCTTGCAGGTGTGTCACGTG
GCAACCTGGACCAGGGATCTCCCCAGCCCAGTGAGGGGCCAGCCTCATCTCCATTCTACAGATGAGAAAA
ATACATACACTATTTAAAAACACTTCTATCCAACACAGCCACACACACACATTCATATGCATACTCACAC
ATGCTCACTCATGCACTCACACATACACTCACACTCATGCACACACACACATTCACACACACACACACAC
ATTCACATGCATGCTTTAACTCGAGGGAAATGAACTCACAGGACTCTAATTTCCTGATGCCATAGGAAGA
ACAACGGGCATTCCTGTCATGTTTTTCATGGGAAGCTGATTCAGAGAGGCTAAGGGGCATCCCTAACGTC
ACACAGCTAAGAGGCAAGGCTGAGGTTTGCACTTCCACTGCTCTGACTTGGAAGCCCAGTGAGGCGCTCA
AATTAAAATTAGAAACCTTTTCCTGTTGTCCAGGCAGAAAACCGGGAGAGGGGAGAGAACAGCCACTAGG
GCGAGGACAGAGCGCAGCCACTGGGCAGCCGGGAAACCGAGGCTTGCGCCTGGCTGCAGACTTCCCTCTT
CAGGCCCTTCTGGCACCATCTGCTCCAGAACCCACAGCTCCCATCCGCCGCTGCCTGGGATGAATGGTGC
CCTGTCAACAGTGATACACCTGCTGTTTAGTGGGAAGAGAGGTGCCAAGAGTGCACGGGATGCCCGGGTG
CACAGAGGGGAGGAGACAGGGCACAGCCGAGCTGCCAGGCCAGGCACAACTTCTTGATGAATAGAGAGGT
GTGGAGCCGCCCGTCGTTCATGTCGATTCTCTCAGTCAATCAAAACGCTGCCACAGCAGGCTTTGGGATG
CGGCGTCTCGATGTGTGTGGGCGTGTGCAGGGCGGTTGCTGATATTAGTCATGAGCGCTGGCCCAGGAAT
CAGAGACGGGGATGAGTCTCAGCTCTGCCATTTCTTGGCTGTGTGACGTCTGGGCCTTGATTTCTCCATA
TGGAGATAGAGAACCCCTTCCTTCTCTGTCCCCAGAAAAAGTGCCGGCCACACTCTCCTTGGCCTGTCAA
CAGCGATGCACCCGCTGTTTAGTGGGAAACCATCTTCCTTCATGAGGTCAAAGCCCTCACTTTTTTCTGG
GAGGGATGACTGTGCACCGTGAGTTAGCCTTAGAGTCAGGCCCAGGAAGACATCAGCCACCTGCACCCCC
CCGCCGCCCCCCACCCCCCTGCCGAGGCCTGGGGAGTCACAGCTCTTCCTGATCTTGCTGTGTGGCCTCG
GGCAGGGCGTTTGCCCTCTCTGGGCCTCAGGCACTCCATCTTGACAATAAGGCCATTGCACTTGCTATTC
ACATTGGGCCACCTGTGCCAGCCATCCATGACAGGGGTTCTTTAGGTAGAGACAAATGGCCTGCAGGGGC
GGGGACCCCCGGGGCATGGCCGTCCACTGCAGGTCCCAGGAGGAGCCCCAGCCTGGCTGGCTGCTTCCAA
GAATAGAGTAGACAGAGTCTGCACGGGGCTGCCAGTGGGCCACTCCTGCACGCAGGGCCCCGGGCCCTGA
GCTCATCAGTGAAGCAGGTAATTATGTGTGTGCTTTGTTCCTGCACAAAGGCACTCTCAGAGAACTTCCA
AGAGGCTCTGCTTCCTTGCTGATTTGATTTTTTCAGAACTGGAGTGTGAAAGTCAAGCTGAACCTGCTTA
ATTGTGCCTGCGAAGGCTATTCAGAGAGACTGCTCTTGGAAAGAAATCAGGACAATTAGCTCTTGGCTGC
AGGGAGAGGGGGAGCGAGCGCTTGATGGTGAATGTCAGCCTGTTCCTGGGAGAACCAAAGGGCAGAGCTG
GGCAGAGTGGGGAAAAGACGGGCGGGGGGGTGGGGGGGCATCTGTAGCTGTCTCTGGGGCTGGGGCCAAG
CCCACCACCTGCAGGGGGCACCAAGCTCCACATGTGGGTGTGGGGCAGCCAGGCTGGGAGCAGAGGCCAG
GTCCGACAGCAAGCTCAGGGCCACTCCCCTCAGTCCAGTGAGGGTTGGTTGTGTGATCTTGGGCAAGTCA
CATGGCCCTCTCGGCCTCAGTTTCCCCGGGTCTGCAAAATGAGTGCTGTGCAGAATGAGCAGATGGAGCC
CACTCATCCCGGGCAGTGGCGGGTGGGAACTATGGGGTCCACAGCAGATGGTGCCAGAAAGGCCTGAAGA
GGGAAGTCTGCAGCCAGGCGCGAGCCTCGGTTCCCCGGCTGCCCAGTGGCCGCGCTCTGTCCCTGCCCTA
GTGGCTGTTCTCTCCCCTCTCCTGGTTTTCTGCCTGGACAACAGGAAAAGGTTTCTAATTTTAATTTGAG
CACCTCATTGGGCTTCCAAGTCAGAGTAGTGGAAGTGCAAACCTCAGCCTTGCCTCTTGGCTGTGTGACG
TTGGGGATGCCCCTTAGCCTCTCTGAATCAGCTTCCCATGAAAAAGATGACAGGAATGCCCGTTGTTCTT
CCTATGGCATCAGGAAATTAGAGTCCTGTGAGTTCATTTCCCTCGAGCTAAAGCATGCATGTGAATGTGT
GCGTGTGTGTGTGTGTGTGCATGAGTGTGAGTGTGAATATGTGTGTGTGCATGAGTGAACGTGTGTGTGA
GTGTGCATATGAGTGTGTGTGTGTGGCTGTGTTGGATAGAAGTGTTTTTAAATAGTGTATGTATTTTTCA
ACCTTTTTTGAATGGAGCCCAGGGTGTATTTCCCAAATTCTTTTTACATTATTTTAAAGAGAGGCAGCAG
AGTGTTATGACAGAGAACCAGCTATGGAGCTGGCCTGAGTCTGCATCTGGGCTCCACCTCACCAGCTGTG
TGTGAAACCACTTTGTTCCTGTTTTCTTATTTGTAAGTAGAGATCATATCATCTATCCACATCTCTGCCT
AGGTGTGTGGACGGCTTAATACACTTGGAAGCCTCAGAACAAGGACTGGCAGTAAGTGCTCAATACATTT
TATCATCATTGTTATTTTTGACGCTGAGGGTCACTGTAACGACCAATGAACCAGCCAGTGAAGTGGGATG
AATCCATTCAGGAGCACACAGGGCTCGGGTGCACTCAGTGCTCACGGGCGAAGTGAGCAGGCTGGCATCT
GTTTGGATTGGTCTCTGCTTGAACCCTGTTAGTTGTTAAACAGTTTGAATGCCATCCCAGGGTAGAAGAG
ACAGAAACTTAAGGGCTACTTACAGATGCAGGGACTAGGGGAGGGATGTGGGTGTGGCCAGAGAATCCAA
CCCTGGGGACCACCCATTTCCTATCTGGGCGGCCAGGGCCCTGCAGGAGGTGCCCCCGTCACTTACAAGC
CAGGGCCCCCACTGCCCCTCCCTGGTCCTGCAGTCCATCTGGGATGTGAGGCTACTCAACCAAGCTCAGT
TTTTGCAGCCAGGCGCACGGGGGAAGGCTGCCACTGGCTGCCATCACCGACTGCGAGGGGTTCCTCATTC
TCAGCTGGCTTTACCCTTTGCTGCTCCTTGAATAAAGTGTTTTGAATTCTGCCGCACAGAGGGGACCCCG
CTTTACTCTTTTGGTTTCCTTTTGCCCGAACACAGCTCACTACAGCCTCAACCTCTTGGGCTCAAGCCAT
CCTCCCACCTTAGCCTCCTGAGTAGCTGGGACCACAGGTGCACATCACCACACCTAGCTGATTTTTTATA
TTTTGTAGAGACGTGGTTTCACCATGTTGCCCAGGCTGTTCTCAAACTCCTGAGCTCAAGCAATCCGGCT
GCCTCGACCTCCCAAAGTGTTGGGATTCCAGGCATGAGCCACTGTGCCCGGCCTCTTTTGGTTACCTTCA
TCGGAACACTTGCAGGGCCTTTGAGAGACCCCATCAAACACGTTTCCCTTTGGGCACCTACACTGAATCC
TCTGGGATGAAATTTCAGCCAGGAGAGGGACTGAAGTAGAGTCACATGTGGGGACCTCCCATTAGTGGGC
TCCGTAGCTGACGGTCCCAGGACAGGGGCCCTGTGTGGAAAGAAGGGCAGACCTAAGGGAAGCCAACAGG
GGTGAGGACCACGGCGCAGGGGCCACATTTAACCGGCTGGGGACCAACTGTTTCTAAGGCTTCCGCCGCT
CAGCTGGGCAAAAGGGAGATAAATATTGTAAAGTTTACGGGAAATAACAGTTTTAAACAAGCTCTGGAAG
TGTAATGTGCCCACAGGCATTCATCATCATCACCGCTCTCATCCTCTCCTGGGTGACCAGGGCGATGAAT
GACCCAAAACACCACACACGAGGAAAGGCAAAGAGATCAGAGACTGGTTCACCCAGAGAGACCGGGATGC
TGCCTTCCAAAATGGAAGAGCTGTGCTGGAAAAGCTGCATTTGACTCACTGGAATAAGTGCAGGGGTCCA
GGGCCTGCTGATTGCTTCCTGGTCCCTGTGCACATGCCCCGTGGTGACTCCGTACAGTGCTTCACAGATC
TTTTGCTGAGGAGCTGGGGATGGGCAGGGAAGGAAGCCTGGACTGGGTGCCTAGAAGCTCTGGGCAGCAG
CCGCATCAGAGGCTTTTGAAGGCAGGAAGGGCAGCCAGCGGCCCGGAGGGGAGCACAGCAGCTGCTGGTG
ACAGCTTGCTGGGTCGTCACAGGGGCTCTGGACGGTCCTTCTGCCCCGAGGTTTGGCTTAGCTTGGTGAG
GGCAGCGTGGAGGACACCGTTCCATCTTAATGCCTTTCCAATAAAGCCTTGAAGACGACAGGCCCCATTT
CCTGAGCAGAGCTGAACAGTTACTTTACACTTCATCTAATTGATTAAAATGACTCCTCTAAATTAACCAG
GCCTTCCAGAGCTGAGTGCGGGAGAGTGAAGTCCAGAGACCAGAGCCGGCTGCAGGGGCAGGCCGGGGGC
CCACAGCCCTTCTCCCGGGCCCTGCTGTCCACCAGCCCTGCTCGCACCCCAGGTTCCTGGTGGCCGATGC
GCTTAGGTCAGCCGACTGGCTTCTGTGCTCTGCCCCTGAACACCAAGCTGCCCTGAACAGAGCCTGGTCT
CTCAGGGTGAGGGTGGGTCAGGGATACCGCGTGTGCTCCCTGCTGTCTGCGTCCGTGTCTGGCAAATTGG
TGAGGGGTCAGGCATCCCAGGTCTCCTTTTATCTTCCACACTGCCCCTGGGGGTAGAGAATTATGGTCCC
ATTTCACAGCTGAAGAAACTGAGGCCCGTCTGCGATGCGTAGCTAGCAGGTGCAGAGGCAGGGATCAAGG
CTCAGCCTGTTGCCTGCAAAGCCCTCGCCCTGTTCCCTATGGCGCACCAGCTGCTTGGATGCTCCCACGC
AGCAGTGCCTTGTCATCACATTCTATTTGCTGAGCGGCGATGGAACAGGCCCTGCGTCCCCGCCTGTGTC
CTAAGCACTCCACAAGCATGAGCGTGTCTAGATGGTGATGGTTTGCTCATGGTCTCTCTCTCAGAGCTTT
TACCCTGGCGGGGTTGGGAGCGTGGCAATGGGTCACCACTGACCCTGTTAGGAAACAAGTGTGTCTGTAC
GTGCCACTTATGATGGGGACAGGCCGTGGTCACTTTGCTGGGGCCAGTGTCAGCAGGATGGCAGATGCTT
TGAGAACACGGCTAAGAACTCTGCCTTCTGGAGCAGGATGTCAAATGCTGTTTGATTCAGGCATGTGACA
CCTGTCAAGAGCCCTGGCACAGCTGCTCTCGAGACACGCCTCGACTGCCACTCTCCCACCCCTGCGTGGC
ACCCTCCTGACTGTTTTATCTGCCTTGGTTTTTCTGGGTCTGGTGTAGCACCTGCCCCATAAGAAGCGCT
CAGTAAATGATTGTTGATAAAAGATTGAACCAATCAAGGAATGATGAGCCAGTGAAGGGCAAATGACTGG
AAGCAAAAGGCCCTCTTGATGGAATTGGGAAGAAGCTGATCCGAGGGCCCAGGACAGCCTTCTCTTGAAT
GCCCTGCTGCTGAGGTCTCACTGCTCGGCTTGGACTCGCCTCTCGCCCTGCCTGGCTGCAGAGGACAAAC
AGTGGGGTGGTGGGTGTGTGTCCGTGCCTGTGCGTAGGATGTGGTAGCTGGTGAGTGTCAGGGCTCTGCA
CGCTGCGCCCTGCCTTGCTTCTTTCTCTCCTCTCAGGCTGTGTGTAGTCCTGGGCCAGGTTTTTTCAGCG
GGCGTGGTGGTGGGAAGGTCCTCTGTCCCTTGCTCATCCTGTGTCCTGGCTCTTCCTCCTTGTGTCTTCT
CTCTGGGCCAGGAGGAAGTGGAGAGGTGGGATCAGGGCCGATGTCCCCAGCCTGGGTGGCAGTGTACTGG
ACCATGGGTTCCCACCCAGAACTCTTCATGGCAGAGACCTATGGCCTGCCTGTCCTCATCTGAAGATAAC
TTGGCCGTTCTGATTTCTCTTCCCACACAAGGCCATGGTGGCTGCCTCTGAGATTTAATAAGCCCTTAGG
TCAAATGTCTGCTTGGCCAGAGGAATTGTCTCCCGGGCTTTCTTGGAGACAGCTCTAAAGCATGCAAACT
ATAGTGTCAGGAGGAAAAGCTCTTCCTCTACCTACTTTGTTCTGTGTTTGGGGGTCTGAGAAATTTAACA
GTCAACAGATCAACAGTAGAAAAGACAAAGGCTTTTCTGTTTTTTTTTTTTTTTTGCCTTAAGCAAAAAT
CCTGCTAGGTTGGTTTAGCCAGAGCCCCCTCATTCCTGGTGTTTCCCCTTAGTAGTTTTTCCTCCACTGA
TCCCCACCCTGCTCCGGCTATTAATTCCTACTTTTCCTTGCTGTATATGTGGCTGAGCCCAACCTCTCTC
CCCAACTGCAGAATCGCATCACAGTGGGTCCTATACCTAAGGCGATAGTTCCTTCCCCCCACCCCTGACA
CCCCCAATAAAATGTGCCTTCTTACCATCATTAACAAGCATCATTGAATAATTTTGATTTAATAGCGGGT
TCGAGAGATCTCCTCCATCTTCATCCTGGCAGGGCTTCCCCTCCACCCCAGAAAGGAATTTATGGCAGTT
TCGCTCTGGGCCTCCTTCCTGGGAGTGAAGCTGCCTCTTCTTGAAGAGGGGGTTTATGGCAGCCTCACTC
CCAGAAGTTTCTGCTTTTAGTCAGATAAGGGAAACTCCAAAAATGCTTCTTTCTGGATCTGTTGAATCTC
AAATGTCTTCAGTTTAAAATAATCTTGATACCAACTCTGGGGGTTGGGGTGGGTCTCACACCACTCTCCT
ATGCATCCCTACCTGCTAAAAAGATGTGCCGGCTCTGGCCTGGCCGGCTCCTCCACCTTCCAGCCACCTG
TCCTGTTTATTCATTTCTTCCCTCTCTTTTCTCCTTTTCTTTTCCATTTATCCAGTCATTCCTTCCTTCT
ACCCACCCACCAACCTGATCACCCACTCATTCTTCTCTCCTTCAATCCAACCTTTCCTCAGTACCCAACC
TGTGCAACTGTACCATGGACTCCTTCAGGCAGTGGCTCCCAACTCCCATCACAGAGCAAATGCTCAACAC
ACACCCGTGTTTCATCTGTTATTCAACAAACACTGATCACTTGCTTCTCTTAATTATTATTATAATATTT
ATTTATTTATTTAGAGACAGGGTCTTGCTCTGTCACCCAGGCTGGAGTGCAGTGGTACAATCACAGCTCA
CTGCAGCCTCCACTTCCCAGGCTCAAGCCATCCTTCCACCTCAGCCTTCCAAGTAGCTGGGACTACAGGC
ACATGTCACCATGCCCAGCTACTTTTTGTATTTTTTTTTTTTTTTGTAGAGATGGGGTCTCACAGTGTTG
CCCAGGCTAGTTTTGAACTCCCGGGCTCAATCCTCCTGCCTCAGCCTCCCACAGTGCTGGGATTACAGGT
GTGCGCCACTGTGCCCAGCTGCTTCTCTTTGTAAGTTCCCAAGTTGAAGCACCCCCTTCAGGTGTAGTTT
TAGACGTGCAACATGAGGGGAAATCTTAGGTTAATTGCTGGGTCCAGAATTGCTCTGAAAGACAAAGACT
GTCTTTGTTCTAGAAAAGTGCTCATAGGCGCTGTGAAGGCCTTGATGTCCTCTGGTGCCAACGCAACAGA
GAAGCCAGCAAAATGCTCTGCATGGGACAGGTTCTGGTTTTTGCTACAGAGACTGTAAGAGATGCAAAGC
CAGGACGGTGAGGGTGAGCTGGGGTTGGGGAGGCGCAGGGGGGCTCCCCAGCAGAGGGAGGGCATTTGAA
GACGGCGGCTGCTCCTTGGAAGCTGGCAGGCACGGCCACAGAATATGCGCCCCTTTCCTGGCCCCCTCGA
GGTCTGCACAGCCCTGAGTGCTCATGCCATGAAAATGAGTGTCTCTGAGTTTGGAGAGCTGGCCTCCGCA
CTGGGCTTCCTGAGGACGGGAGCAGGAGCAGGAGGCTGAGGCCAGGGGGGATGGCAGCCCCCGGGGATCT
GAGGAAGTCTGGGAGCTGGCTTTGAACTTCTGCCCACGCTGGAACTTGGGGAGAAGAGGGAGATGTGAGT
CAGGTTTTCCTAGTTCCTGAGGGCCTGGGAGGCCTTGGCTGGCATCCCGTTTACATAGCGAGTGGCCCCA
CCTGATGGTTCTGATCCAGATTCTGGTACCCGAGTGAGCTAGGCCACGGGAGCAGCTGATGCCCCTGCTG
TCCCTGAAGGCAGCATGGGAGGCCCCCGGGACTGGGGCTCAGGCTTTCCAGAGAGAAGGAGGTGCCTTTC
CCTGCCAACTTCCTAGCTTTAAGTCACGCTCCGGACATCCAGAGTGAGCACCTGCACCTGCCAGAACCCC
TTGAGCCGCCTCTACCTTCCAGGATGACCCATTCCCAGTTTTCCCAATTACCTTTCATATTTCCTCTTTG
GAAATGGAATTTGATTTTGAGGCTGAGCAGGTGGCTGGAGACATAGAACATAAATATATCATGACTTGGG
AGTTGACTTGCTTATCATGGAAATAAATGTCCTCCCTCCTCAAGAAGCGGTCACTTGGCCAGGTCACTTT
GATCTTGCCCCGTGGTGAAATCAGACGGTCCCTGGCTCGTGGGCGCTGGAACCAGGGGGGAATTTCAGGG
TGAGTCATGAATGCTAGGGGGCAGGGGTGAATGGGCTGTAATGTCAGCTTTGTTTTCCTGTCAGGCGAGA
GAGAGAGAGAGGAGGGGAACAAGAGAGAATGATGCCAAGGAGGCGCCTGGGCAGCTGATGAGCTCAGCCC
CAGGGTGACTGCGGTCATTTGGGTCGGCAGTGGGGACAGACATCAGCCCCAGCTGGGTCATGGGTCATTT
ACGGCCTGGGTCTCCCCCTCTCCTCCCTCCTCCTTCTCTCCAACCTGCTTTCTTGCTGCTGGAAGGGGGT
GTGCAGGCTCCCGTGTGCCTGGCACAGCAGGGGCAGCACAGGAGACTCCATGGGGCCTGTGTCCCGCACC
CCCACGCCCCCGCCCCCAGCCACAGCCCTTTGAGAGCACAGCGGAATGAACGCCTGATTAGGAATCAGAT
GCCAGAAAGAAGCTGTGTCTGTTCCAAAATCAAGAGACCAGGAGAAGTGACGTCCTTTAAAAGGAGAGAG
CTAAGTGACAGAGAGGGGGCTTGTCCCCAGCCCAGGCACCTCGGGGCTGCAAGCTCCTTTGAGATGCGGG
GCTCAGGCCTCATCAGGGGAAACGTGGGCATCTAGATGACCCTCCACACTGAGCAGACCCAGCACAGGCT
GCGCCACTCATCATTATAGGCTGCGCCACTCATTATAGGCTGCGGGGCCTGCACAGACCCCAGGCATCGA
GCCTGCGATGGGGCAACGTGTGGTGTGGAGGCCATACCCCTCCTCTGCTGGCTGGCAGCTTGGCCCAGCC
TCCTCACTGGTGAACAGTCAGCTCTGCCCCCTGGGCTGTCAGGAGGATTAAATGAGATAAGACAAACACA
GAGCAACTCCTTGCTAACAGCCATGCAGTACCAAGCAAGGCACAGCAAAGGGAAAGAGAAATCCAGTGGC
ACCAGTGGGCTGAGAAACTGTCGGGTTTGGCTGCTGCTGTTGTCTGCATCTCCCGCCTTCCTCCTGAGCC
GTGCTTCCAGGGCAGCCCCGGCCCTTCATGTCAGCTGGTGACATTTCTGCAATTGTTCCTAAAGAGGGGC
CTGTGGGTCGAGTGAGCTAAGCAGGGCTTGGCAAGGTGGTGTCCCCCGGTCAGTGCCAGAGTCTCCCTAA
TGTGGCCTGTGAGACAGAGCTGATGCTGAACAAAGGAACAGGGTGATCTGGGAGCAGCAGCAGCCGCTGT
CCTGAGCATCTGGGTCAGGACGACACGGATGCCACCCCAGGACCCCTGGCATAGGGAGCCGAGCAGGGCA
GCCGGGCAGAGGCAGGAATGAATCCTACTCAGCTGCAGAATAGTCACAGACACCTCTCCACCAGCAACGT
CCCTGAAACAGTCTTTCTTATTAAAAAATATTGAGTGTCAGTTATGTGTCAGGCACTGTTCTGGACACGA
GGTCTCAGGAACAAACAAAATATACAGAAACTTGGCCCTTGTGTTGCTCAGGATGAAGAGGAGAAGACAG
GAGCTGTGAGGTGTCCCCACCTGTCCAGAGGCTGGGAAAACTTAGGAGACGGTGCTGCTTGTTTAGAAAA
CCGGGCAGAGGAGCCCTGCTAGTCCAGGCCGGGCCAGCGCCCAAGGATGAGGGGAAGCACAGCTCCTTTG
GGGTCTGCGTGCATTTGAGCATGAGTGCATTAACAGGGGGAACGTCTGTGTTTACAGGGCATGCGTTTGG
GAGCCAGACAGACCTGGGCTGAGTCCTGCCCTACATACTAACTAGCCTGCTGTGTCGCCTTGCAAGGGGT
GAGATTTCTCTGCTCTAAGTCATGGGTGCTGAGGCCCACCTGTCTGGCGAGGGTCTGGGGGAGGTCGGAT
GAGATGAAGGAGGCTGGGGCTGGCACCGTGGTGGTCCCTGGGCTGTGCCTGTTTCCCTCCCACTTCTGCT
GCCTCCATCTGACCCTGCTCCGGAGGGACCAGGGGAGGGGCGGACGGAGGACACTGCTTCTGCAGCGCTC
CTTGTCTCCGTCAGTGATTTATAGGAAGCTGGCTCAACCGAGAGCTGAGGGAAGACGGTGAAGGCCCTTT
TGTTTTTAGTTCAATTTTGTGAGCAGAATGCTCCTGGGGCCAATAGATTTTTCCAGTAATGAAATTGTGA
AAATAATTGAATTATGCCTTTCCAGCTTCAAAGACAACCTGTGCCCTTTCCCCAGCCCTCCGCATGGCAT
TAGAGAACATTCGCCTCCTGCTGGGTCGGCCCCTTCGCCTCTTCCTTCTGTCTGTCGGTGCAGCCTCTGA
ATAGAATCCTGGCGAAGGGAGGGGCTGGGTGTGTTGTTGCTGCACCCAGGACTGCCCACATCAGTTGCAA
AATGCAATGTGGGGCCCTTTGTTGAAAAACAATGAAGAATTTCAAGATGGTGGCAGCAGAGCCTGAAACC
GAGCTCAGGGCCCATCTGAGCAGGGGCCCGGTGTGACCACACGGATCCCATACCGGTGATGCCAACCCCC
TGAGTCCCGGGGAACAGTGGCAAATCAGCACTCAGGAAATATCTGTTGAATGGAAAATCCAGTTTCCTTC
GCCGTCTTCCTTGTCTGTCCTCTCCTGCCGCCTCCATTCAACTTTCTCCCTTCTTCTCACAGCACTCGGC
AGTGTGGACTCTGGCTGCCTGGGTTCAAATGAGCTCACCACCTCCTAGTTGTAAGCTCTTCGACAAGTGA
CTTAAACACTCTGTGCCCAGTTTTCCGCATCTGCAAAATGGGGAGATAAATAGCCCCTACCTCCTAGGAT
CATCATGAGAATGAGGTGTGCGAAGCTTGGCCGGCATGGGTTCCATAGCAGGCACTCAGGGGTGTCGGCC
ACGAAGATTATTCTTTCTCTTCTCTCTTTGCCGTCTTATTTCATCTCTCTCCGTTATTTGGTTCCCCTGT
CCTTAGTCCCCTTTCTCCCCCAATGGCATCCCAAGATGCACAATAGTGGCAAGTGCCCAGCCTGTTTCCA
CAGCCTGATCCCCACCACTGCGTTGGCCAGTCACCCAAGAAGCAGCTGGACCCATCATCTGGCTCTAGGG
ATGACCCAGTTCCAGCACCCCCGCAAACCTCCGTCTGTCCCCCTACCTCCCTCAGCAGAGGCCCAGCCCA
ATGCAGGCCCGTGGCTGGATGGGAGTAGCTCTTCCCACCACCCCTGGGCAGGGCTCTGCGGAGCTTGGGA
GCCTCACCTGGAATCGGCCCTCATGCCTCAGTAGAGAAGGAGAGCGAGGAGAGAGGTGATGGGGCTCCGC
GGGCACCCCCGATGCACAGTCTCCTTCTGGGCTTCTGATGGCCACAAGGCCAGAAGACCTGCCCAGAAGA
ATTCAGTATAACCCAGTTCAGTGAAATTGGAGAGAACGAGGGCCTGCGTCTTCCGGGCAGAAGGCAGGGT
TCCTGCCCTCTGGAGCCCTTGGCCTGGCGCGGGCTGATTAGGACCTAGATCTGCCTGGGTGGCTGGGTGG
CCGAGTGGCGATTGGGCTGGTTCTGTACCGGGTGTGCTCCGTGGGGGGCGTGATCTGGCAAAGCCTTGGA
GGTGGGACTGTGGAGGCACCATTGATTGAACTGTGTCCCCTGCAATTCACATGTTGAGGCCCAAACCCCC
AGTGTGGCTGCATTTGGAGTAGGGCAGTAATTATGGTTAAATGAGGTCGTATGGGCGGGTGCTGATCCAC
TAGGATTAGGATCCTTATAAGAACCTGCCACCTTCTCTCTGCCACGTGAGGACATGGGGAGGAGGCGGCT
GCCTCCCACCCAGGAGGAGCCCTTACTGGACACTGGGCCCTGGCTGCACCTTGACCTTGGACTTCTAGTC
CCCAGAACTGTGAGAAGTAGATTTCTGCTGATTACGCTTTCCTGTCTGCGGCCTGAGCTAAGACAGCAGC
GCTTGGGGAGAAGCAGAATTTGAGGAGCTCCTCAGTGGCAGGCTGCCCTGGCCCTGCTGTCAGCAGAGGG
GAATGGCCATCCATGCTGGCCCCTCACCAGCCGGGCCTTCAGTGAGCTCCCCGGGTAGGTGAAGCTCTCC
CAGCTCTGTGTCCCCCGCCAAAGCAGGCCCACAAGCGAGCGCCTATGGGGTGGAGTGAGAGTGAGGAAGA
AACATTACCCGAGGGGTCACTCTCTTCAGAAGACCTCAATGACTGTAGACTACTGAATTATTTCCTTAAA
AAAAAAAAAAAAAGGCTAGGTATGGTGGCTCAGGCCTATAATCCCAGCACTTTGGGAGGACAAAGGACCA
CCTGAAGCCAGGAGTTCCAGACTAGCCTGGGCAACACAGCAAGACCCCATCTCTACAAAAAATTTAAAAC
TTAGCCAGGCGTGGTGGCACATGCCTGTAATTTCAGGTATTTGGGAGGCAGAGGCAGGAGGATCACCTGA
GCCCAGGAGCTAGAGGCTGCAGTGAGCTATGATTGCACCACTGCTTTCCAGCCTGGGTGACAGAGTGAGA
CTCAAAAATGGTTAAAAAAAAAAAAAGAAAAAATGTTGATAGCTACTATAAAGTTTCTCTTATGCAGTAC
CTCCTCATTTTACAGGAAATTTGGAGATAGGGAAAATAGAAAGAAGAGGAAAAGATGCCCAAATATACCC
AGAAAGTCCCTGCTAACATGCTGCTGTCGTCCTTCTATTCCTTAATCTAGGCATGTGGGCTTTTTTCTTA
TTTAAAAATGTTGATTTAGATATAATTACATGCAGAAAAGTGCACAAATCTGAAGTCTGCAGTTTGAGAA
GTTTTAGACATGTGCATACTCTGCACCGACCACCTCTGCTAGGATATAGAGCATGGCCAGTGCCCAGAGG
GCACCGCAGGCCCTGCCTAGCCACACTCACACTCTCTTCAGTAACCCCTCATTCTGATTCTATTGCCATA
GAATAGTTTGGTCTTTCTTAAACCTCATATAAACGAACCATGTTGTATGTGGTCTTTGTGTCTGGCTGTT
TTTTCCCCTATTTTAAAAATTGTGTTAAAATACACATAAACTTTATCATCTTGACCATTTTTAAGTGTAC
AGTTCAACGTTATTAAATACATTCATAATGTTGTATAACCATCACTGCCATCCGTCTGTAGAACTCTTTT
CATCTTGGAAAACTAAACTCCATACTCATTAAACACTAACTCTGTATTCCCTCCTCCCCGCAGCCCCTGG
TAAACACCATCCTACCTTCTGTTCATATGAATTAAAAAAAAATTTTATTTTAGTTTTTGAGACAGAGTCT
CGCTATGTACCCCAGGCGGGAGTGCAGTGGCGCAATCTCGGCTTACGGCAACCTCCGCCTCCCCGGTTCA
AGTGATTCTCCCCATCAGCCCTCTGAGTAGCTGGGATTACAGGCACACACCACCACGCTCAGCTAATTTT
TTTTTTTTGATATTTTTTAATAGAGACAGGGTTTCACCATGTTGACCAGGCTGGTCTCAAACACCTGACC
TCAAGTGATCCACCTGCCTCAGCCTCCCAAAGTGCTGGGATTACAGGCATGAGCCATTGTGCCTGGCTGT
GTTTCCTATGAATTTGACCATTCTAGGTACCTCCTATGGGTGGATTCATACAGTACTTGCCTTTTTGTGT
CTAGCTTATTTCACTTAGCATAATGTCTTCAGAGTTCATCCATATCTGTAGCATGTCTGAATTTCTTTCC
TTTTCTTAGGCTGAATAATATTTCATTATGGATATCATGGCATGTTGCTCATCCATTAATCAATCAGTGG
ACACTGGGTAGCATCTGCCCAAGTTTTAGACATTGGGAATAATGCTGCTGTGAACATGTGTGCACAAAAT
AACTCTTCAAGACCCTGTTTTCAGTTTTTTTGGGCATATACCCAGAAGTGGAATTCCTGATCATATGGTA
ATTCTACTTCTAATTTTTTTTCTTTTTTTAGATGGAGTTTTGCTCTTGTTGCCCAGGCTGGAGTGCAATG
GCGCAATCTTGACTCACTGCAACCTCCGCCTCCCGGGTTCAAGTGATTCTCCTGCCTCAGCCTCCCAAGT
CGCTGGGATTACAGACATACGCCACCATGCCCGGCTAATTTTGTATTTTTAGTAGAGACGGGGTTTCTCC
ATGTTGGTCAGGCTGGTCTCGAACTCCCAACCTCAGGTGATCTGCCCGCCTCGGCCTCCCAAAGTGCTGG
GATTACAGGCGTGAGCCATCGTCTACTTCTAATTTTTTAAAGGAACCACCACACTGTTTTCCACAGTGGC
TGCATCATTTTACATTTTGTATCTGGTTTCTTTTGCTTAATATTGGTCAGGGAGATTCATCAATGTGTAC
AGCACAAGTTTGTTTCTTTATAATTGCTGTGTTAAGGATTCCATTGTATGAACAAAACACAATCTATTAA
TTCCCCTATTGATGGACATTTGGATTGTTGCCCACTGTGGCTATTATGAATAATGCTGTCATTAACCGTC
TTGTACACATTTTCTGGTGGTCATAGGCACCTGTTTCTCCTGTGGGTATATTTGGGAATGGAATTGCTGG
GTCATAGGGTAGGCAATTGTTTCACTTTAGTAGATATTAGGTGTAAATTTTGATGTCTTTAAAATAGTAC
TGATTGAGCCGGGTACAGTGGCTCATGCCGGTAATCCCAGCATTTTGGGAGGCCGAGGCGGGTGGATCAC
CTGAGGTCAGGAGTTTGAGACCAGCCTGACCAACACAGTGAAACCCCATCTTTACTAAAAATACAAAAAT
TAGCTGGGCGTGATGGTGGGCATCTGTAATCCCAGCTACTCTGGAGGTTGAGGTAGGAGAATCGCTTGAA
CCCGGGAGTTGAAGGTTGCGGTGAACCAAGATCGCACCATTGCACTCCAGCCTGGGCGACAGAGCGAGAT
TTCGCTTCAATAAAATAAAATAAAATAATAGAATAAAATAGAATAAAAAAAAATATGGTACTGATCATCA
CATAAAGTTTTGAGGTCTGCCTTTTTCACTTAACATTAAATCATTACTATTTTTAAGTTAGAACTTTTAT
TTTGAGATAATGATAGATTCCCATGTAGTTGTATGGAGTAACACAGAGAGATTCAGTGCACCTTGTACCC
TTTTCTCCTGTGGTAACATCTTGTGAACCTAAAGGCAATATCATAATAACTATATTGATGTTGATTCAAT
GTCCTCAGATCTCCCTAGCTTCCTTTGCATCCATTCGTGTGTGTGTGTTCCTCTAGTTCTACACACTTTT
ATCACCTGCACATATTTGTGGGTCCACCACCACAGTTCCAATGCTACAAGGATCCCTCCTGTTTGCTTCT
TTCCTTTTCCTTCCTTCCTTCCTTCCTTCCTTCCTTCCTTCCTTCCTTCCTCTCTCCCTCCCTCCCTCCC
TCTCTCTCTCTTTCTTTCTTTCTAGCTGAGATTACAGGCATGCGCCATCACGTCTGTTTAATTTTTGTAT
TTTTACTGGAGATGAGGCTTCACTATGTTGACCAGGCTGGTCTCGAACTTCTGACTTAAGGTGATCCACC
CGCCTCAGCCTCACAAAGTGCTGGGATTACAGGCATGAGCCACTGCGCTCAAGCTTTTTTTTTTTTTTTC
TTTAGACGGAGTCTCGCTCTGTCACCCAGGCTGGAGTGCAGTGGCGCGATCTCGGCTCACTGCAAGCTCC
GCCTCCCAGGTTCACGCCATTCTCCTGCCTCAGCCTCCTGAGCAGCTGGGACTACAGGTGCCCACCAACA
CGCCCGGCTAATTTTTTTTTTCGTATTCGTAGTAGAGACGGGGTTTCACCGTGTTAGCCAGGATGGTCTC
AATCTCCTGACCTCGTGATCTGCCTGCCTCGGCCTCCCAAAGTCCTGGGATTACAGGCGTGAGCCACCGT
GCAGGCTTTTTTTTTTTTTTTTTAATGTGAAGTCTCACTGTGTTGCCCAGACTGGAGTGCAATGGCACCA
TCTCAGCTCACTGCAACCTCCACCTCCTGGGTTCAAGCAATTCTCCCGCCTCAGCCTCCCAAGTAGCTGG
GACTACAGGCACCTGCCACCATGCCTGGCTAATTTTTGTATTTTTAGTAGAGACAGAGTTTCACCATGTC
GACCAGACTGGTCTCGAACTCCTGACCTCAGGTAATCCACCTGTCTCGGCCTCCCAAAGTGCTGGAATTA
CAGGCATGAGCCACTGTGCCTGGCCACCCCTCCTGTTTGTTTCTAACACAATCATCTACCCCCAGCCTCT
GGCAACCACCAATCTGTTCTCCATCTTTATACCTTTGTCACCATAAGAATGGTCTGTACATGGAATCAAG
CAACTAGTAACCTTTTGGGATTGGCTTTTTGCAGTCGGCATAGTTCTCTGGAGAGTCATCCAGGTTGCTG
TGTGCTGTGATTTGAATGTTGGTGCCCTCCTAAATTCATATGTTGGAATCTCACACCCAATATAATAGTT
TTAAGAGGTGGGACCTTTGGGAAGTGATTAAGTCACGAGGGCTGTGACCTCATGAGTAGGATTAATGCCC
TTATAAGAGAGGTGAAGGGAGTGCCACTGCCCCTTTAACCACGTGAGGACACAGCAACAAACTGCCGTCT
ATGAATCAGAGAGCCCTCAACAGACACCGATTCTGCTGGCACCTTGATCTTGGACTTCCCAGCCTCCAGA
GCTGTGAGCAATAAGTTTCTGCTCTTTATAAATTATGCAGTCTAGGGTATTTTGTTATAGCAGCTGGAAC
AGACTAAGACACTGTGTACCGATGATTTATTCTTTTCGTTACTGAGTGGTGTTTCTGGATTTGGACATAC
CGCAGTATGTCTGGCATTCATCCTTTTTTTGTTTGTTTGTTTTGAGACAGAGTCTCACTCTGTCACCAAG
GCTGCAGTGTAGTGGCGCGATCTCAGCTCACCGCAACCTCCACCTCCTGGGTTCAGCTATTCTCATGCCT
CAGCCTCCCAAGTAGCTGGGATTACAGCCACATGCCACCACGCCCGGCTAATTTTTGTATTTTTAGTAGA
GACAGGGTTTCACCACGTTGGCCAGGCTGGTCTCAAACTCCCGACCTAGGGCATCCACCCGCCTCGGCCT
CCCAAAGTGCTGGGATTACAGATGTGAGCCACCACACCTGGCCTGACATTCACCCTTAAAAGGACATTTG
AGTGCTACCAGTATTTGCCTGCTTTAGTAAAGCTGCTATGGAGACTAGTGTATAGATTCTTTTGTGAACT
TACATTCACACAAAAATGCAGTTGCTGGGTTGTGTGGTATTTGCCTCATAACTTCAGATGCTTCTTGACT
CACAACATGGTTACTTCCCGACGAACCCATTGTAAATTGGCAGCGTGGCTGAGTGGGAGCTGCATCTCTC
TGCCTCTGTCCAGCACTGTGAGGGAAGTACAGTTTCTATTGAATGTGTATCACTTTCACACCATCATAAA
GTCAAAAAATCCTGAGTTGAACCATCATAAGCCAGGGATTGTCAGTATTTTTAGAGGAGCTCAGGGTCAT
AGGTTACTCTGCCAGTTACATATTCTGATGCAAACGTTTTCTGAAGCCTTTATTATTATGACATTCTGGG
GACCTCCATACCCCTTATTTTTGGGGATTAGAGCTTCCCTCTGTCTAATCCAGCTCTAGAGCCACCTCCT
CGGGAAGCCCTCCACCCAGAGCCACCTCTCTGTGAGAGCGGCCCCACCAATGACTTTCCTGTGGGATGAT
GGAGCCCTCATGCTGAGCAGAGGTTAATGCCATCCCACGGCTCCCTTCCCCCAGAGGAGGTCTGGGTCTC
AGCTGAGGCTTACAGCCTAACACATCTGGCTCCGGACATACTGGGAGCTCTTTACCTGAGGGTGTCCCTG
TCATGTAGACAGAGGCTTTTGAGAAAATAAACAGTCCTGAGAGAACCTCTTCGTCCTGGGGGAAGCTGGC
TTGGTCTGGTGAAGACTTGTTCACTTGCTTCCAGCAGAAAATAACTTCGCTGTGAGCCTGGAGGTTATTA
TAACTAAATCATTCTGTGAACTACACAGACAGGCTGGGCATGGCCATTGTATTTCCGTCACCCTGGAGAG
AAATCTGTGACCAAATGGTTAAAAAAAATTCCAAGTCATTCATTATTCACCCTCGCTGCATAATGCATGA
AGGCTGCGGGCCAGGCCCATTTATGTTTTCAACATTGGCATCAAATGTCAGCCACTGCCTCAGAGAGGCC
CACGAGGGCCTTGGCACTCGTGGTTGGCCCCTTCTCCCCTCTGGATGAACCTCAGGCCTCCAGGGCTCCT
GCCTCCTGCTCACTAGGCTCTCAGCCTGGATGGTCCTTCTTTCTCTTCTTCTATGAAGCCCTCTCCATCC
TTTAGGGCTTAGAAATCATCTCCTCCCTGTCACCTGCCAGGGCAGGAAGTCATCCTCCCTCTCTGTTCCC
CCTGTAACACTTTATTTTTATTTATTTATTTATTTATTTTTTTGAGATGAGACGGAGTCTCACTCTGTCG
CTAGGCTGGAGTGCAGTGGCATGATTATGGCTCACTGCAACCTCTGCCTCCCAGGTTCAAGCGATTCTTG
TGCCTCTGCCTCCCGAGTAGTTAGGACTGCAGGCACGCACCACCATGTCTGGCTGATTTTTGTATTTTTA
GTAGAGATGGGGTTTCAACATGTTGGCCAGGCTGGTCTTGAACTTCTGACCTCAGGTCATCTACCTGCCT
CAGCCTCCCAAAGTGCTGGGATTACAGGTGTGAGCCACCGCACCCAGCCCCCTGTGGCACTTTAGAGTAT
TGATAACCACCACCACTGTGATCATAACTAGTATGATTTATATAGTTTGTATTCTCTGCTGGACACTTAA
CTCACACTATCTCATTTAATTCTCATAATAGCCAGTGAGGCAGATATTCTATTTCTCAATTTTAAGATGA
GGAAACAGGCTTAGAGTGGTTAATTAATTTGCCCAAAGACATGCAGCTAGCAAGAGGCAGCATATTGCAA
TTTGCCTGCAGGATGGTAGCAATTGTCATCCCTTTTCTTCTCAATCATTCTCACCAGAAATGTATTAGTT
TTTGCAGCCTTTTCAAGGAACCAACTTTAGGTTTCGTTGATTGTCTCTGTTTCTTAGTTCATTACATGTG
TATTTTTATTTCTAGTCCTGCTTGCTTAGGATTTATTTTGCTGTTCCTTTTGTAATTTCTTGAGATAGAT
CTTAGTGCATTTGTTTTTAATCATTCCTCCTTTCTCCAGTTCAGGCTGGTCTTGAACTCCTGGGCTCAGG
CAAGCATTTCAGGCTAGAAATTTCCCTCTGAGTACTGCTTTGGTTGCATCCCTGCACATACAAGTTTTGA
TTGTAGGAGTTTATTATTCCATTGAAAATATTTTCCGATTTTCCTGGTGATGTCCAGGTGGGATTGTGCT
CAATCTGACAGACTGCACAGCTCTCTCTCTCTCTCTCTTTTTTTTTTTTTTTTTTTTTTTTTTTAAGACA
GGGTCTCAGCTCTGTCACTCAGGCTGGAGTGCAGTGTTGCGATCATGGCTCACTGCAGCCTTGACCTCCA
GGGCTCAAGGGATCCTCTCACCTCAGCCTCCCGAGTAGCTGGGACTACAGGAGTGAGCCACCACATGCAG
CTAATTTAAAAAAAAAATTGTAGAGATAAAGTCTCACTATGTTGCCCAGGCTGGTCTTGAACTCGTAGGT
TCAAGCAATCCTCATTCCTCAGCCTCCCAAAGTGCTGGGATTATAGGTGTGAGCCACTGTGCCTGGCTCT
TTTTATGATACTCTTTTCTGACATTGAGCAAATCATTTTCATGTCTGTCCCTCCCCCAGGATCAGTTTGA
GACCAGCCTGGCCGACATGGTGAAACACTGTCTCTACTAAAAATACAGAGACTGGCTGGGTGTGGTGGTA
CATGTCTGTAATCCCAGCTACTTGGGAGGCTGAGGCAGGAGAATTGCTTAAACCCGGGAGGCGGAGGTTG
CAGTGAGCTGAGATAGTTCCACTGCACTCCAGCCTAGCAGCCTGGACGACAGAGGAAGACTCTGTCTCAA
AACAAACAAACAAACAAACAAAAAAAAACCCAAAAAAAATCCCACAAAAAACAAAACTATGAACTAGGTG
GTCTAGAAACAATTATTTCTCACAGGTCGTTTTAAGCCACTGAGTTTGTGGTAATTTGTCACAGCAGCCC
TAAAAAATAAATATACACAGTTTTAATTGAAGTTGGGCTAAGAAGCTGGACGCTAGTCCCAGCTACTCAG
GAAGTGGAGGTGAGGGGATCGCTTCAGCCCAAGGAGTTCGAGGCTAGCCTGAGCAACATAGCAAGGCCTC
ATCTCTAAAAAATAAAAATTAAAAATTGGGTTAAGAATTTAGCAGCTAGTCCTTAATAGGATGAGCCATG
AGGACCGGCAGCTACATATCAGTGGTTTGAAGCAAATGGTTGCCAAGTCGCTCCTCTCCCTCCCAGGTCA
TCACCGTGACATCCTTTTGTCCAGTGTCCTTCCCTCCAGGGTAGCTCAGGTTAGGGGAAACAGCCAACAC
ATCCATGCAAGGAAATAAATGGCCACTCAAACGCAGACTCTTTTAAAAGTGGGAGAGTGGCTGGGTGTAA
TGGTTCATACCTGTGATCCCAACACTTTGAGAGGCCGAGGTGGGAGGACTGCTTGAGCCCAGGAGTTGGA
GACCAGTCTGAGCAACATTAGGAAGACCCTGTCTCTACAAAAATTACAAAAATGAGCCAGGTATGGTGGC
GCGTACCCAGCTACTTGGGAGGCTAAGGCGGGAGGATGGCTTGAGCCCTGGAATTCGAGACTGCAGCGAA
CTATGATGGCACCACTGCACTCCAGCCTGGGCCACAGAGCAAGACTCTGTCTCTAAAACAAAACAAAATA
AATAGAAGGAGGAGAGGCGTGAGCATTGAGAGCATGGCCAAAGAGCGGCAGCACTGGCTACCCAGCACTT
ACTAGCCACATCTGGGCCTAAGGATTTTATGTCCCTCAGGACTCACGGGGCTGATGTGCCATCTGACCCC
TCTGAGACCTGTGGGACTGGGCTCCGAGCCTCTGGGACACTGGAGGGGTGGAGGCAGGTGTCTGGGCAAC
ATCCAATAAGAAGCCTGTGACAGAGTCAACAGTGGCAAGAGCCCTTTGGGCCAGCCCGGCTCATCCCCTC
CTGGCTGCCTGCCCCTCCATGGCAAGGTCATTTTCCTTGATTCCGATCACAGCAGACCTCTGTTGCTCTG
TGGCTGATTCATTCAGAGAAGCAGGGTTCTGGCTGGGTATGGTGGCTTATGCCTGTAATCCCAGCATTTT
GGGAGGCCAAGGTGGGCAGATCACTTGAGGTCAGGAGCTCGAGACCAGCCTGGCCAAAATGGCAAATCCC
TGTCTCTACTAAAAATAAAAAAATTAGCTGGGCATGATGGCACGTCCCTGGAGGCTGCAGTGAGCTGAGA
TTGCGCCACCGCACTCCAGCCTGGGTGACGGAGTGAGACTCTGTTTCAAAAAAAAAAAAAGGCCAGGTGC
GGTGGCTCACGCCTGTAATCCCAGCACTTTGGGAGGCCGAGGCGGGCGGATCATGACGTCAGGAGATCGA
GACCATCCTGCCCAACATGGTGAAACCCTGTCTCTACTAAAAATACAAAAACAAAATCAGCCAGGCGTGA
TGGCAGGCGCCTGTAGTCCCAGCTACTGGGGAGGCTGAGGCAGGAGAATGGCGTGAACCCAGGAGGTGGA
GCTTACAGTGAGCCAAGATCGCACCACTGCACTACAGCCTGGGTGACAGAGCGAGACTCCATCTAAAAAA
AAAAAAAAAAAAAAAAAAAAAAAGCAAAAAAGCAGGGCAGCAGGGTTCCTAGCCCCGGCTCCACTGCCTT
TTCTGGCTTGGGGAGACAAAGCTTGGTAACATCAGGACTGCAAACATCCTGCCATTAGATCTTGGACCAG
CACCGGCCTGGTAGTTTGCTGTTTCATCAGGGGGCTTGCAGTTCATAAGCAATCCCTTCTTTTCTTTTCT
CCCAAAGCTGGGAAATCAAAACAAAACTTCCTTCCCTTCCGATATTCCTCTTTACTAAAAATTTGCGTAG
CCAACTCTCCTGGGAGTGGGGGGACCAACTTGGCTCAGGTTGGCTGGGATGGTCCTGTTTTTAGCATTGA
AAACCACAAAAGCAATGAAAGTCTCACGTTGTGGGAGGCCCTGAGTCCTGGGCAAACTGGGACTGTTGGT
CCAAACCGTCCCTTCCAGAAGAGGCCCACATGGCAGGCCCTTTGAGGCCTGGGGCAGGCGGGACAGAGGC
AGCACCCGCCTGGCATGGGGCTGAGGCCTCCTTGCAGCTGCCTCCCGGCCCCTTGTTCTCCTTCATGGCT
CCATGCACAGCTGAGGGGAGACATGAGGTCTCTCGGTCTCCATGGGTTTGGGGCAGGCCCAGAATGGCCC
TGAATCCAGTAACACCAACTGTCCCTGGAAGTGTCACCACCCTGGTCTGGGAGGAGCTCCATGAACATCT
CAACCATCCACTTCTTGTGCAGATGGGGAAACTGAGTCAGAAAGGGAGGAGACTGCCTTAAGAAAGATTC
TTGCAACTTGACTTTGATCCTGTAGAGTGTTTGTGGGTTTAGAAGAGCACAGGTCTGAAGATGGAAGACT
TGGGTTTCAGACCCGTCTCTGCCCTGTGCACCAGCATGCCCAGAGCAGCACTAACCCAAAGCCAATCTGA
CAAATGAACAAGTCATTCTATTTACTGGTTTCTTTGATCTATTTATAAAAATGAATATTTATAGCAAATC
CTGTAGGATGCGGTAGATTTGGAAGATTTCTGTGATGTTTTGTTTGACTAATGCTTATTTTCCCTTTTGT
ATCAGTATTTTAACAAATGCTTTGGATTTCCTCCACGCAGAGGAGAATCAGTATGCAGCTGTTAATTTTG
GCTGCTGACTCCAGGACAAGAGACAGAGAGAGAGCGTGAAAGCGAGACAGAGCGTGAGAGTGAGAGAGAG
AGAGAGAGGCAGGAGAGGCTGAGCGTGGGAGGGAGACAGACGGCATCGATTCATTAATATATTCAAAACT
ATATTCAAAAACTATTCATTTAAAAATTCTGGAATATATTGACTACATACTTTAAAAGACTTTTTTATAA
TGGGAAACTTTGAACATAGGCAAAAATAGAATAATAGAAAAGCCCAGCGTGCGACATCAGTGTCAACAGA
GGTCCATATGTGGCCAGTCTTGTCTCAGCCTTTTCCCCACCCATTCACTCCTCTCCCAAATCCGGATGGT
TTTAAGGTGATTTTAGCCATCGAATTCCATATGACTCTGTTCTCAAGTGTACTGGATACATTCTTGAAAA
TACTCTTGAACATATTCTTGAGGATGAGAAATTCTTACAAATGTATATTTTTTGTGCCCCTGTGTCTCCC
CTTCCTTGCCTTTCAAACTCCTCTCTCTGTCCCTTTGTCTCCTTTCCCCCACCCTTCACCTCCCCTCTCT
GTTCTCCCCAGTCTCCCCTAAACTCCAAATCTCTACTCTGACCCTCAGTCTCATCTTCTGGGTGACCGAA
GATGGGGCAGTGCAGCCCCTGCTGAGCTGACCCTCCTTTCTGTCCTTCCTCGTGCCGAGGTTCTCTCTCC
TTGACTGTTTCTCCTAGCTGAAGCAGTAGGCTGCTGGACAGAGGCAGGTCCAGCATACTTTGAATGTTGT
TATGAAGTCCAGTGGAGAAGAGCTAGCCTAAATTCGGCAGAAATCTTCAATATCTTTAAAAAAGAAATCC
CTCCATTATGGATCACTGTAAACTTGTATCAGTTGTTATAATCAATGAACACATTGACTGAATTGGCCTT
TTGATATATTGACTTAGAGCTGTGTGATTTTGGATGAGCCATTTTACCTTTTAGAGCCCCAGTTTCCATC
TCTGTGAAGTGGGGAAGTGTGGACCATAGCAGTGGTTTTCTTTTTTCTGAGGCAATTCTCCTGCCTCAGC
ATCCCGAGTAGCTGGGATTACAGGCGCGCACCGCCATGCCCAGCTAATTTTTTTTTTTTTTGTATTTTTA
GTAGAGACGGAGTTTCACCGTGTTGGCCAGGATGGTCTCGATTTCCTGACCTCGTGATCCACCCACCTCG
GCCTCCCAAAGTGCTGGGATTACAGGCGTGAGCCACCGTGCCCGGCCAGCAGTGGTTTTCAAACTGGTTT
CGGAAGCAGATTCATTCTTTTCCTAGCCAAAGCCTGGTGAACCGTAGTGTGTACAACAGTTAAAAGCAGA
GACCTGCAGGTCCACAGGAGCAGACGCTGAGACCAGCCTGCCTGGGGTCAATGCCCTCTAACCCCTCTGT
GCCTCTGCTGTTCATCTGCTGAATGGGTCTCATAGTTGAGCAGTGGTCGAGAGATTTCCTGGCATCAAAT
ACATGGAAGGGACTTAGCGTAGGTCCTGCTTAGCACATAGTAAGTGCTCAGCGAACTTACTTACCACTTA
TTACTTACCACCCCTGCCTGAAGCAGGGTGGGGCCAAGGGATTGGCTGGACCTCAGCCACTACCCCTCCC
CTCACATGGAGCCAGGCCCCACCTCCACAGATTGGGGGCTTGGGGACACGTCTTCTGTGCTCCATGCTCT
CAGGTCCCAGTGAGGCTGAGCGCTGAACCATCCAAGTTGGCAGGGCCAGGACAAGGACCCAGGAGCTCTG
GTGCCCAGGAGTCCCCCGAAGGGGGGCTCAGAGGAGGTGCGTGCGGGGCTGGTGCCGGTGCCCCCGAGGC
CCACGTGCAGCCCCGCTCTGTGGGGCCTTTGCCCAGCCTCCTGGTCCTTGCCAGCGCTGCATGGCGTTTC
CTGCCGGAGTGCTGGTCACTGAGGGCTCTCAGACAAGGAATGACCCAGGGGCTGGGCCCAGCCTCTCCCT
TGTTCCCTGATGCTGCTGGGGCCGCTCCTCTCTACCCAGCAGAAGGGTCCCGCAGGAAGATCAGCTCTGA
GGTTAATTGTCCTTCTGGATAGCTGCAGCCCAGTTGTCATGGAAACCGGTCACGCAGGGCTCGGTAGCAG
GACCCTCCTTCCTACCTCTGCCACGGTGCCGCAGGCTCAGAGGGGGCGTGGGAGCTGCATCAGGTGATGG
TAAGATACTACAGCCATTCTCCAACTGCAGGCACCTGGAAGGAGAAGGTGGCCCGGGGCCAACTGTGCAG
TGAGAGAGGCTTTTTCTGCGTCTCCCCGCAGCTGTCTTGTTTTCAGCAGTGGCCCTGGGGGAGATGTGGA
CGCTCTGAGGCCTGGGGCTGGCTCTGTGTGTCCAGGTCTGTGTGGGGGTCACTGAGAGAAGGGAAAGGCA
GGGGATCTGGCCCCATGTGGATCTTGGTGGCCCTGGCACTTTTTTACTCTGAGCCCAGCCTTCCTTGTGT
GTAAAACAGGGATTGTGGTGCCTACCTCTTGGGCTTGCCCTGCAGATGGGCCGGGCTGAGCCACACCACA
GCGGCTGGGCAGGTTCAGGCCCCTTCTCACCTCTGCTGTCCTCCAGCACCATGATTCGGTTGGGAACAGA
GGAAGGCGAGGAGAGCCATCCGCTTCCTGCTTCTGCAGCATTCGGCCATTTGCCCAATGCTCCCTGATGC
CACGGTTCTCAAAGTGTGGTCCCTGTCCCTGGCCTGGGAGCATCAGCTTCCTGTGGGGACTCTAGAAATG
CAAGTTCTCCGGCCCCACACTAGACTGACTGAATCAGAAACTCGGGGCGGTGGGGTCTAGCGATCTGTGC
GTTAACACATACTCTAGGGGGTTCCGATGCCCCTCGAGTTTGAGACCCTCTGGTCTAATGATTTATGGGT
GAGACACTCTGCGCCTGCTTCCCTGAGGACCCCGAGCTGCAGCCCCAGGGCTTCCCCCTCCCACTTTGGG
TGAGTATGAGCAGGGCCCAGGTGGCTGGGGTGCAGTGGGCAGCCCGGTGGTCCTGCCTGGGGTCTGGTGC
TCCCTCGGTGTCTGTCAGCTCGGGCTGCTCTAGCAGAAGGCCACAGAATGGGGGCTCACACCGCAGGAAT
ATATAGTCTCATGGTTCTGGTGTCTGGAAGTCTAAAATGAAGGCGTTGGTAGACTCAGTTTCTAGTGGGG
CCTCTCTTCCTGGCCAGCAGGTGGCCGCCTTCTTGCTGTGTCCTCACATGGACTTTCTCTGTCCATGCAT
CCCTGGAGTCTCGCCCTCATCTGGTGGTCCTTTGTAAGGACACCAGTCCTACTGGATTAGGGTCCCACCC
TTAGGACCTCATTTCACATGAATCACCTCCTTAAAGACCTCATCTCCAAATACGTCCTGTTGAGGAGTTG
GGCTTCAGCATATGGATTTGGGGGGACACTCAGTTTCCTTGGGCCTCTCAGCTCCTGAGGGGGTCATCAT
GAGCCACCTTCCTGCTTGAGGGGGATAAAGGGCAGCTGGGACCCAGGGCCTGGGGCCTTGGGCCGAGGCA
TGGGGGGCATTTGTGGGCCGGTGTTTGAAATGCTCCACCAGCACTGCAAACACACCAGACCCCTGAAGAG
AGGGTCAGGCGCTCAGCTAGGAAGAGGGCGCTGCGGAGAGAGTGGTGTGTGTCTTCCTGTGTCCTCCTAG
GAGCTTCTGTCTGAGAAGTCAGAAGTCATGAGTTCCCAGTCCTGGCCGCATTTGGAGGCTGGATCATTGG
AGGGAGGCGCCGGCGCCCCATGTAGCTGATGTTGGTGGAGCATTGGCTGAGGGCCCAGAGAGCCATGCTG
CTGCCTCCCACACATCAATCACTCAGGCCACACAGTCACTGGTGAAAGAGGGCACACAGGTAATCCTCCA
GTTCTTGAATGGGGAATGGAGGCACAGGAGGGCTAAGCGAGGGCCCATGGCCTCAAGATTCAAACACAGG
CACTGTCTGGCCCCAGAACACACGCCCTTCCCACCTGGCCGAGGAGGTGCTCACAAGGGGCACTCCAGGT
GGATGCAGAGTGGGCCAGCCCCCTCCCTTCCTGCGGGCAGTGGCCCCTGCTCTTCAGGGGAGTCAGATGC
ATTGATTCTGGAGGCTGAAGCCAGGTGGAAGTGATCTAGAGGGGAGATGAATGTGAGGCGCTGCCCCGGA
GGAGACAGGAATGGCTCATCAGCCACGAGCCAGTGCCATTTAGAAACTGGCAGCCAAAGTGACGAGGCCA
CACAGGGCAGGAGGAGCAAGAGCACACGCTTGGAGGTGATGGTATTCCAGCAGTGGCATCAGCTCAGCCT
GGGCCAGGCCAATGAGCCCACCAGCAGCCTGCCCCTTGCTGGGGTGGAAGCCTGAAAGCAGACCACAGTG
GCTGCTTTCTTTGGTCTGGGGGAGCTCAGGCCAGGGGCTGGTTAGAGTCCCCAGCACAGCCCCATCCTCC
ATCCCACAGTGGGCAGCAGTGTGAAACTGGGCAGGGGACTTCAACTCTCCTAGTCTAGTTTTTTCATTCA
TGTCAAGGGAACACCACTACTGACCTCACAGGGTGAGGGTTAAGTCAGATGATGTGCTTAAAAGTCTTAC
ATGAGGCATGGTGACACTAGATGGGCCAGGGCTTCCTGAAGGCAGGGTCAACTCTGATTCATCTTGACAT
CTCCAGTTCCTGGCCCAGAGCCTGGCACATAGTGGGCACTTGAGAAGTGTCTGTTCCCTTCCCTCGAGAG
GGTCTTCAAGTGGTGAATGGAGATGATGGTGATAGTGATAATGGTGGTGATGGTGGTAGTGATGGTGATG
ATGGTGGTGATGGTACTGATGATGGTGGAGATGATGGTGATGATGGTGGTGATGGTGGTGATGGTGGTAT
TGGTGGTGATGGTGGTGGTGATGGTTATGATGGTGGTGGTGATGGTACTGATGATGGTGGAGATGATAGT
GATGGTGATGATGATGGTGATGGTGGTGATGGTGGTATTGGTGGTGATGGTGGTGGTGATGGTTATGATG
GTGGTGGTGGTGGTACTGATGATGGTGGAGATGATAGTGATGGTGATGATGGTGGTGATGGTGGTGGTGG
TGGTGATGGTGGTGGTGGTGGTGGAGGTGATGGTGGGGTTGGTGGTGATGAGTGTGATGATGTGATGGTG
GTGGTGATGATGGTGGTGAGGATGGTGTTGGTGGTGATGAGTGTGATGATGTGGTGATGGTGATGGTGGT
GGTGATGGTGGTGATGGGGGGGTTGGTGGTGATGAGTGTGATGATGATGATGTGATGATGGTGGTGATGG
TGATGGTGGTGGCGATGGTGGGGTTGGTGGTGATAAGTGTGATAATAATGACGGTGGTAATGATGGTGTT
GATGGTGGTGGTGAGGATGGTGGTGGTGATGATGGTGATGATCATGGTGATGGTGGTGATGTTGGCGATG
ATGGTGGTAATGATGGTGGCAATGGTGGTGGTGATGGTGGGGTTGGTGGTGATGAGTGTGATGATAATGG
TGGTGATGATGGTGATGGCAGAGATGGTTACAATGCTGAGAGTGATGATCTTGGCGGTGATGGTGGTGAT
GTTGGCGATGATGGTGGTAATGATGGTGGCAATGGTGGTGGTGATGGTGGGGTTGGTGGTGATGAGTGTG
ATGATAATGGTGGTGATGATGGTGATGGCAGAGATGGTTACAATGCTGAGAGTGATGATGGTGATGGTGA
CGATGGTGATATAAGTCCCCATGCGCACTTCCTCTTCTCTAGCTCTGTCTCTCACCTGCTCTTGCCTGAG
CTCCACTGTGGCCACCCACCTGTGACCTGTGGATGGAAAGTAATTCTAATTATGACATTTTCCTACAAAG
AACAAACCACTGGGCTTCTCTGGAGCACAAAGGCATGGCGAGATGAGCAGTTTGCCCCACAGGACGCTGG
CATTGCCCTCTGAACTTTTTCCCAGATGTGCTGAGGACAAGGAAGTCAGGAGTGTGGCTTGTTTTCCATC
TGCTCAACACTGCTCGTGCCTGGAGCCCCCCAGCTGTGCCTATCAGCCCCTAGGGGTTGGCTGTCAGGGG
AGCAGAGAGACTCTTGGGGCAGCGTGGCCTTGGTCACAGCAGAATGCTGGGTTCTGACATTTATGAGCCA
TGTGACCTTGGCATGCTCCTTGACCTCTCTAAACCTTGCATGACACTTAGGAAGGTTAATAAGATCACGT
CTGTGAGAGGTCTTAGTGCCACAGTAGATCAGTGTCAGTGAGGGTAGGGACTTAGCTCTGTGGCTTGGGA
GGCGCAGGTGGCAGTGAGTAGCCAGTGGGTGAGACCCAGAGAGGCTGTGCCTGTGAGCGGCTGGGCAGAG
TGGGAATGGGTGCTGTCAGCTGGGTGCACAGGCAGAGGAGGTACCGCTGCCCACAAAGGGAAGTCTCTGA
GTTGGAGCGGAGCAGGGGCGGGAGAGAAGGCAATTGGGGCCTTCCTGGGTTTGGGAGGCTCAGCACCAGG
AGCAGCCCACAGCCAGGGCTCAGGACCCAGGGGCTCACCCAGCCCTCCTGACCAGGCTAACCTGCGGGGT
CTGGAGGGAACACGGGCAGCCACGGCCATGCGCACTGGGAATAAGTGAGAGGCTCCCGCTATCTCAGACC
TGGCGTGGGAGCTGTGGGCCTTTCCAGGGCCCGGCTGCTGCGGAGGTAGGAGTTTGGTGTTGGCAGCAGG
AGCAGCAAGGACACACTTTAGGACCTACAGCGTGGAGGCAGGAGGACAGGGAAATGGCCTCGAATGAGTC
CGTGACCCCTTGAGGAATCTGCACACGACTCCTGGCTGTCTTGTCACTTGATGTAAGGCCTCAAGCGGCA
TCTCCTGAGGATTTCATGGAGGCCAGAGGGGACTCACCCAGAGGTGGAGGGATGCAAGCTCAGAGGCCAG
GGTGGGCGGGGGCTGGAGCTGCTCTGTGACAGCCCAGAGCTCCCGGAGCCGCAGCCACCGTCCCAAGAGA
GCTCAAAAGAGGAGGGGGTGGGAAGGGGGAGGTAAAAGCTGTTCAGCCAAGGCTTTCTCAGCCTGGGGAC
TGACCTGGGTTTGTCCTATTCTGTGAACCAACCCAAACATGTAAATGTCCTGGCACTGAGCAAAGCCTTC
CCCAGGGGCGTTTTCCCTGAAGGACTTCTCAGAGCCTTTAATGGGCCAGGGAATCTTGTCACGCTCAGGT
GATGGTAGAGGAGGAAAGGACTTCTCCAACCGTGGTGACTCTAGAACCTCTCCCTACAGCAGCTCGGGGA
GCTCCAGCTCTGGAGAACACACTTTGGGAAGCCTTCCCACGCAGGACAGAGGTCTCTCTGCCGGGCTGCG
AGCCCTGCCCACTTGTGACCAGGGTTTTCAATCCCCTGGTGTGAGAGCAGTCTCAGGTAGCCCAAGCCTA
AGTGGAAAAGGGGCCGGGAGAGGCGGCTGGGCACCCCCTCGCTGGGGCTGGTCTCCTGGGTCCCTGTTTC
CTGTCTCAGGAACTGGGAGGAACCCACTTCCACCACCCAGCAGGGGAAGACTGAAAGTATTAGGGGGCCT
GTCTGGGGTCAGTCCCACTGTGGACTTGGGCACTGCCACTCCACCCCCCCCTGCTGCCACAGGCAGTACC
CAAAGGTGGTGCATTCGGACCCTGAACCCCCAGCTTCATGGGTTCTAGGACCCTCCTCTCCATGGCTCAG
CTTCCAACTGATGACCCCTCTCCAGAGCCAGCCTCTGTCCTCCAGCAGGAGGTAGCTTTGGCCTCTCTTG
GTCTCTGACCCGCATGTCCCTTCTGCGCTGCAGTCTCCTGGGCTCAGGGTCCTCCTTCAGACTCCTCCAG
GGAGGCCACACCCTCACACAGACCTGCCCTGGGCTCCCTTCTCTGCTCTGAGCAGCCCCTAGTCTTACAG
GAGCTCTGCAGAGGCCCTTCCGCTGCAGAAGAGCTTAGGTGACAAGCCAGAAGCCTCAGGTGGCCTCCTT
GTTTTGCCAGCCCTCCCCCGTCGGTTCCTTGCTTTTTCTACCCAGCTTGGATCTCGGGGGGCTGGCCTGG
ATGGATTTTGTCAATGACTCACTTGCCCTTTAATGATCATTGCATTTGGGAAGCCCTGGCCAGAGATGCA
AGTGTAGCAGGAGAGGTTGGAATGTTCCTGTGCCTCCTCCCGGAGCTGCTGCTGTGGGGCAGGGAGACTT
CTCCCTTCTGGTGGCCTCCTCTCCTCCAGCCCCTCCTTCTCACGAGGCTCTGTGAGACTGTTTTGGGCCT
GGAGGCGGCAACAGCTTTCCACGGGGCTGGGTGCCGGGTGCCTCAGTGTCCCCCACTGTCCCTCCATTCT
GCCCACTCCTCTGCAAACAGCCTCTGCATCACGTGTTCTTCAAGATGCCAGCCGAGCGCGCCTGCAGGAG
CCTGTCCTGCTCAGGGGCGGAGGGCTCTGCTCGTGTGGGCACAGCTCTGAGGCGGTCCTTGTAGCAAACA
TCGCTGACTGCCTACCACACGCCCGGGGACCTCCTGGGCACCTGTCAGTCATGAACGTAACAGGTTCAGG
TTACCCAAGTCCCATGCCCTAACTGGGCAGTTGCACCATGGGCAGCAACAGACCCTGGGCTTCAGGATGT
CCCAGCAGAGCCGCAGCAGCCACAGAGCAACCCCTCTTCACCCTGGGGATGGGGCTGGGCCTCTGAGAGC
AAGCATGAGATTGCAGAGCCAAAACCCAAGCTGTCAAAACGACCAGCAAGAAAGACAGTTTGGATTAATT
GGCACAGGGAGGTCCCTAGACATTTAGATTATGGCTGCTCCTGGGCTGGAGCGTGGAACCCAGAGACATT
TCACAAATTAACATAAATCCTGTGAACCCTCTGTCCATTTCAACGCTTAATGCCAAACATAAACAAATGA
AAGTCATTTCAGAAGCTGGAGGAGTTTGAATAATTCAGTCCCATCATCATTTACTCCAAAACGCCTCATT
TCATCTTATGAGTCATCGTTGAAAGGAAAAGAAGATTTTACTTGGTATTAGAACCGGGAGAGCTGGAAAA
GCTTCGAAGAGGAAGGAGGCTGCCAGCTTCCTGGGTGCACAAGGCCGTGGCCAGCTTCCGGGCTCAGTCG
TGGACAAAGGCCTCCTGGGACAGAGAAGTGGCCTCTAAGCAAAGGCGGTGGTGTGACGCTAGCAGGCCTG
TGTCCACCTCCTGGTCAGGCTGCTGACTGGCGGGGCTCTGGGGGTAAGGCCTTGGTCCTGCCGTGCCTCC
TCCTGGCTCATCTGTGACACAGAAATGACAGTTCCTACTCCCCAGCTTGTGCGGGCCCAAATGGGATGGG
GTGTGTGTGTGAGGACTGGGGCCTAGGCCTGTGCAGGGCAGGTTGTGTTACTAACGTCCCCCCGTGGCTC
CTGGGCTAACGGTGCACTTCTCCGTCCTGCTTCACTGCCTTATGGCACGTGTCCATTTCCCCAACCACCG
GTGGGCCCCGGGTGGCTTGGCCGGCCTGGTGTCCCGTGCCCAGCATGGGCTCTGGCCCAAGTTAGCACTT
GCTGCCTAAGTGCCTCTGCCTTTTACTCACCTCCATCTGGAGCGGGCTCAGTGGGGTTCCTGCTGGGGCT
CTACGGGGGCATCCAGACTTTGCCCTCTGACCAGCCCCACTGTCCTGTCCTTCCCTCCCCTGCGTCCTCA
TCTGTCACTCCTTGGTCACAGCCGCCCCCTGACTTTCCTCCCTGCCTCCTTGGCATGGTGGGAAACATGC
GAGTTCCATTTCTGAGGCTGCCTTGTGCCTGATCTTGGGCAAGTTGCTTGCTAGACCTCTCGGCGTCTCC
GTTCTCAGCGGTGCGGGTAACCTGGCCCTGTGCTACTGTGTGTGGGTTTACAGCCAAGGCCTGTGAGGCA
CGTGACCACCAAAACCCTCACGAGGCCATGTGGTCGTGGCTACGGGTTTAATCTAAAATTCATTCTGATC
CCTTGGCCTTGGAAAAGGCACTTAGCAGGGTGGTGAGGGCCACAGGTGTGGTCTGAGCTCCTGGGGAGCT
ACCGAGGGCAACAGGCATGTTCTGGAAGACCCACCGTCTGAGGGGTCATGTGACAGGTGGGCTAGGTGGG
CACAGAATGGGAAGAGAGGCAGTGAGGCAGCCTCTCTGCAGAGGAGGGGCTTCACAGAGGAGGCAGCGGC
TCAGGTGGTCCCAGGAGGATGGGTGGGGTTCGGAGGCAGAGGGCTGGACTTGGAAGGAAGGAAGAGCGGA
CACAGGGCCTCTGAGCGGACAGTGCTGGCGGAGCCCTGGGGAGCACCGAGTCCCCGCCGTGATGGGTCCC
TGAAGCACCGGAGCCAGCAGCAGGAGCATGCGGGGGAGACCTTGGCCCAGCCCTGCAGTCAGGCAGTAGG
TGAGCCCTGAAGGGTGAGGTTGCTTCAGCTGGATGAGAGGGTGTGGGAGAGAGGAGCATCCCAGGCAAAG
TGAGCCGCGTTTACCTGGAGACCAAGGGTGGGAAGCGAGGGAGAAGAGGTCGCGGTGAGCCTCGGGTGGA
CCCATGTGGCAGACCTGTGAGTTGGTCCCCGACAGCCTCCTGCCTTTCTCCCTGACTGCCCAGCAGCAGT
GTTCTTGGGCCTCAGGGACGTGTCGTGACTGGCTGAAGGCTGTCACGGTGGTCCTGTGTAGGACTGGTTG
CCACAGGTAAGTAGTGACCTGGCCTGGCCGATGCTGTGTTAGGATAAACGTGCTAAGGAGGCTCTGGAAA
AGAATTTTCCTGCTTGGACAAGACAGAGACTCACACGAGGAAATTCCTTTTGTTCCTACCCTCACTTCCA
GTGTTGAACACAGTCATGCTAGAAATGGTGCCTGGTGCAGCTGCAGCCGTCTTGCTACCAGCAGGGCAAG
GCAGAGCCGGCCGCCCAGGGACCTGCCACTGTGGGGTGGCCGAAGCAGCCCTGGCACCACACAGCTGTTA
TATGCTGTGGTCAGAAGTCCTTATTAGGTGAGCAATTAATCAGATGTGTGGCTTCTCACCCTGTATTTCC
TCTGGCAAGACCCTGCGGCACTGCAGACCCCTGTCCTCATATGAGGCCTAAGCAGTGAGGAGCTTCAGAC
CCGTGTGAAGACAGAAGGCGCGGGGAGCAAAGGTGGCCAGAGAGGGGTGGGGTGGGCCACGAGCCCCCAC
CAACTGCAGGCCCACGGCTCAGCAGCAATCTGAGGTCTCCCAGGCAGCCTTCTTGCCTTCCTTCCATGGC
TCCTTGGAAAGAGCTGGGTTTCATCTCGGGACACAGTGGCATTAGGGATCGGCAGAGACCAGCAGGAGCC
CTGGGTCCAGACTGTGGCTGTTTCTGTGGTGACCAGCAGCCGGCACTGCCTGGCAGTGCCTGGGAGTGGA
CGTTGAGGACACTGTGCCCAGGAGGTCAGTCTGCCCACCCAGGCTGTGTCCTGGTTGCTGGGTGGGGGTG
TGAAGAGTCCAGGACCAGCCGTTGTCCCACCCAGCAAAACCCTCTGTGTGTGGATCCCATGGGGGGCCGG
GCTCCGGGGATGGACAGCCAGCAGCCCTGGCCTGAGCAGCCATCCGGAGGAGGGAGCAGACAAGAAACAG
GCAGTGACCGCACAGGGCGATGGGGCTGGGCAGGGAGGAGGCGGGTTCGCTGTGGCCAGATGCCAAGCCA
GAGCCGGGAGGCCAGAGGGAGGGCCGCAGCCGGGACAGGAGTGACTGATGCCAGGGCCTCAGAGTGAAGA
CAGGGCTGGGGTCCCCTGCCCCCTGATGGGAAGGAGACTGCGTGGCTGGGGTCCCTGGGAGCGCATCCCT
TTTGAGACCACACTCCTCAGCAGCACCTCCCTCTCTCAAGCACCAACACTGGCCTTTTGGCTGCCTGAGA
AGGGGCTCTGGGGTCCAGGGGAGCCAGCTCAGCTCACCCCAGTGAGCTTCCCACACTCAGACAAGGCCCT
ACTCCAGGGCAGCTTGGCCCGTGCCTCTGCCCCTACCCCTGCCATGGCGTCTCTCCAGGCCCTAGGTCTG
TACTCACATAACCCAGTCCTGCATTGTGCTGACTCAGCCTCTGAGCCTCAACAATCCCTCCTTCGAGTGG
GGACAACATCCCTGCCTCCGAGGGCTGGTGTGCGGATCGAGGGAAGTCACAGGTAGTCACAGCTCTGTGA
AGGGTAACGCGAGCGCCTAGGCTGGGAGGCTGGGTCTGCAGCTGCCGGCTCCACGGGCTGTCTCCACCTC
TCCCTGCCTGGCTCTCCTGGCTCTTCGTTTCGTCATGTTCCTTCTGCCCAACCAGTCCTAGGGGGACAGG
GAGACTCCTGGGCACAGGGGCTGCTCAAACTGCCCTGGGACAGATGGCCTCCTGCCCTGCCCCAGGCAGC
ACTGGGAAGCAGAAGGGCTGGGGGAACTCCAGCAAGGCCTGGGGGGCATTGAGCAAAGCCTGGAGAGAGC
ACACTTGTGGATGGGTGCACCTGTGGGTGGCCATACCTGCGGGGCGGGCACACCTGCAGGCTAGGAGTAG
GGGGGCTGGGCGGTGGGCCCAGGGGAGGGTGTTACTGAGTTACGTGGCCCCCAGTGCTCCTGGTCCTTTC
AACCTTTAAGTTGAAGATAAAGTCACTTTTGCAATACTAAAGACCAACAGATTCTAATATTCACTCCCAT
GCAGGAACTAAGTGCCTGTTACCTGCTGTCCTGCTTGTCCCATCTTAGGGTTTCCCAGGGCCCAGCAGAG
CCTTTGCCATACCTTCTCTAAGGCCCAGGCTCTGGGAGGAAGGTCACTCAGACTTTCAGCACTGCTGCTG
GTGGATGGGGCCCCTCTCAGAGGCCCTGGATCCTGCTGGGCCTGTCTGCCTGTCTGACCATGGAAACCTC
CAATGGGGCAGGGTCCAGGTTTCCCTCCCTACAAGCACGCTCCTAAGCACGTCCGCTCTGTCGTTGTAGG
TGTAGAACTCAACAATCACACCCTGCAAAGGCGGAGGAAACCCTCTTCCCTCCCCAGGCCCATTCTGTGT
CTCAGGCCCTGCCTCTCCCCTGTATGTACCGCATACATCCCTTTCGGGGCTATGCACAGGCTTCAGGGGA
GCCCCACCCATGGGGTGCATCTTCAGCAGGACAATCATCAGAGAATGTTGTGGTCCAGGATAGGGCAGTC
CTCCACTTCTGGTCCTCCAGAAGTGGACTAACTAACATCTCCTCCAAAGTCATTCTTCTTCCATGTTTAC
GGTGACCTCTGTGTTCTAATCCTGGGAGAAGCCCTAAGCTGAGCTCACGGGAGCTGGGAGACCCTGCTGC
CAAGCTGAGTCTCACCATCTTTGGCCCATTTTTTTTTTTTTTTTAGACAGGGTCTCGTGCTGTTGCCCAG
CCTGGAGTGCAGTGGTGCGCTCTTGGCTCACTGCAGCCTCACCTTCTCTGGCTCAAGCAATCCTCCAGCT
TCAGCTTTCCAAGTAGCTGAGGATACAGACGTGCATCACTATGCCTGGCTAATTTTTTGTATTTTTTTTT
GTAGAGATGGGGTTTTGCATGTTGGCCAGGCTGGTCTTGAACTCCTGAGTGTAAGAGTTGAAGAAAGAAG
AAAGAAACACGAAAAGTGGCTCAACAGTCCAAGACAGGTTTATTTTGGAGAATAAACCTGAGAGGGGCTT
CTGGCCGATTGCTGTCAGGAGCACTCTCTCTTACAGACTAAGGGTATTTAAGGGTTTAGGGAGGGAGAGC
TTATCGCAGGTTGGGAATGTTTCTGGTCAGAGGAGTGTTTGATTTCGGGGTAGGAATGTTTCTGGTTGGA
GGGCGCTTTATCTCAGGGTTGGAATGTTTCTGATTGGAGGTGTCATTTGTGGTTTATGGTCATGCTGACA
GCCATTAGGCTGATTTTTTGGGGGCTGGATTTAGGCGGTTTTTAATCAAGGGGAACTTAAAATGCTGCTG
TTTGTCCAAAATGTTGATGCTCCTGCTTTGTCAATCCAGACCCTATAGTTATAAAAGGATGAGGGGCGAC
GTGCTCTTTCTGGCTACTTCCTGCTGAGAGAGGGTTGTCGTTATGGGACACTGAACATGGTGCTGGAGTG
GAAGAGGTCGATTTGTTCTGGGTAGCACACTCTGCCTCAGAGGCCCAGAGGCAGCGCCCACTGAAACATC
TAATTTTCAGCTCACAGGGCTTCAAGAAAGCACAGCTTAGGTTTTAGTGATCTCCAGCTAGAAAAAAAAA
AGGGGGGGGGGAAGGAAAAGAAAAAGGAAAAATTGAAAACATTATTTTGGAGACTTGTAGCCAGAAAAAT
TAGAATTTAATCCAAACTGTAGAAAACAATAAAAATTGAAAAACCTCAGACAAGACTAGAATTTAACAAC
AGGTGTGCTACAGTTTTTGAAACACAATTCTCTCTCTCCAGTTTTCCATTTATATTAAAAGACAAATCAT
GGGGCCAGGTGTGGTGGCTCACACCTGTAATCCCAGCACTTTGGGAGGCCGAGGTGGGCGGATCACAAGG
TCAGGAGATCAAGACCATCCTGGCTAACACAGTGAAACCCCGTCTCTACTAAAAATACAAAAATTAGCTG
GGTGTGGTGGCGGGCGCCTGAAGTCCCAGCTACTCGGGAGGCTGAGGCGGGAGAATGGCGTGAACCCAGA
AGGTGGAGCTTGCAGTGAACTGAGATCGTGCCACTGCACTCCAGTCTGGGTGACAGAGTGAGACTCAGTC
TCAAAAAAAAAAAAAAAAAAAAAAAAAAAAAACAAATCATGGTATGACTAGTTTGCTTTGCCAGATTTTT
TTTTAGGTAAAGTTTCACTCTTGTTGCCCAGGCTGGAGTGCAATGGTGCAATCTCAGCTCACTGCAACCT
CCACCTCCTGAGTTCAAGCGATTCTCCAGCCTTACTTCCTGAGTAGCTGGGATTACAGGCATGTGCCACC
ATGCCCAGCTAATTTTTGTATTTTTAGTGGAGATGGGGTCTCACCATGTTGGCCAGGCTGATCTCCAACT
CCTGACCTCAGGTGATCCACCCATGTCAGCCTCCCAAAGTGCTGGGATTACAGAAGTGAACCACCGCACC
CAGCTGTGGCCAGATTATTTGTATAAGGTGCAGCAAGAATAATTATTTTTACATAGGCCTTTTAAGTTGG
CTTCAAAAAAACTCTGTTTCATGGAAGGAATTTGAGATAAGACCTTTTTAAAGCCAATCCCAGCCATGGA
AGTGCACCATCAAATACCTGTGAGTTGGGTGAATTCTTCCACTCTTGAGGCTCCAAGATAACCTGGGGTT
CCTGGCCTGTGAGAAAGTGACATTCCTTTACTTACCTCAGGTCAGAAACCTGCACAGGGACTGCGCGCAC
AAAATATGAGGCCCACAGGGACCGCGCGCGCAAAATATGAGGCCCACAGGGACCGCGCGCGCAAAATATG
AGGCCCACAGGCACCGCGCACACAAAATATGAGGCCCACAGGGACTGCGCGCACAAATTATGAGTCCCAC
AGGGACCGCGCGCGCAAAATATGAGGCCCACAGGGACCGCGCGCGCAAAATATGAGGCCCACAGGGACCG
CGCGCGCAAAATATGAGGCCCACAGGCACCGCGCACACAAAATATGAGGCCCACAGGGACTGCGCGCACA
AATTATGAGTCCCACGGGGACCGCGCACACAAAATATGAGGCCCGCCATCTAAGGGCTCTACTGGCTTCA
CAAGTCAAGTTTGATTCCTTAAAGGAGAGCACACCATTCCAGTCAAAGCCTTGCTAAAACAACCAGTTCT
TCCAATTGTGTCCTGCCATAAAAGAAAACAGACTTTTGGTCGGGTGCCATGGCTCACACCTGTAATCCCA
GCACTTTGGGAGCCCGAGGCGGGCAGATCACCTGATGTCAGGAGTTCAAGACCAGCCTAGCCAATGTGGC
GAAACCCTGTCTGTACTAAAAAACACAAAAGTTAGCTGGGCATGGTGGTGCATGCCTGTAATCCCAGCTA
CTCGGGAGGCTGAGGCAGGGAGAATTGCTTGAACCTGGGAGGTGGAGGTTGCAGTGAGCCGAGATTGCAC
CACTGCTCTCCAGCCTGGGGAACAGAGCAAGATTCCATCTCAAAAAAAAAAAAAAGAAAGAAAGAAAGAA
AGAAAACAGACTTGTATTGCACCTTTGCAAATAACCATACTGCCATAATTTAAGGATACTCACAGGTAGT
TTCCAGACTCGGGAGAAAACCAGGCAGAGAGAAATAAGCATGCCTCAAATTTTGTTCATGGGAGCACACC
AAACTGTCAAAAGCTGTTGATAGCTCAAAAGAAAAGCCTCTTTGACTCTGAAAAGCAAACAAAGGACCAA
CAATATTCCGAGCAAAACATCAAAAAGATCACTCCAGTCTGTTAGTTCAGTTCACGCAGTCAGTTCCTGT
CCTGCCTGATATCAATGAACATTCCAGCTCTTCAAGAGTCCTGAACGTCCTTCCTCTATTCTGATGTCAC
AATCTGCAAAGTTATCAGAAACCTGCATTCAAGAGCACCTGTCAGAACTTTATAGCTGATCATAAAACCA
CCTTCTAAAGAGGACCAAAACAAGACAACAATTGTTCATGGATGACAAAAAGTTTTAGGGTGGCCGCAGT
TAAAGACACAATTGATGAGGAAATCTGTTACCTACGTGGCACACAACAATTTTAACATAACAATTATAAT
TATTACTGATAATGTACACTAAAACATATCAGGATTGTAGGAGTCTCCCACAACCTTGGAACACATACCA
AAAACATATCTACACAAATATAGCCCAAAGAAAGCCAAGCACCATTTCGTATTTGACAATATTTTCTGTA
TAATTTTTATACCAAATAAGCCAAATTTCACCTTTACATTAGTGTACTATGAATGTTAAACCGAATTAAT
AAATCCTTATAGACATATTTACTCAATTTTAATATTTGACCACAAGGTAAGATTTTTATAGACTTTTTAT
AGCCCTTTACAATTTTTGTTAAAGAGCAGGTTAGTGCTCTAAGAGAAACCCACTGTGCTTTTATTTTAAT
AAAATTTAATTTACAGAAAAACTGGAGGATACCCCTTTTAGCCAATATGTTTACACACAGAACTGCCTCT
AAAATCAGCCTTTCACAACTAGCCCAAACCTTCATTTTTATTTTATCCAACTGAAAAAAAAATCCTTTAA
CCTTTCAAACTTGGCAAAAATCCACATTCTCATGCCTCCCTGCAATCTTTCTACCAAAACTATATTTTAC
TTTTCCTACATACCTTGCATGTAAGGACAGTGGCTTGGAATGTTGGAACCTTTCCTTGGAATGTTCTGGG
TTTCAGCACCAAATGTAAGACTTGAAGAAGGAAGAAAGAAACATGAAAAGCGGCTCAACAGTCAAAGACA
GATTTATTTTGGAGAATAAACCTGAGAGGAGCTTCTGGCCGATTTCTCTCAGGGGCACTCTCTCTTATAG
ACTAAGGGTATTTACGGGTTTAGGGAGGGAGAGCTTATCACAGGTTCAGAATGTTTCTGGTTGGAGGAGA
GTTTTATTTTGGGGTGAGAAGGTTTCTGGTCGGCAGGGAAGTTATCTCAGGTTGGCATGTTTCTGGTTGG
AGACAGGTTTATCTCAGGGTTGGAATGTGTCTGGTTGGAGGTGTCATTTGTAGTTTATGATCATGCTGAC
ATTAGCCATTAGGCTGCTGTTTTTGGGGTGGATTTAGGTGGTTTTTAATCAAGAGGAACTTAAAATGGCA
GTGTTTGTCCAAGATGGTGATGCTCCTGCTCTGTGACTGAGCTCAAGCGATTTGCCTGCCTCGGCCTCCC
AAAGTGCTGGGATTACAGGCGAGAGGCACTGTTCCCAGCCGTTGGCCCGTTTTCTAAAGCCACGTCTTCC
TCTGCGATCATCCAGAACAACACAGATTCTCCACCTCCTCTTTTTCTAAGCTCTTGCTGCAAATGCTGGA
GAAAGAACAGTGAGCGTTCAGGCTGCAGCTTGGCCAAAAGGCCAGCGAGGGAGAAGTAATTACCTCGGCA
ATGACAGGTGTTCCATCCTTTCTTCTCCCCTGGAAATATCAGCCATCCATCAGCCAGGGCCAAACACCCA
CCCACATCTGGCTCGGAAAGCAGTAATGTACCAGGAAGCAGCTGTTTTCGAGAGAAGCCAGCCCTCTGTC
AGTTTACTAGCTTATTCTCTCACTCATTCAACATTCCTGTGTTTATCATACCATCCCAGGGGTGGTGTGG
CTTAAGGATGGGTCTCGGGCTGGCCTGGCTGCTCCTGTTTCCCGACTGCCACCCACTAGCTGTGTGACCT
CAAGCAAGCTGCTTAACCTCCCATGCCTTGGTTTCCTCAACCATTAAGTGGGAGGTAACAATAGTGGCAC
CTACGCATAGATTGTTCTTGGGGGGTAAATGAATTAATACATGTGAGGGTTGGCCAGGCACAGTGGCTCA
CGCCTGTAATCCCAGCACTTTGGGAAGCTGAGGCGGACAGATCACAAGGTCAGGAGTTCGAGCCCAGCCT
GGCCAATAAGGTGAAACCCCATCTCTCTACTAAAAATACAAAACTTAGCCAGGCGTGGTGGTGCAAGCCT
GTAATTGCAGCTACTCGGGAGGCTGAGGCAGAAGAATAACTTGAACCCGGGAGGCGGAGGTTGCAGTGAG
CAGAGATTGTGCCATCGCACTCCAGCCTGGGTGACAGAGTGAGACTCTGTCTCACAAAAATAAATACATA
CATACATACATACATACATGTGACGGTCTCCCAGACATGCACTCCGGCTCCACCTTGACCAAGGGGATGG
GGCTCACGGTTAAGTCAAACTCTCAGGCTCTTTCTCCAGAGAATTTGAACTCTGAGCCTTGGGCTGATGA
CACAAAGACTCAAATGGTGGCTGCGCCTTTCTCCCCCAAGTGCACCCCCAGAGACTGCTGGTGCTTCCTG
CTAGCTGGATCCCCAGAGCTGCTTGGTCCCTGTTCTAGGTGAGGCCATTCAGCAGTCCTTGTGATTTTCT
GAGCATACTTTAGCCTTCTAGCACACTCCTCTCTTCCCCTAAAATTAGCCAAGAGTGGGTTTCTGTTGCT
TGCATCCCAACTACCATCATTGGGACAGAGCCCCTGTGCTTGAGACAAGCAGAAGATAGACTTCTTTCAC
CTGGGGCCTGGCTTCGTCCAACAGCAGAGCCCAACCTCCAGGGCAGACTGACCCGTGATGGGCATGGGAG
CCCAGAAAAAGGCCCCATCCAGCCTGTGGGATCAGAGGAGCCTTCTTGGAGGAGGTGATGCTGGGCGAGT
GTTAAAGGATACCTAGGCATCAGCTAGGTGAAGAGAGCAGGGAAGGAGACTCCAGGGAGAGGAATGTGTG
AGAAAATGGGGAGGAGAGGGAGGAACGGTTGGTGGGCAGCTGCCACTCTGTCCACTGGCAGAGAAGCAGC
CAAGTCTCTGATGGAGCCCACCGGAGCAGCCTACCTGGCTTCTGTGGTCACTGGCTGTGACCCCGCCAGC
CCTGCTCAGCGCTGTGCCCAGCCCTGGGTGCAGGGAGGTGCGGTTTGCTCTCAGAGGAGCAGCTAGCTGG
GAGCATCCGAGGCCATTAGGGACAGGACGCTAATGCATCGGCGCCCCATTGATTCTGCCTGGCTTTTGTG
AACACGTCTGCGCTGACTAATTTGTTTAATTACTCATTGCCGCATCTGTTCTCAATTGCCCTATGCAGAT
ACTTAGTCTGTGGCTGGGAGCCAAGCCTCAGGGTCCCTTTTCCTCTTCCAAGATGGGTGGCACTGAACGC
CGAGGCCACGGCCCGTCCTGATGTGGTCAGAGATGTCGTCATGTGCCACTAACAGGCGTCGCCAAGACCA
GCCAGGTGCAGACCTTGGGTGGGTCCTATGGGGTCCTACAGGAAGCATAGGCATGGTCCCTGGTGGTCCC
CAGGAGCACGGCTCTAACACAGCCGAGGCATAGTGTGAGCAAAGTCAGACGCAGTGGTTACAAGCAACCC
CACGTGGTCTGCTTCGAATCCAGCAAACTTTTGTTTTCAGTGCCCAGCTCCCTAAGCCTTCTTTGTCCTT
ACCGTCTCTTCAGAATCTGTCTGCTTCACCCTGAATCTTGTCTATTGTCCTGGCTAGTTCAGCTGGAGGC
CAGGGGTCGGAGACTTAGAGAAATGAGGAGGGGGCGTGGAGCAGGGGCTGAGGCCTGAGCGGTGAGTGGG
GCTCGGTATTGACGATCAGCGAACAGTCTCCTGGGGAGTCAGCTTGAATGGGGCCTGTGATATCTGCGGC
CAGTGCCCTCGGTATGTCCCACTCAGCATCCTCCCCAGGTCAGAACACATTTTGGGGCAGAACCGAGTTT
TCTCTTCACTTTCAGACTCAAGTCCAGCTTATTGGGGCTTATGAAGACTTCCACCTCATACAGGATGAAT
GATTCTCTTCTCCATTCCCTCTGCTGCTCGTGGACACTCGGAGGTGGGGGAAGGCTCTGTTCTTTTACTT
TTCTGGTTCTAGTTTCTCAGGGAATGAGGCAGGAGGAGAGACATGGAAAGGGAGAGAGATACCTTCCGTT
TCAAGGAATATGCAGTTTGGATGTCTGAAAAAGATTTATGGCAGAGATGCTGGATGGAAAACAAACACAC
AGTTTGAGAGGGTCCTTTTTATTTTTATTTTATTTTATTTATTTATTTTTTTGAGATGGAGTCTTGCTGT
GTCACCCAGGCTGGAGTGCAATGGTGCAATCTCCGCTCACTGCAACCTCTGCCTCCTGGTTTCAAGTGAT
TCTCCTGCCTCAGCCTCTGGAGTAGCTGGGACTACAGGCACCTGCCACCACCCCTGGATAATTTTTGTAT
TTTTAGTGGAGACGAGGTTTTCACCACGTTGACCAGGCTGGTCTCGAACTCCTGACCTCATATGATCCTC
CCGCCTTGGCTTCCCAAAGTTCTGGGATTACAGGCATGAGCCACTGCGCCTGGCCAAGAGGGTGTCTGGG
AGGCCGAAGCGGGAGGATCATGAGGTCAGGAGATCGAGACCATCCTGGCTAACATGGTGAAACCCTGTCT
GTACTAAAAATACAAAAAATTAGCCAGGCATGGTGGTGGGCACCTGTAGTTCCAGCTACTCAGGAGGCTG
AGGCAGGAGAATGGCGTGAATCTGGGAGGCGGAGCTTGCAGTGAGCTGAGATCGCACCACTGTACTCCAG
CCTGGGCAACAGAGCAAGACTCCGTCTCAAAAAAAAAAAAAAAAAGTTAAACACATGTCTAACCTATGAC
CCAGAAATTTCAGTTCTGGGTATTTATGCACAAGAAATGAAAAGGTATCTATCTGCATAAAAAGACTTGA
ATAAGATTTTTCATAATAGTTGTATTCACCATAACTAAAAATATAAGCCACCTAAACATCTATTATGTGA
AAGAGTACCCATGGAATGTATAGCTTGGATACTACTCAGCAATAAGAGAAGTATGAACAAGAGGTAGAAT
GCAGTTCAGAGACAAACACATGAAAAACACAAAAGAGAGGCAAAAAAAGGTGGCAGTGGAAGGAGGAATT
CTGACACATGCTTAACCGGAGTTCCAGAAGAAGATAAGACAGTGAAGGGGACATGTGATAGTCAAAGGCA
GGCCCCAATCCTCAGATCAGGAACCATAACAATTCTCAGAGAGAATAAATGAACAGAAATCCATATTTAG
ATCCATCAGAGTGAAACTGCAGAACACCAAAGACCACAAGAAGATTTTAAAAGCAGCAAGTACAAGAGAG
ATTACCCTCAGAACACCAACAACAGATTGACAGTTGACTTCACAACAGCAATAATGGAAGACACAGAGCT
GGATACACAAAGGAAATTGACTACAATCTAGAATCCAAAAGATTCTTGAAGGATGAGGGCAAAATAAAGC
CATTTTCGGATACACAAAAGCTGAGAGCAGCTATTGCAAAGAGACTCTCACTAAAGGGCCTCCACACCAA
TGCACTTCAGGAAGAAGGAAAGAGGTTTCGGACGGAAGGTCTGTGTGCAGAGGGAATGGCTGTCTCAGAG
GCGACTCAAACTCAGTATGTCCAGAAAGGACTCACAATCTGCTCTCCCAGCCCAGCGCCTCCTGGGACTT
TGGCCTCTGTGGATGATGACCACAGTCACTTGGGCCAGAAACATAGACATCATCTTGATACCCCTTCTCC
CTCACCTCCCCCATATCCAGTCACCTCCAGTCCTGTCATCTTTGCCTCCTAATCTCTTTCATCTCTGCCC
TTTTCTCTCCATCTCCATGGCCACCCAGCTGCCACTGCATTTTGCTCACCACCTGCAATTGGCCTTCCAG
CCCCTGAACCCCTCTGATCTGCTCAGCCCGCTCTCCACACCGAACCCGAGGCTCTCTCTTTGAATGGCAA
TCTGATAGTCTCCCCCCTGTTTAAAACATGAAATGGTATCCTACTACCCTGGGGATAAAAACAAGACAAA
AACGTTTAAGAGGTTAGAGAGGTCTTCATGATTCAGTCCCCACCCACCTCTCAGACTCACCTCCCGCCCC
AGCCTCTCTGGACTCTTTTGGCCCCTCATGGGTGCCATCATTTTGCCCACATAGGCTCTTGTCTGTGCTG
TTTTCACTATAGAGTGGGTCCTTCCCTTCTTTCTTTGTCTAATTCATGACACTCATCGTTTAGTGCATTT
GGCACTTCCTCCAGGAAGCCTGCCTTGACCTCCCTGACTATATCAGCTCTCCCCCATTAAATGTTCCCCT
CGTGCCATGGTCCCCTCATCTGTGACATTGATCACATGAGCAATTTCACATTTATTCATGTAGTTAGTTA
TAGTCTGGATCCTCCTTTGGATTTTAAGTTCCTATAGGTAAAAGCCTTCCTCTCCTTTGGCCACCGAGAT
ATCTCCAGGACTACCATGGACATGCAGTAAACATTCACCAGTTGTTGAATTTATGAGTGGGTGAGTGGTG
CCTGCCACCCTAGAATTTCCCTTATTTGAAATTCTAGAATAAGCAGATTCACTTCTTTTTCACATCCATG
TGAATTTTTGTTCTTGTATAAATGAATAGTAAGTAAGAGCCATAAGAAATAATTGGAGAAAGAAGGAATT
GGGAATTGGGAAGAGAAAAGAGAGAAAAACAATAGAGGCAGAGCAGGGGCTTTTTGCAGTGATGCTTGGG
GAGAAGAAGGAAGCCGAAGGGACAGCAAGTGGGTAGATGGCAGGTGACTGGCCCCAGGTTATCCAGAGCA
GAGCTGAGACCACCCAGTCCTAAGGGGAAGGGGCTGGGAAGGAAGCCACTCAGCTCTGCCAAGCAGATTA
AATACAGATCAACAAGAGCCTTTCCCATTTTGAAAATGTACATTGCTCTGTGTTGCTGGGACTGCAGTGA
GACCCAGGAGGCAGGAATAAACAATTCTGAGTCATAAAATCATTAAGGCATCAAACATTTGGATCGATGA
GCAGACACAAAAGAATATGCTTCCTATACACATGTTAAAAGCCTAACTCTTGACAAGGATCTCACCGAAT
CTCTTGGGCAGAAGGATTCTGTAATGGAAAGTAATTGCACGCCACTCATCTTTGTTAATTTAAGATGAGT
AAAGGGTTATTGATTCACTCTTTGTTTCATGAGCTGTTTTCATATTGAATTTCAGTGAACAAAAATATTT
TTAATAAGGATTTCATGGGCTTATTTGGCATCTTTCTCTCCCAAGCTGCAGAGCTATAGATCCAACTGCC
AGCTGGACAAACCCAACTCATCTCCAGCCATGGCCCCTCCTGATGTGAGCCTATCTCAGGTTCTGGTACC
ACAATCCACCCAGTGCCAAGCCAGACACTCACGTATCACCCTGGATGCCCTCCGCAGCCCCTCACCCATC
CAACAGATGCCCCGTCCCTGTGCCTGCTAACTCCCTCCCACCTCTCCAGCTCAAGGCCTCCCTCCCACGT
GCTGGATCCTGCCTGTTCAGACCTGTCCTGTCTCCTGGTGGCCAAACTGGCCTCCCCAGTCAGTCCCTGC
TGCAGAAAGTGGTTTCTTTCTTTCTTTCTTTCTTTCTTTCTTTTTTTTTTTTTTTTGAGATGAAGTCTTA
TTCCATCGACCAGGGCCTGGAGTGCAATGGTGCAATCTTGGCTCACTGCAACCTCTGCCTCCTGGGTTCA
AGCAATTCTTTTGCCTCATTCCCCTGAGTAGCTGGGATTATAGACATGCACCACCACGCATGGCTAATTT
TTGTATTTTTAGTAGGGGTGGGGTTTCACCATGTTGGCCAGGCTGGTCTTGAACTCCTGACCTCAAGTGA
TCCACTGATCTTGGCCTCCCAAAGTGCTGGGATTACCCCTGTGAGCCACCGCACCCAGCCTGGAAAGTAG
TTTCTATAGTACAAAGCCGGGGGTGCTGCTCCCCTGCCCCACACACCTCAGCACCTCCATGTTGCTTCCT
GTGGAGTAGACACTATTGTTTTGTCTGCCCAGTCTCTTCTTTTCTTGTGGGAATATGCTTCTGGAATATT
TGTCTGGAAAACCCTCTCCTTTTGATAACTGTCTTTGCCTCCCTGGGACTGCAGCTGACGTGCTTGCATG
TGTGTGTGTGTGTGCGCGCGCGTGTGTGTGCGTGCATGCGTGTGTGTGTGTGTGTGTGTGTTGGGATCGG
CTTCCAAATCTTCCTACTGGGGGAATGCCCATTATCCATTAGATGCCACTCAGTGGGAAGTGGTGCCCCA
CCCGTCCGTGGAGACCTAAGGGGCTTAATAGATCCTCCCTGCATAGCCAGAGACGGATACAATCTGGGCT
CAGTGAATCGGTGCTTTGACTGCTCACAGCGTAGCTGAGAGCTTACTCCCAGAATGGCGAAGGTGGCAAA
GAGTCCCCATAGCTGGTGTTGTTGGCTGCCATCACCCCTTTTATCCAGGGGCACAGCTGAAATCTGTGCC
ACTCGGCTCTGCTGGGCAGGTGGGATAAATCTGCCCCAGCCTCTGTGCTGTCCCCATCCCTGCTCCTGCT
GCCCAGCAGCCTGGAGTCCTTCTCCAGACTTCCTGTAACTCTCTCCCCAAGCCAATGCCCTCCCTCGGGT
GAACAAGGGCCCCTCCTTGTGACCCATGGTGCCTGGCTTGCCTTGGCCCCAGCCCTCCTCCCTGTGCCGA
AATCATCTCTCTGTGGACCTTTCTCCCTCATGGACTCCCACCTCCTTGTGGGCAGGAACCAGCCCTTGGC
CATGTTCCGGCTCCCTAGTGCCCAGTATGTGTTTGGTGAATTTGGGGGCATGGGTTGGATGATCTTGGTT
CAGTTACCAAATCTTTCTTTCCTGTCCAATCAGTCTTCCAGGATCTCAACCAGTTCTCTCACTGCAGAGT
CTGGAGAAGCCCAGCCCTCATTGTGAAGGGAGAGCCACCGTGGTGCTCACAGCAAGACACCCAGAGTCCC
TTCCAGACAGGGACACCCCTGGAGCGACCCTGTTTCAGAATGAAGTTATCTGGGTGGGAACTCACCAAGA
ATGCACAGAGGGCGCTGGGCTCCAAGCTTCAGCATATCCTTTCACTGGACTCCACACAGGCCTGCGGAGC
AGGTGGACCCACCATTCTCAGACCCCCCAGGGCCCCATAGACAGCAGATCTGCTGCTTTCAATCACACCC
ACTGCCTCTCATGCCGGAGTGGGGGAAATGGAACCCGCAAAGCCTGCAGGCCAGGGAGGGTGGGAAACTG
GGGTGATGTGAGCCCACAGGGTGGAGCACTGTGGACTGAATGCCTGTGTCCCCTGGGTTCATATGTTGAA
CCCCTAACTCCCAATGTGATGGTGTCAGGAGGTGGAGCCTTTGGGAGGTGGTTAGGCCGTGAGGGTGGAG
ACCCATGCTGGGGTTGGTGCCCTTATGGTTCTCCCATGTGAGGACACAGAGGGCAGCCATCTACAAGCCA
AGAAGAGAGGCCTCACCAGACACCAAACCTGCCGGCACCTTGCTGTGAGATTTCCAGCCCCCAGAGCTGT
GAGAAATAAATTTCTGTTGTTTAAGGCACTCAA
SEQ ID NO: 14 is the amino acid sequence for human RRM2. isoform 2.
>NP_001025.1 ribonucleoside-diphosphate reductase subunit M2 isoform 2
[Homo sapiens]
MLSLRVPLAPITDPQQLQLSPLKGLSLVDKENTPPALSGTRVLASKTARRIFQEPTEPKTKAAAPGVEDE
PLLRENPRRFVIFPIEYHDIWQMYKKAEASFWTAEEVDLSKDIQHWESLKPEERYFISHVLAFFAASDGI
VNENLVERFSQEVQITEARCFYGFQIAMENIHSEMYSLLIDTYIKDPKEREFLFNAIETMPCVKKKADWA
LRWIGDKEATYGERVVAFAAVEGIFFSGSFASIFWLKKRGLMPGLTFSNELISRDEGLHCDFACLMFKHL
VHKPSEERVREIIINAVRIEQEFLTEALPVKLIGMNCTLMKQYIEFVADRLMLELGFSKVFRVENPFDFM
ENISLEGKTNFFEKRVGEYQRMGVMSSPTENSFTLDADF
SEQ ID NO: 15 is the mRNA sequence for human RRM2, isoform 2.
>NM_001034.4 Homo sapiens ribonucleotide reductase regulatory subunit M2
(RRM2), transcript variant 2, mRNA
GTGCACCCTGTCCCAGCCGTCCTGTCCTGGCTGCTCGCTCTGCTTCGCTGCGCCTCCACTATGCTCTCCC
TCCGTGTCCCGCTCGCGCCCATCACGGACCCGCAGCAGCTGCAGCTCTCGCCGCTGAAGGGGCTCAGCTT
GGTCGACAAGGAGAACACGCCGCCGGCCCTGAGCGGGACCCGCGTCCTGGCCAGCAAGACCGCGAGGAGG
ATCTTCCAGGAGCCCACGGAGCCGAAAACTAAAGCAGCTGCCCCCGGCGTGGAGGATGAGCCGCTGCTGA
GAGAAAACCCCCGCCGCTTTGTCATCTTCCCCATCGAGTACCATGATATCTGGCAGATGTATAAGAAGGC
AGAGGCTTCCTTTTGGACCGCCGAGGAGGTGGACCTCTCCAAGGACATTCAGCACTGGGAATCCCTGAAA
CCCGAGGAGAGATATTTTATATCCCATGTTCTGGCTTTCTTTGCAGCAAGCGATGGCATAGTAAATGAAA
ACTTGGTGGAGCGATTTAGCCAAGAAGTTCAGATTACAGAAGCCCGCTGTTTCTATGGCTTCCAAATTGC
CATGGAAAACATACATTCTGAAATGTATAGTCTTCTTATTGACACTTACATAAAAGATCCCAAAGAAAGG
GAATTTCTCTTCAATGCCATTGAAACGATGCCTTGTGTCAAGAAGAAGGCAGACTGGGCCTTGCGCTGGA
TTGGGGACAAAGAGGCTACCTATGGTGAACGTGTTGTAGCCTTTGCTGCAGTGGAAGGCATTTTCTTTTC
CGGTTCTTTTGCGTCGATATTCTGGCTCAAGAAACGAGGACTGATGCCTGGCCTCACATTTTCTAATGAA
CTTATTAGCAGAGATGAGGGTTTACACTGTGATTTTGCTTGCCTGATGTTCAAACACCTGGTACACAAAC
CATCGGAGGAGAGAGTAAGAGAAATAATTATCAATGCTGTTCGGATAGAACAGGAGTTCCTCACTGAGGC
CTTGCCTGTGAAGCTCATTGGGATGAATTGCACTCTAATGAAGCAATACATTGAGTTTGTGGCAGACAGA
CTTATGCTGGAACTGGGTTTTAGCAAGGTTTTCAGAGTAGAGAACCCATTTGACTTTATGGAGAATATTT
CACTGGAAGGAAAGACTAACTTCTTTGAGAAGAGAGTAGGCGAGTATCAGAGGATGGGAGTGATGTCAAG
TCCAACAGAGAATTCTTTTACCTTGGATGCTGACTTCTAAATGAACTGAAGATGTGCCCTTACTTGGCTG
ATTTTTTTTTTCCATCTCATAAGAAAAATCAGCTGAAGTGTTACCAACTAGCCACACCATGAATTGTCCG
TAATGTTCATTAACAGCATCTTTAAAACTGTGTAGCTACCTCACAACCAGTCCTGTCTGTTTATAGTGCT
GGTAGTATCACCTTTTGCCAGAAGGCCTGGCTGGCTGTGACTTACCATAGCAGTGACAATGGCAGTCTTG
GCTTTAAAGTGAGGGGTGACCCTTTAGTGAGCTTAGCACAGCGGGATTAAACAGTCCTTTAACCAGCACA
GCCAGTTAAAAGATGCAGCCTCACTGCTTCAACGCAGATTTTAATGTTTACTTAAATATAAACCTGGCAC
TTTACAAACAAATAAACATTGTTTGTACTCACAAGGCGATAATAGCTTGATTTATTTGGTTTCTACACCA
AATACATTCTCCTGACCACTAATGGGAGCCAATTCACAATTCACTAAGTGACTAAAGTAAGTTAAACTTG
TGTAGACTAAGCATGTAATTTTTAAGTTTTATTTTAATGAATTAAAATATTTGTTAACCAACTTTAAAGT
CAGTCCTGTGTATACCTAGATATTAGTCAGTTGGTGCCAGATAGAAGACAGGTTGTGTTTTTATCCTGTG
GCTTGTGTAGTGTCCTGGGATTCTCTGCCCCCTCTGAGTAGAGTGTTGTGGGATAAAGGAATCTCTCAGG
GCAAGGAGCTTCTTAAGTTAAATCACTAGAAATTTAGGGGTGATCTGGGCCTTCATATGTGTGAGAAGCC
GTTTCATTTTATTTCTCACTGTATTTTCCTCAACGTCTGGTTGATGAGAAAAAATTCTTGAAGAGTTTTC
ATATGTGGGAGCTAAGGTAGTATTGTAAAATTTCAAGTCATCCTTAAACAAAATGATCCACCTAAGATCT
TGCCCCTGTTAAGTGGTGAAATCAACTAGAGGTGGTTCCTACAAGTTGTTCATTCTAGTTTTGTTTGGTG
TAAGTAGGTTGTGTGAGTTAATTCATTTATATTTACTATGTCTGTTAAATCAGAAATTTTTTATTATCTA
TGTTCTTCTAGATTTTACCTGTAGTTCATACTTCAGTCACCCAGTGTCTTATTCTGGCATTGTCTAAATC
TGAGCATTGTCTAGGGGGATCTTAAACTTTAGTAGGAAACCATGAGCTGTTAATACAGTTTCCATTCAAA
TATTAATTTCAGAATGAAACATAATTTTTTTTTTTTTTTTTGAGATGGAGTCTCGCTCTGTTGCCCAGGC
TGGAGTGCAGTGGCGCGATTTTGGCTCACTGTAACCTCCATCTCCTGGGTTCAAGCAATTCTCCTGTCTC
AGCCTCCCTAGTAGCTGGGACTGCAGGTATGTGCTACCACACCTGGCTAATTTTTGTATTTTTAGTAGAG
ATGGAGTTTCACCATATTGGTCAGGCTGGTCTTGAACTCCTGACCTCAGGTGATCCACCCACCTCGGCCT
CCCAAAGTGCTGGGATTGCAGGCGTGATAAACAAATATTCTTAATAGGGCTACTTTGAATTAATCTGCCT
TTATGTTTGGGAGAAGAAAGCTGAGACATTGCATGAAAGATGATGAGAGATAAATGTTGATCTTTTGGCC
CCATTTGTTAATTGTATTCAGTATTTGAACGTCGTCCTGTTTATTGTTAGTTTTCTTCATCATTTATTGT
ATAGACAATTTTTAAATCTCTGTAATATGATACATTTTCCTATCTTTTAAGTTATTGTTACCTAAAGTTA
ATCCAGATTATATGGTCCTTATATGTGTACAACATTAAAATGAAAGGCTTTGTCTTGCATTGTGAGGTAC
AGGCGGAAGTTGGAATCAGGTTTTAGGATTCTGTCTCTCATTAGCTGAATAATGTGAGGATTAACTTCTG
CCAGCTCAGACCATTTCCTAATCAGTTGAAAGGGAAACAAGTATTTCAGTCTCAAAATTGAATAATGCAC
AAGTCTTAAGTGATTAAAATAAAACTGTTCTTATGTCA
SEQ ID NO: 16 is the nucleotide sequence encoding mouse RRM2, isoform 2.
NCBI GeneID: 20135
SEQ ID NO: 17 is the amino acid sequence for mouse RRM2, isoform 2.
>NP_033130.1 ribonucleoside-diphosphate reductase subunit M2 [Mus musculus]
MLSVRTPLATIADQQQLQLSPLKRLTLADKENTPPTLSSTRVLASKAARRIFQDSAELESKAPTNPSVED
EPLLRENPRRFVVFPIEYHDIWQMYKKAEASFWTAEEVDLSKDIQHWEALKPDERHFISHVLAFFAASDG
IVNENLVERFSQEVQVTEARCFYGFQIAMENIHSEMYSLLIDTYIKDPKEREYLFNAIETMPCVKKKADW
ALRWIGDKEATYGERVVAFAAVEGIFFSGSFASIFWLKKRGLMPGLTFSNELISRDEGLHCDFACLMFKH
LVHKPAEQRVREIITNAVRIEQEFLTEALPVKLIGMNCTLMKQYIEFVADRLMLELGFNKIFRVENPFDF
MENISLEGKTNFFEKRVGEYQRMGVMSNSTENSFTLDADF
SEQ ID NO: 18 is the mRNA sequence for mouse RRM2, isoform 2.
>NM_009104.2 Mus musculus ribonucleotide reductase M2 (Rrm2), mRNA
TTTAAAGGGCGCGGGCGCTGGCAGTCGGCGGTGCACCGGATTCCAGCTGTTTTCGCCTGCTCCTCGCCGT
CTCCGCCGCTGCCCTCGTTCGCCATGCTCTCCGTCCGCACCCCGCTCGCCACCATCGCTGACCAGCAGCA
GCTGCAGTTGTCGCCGCTGAAGCGACTCACCCTGGCTGACAAGGAGAACACGCCCCCGACTCTCAGCAGC
ACCCGCGTCCTGGCCAGCAAAGCTGCGAGGAGAATCTTCCAGGACTCCGCCGAGCTGGAAAGTAAAGCGC
CTACTAACCCCAGCGTTGAGGATGAGCCGTTACTGAGAGAAAACCCCCGCCGCTTCGTTGTCTTTCCCAT
CGAGTACCATGATATCTGGCAGATGTACAAGAAAGCCGAGGCCTCCTTTTGGACTGCCGAGGAGGTGGAC
CTTTCCAAGGATATTCAGCACTGGGAAGCTCTGAAACCCGATGAGAGACATTTTATATCTCACGTTCTGG
CTTTCTTTGCAGCGAGTGATGGCATAGTCAATGAGAACTTGGTGGAGCGATTTAGCCAAGAAGTTCAAGT
TACAGAGGCCCGCTGTTTCTATGGCTTCCAAATTGCCATGGAAAACATACACTCTGAAATGTACAGTCTC
CTTATTGACACTTACATTAAAGATCCCAAGGAAAGAGAATATCTCTTCAATGCTATTGAAACAATGCCTT
GTGTGAAGAAGAAGGCTGACTGGGCCTTGCGCTGGATTGGGGACAAAGAGGCTACGTATGGAGAACGCGT
TGTGGCCTTTGCCGCCGTAGAAGGAATCTTCTTTTCCGGTTCTTTTGCATCGATATTCTGGCTCAAGAAA
CGGGGGCTGATGCCGGGCCTTACATTTTCCAATGAGCTTATTAGCAGAGACGAGGGTTTACACTGTGACT
TTGCCTGCCTGATGTTCAAGCACCTGGTACACAAGCCAGCAGAGCAGAGGGTCCGAGAGATAATCACCAA
CGCCGTTAGGATAGAGCAGGAGTTCCTCACGGAGGCCTTGCCCGTGAAGCTCATCGGGATGAACTGCACT
TTGATGAAGCAGTACATTGAGTTTGTGGCCGACAGGCTTATGCTGGAGCTGGGTTTTAACAAGATTTTCA
GAGTAGAAAATCCGTTTGACTTCATGGAAAATATCTCACTAGAAGGAAAGACAAACTTCTTTGAGAAGCG
AGTAGGCGAGTATCAGAGGATGGGAGTCATGTCGAATTCGACAGAGAACTCTTTTACCTTGGATGCTGAC
TTCTAAGTAACTGATCGTGTGTTCTTCGCTGATTTTTGTCCCCTTGCCATTAAAAGAAACCAGCAAAAAC
AACCAACTGGCTACACCATGAATTGTCATTAAATTTGCTAAACAGGTGTCTAAAAAGCTGTGTAGCTACC
TCAGTCCTGTTTGCCAGGCTGGTCACTAGAAGAAAGTATACTTCAAACAATGGGTACTTGGATCCTTAGG
GAGATCCTGTCCTTGGCTTTTACAAGTAGTGTGGTCACCTTTGACCTCATCAAAGTACTAACAGCACTGG
GCCAGGTTTTAGGAGCAGTGACCATCAAGCAAGCAGGTTTAAACATTTAGATGCTGTTTAGGGCTGTTTA
AAGATGTCGGACTGCTTCCTGCAGGCATGCAGGGTCTACTTAACAAGTTTGTAAATAAAATTGGCACTTT
GCACACACACACACATAGTGCTGTCAGGCGATTAAACTATACATTTTATGAGGTAGTACCTCTATGCTTT
TTTTTTTTTTTTTTAATGCTCAGTATTATCTTGAAGTTTGCAAATGCTATGATGGTACAGTAAATTCTGA
CATTTGCCCTAATAGTGTCACTTTTTTTTTTTCTTCGAGACAGAGTTTCTCTGTATAGCCCTGGCTGTAC
GGAATTCACAAGTGAGTTTGAGCCCAGTGGTGGGTACACCCGTGGGACTCTTACAAACCAAAACAGGAAA
AGCAAGTGTTCCCTGAGGTAGTTTACTGTGATCTAGCTTCCTCATGAACTGACATAACCCTGATCAGTTT
CCTTGATTATTGTATAGATGTTTTTGTAATATGAAAAGCCTTTGTACCTTTTAAATTATTGTTACTTAAA
ATTAATAAACTCTTGAATTAACAGTCTTGAACTTTCATGGCATACAAGTATTAAATGATTTAACTAAAAC
CTTAATGTCAAAAAAAAAAAAAAAAAAAA
SEQ ID NO: 19 is the nucleotide sequence encoding rat RRM2, isoform 2
GeneID: 362720
SEQ ID NO: 20 is the amino acid sequence for rat RRM2, isoform 2.
>NP_001020911.1 ribonucleoside-diphosphate reductase subunit M2
[Rattus norvegicus]
MLSVRAPLATIADQQQLHLSPLKRLSLADKENTPPTLSSARVLASKAARRIFQDSAELESKAPTKPSIEE
EPLLRENPRRFVVFPIEYHDIWQMYKKAEASFWTAEEVDLSKDIQHWEALKPDERHFISHVLAFFAASDG
IVNENLVERFSQEVQVTEARCFYGFQIAMENIHSEMYSLLIDTYIKDSKEREYLFNAIETMPCVKKKADW
ALRWIGDKEATYGERVVAFAAVEGIFFSGSFASIFWLKKRGLMPGLTFSNELISRDEGLHCDFACLMFKH
LVHKPSEQRVKEIITNSVRIEQEFLTEALPVKLIGMNCTLMKQYIEFVADRLMLELGFNKIFKVENPFDF
MENISLEGKTNFFEKRVGEYQRMGVMSNSTENSFTLDADF
SEQ ID NO: 21 is the mRNA sequence for rat RRM2, isoform 2.
>NM_001025740.1 Rattus norvegicus ribonucleotide reductase regulatory
subunit M2 (Rrm2), mRNA
TCCAGCTGTTCCCTCTTCTCCTCGTCCTCTCCACCTCTGCCTTCGTTCGCCATGCTCTCGGTCCGCGCCC
CGCTCGCCACCATCGCTGACCAGCAGCAGCTGCACTTGTCGCCCCTGAAGCGACTCAGTCTGGCTGACAA
GGAGAACACGCCCCCAACCCTCAGCAGCGCCCGCGTCCTGGCTAGCAAGGCTGCAAGGAGAATCTTCCAG
GACTCTGCCGAGCTGGAAAGTAAAGCACCCACTAAGCCCAGCATTGAGGAAGAGCCGTTACTGAGAGAAA
ATCCCCGCCGTTTCGTTGTCTTTCCCATCGAATACCATGATATCTGGCAGATGTACAAGAAAGCTGAGGC
CTCCTTTTGGACTGCCGAGGAGGTGGACCTTTCCAAGGATATTCAGCACTGGGAAGCTCTGAAACCAGAT
GAGAGACATTTTATATCTCATGTTCTGGCCTTCTTTGCGGCGAGTGACGGCATAGTCAATGAGAACTTGG
TGGAGCGATTTAGCCAAGAAGTTCAAGTCACAGAGGCCCGCTGTTTCTATGGCTTCCAAATTGCCATGGA
GAACATACACTCCGAAATGTACAGTCTCCTTATTGACACTTACATTAAAGATTCCAAAGAAAGAGAATAT
CTCTTCAACGCCATTGAGACAATGCCTTGTGTGAAGAAGAAGGCTGACTGGGCCTTGCGTTGGATTGGGG
ACAAAGAGGCTACGTATGGAGAACGAGTTGTGGCCTTCGCTGCGGTAGAAGGAATCTTCTTTTCTGGTTC
TTTTGCATCAATATTCTGGCTCAAGAAACGGGGACTGATGCCGGGCCTTACATTTTCCAATGAGCTTATT
AGCAGAGATGAGGGTCTGCACTGTGACTTTGCCTGCCTGATGTTCAAGCACCTGGTACACAAGCCCTCGG
AGCAGAGAGTAAAAGAAATAATTACCAACTCGGTCAGGATAGAGCAGGAGTTCCTCACAGAGGCCCTGCC
TGTGAAGCTCATCGGGATGAATTGCACCTTGATGAAGCAGTACATCGAGTTTGTGGCCGACAGGCTTATG
CTGGAGCTGGGTTTTAACAAGATTTTCAAAGTAGAAAATCCATTTGACTTCATGGAGAATATTTCACTAG
AAGGAAAAACAAACTTCTTTGAGAAGCGAGTAGGCGAGTACCAGAGGATGGGAGTAATGTCAAATTCGAC
AGAAAATTCTTTCACCTTGGATGCTGACTTCTAAGCAACCGATCCGTGTGCTCTTTGCTGATTATTCTCC
CCTTGTCATTAAAAGAAATCAGCAAAACCAAACAACTGGCTACACCACGAATTGTCGTTAAATTTGCTAA
CTGGTGTCTAAAAGCCGTGTAGCTACCTCGGTCCTGCTTGCTAGGTTTGCCACTAGAAGGAAGCATACTT
AAAACAATGGCTACTTGGATCCTCAGGGAGATCCTGTCTGCAAGTCGCGTGGTCACCCTTAGCTTCATCA
AAGCACTAACAGCTCACCCGGCCAGGCTTCATGAGCACTGACCCTCAAGCAAGCAGGTTTATTAAACATT
TAGATGCCAACCTCACTTACTGTTTCCTGCAGTCATGGAGAGTTTACTTAACAAGTTTGTAAATAATAAA
ACTGGCACTTTGCACACAGACTTGGTACTATCCTAGGGGAAGGCCTGCTTTATTTGGTTTCTAGACCGAG
TAGGAAGTGATCCATTTACCACTGAGGGCAGCCCCATTCAGAGTCTTAAGTGACTAAGCCAGTGTTGAAC
AAGCAATTTCCAGGCTTTGTTCTTCAGGGAACTTCCCATCAGCTTTGAAGTCGGTCCTGTGCACCCTAGG
CACATGGATCAGTTCACAAGTGGGGTTCAGTGGAGAGAACTTCCCCCTCAGAAGTCACTTGAAACTTAGA
TGAGATTTGGGACACTTGCTGGTTGACTCTGTCTCATTTGTGTAAAAAGTAGTTTTTTTTTTTTTTTTTT
TCCAAGTTATACTTTGTCCCATTCCTAGTTAGTACAAAGTCTTGAAAGGGCCTTTGTAGGGCTTTTTAAG
TCAGGGTCTTAACTATGTAACTCTGGCTTGGCCTGGAACTTGCTATGTAGACCAGGTTACCCTCAAACTT
GCCTGTCTTCCCAAATACTGGGATTAAGGTTTCTGTGACCATACCTGGCTTTACCTGATTAATTCCTAAA
CACCAGAAAACCAGTACTGTATGAGATGTTAATGTGTGTTCCTTTCAGACTGGAGTACAGACCAGTAGAT
AACAGATAACAGCTGGTTCACCTTAATCTGCCTTTTTGTGTATTAATCTGTGTTTAGAGAACGGAACAAT
AGCCAGAATTCACCTAGCGAGTTCGAGGCCAGTTGGTGTATATGTGGGACTCTTAACCAAAACAGCAAGC
GTTCCCTGGGGTAGTTCACAATGATCTCCAGCTTCCTTGTTAACCAGATAACTGCAAGTCAGATGTATGA
CCCTGGTTGGTTTATTGTATTGATATGTTTCTGTAATATGAGTAAATTATTGTTACTTAAAAGTAATAAA
CAAAATTGAAAAAAAAAAAAAAAAAAAAAAAAAA
SEQ ID NO: 22 is the nucleotide sequence encoding canine RRM2, isoform 2.
NCBI Gene ID: 482963
SEQ ID NO: 23 is the amino acid sequence for canine RRM2, isoform 2
>XP_540076.2 ribonucleoside-diphosphate reductase subunit M2 [Canis
lupus familiaris]
MLSVRVPLATIADPQQQQQQQLQLSPLKGLSLADKENTPPALSGTRVLASKTARRIFQEPAEPKTKVLAP
SAEEEPLLRENPRRFVIFPIEYHDIWQMYKKAEASFWTAEEVDLSKDIQHWESLKPEERYFISHVLAFFA
ASDGIVNENLVERFSQEVQITEARCFYGFQIAMENIHSEMYSLLIDTYIKDSKEREFLFNAIETMPCVKK
KADWALRWIGDKEATYGERVVAFAAVEGIFFSGSFASIFWLKKRGLMPGLTFSNELISRDEGLHCDFACL
MFKHLVHKPSEQRVKEIIINAVRIEQEFLTEALPVKLIGMNCTLMKQYIEFVADRLMLELGFSKVFRVEN
PFDEMENISLEGKTNFFEKRVGEYQRMGVMSSPTENSFTLDADF
SEQ ID NO: 24 is the mRNA sequence for canine RRM2, isoform 2
>XM_540076.6 PREDICTED: Canis lupus familiaris ribonucleotide reductase
regulatory subunit M2 (RRM2), mRNA
TGCGCCGCCGCCGCCGCCGAGCCCGCCTGCCGCCGCCATGCTCTCCGTCCGCGTCCCGCTCGCCACCATC
GCGGACCCCCAGCAGCAGCAGCAGCAGCAGCTCCAGCTCTCGCCCCTCAAGGGGCTCAGCCTGGCGGACA
AGGAGAACACGCCCCCGGCCCTCAGCGGCACCCGCGTGCTGGCCAGCAAGACCGCCCGGAGGATCTTCCA
GGAGCCCGCCGAGCCGAAAACTAAGGTACTTGCCCCCAGCGCGGAGGAGGAACCACTGCTGAGAGAAAAC
CCCCGTCGCTTTGTCATCTTCCCTATCGAGTACCATGATATTTGGCAGATGTATAAGAAAGCGGAGGCTT
CCTTTTGGACAGCCGAAGAGGTGGATCTTTCCAAGGACATTCAGCACTGGGAATCCCTGAAGCCTGAGGA
GAGATATTTTATATCCCATGTTCTGGCTTTCTTTGCAGCGAGCGATGGCATAGTAAATGAAAACTTGGTG
GAGCGGTTTAGCCAAGAAGTTCAGATTACGGAAGCCCGCTGTTTCTATGGCTTCCAAATTGCCATGGAAA
ACATCCACTCTGAGATGTATAGTCTCCTCATTGACACTTATATTAAAGATTCCAAAGAAAGGGAATTTCT
CTTCAACGCCATTGAGACGATGCCTTGTGTAAAGAAGAAGGCAGATTGGGCCTTGCGTTGGATTGGGGAC
AAAGAAGCTACCTATGGAGAACGGGTTGTGGCCTTTGCTGCCGTGGAAGGAATCTTCTTTTCGGGTTCTT
TTGCGTCAATATTCTGGCTCAAGAAACGGGGCCTGATGCCTGGCCTCACGTTTTCCAATGAACTTATTAG
CAGAGATGAGGGTTTACACTGTGACTTTGCCTGCCTGATGTTCAAACACCTGGTCCACAAACCTTCAGAG
CAGAGGGTGAAGGAAATAATTATCAATGCCGTTAGGATAGAACAGGAGTTCCTCACGGAGGCTTTGCCAG
TGAAGCTCATTGGGATGAATTGCACGTTAATGAAGCAGTATATTGAATTCGTGGCAGACCGACTTATGCT
GGAGCTCGGTTTTAGCAAGGTTTTCAGAGTAGAAAATCCATTTGACTTTATGGAGAATATTTCACTGGAA
GGGAAGACTAACTTCTTTGAGAAGAGAGTAGGCGAGTATCAGAGGATGGGAGTGATGTCAAGTCCAACAG
AGAATTCGTTTACCTTGGATGCTGACTTCTAAGTGAATGAAGATGTGCTCTTTGCTGATTTTTTTTTTTC
CCTTTCTCATCCAAAAAAAAAAAAATCAGCTACTTGAAGTGTATCAAACCAGCTACACCATGAATCATCC
ATAACGTTCATTAATAGTATTGTTAAAACTGTGTAGCTACCTCATAAGCAAGCCTGTTGATCAGTTAATG
CTAGTCGTCTCACCCAGAAAGAAGCATAGACAAAAAGCTACTCGGATTCTTAATGAAAGATATTGGCCGT
GTTTGGCTTTTGCGGGCAGGCTGGCTGTCCACTGACTTCACAGTGGCTCTTGGTGGCAGTCAGGTCTCAA
AAGTGTGGGGACTCAAGTGAGTCTGATCTAGCACGATTAATTAGTTAGTCTAAGGCCCTTGGGCGGTGTC
AGCCTCTGTGCTTCAAAGCAGATTTTTAAGTTTACGTACCGATTTTTATATAAAACTGGCACTTTACACA
CAAATAAACATAGTTTGTACTGTTGAAATAAAGGCTTGATTTAACTTAATCTGGTTTCTAGCCCAAATGC
AGAGCATTCTATTGACCACTAATGGGAGCCAGTTTGCAATTTACTAGGTAACCAAAAAGTCCATCAAACC
TGTGTGAATCAAGCATGTTATTTCTGTTTATTTTCTATAATGAATTGATGTTCTCTTTAATCAACTTTAA
AGTCAATCCTTCATATACCTAGGTATTAGCCACTTGGTGCCATGAAGAAACGCAGGTTGTGTTTTATATT
TTGGAGGCCAGGTCAAGTATTGTGGATAAGAGGGGAAAGGAGGTTCCAATTAAATCATTAGAGCTTGAAG
TGTGATGTAGGCTGACTGCTGGTCGCCTGGGGGTGTGCGAGGATCAGCATCCTTTTATTTCTCAAACCAC
ATTTTCCCCCACCTTGAGTTCTTATAGAAAGAAGATCCTTAGATCCTTAGCTGTAGGGTCTGAGATAATA
TTGTAAATTGATTTTGAAATCAATCCTTGCACGAATTGACCCGCTTAGGATCTTGCTCCAATTAAGTGGC
ACAACCAGAACTGAAATTGGCTCCCCGGAAAGTTGAGCATTTTCTCTGATTTGGTCTAATTTGTAAGTAG
GTAATGTTGACCTAATCCATTTGTGTCTACTACATGTTTTTTCAATTAGATATTTCTTCTGTTTTTTTGT
TCTTTTATATCTGGTTCATATTTTGAAATAATTGCTCAGTTAGTGCAGTTCATGATTGGAGCAGATAGTC
TTCAGGGCACTTACTTCCAGCTTTTGCCTCAATCTGAGCATTACCTTGTTGGATTCCTGACCTGCAGTAG
AAAACTAGAGTTGCATGAGCTATATTAATACAGGTTCTGTTCACACAGTAATTTTAGAAAGAAGTATAAA
ATAATATACTTAATAGGATTAGTTTGAATCAACCTGTCTTTGTGTTACCCCTGCTTTCTCCCTCCCCATC
AAAAAAAAAAAAAAAGAAAAAAAAACAAAAAACCCAGCCAGGAGGTTACGAGAAGGTGGTGGATGATACG
CACTGATCCTTTGGCCACATTTGTTAACCTGTCTTTTTGTGTTGGGTGATCACTGACCTGTTTTTTTGTC
AGTTTTCTTCATTTATTGTATAAATTGTCAAATAGTCAATTTAAAAATTTCTGTAACGGTGGCTGTCTTT
TAAATTATTGTTACCTGAAGTGAATCTAGATAATGTGGTTCTTACCCTTGTGCAACACAAAGGTGAATAA
ACGTTTTTGCCTCGCGTGTCGGGTGCAGACGGAA
SEQ ID NO: 25 is an exemplary nucleic acid sequence comprising a Kozak sequence, RRM1,
P2A, and RRM2.
GCTAGCGAATTCGCCACCATGCACGTCATCAAGAGAGACGGGAGGCAGGAAAGAGTCATGTTCGATAAAATCACTTCAAGAATCCA
GAAACTGTGTTACGGGCTGAACATGGACTTCGTCGATCCTGCCCAGATTACCATGAAAGTGATCCAGGGACTGTACTCTGGCGTCA
CCACAGTGGAGCTGGACACACTGGCCGCTGAAACCGCAGCCACACTGACTACCAAACACCCAGATTATGCAATTCTGGCTGCACGG
ATCGCCGTGAGTAATCTGCATAAGGAGACAAAGAAAGTCTTCTCAGACGTGATGGAGGACCTGTACAATTATATCAACCCTCACAA
TGGGAAACATTCACCAATGGTCGCTAAGAGCACTCTGGACATTGTGCTGGCCAACAAAGATCGGCTGAACAGCGCTATCATCTACG
ACCGGGATTTCAGTTACAACTACTTCGGCTTTAAGACACTGGAGAGATCATATCTGCTGAAAATCAATGGGAAGGTGGCCGAACGG
CCTCAGCACATGCTGATGAGAGTCAGCGTGGGCATTCATAAGGAGGACATTGATGCCGCTATCGAAACTTACAACCTGCTGAGCGA
GCGCTGGTTCACCCACGCTTCCCCTACACTGTTTAACGCAGGAACCAATCGACCACAGCTGAGCAGCTGCTTCCTGCTGAGCATGA
AGGACGATTCCATCGAGGGCATCTACGACACCCTGAAACAGTGCGCACTGATTTCTAAGAGTGCCGGCGGGATCGGAGTCGCTGTG
AGTTGTATTCGGGCAACCGGCTCATATATCGCCGGCACAAACGGCAACAGCAACGGGCTGGTCCCCATGCTGAGGGTGTACAACAA
TACAGCCCGCTATGTGGATCAGGGAGGCAACAAGAGACCAGGAGCATTTGCCATCTACCTGGAACCCTGGCACCTGGACATTTTCG
AGTTTCTGGATCTGAAGAAAAATACTGGCAAAGAGGAACAGAGGGCTCGCGACCTGTTCTTTGCACTGTGGATTCCCGACCTGTTC
ATGAAGAGGGTGGAGACCAACCAGGACTGGAGCCTGATGTGCCCCAATGAGTGTCCTGGGCTGGATGAAGTGTGGGGAGAGGAATT
TGAAAAACTGTACGCCAGTTATGAGAAGCAGGGCCGAGTGCGGAAAGTGGTCAAGGCCCAGCAGCTGTGGTACGCTATCATTGAGA
GCCAGACAGAAACTGGCACCCCCTACATGCTGTATAAAGACTCTTGCAACCGCAAGAGTAACCAGCAGAATCTGGGGACCATCAAA
TGCAGCAATCTGTGTACAGAGATTGTGGAATATACTTCCAAGGATGAGGTCGCCGTGTGTAACCTGGCATCACTGGCCCTGAATAT
GTACGTCACAAGCGAGCACACTTATGACTTCAAGAAACTGGCTGAAGTGACCAAAGTGGTCGTGAGGAATCTGAACAAGATCATTG
ACATCAACTACTATCCCGTGCCTGAGGCCTGCCTGAGCAATAAGAGACATAGGCCCATCGGGATTGGAGTGCAGGGCCTGGCTGAC
GCATTCATCCTGATGCGCTACCCTTTTGAGTCCGCCGAAGCTCAGCTGCTGAACAAGCAGATTTTTGAAACAATCTACTACGGGGC
TCTGGAGGCATCTTGTGACCTGGCCAAAGAACAGGGACCCTACGAGACTTATGAAGGCTCCCCTGTGTCTAAGGGCATCCTGCAGT
ACGATATGTGGAACGTCACACCAACTGACCTGTGGGATTGGAAAGTGCTGAAGGAGAAAATTGCAAAGTATGGCATCCGGAACAGC
CTGCTGATCGCCCCAATGCCCACTGCCTCTACCGCTCAGATTCTGGGCAACAATGAGTCCATCGAACCATACACTTCTAACATCTA
CACCCGGAGAGTCCTGAGCGGGGAGTTCCAGATCGTGAATCCCCACCTGCTGAAAGACCTGACCGAACGGGGACTGTGGCATGAGG
AAATGAAGAACCAGATCATTGCCTGCAATGGCAGTATCCAGTCAATTCCTGAGATCCCAGACGATCTGAAACAGCTGTACAAGACA
GTCTGGGAGATCAGCCAGAAAACTGTGCTGAAGATGGCAGCCGAAAGAGGGGCTTTCATTGATCAGTCACAGAGCCTGAACATCCA
CATTGCCGAGCCCAATTACGGAAAGCTGACCTCCATGCATTTTTATGGGTGGAAACAGGGACTGAAGACTGGCATGTACTATCTGC
GCACCCGACCAGCTGCAAACCCCATCCAGTTTACCCTGAATAAGGAGAAACTGAAGGACAAAGAAAAGGTGTCCAAAGAGGAAGAG
GAAAAGGAGAGAAACACAGCCGCTATGGTGTGTTCTCTGGAGAATAGGGATGAATGCCTGATGTGTGGCAGTGGAAGCGGAGCTAC
TAACTTCAGCCTGCTGAAGCAGGCTGGAGACGTGGAGGAGAACCCTGGACCTCTGAGTCTGAGGGTCCCACTGGCACCTATCACCG
ATCCACAGCAGCTGCAGCTGAGCCCACTGAAAGGCCTGAGTCTGGTCGATAAAGAGAACACACCACCTGCACTGAGTGGCACTCGG
GTGCTGGCATCAAAGACCGCCCGGAGAATTTTCCAGGAGCCAACCGAACCCAAAACAAAGGCCGCTGCACCTGGGGTCGAGGACGA
ACCACTGCTGAGAGAGAATCCCAGGCGCTTCGTGATTTTTCCTATCGAATACCACGATATTTGGCAGATGTATAAGAAAGCTGAGG
CAAGTTTCTGGACAGCTGAGGAAGTGGACCTGAGCAAAGACATCCAGCACTGGGAATCCCTGAAGCCAGAGGAAAGGTACTTCATT
TCTCATGTGCTGGCATTCTTTGCCGCTAGTGACGGGATCGTGAACGAGAATCTGGTCGAACGCTTTAGCCAGGAGGTGCAGATCAC
TGAAGCCCGATGCTTCTATGGATTTCAGATTGCTATGGAGAACATCCATTCAGAAATGTACAGCCTGCTGATTGACACCTATATCA
AAGATCCTAAGGAGCGCGAGTTCCTGTTTAATGCCATTGAGACAATGCCATGTGTGAAGAAAAAGGCAGACTGGGCTCTGCGATGG
ATCGGCGATAAGGAGGCTACTTACGGGGAAAGAGTGGTCGCATTCGCAGCCGTGGAGGGAATTTTCTTTTCTGGCAGTTTCGCTTC
CATCTTTTGGCTGAAAAAGCGAGGCCTGATGCCTGGGCTGACCTTTTCCAACGAGCTGATTTCTCGCGACGAAGGCCTGCACTGCG
ATTTCGCCTGTCTGATGTTTAAACACCTGGTGCATAAGCCCTCTGAGGAACGAGTCCGGGAGATCATTATCAACGCAGTGAGGATC
GAGCAGGAGTTCCTGACAGAAGCCCTGCCTGTCAAACTGATTGGCATGAATTGCACTCTGATGAAGCAGTACATCGAGTTTGTGGC
CGACAGGCTGATGCTGGAACTGGGATTCTCAAAGGTGTTTCGCGTCGAGAACCCATTCGATTTTATGGAGAATATCAGCCTGGAAG
GCAAAACAAACTTCTTTGAGAAGAGAGTCGGGGAATATCAGAGGATGGGCGTGATGAGCAGCCCCACTGAGAATAGCTTCACCCTG
GACGCCGATTTTTGAGCTAGC
SEQ ID NO: 26 is an exemplary Kozak sequence (as found in SEQ ID NO: 25).
GCCACC
SEQ ID NO: 27 is an exemplary RRM1 sequence (as found in SEQ ID NO: 25).
ATGCACGTCATCAAGAGAGACGGGAGGCAGGAAAGAGTCATGTTCGATAAAATCACTTCAAGAATCCAGAAACTGTGTTACGGGCT
GAACATGGACTTCGTCGATCCTGCCCAGATTACCATGAAAGTGATCCAGGGACTGTACTCTGGCGTCACCACAGTGGAGCTGGACA
CACTGGCCGCTGAAACCGCAGCCACACTGACTACCAAACACCCAGATTATGCAATTCTGGCTGCACGGATCGCCGTGAGTAATCTG
CATAAGGAGACAAAGAAAGTCTTCTCAGACGTGATGGAGGACCTGTACAATTATATCAACCCTCACAATGGGAAACATTCACCAAT
GGTCGCTAAGAGCACTCTGGACATTGTGCTGGCCAACAAAGATCGGCTGAACAGCGCTATCATCTACGACCGGGATTTCAGTTACA
ACTACTTCGGCTTTAAGACACTGGAGAGATCATATCTGCTGAAAATCAATGGGAAGGTGGCCGAACGGCCTCAGCACATGCTGATG
AGAGTCAGCGTGGGCATTCATAAGGAGGACATTGATGCCGCTATCGAAACTTACAACCTGCTGAGCGAGCGCTGGTTCACCCACGC
TTCCCCTACACTGTTTAACGCAGGAACCAATCGACCACAGCTGAGCAGCTGCTTCCTGCTGAGCATGAAGGACGATTCCATCGAGG
GCATCTACGACACCCTGAAACAGTGCGCACTGATTTCTAAGAGTGCCGGCGGGATCGGAGTCGCTGTGAGTTGTATTCGGGCAACC
GGCTCATATATCGCCGGCACAAACGGCAACAGCAACGGGCTGGTCCCCATGCTGAGGGTGTACAACAATACAGCCCGCTATGTGGA
TCAGGGAGGCAACAAGAGACCAGGAGCATTTGCCATCTACCTGGAACCCTGGCACCTGGACATTTTCGAGTTTCTGGATCTGAAGA
AAAATACTGGCAAAGAGGAACAGAGGGCTCGCGACCTGTTCTTTGCACTGTGGATTCCCGACCTGTTCATGAAGAGGGTGGAGACC
AACCAGGACTGGAGCCTGATGTGCCCCAATGAGTGTCCTGGGCTGGATGAAGTGTGGGGAGAGGAATTTGAAAAACTGTACGCCAG
TTATGAGAAGCAGGGCCGAGTGCGGAAAGTGGTCAAGGCCCAGCAGCTGTGGTACGCTATCATTGAGAGCCAGACAGAAACTGGCA
CCCCCTACATGCTGTATAAAGACTCTTGCAACCGCAAGAGTAACCAGCAGAATCTGGGGACCATCAAATGCAGCAATCTGTGTACA
GAGATTGTGGAATATACTTCCAAGGATGAGGTCGCCGTGTGTAACCTGGCATCACTGGCCCTGAATATGTACGTCACAAGCGAGCA
CACTTATGACTTCAAGAAACTGGCTGAAGTGACCAAAGTGGTCGTGAGGAATCTGAACAAGATCATTGACATCAACTACTATCCCG
TGCCTGAGGCCTGCCTGAGCAATAAGAGACATAGGCCCATCGGGATTGGAGTGCAGGGCCTGGCTGACGCATTCATCCTGATGCGC
TACCCTTTTGAGTCCGCCGAAGCTCAGCTGCTGAACAAGCAGATTTTTGAAACAATCTACTACGGGGCTCTGGAGGCATCTTGTGA
CCTGGCCAAAGAACAGGGACCCTACGAGACTTATGAAGGCTCCCCTGTGTCTAAGGGCATCCTGCAGTACGATATGTGGAACGTCA
CACCAACTGACCTGTGGGATTGGAAAGTGCTGAAGGAGAAAATTGCAAAGTATGGCATCCGGAACAGCCTGCTGATCGCCCCAATG
CCCACTGCCTCTACCGCTCAGATTCTGGGCAACAATGAGTCCATCGAACCATACACTTCTAACATCTACACCCGGAGAGTCCTGAG
CGGGGAGTTCCAGATCGTGAATCCCCACCTGCTGAAAGACCTGACCGAACGGGGACTGTGGCATGAGGAAATGAAGAACCAGATCA
TTGCCTGCAATGGCAGTATCCAGTCAATTCCTGAGATCCCAGACGATCTGAAACAGCTGTACAAGACAGTCTGGGAGATCAGCCAG
AAAACTGTGCTGAAGATGGCAGCCGAAAGAGGGGCTTTCATTGATCAGTCACAGAGCCTGAACATCCACATTGCCGAGCCCAATTA
CGGAAAGCTGACCTCCATGCATTTTTATGGGTGGAAACAGGGACTGAAGACTGGCATGTACTATCTGCGCACCCGACCAGCTGCAA
ACCCCATCCAGTTTACCCTGAATAAGGAGAAACTGAAGGACAAAGAAAAGGTGTCCAAAGAGGAAGAGGAAAAGGAGAGAAACACA
GCCGCTATGGTGTGTTCTCTGGAGAATAGGGATGAATGCCTGATGTGTGGCAGT
SEQ ID NO: 28 is an exemplary P2A sequence (as found in SEQ ID NO: 25).
GCTACTAACTTCAGCCTGCTGAAGCAGGCTGGAGACGTGGAGGAGAACCCTGGACCT
SEQ ID NO: 29-CK8 promoter

| | |
|---|---|
| ctagactagc atgctgccca tgtaaggagg caaggcctgg ggacacccga gatgcctggt | 60 |
| tataattaac ccagacatgt ggctgccccc ccccccccaa cacctgctgc ctctaaaaat | 120 |
| aaccctgcat gccatgttcc cggcgaaggg ccagctgtcc cccgccagct agactcagca | 180 |
| cttagtttag gaaccagtga gcaagtcagc ccttggggca gcccatacaa ggccatgggg | 240 |
| ctgggcaagc tgcacgcctg ggtccggggt gggcacggtg cccgggcaac gagctgaaag | 300 |
| ctcatctgct ctcaggggcc cctccctggg gacagcccct cctggctagt cacaccctgt | 360 |
| aggctcctct atataaccca ggggcacagg ggctgccctc attctaccac cacctccaca | 420 |
| gcacagacag acactcagga gccagccagc | 450 |

SEQ ID NO: 30-hum-cTnT455

| | |
|---|---|
| ctgctcccag ctggccctcc caggcctggg ttgctggcct ctgctttatc aggattctca | 60 |
| agagggacag ctggtttatg ttgcatgact gttccctgca tatctgctct ggttttaaat | 120 |
| agcttatctg ctagcctgct cccagctggc cctcccaggc ctgggttgct ggcctctgct | 180 |
| ttatcaggat tctcaagagg gacagctggt ttatgttgca tgactgttcc ctgcatatct | 240 |
| gctctggttt taaatagctt atctgagcag ctggaggacc acatgggctt atatggggca | 300 |
| cctgccaaaa tagcagccaa cacccccccc tgtcgcacat tcctccctgg ctcaccaggc | 360 |
| cccagcccac atgcctgctt aaagccctct ccatcctctg cctcacccag tccccgctga | 420 |
| gactgagcag acgcctccag gatctgtcgg cagct | 455 |

```
SEQ ID NO: 31-Homo sapiens dystrophin (DMD) gene
GeneID: 1756
SEQ ID NO: 32-Homo sapiens dystrophin mRNA-transcript variant Dp427m, mRNA
>NM_004006.3 Homo sapiens dystrophin (DMD), transcript variant Dp427m, mRNA
ATCAGTTACTGTGTTGACTCACTCAGTGTTGGGATCACTCACTTTCCCCCTACAGGACTCAGATCTGGGA
GGCAATTACCTTCGGAGAAAAACGAATAGGAAAAACTGAAGTGTTACTTTTTTTAAAGCTGCTGAAGTTT
GTTGGTTTCTCATTGTTTTTAAGCCTACTGGAGCAATAAAGTTTGAAGAACTTTTACCAGGTTTTTTTTA
TCGCTGCCTTGATATACACTTTTCAAAATGCTTTGGTGGGAAGAAGTAGAGGACTGTTATGAAAGAGAAG
ATGTTCAAAAGAAAACATTCACAAAATGGGTAAATGCACAATTTTCTAAGTTTGGGAAGCAGCATATTGA
GAACCTCTTCAGTGACCTACAGGATGGGAGGCGCCTCCTAGACCTCCTCGAAGGCCTGACAGGGCAAAAA
CTGCCAAAAGAAAAAGGATCCACAAGAGTTCATGCCCTGAACAATGTCAACAAGGCACTGCGGGTTTTGC
AGAACAATAATGTTGATTTAGTGAATATTGGAAGTACTGACATCGTAGATGGAAATCATAAACTGACTCT
TGGTTTGATTTGGAATATAATCCTCCACTGGCAGGTCAAAAATGTAATGAAAAATATCATGGCTGGATTG
CAACAAACCAACAGTGAAAAGATTCTCCTGAGCTGGGTCCGACAATCAACTCGTAATTATCCACAGGTTA
ATGTAATCAACTTCACCACCAGCTGGTCTGATGGCCTGGCTTTGAATGCTCTCATCCATAGTCATAGGCC
AGACCTATTTGACTGGAATAGTGTGGTTTGCCAGCAGTCAGCCACACAACGACTGGAACATGCATTCAAC
ATCGCCAGATATCAATTAGGCATAGAGAAACTACTCGATCCTGAAGATGTTGATACCACCTATCCAGATA
AGAAGTCCATCTTAATGTACATCACATCACTCTTCCAAGTTTTGCCTCAACAAGTGAGCATTGAAGCCAT
CCAGGAAGTGGAAATGTTGCCAAGGCCACCTAAAGTGACTAAAGAAGAACATTTTCAGTTACATCATCAA
ATGCACTATTCTCAACAGATCACGGTCAGTCTAGCACAGGGATATGAGAGAACTTCTTCCCCTAAGCCTC
GATTCAAGAGCTATGCCTACACACAGGCTGCTTATGTCACCACCTCTGACCCTACACGGAGCCCATTTCC
TTCACAGCATTTGGAAGCTCCTGAAGACAAGTCATTTGGCAGTTCATTGATGGAGAGTGAAGTAAACCTG
GACCGTTATCAAACAGCTTTAGAAGAAGTATTATCGTGGCTTCTTTCTGCTGAGGACACATTGCAAGCAC
AAGGAGAGATTTCTAATGATGTGGAAGTGGTGAAAGACCAGTTTCATACTCATGAGGGGTACATGATGGA
TTTGACAGCCCATCAGGGCCGGGTTGGTAATATTCTACAATTGGGAAGTAAGCTGATTGGAACAGGAAAA
TTATCAGAAGATGAAGAAACTGAAGTACAAGAGCAGATGAATCTCCTAAATTCAAGATGGGAATGCCTCA
GGGTAGCTAGCATGGAAAAACAAAGCAATTTACATAGAGTTTTAATGGATCTCCAGAATCAGAAACTGAA
AGAGTTGAATGACTGGCTAACAAAAACAGAAGAAAGAACAAGGAAAATGGAGGAAGAGCCTCTTGGACCT
GATCTTGAAGACCTAAAACGCCAAGTACAACAACATAAGGTGCTTCAAGAAGATCTAGAACAAGAACAAG
TCAGGGTCAATTCTCTCACTCACATGGTGGTGGTAGTTGATGAATCTAGTGGAGATCACGCAACTGCTGC
TTTGGAAGAACAACTTAAGGTATTGGGAGATCGATGGGCAAACATCTGTAGATGGACAGAAGACCGCTGG
GTTCTTTTACAAGACATCCTTCTCAAATGGCAACGTCTTACTGAAGAACAGTGCCTTTTTAGTGCATGGC
TTTCAGAAAAAGAAGATGCAGTGAACAAGATTCACACAACTGGCTTTAAAGATCAAAATGAAATGTTATC
AAGTCTTCAAAAACTGGCCGTTTTAAAAGCGGATCTAGAAAAGAAAAAGCAATCCATGGGCAAACTGTAT
TCACTCAAACAAGATCTTCTTTCAACACTGAAGAATAAGTCAGTGACCCAGAAGACGGAAGCATGGCTGG
ATAACTTTGCCCGGTGTTGGGATAATTTAGTCCAAAAACTTGAAAAGAGTACAGCACAGATTTCACAGGC
TGTCACCACCACTCAGCCATCACTAACACAGACAACTGTAATGGAAACAGTAACTACGGTGACCACAAGG
GAACAGATCCTGGTAAAGCATGCTCAAGAGGAACTTCCACCACCACCTCCCCAAAAGAAGAGGCAGATTA
CTGTGGATTCTGAAATTAGGAAAAGGTTGGATGTTGATATAACTGAACTTCACAGCTGGATTACTCGCTC
AGAAGCTGTGTTGCAGAGTCCTGAATTTGCAATCTTTCGGAAGGAAGGCAACTTCTCAGACTTAAAAGAA
AAAGTCAATGCCATAGAGCGAGAAAAAGCTGAGAAGTTCAGAAAACTGCAAGATGCCAGCAGATCAGCTC
AGGCCCTGGTGGAACAGATGGTGAATGAGGGTGTTAATGCAGATAGCATCAAACAAGCCTCAGAACAACT
GAACAGCCGGTGGATCGAATTCTGCCAGTTGCTAAGTGAGAGACTTAACTGGCTGGAGTATCAGAACAAC
ATCATCGCTTTCTATAATCAGCTACAACAATTGGAGCAGATGACAACTACTGCTGAAAACTGGTTGAAAA
TCCAACCCACCACCCCATCAGAGCCAACAGCAATTAAAAGTCAGTTAAAAATTTGTAAGGATGAAGTCAA
CCGGCTATCAGATCTTCAACCTCAAATTGAACGATTAAAAATTCAAAGCATAGCCCTGAAAGAGAAAGGA
CAAGGACCCATGTTCCTGGATGCAGACTTTGTGGCCTTTACAAATCATTTTAAGCAAGTCTTTTCTGATG
TGCAGGCCAGAGAGAAAGAGCTACAGACAATTTTTGACACTTTGCCACCAATGCGCTATCAGGAGACCAT
GAGTGCCATCAGGACATGGGTCCAGCAGTCAGAAACCAAACTCTCCATACCTCAACTTAGTGTCACCGAC
TATGAAATCATGGAGCAGAGACTCGGGGAATTGCAGGCTTTACAAAGTTCTCTGCAAGAGCAACAAAGTG
GCCTATACTATCTCAGCACCACTGTGAAAGAGATGTCGAAGAAAGCGCCCTCTGAAATTAGCCGGAAATA
TCAATCAGAATTTGAAGAAATTGAGGGACGCTGGAAGAAGCTCTCCTCCCAGCTGGTTGAGCATTGTCAA
AAGCTAGAGGAGCAAATGAATAAACTCCGAAAAATTCAGAATCACATACAAACCCTGAAGAAATGGATGG
CTGAAGTTGATGTTTTTCTGAAGGAGGAATGGCCTGCCCTTGGGGATTCAGAAATTCTAAAAAAGCAGCT
GAAACAGTGCAGACTTTTAGTCAGTGATATTCAGACAATTCAGCCCAGTCTAAACAGTGTCAATGAAGGT
GGGCAGAAGATAAAGAATGAAGCAGAGCCAGAGTTTGCTTCGAGACTTGAGACAGAACTCAAAGAACTTA
ACACTCAGTGGGATCACATGTGCCAACAGGTCTATGCCAGAAAGGAGGCCTTGAAGGGAGGTTTGGAGAA
AACTGTAAGCCTCCAGAAAGATCTATCAGAGATGCACGAATGGATGACACAAGCTGAAGAAGAGTATCTT
GAGAGAGATTTTGAATATAAAACTCCAGATGAATTACAGAAAGCAGTTGAAGAGATGAAGAGAGCTAAAG
AAGAGGCCCAACAAAAAGAAGCGAAAGTGAAACTCCTTACTGAGTCTGTAAATAGTGTCATAGCTCAAGC
TCCACCTGTAGCACAAGAGGCCTTAAAAAAGGAACTTGAAACTCTAACCACCAACTACCAGTGGCTCTGC
ACTAGGCTGAATGGGAAATGCAAGACTTTGGAAGAAGTTTGGGCATGTTGGCATGAGTTATTGTCATACT
TGGAGAAAGCAAACAAGTGGCTAAATGAAGTAGAATTTAAACTTAAAACCACTGAAAACATTCCTGGCGG
AGCTGAGGAAATCTCTGAGGTGCTAGATTCACTTGAAAATTTGATGCGACATTCAGAGGATAACCCAAAT
CAGATTCGCATATTGGCACAGACCCTAACAGATGGCGGAGTCATGGATGAGCTAATCAATGAGGAACTTG
AGACATTTAATTCTCGTTGGAGGGAACTACATGAAGAGGCTGTAAGGAGGCAAAAGTTGCTTGAACAGAG
CATCCAGTCTGCCCAGGAGACTGAAAAATCCTTACACTTAATCCAGGAGTCCCTCACATTCATTGACAAG
CAGTTGGCAGCTTATATTGCAGACAAGGTGGACGCAGCTCAAATGCCTCAGGAAGCCCAGAAAATCCAAT
CTGATTTGACAAGTCATGAGATCAGTTTAGAAGAAATGAAGAAACATAATCAGGGGAAGGAGGCTGCCCA
AAGAGTCCTGTCTCAGATTGATGTTGCACAGAAAAAATTACAAGATGTCTCCATGAAGTTTCGATTATTC
CAGAAACCAGCCAATTTTGAGCAGCGTCTACAAGAAAGTAAGATGATTTTAGATGAAGTGAAGATGCACT
TGCCTGCATTGGAAACAAAGAGTGTGGAACAGGAAGTAGTACAGTCACAGCTAAATCATTGTGTGAACTT
GTATAAAAGTCTGAGTGAAGTGAAGTCTGAAGTGGAAATGGTGATAAAGACTGGACGTCAGATTGTACAG
AAAAAGCAGACGGAAAATCCCAAAGAACTTGATGAAAGAGTAACAGCTTTGAAATTGCATTATAATGAGC
TGGGAGCAAAGGTAACAGAAAGAAAGCAACAGTTGGAGAAATGCTTGAAATTGTCCCGTAAGATGCGAAA
GGAAATGAATGTCTTGACAGAATGGCTGGCAGCTACAGATATGGAATTGACAAAGAGATCAGCAGTTGAA
GGAATGCCTAGTAATTTGGATTCTGAAGTTGCCTGGGGAAAGGCTACTCAAAAAGAGATTGAGAAACAGA
AGGTGCACCTGAAGAGTATCACAGAGGTAGGAGAGGCCTTGAAAACAGTTTTGGGCAAGAAGGAGACGTT
GGTGGAAGATAAACTCAGTCTTCTGAATAGTAACTGGATAGCTGTCACCTCCCGAGCAGAAGAGTGGTTA
AATCTTTTGTTGGAATACCAGAAACACATGGAAACTTTTGACCAGAATGTGGACCACATCACAAAGTGGA
TCATTCAGGCTGACACACTTTTGGATGAATCAGAGAAAAAGAAACCCCAGCAAAAAGAAGACGTGCTTAA
GCGTTTAAAGGCAGAACTGAATGACATACGCCCAAAGGTGGACTCTACACGTGACCAAGCAGCAAACTTG
ATGGCAAACCGCGGTGACCACTGCAGGAAATTAGTAGAGCCCCAAATCTCAGAGCTCAACCATCGATTTG
CAGCCATTTCACACAGAATTAAGACTGGAAAGGCCTCCATTCCTTTGAAGGAATTGGAGCAGTTTAACTC
AGATATACAAAAATTGCTTGAACCACTGGAGGCTGAAATTCAGCAGGGGGTGAATCTGAAAGAGGAAGAC
TTCAATAAAGATATGAATGAAGACAATGAGGGTACTGTAAAAGAATTGTTGCAAAGAGGAGACAACTTAC
AACAAAGAATCACAGATGAGAGAAAGCGAGAGGAAATAAAGATAAAACAGCAGCTGTTACAGACAAAACA
TAATGCTCTCAAGGATTTGAGGTCTCAAAGAAGAAAAAAGGCTCTAGAAATTTCTCATCAGTGGTATCAG
TACAAGAGGCAGGCTGATGATCTCCTGAAATGCTTGGATGACATTGAAAAAAAATTAGCCAGCCTACCTG
AGCCCAGAGATGAAAGGAAAATAAAGGAAATTGATCGGGAATTGCAGAAGAAGAAAGAGGAGCTGAATGC
AGTGCGTAGGCAAGCTGAGGGCTTGTCTGAGGATGGGGCCGCAATGGCAGTGGAGCCAACTCAGATCCAG
CTCAGCAAGCGCTGGCGGGAAATTGAGAGCAAATTTGCTCAGTTTCGAAGACTCAACTTTGCACAAATTC
ACACTGTCCGTGAAGAAACGATGATGGTGATGACTGAAGACATGCCTTTGGAAATTTCTTATGTGCCTTC
TACTTATTTGACTGAAATCACTCATGTCTCACAAGCCCTATTAGAAGTGGAACAACTTCTCAATGCTCCT
GACCTCTGTGCTAAGGACTTTGAAGATCTCTTTAAGCAAGAGGAGTCTCTGAAGAATATAAAAGATAGTC
TACAACAAAGCTCAGGTCGGATTGACATTATTCATAGCAAGAAGACAGCAGCATTGCAAAGTGCAACGCC
TGTGGAAAGGGTGAAGCTACAGGAAGCTCTCTCCCAGCTTGATTTCCAATGGGAAAAAGTTAACAAAATG
TACAAGGACCGACAAGGGCGATTTGACAGATCTGTTGAGAAATGGCGGCGTTTTCATTATGATATAAAGA
TATTTAATCAGTGGCTAACAGAAGCTGAACAGTTTCTCAGAAAGACACAAATTCCTGAGAATTGGGAACA
TGCTAAATACAAATGGTATCTTAAGGAACTCCAGGATGGCATTGGGCAGCGGCAAACTGTTGTCAGAACA
TTGAATGCAACTGGGGAAGAAATAATTCAGCAATCCTCAAAAACAGATGCCAGTATTCTACAGGAAAAAT
TGGGAAGCCTGAATCTGCGGTGGCAGGAGGTCTGCAAACAGCTGTCAGACAGAAAAAAGAGGCTAGAAGA
ACAAAAGAATATCTTGTCAGAATTTCAAAGAGATTTAAATGAATTTGTTTTATGGTTGGAGGAAGCAGAT
AACATTGCTAGTATCCCACTTGAACCTGGAAAAGAGCAGCAACTAAAAGAAAAGCTTGAGCAAGTCAAGT
TACTGGTGGAAGAGTTGCCCCTGCGCCAGGGAATTCTCAAACAATTAAATGAAACTGGAGGACCCGTGCT
TGTAAGTGCTCCCATAAGCCCAGAAGAGCAAGATAAACTTGAAAATAAGCTCAAGCAGACAAATCTCCAG
TGGATAAAGGTTTCCAGAGCTTTACCTGAGAAACAAGGAGAAATTGAAGCTCAAATAAAAGACCTTGGGC
AGCTTGAAAAAAAGCTTGAAGACCTTGAAGAGCAGTTAAATCATCTGCTGCTGTGGTTATCTCCTATTAG
GAATCAGTTGGAAATTTATAACCAACCAAACCAAGAAGGACCATTTGACGTTAAGGAAACTGAAATAGCA
GTTCAAGCTAAACAACCGGATGTGGAAGAGATTTTGTCTAAAGGGCAGCATTTGTACAAGGAAAAACCAG
CCACTCAGCCAGTGAAGAGGAAGTTAGAAGATCTGAGCTCTGAGTGGAAGGCGGTAAACCGTTTACTTCA
AGAGCTGAGGGCAAAGCAGCCTGACCTAGCTCCTGGACTGACCACTATTGGAGCCTCTCCTACTCAGACT
GTTACTCTGGTGACACAACCTGTGGTTACTAAGGAAACTGCCATCTCCAAACTAGAAATGCCATCTTCCT
TGATGTTGGAGGTACCTGCTCTGGCAGATTTCAACCGGGCTTGGACAGAACTTACCGACTGGCTTTCTCT
GCTTGATCAAGTTATAAAATCACAGAGGGTGATGGTGGGTGACCTTGAGGATATCAACGAGATGATCATC
AAGCAGAAGGCAACAATGCAGGATTTGGAACAGAGGCGTCCCCAGTTGGAAGAACTCATTACCGCTGCCC
AAAATTTGAAAAACAAGACCAGCAATCAAGAGGCTAGAACAATCATTACGGATCGAATTGAAAGAATTCA
GAATCAGTGGGATGAAGTACAAGAACACCTTCAGAACCGGAGGCAACAGTTGAATGAAATGTTAAAGGAT
TCAACACAATGGCTGGAAGCTAAGGAAGAAGCTGAGCAGGTCTTAGGACAGGCCAGAGCCAAGCTTGAGT
CATGGAAGGAGGGTCCCTATACAGTAGATGCAATCCAAAAGAAAATCACAGAAACCAAGCAGTTGGCCAA
AGACCTCCGCCAGTGGCAGACAAATGTAGATGTGGCAAATGACTTGGCCCTGAAACTTCTCCGGGATTAT
TCTGCAGATGATACCAGAAAAGTCCACATGATAACAGAGAATATCAATGCCTCTTGGAGAAGCATTCATA
AAAGGGTGAGTGAGCGAGAGGCTGCTTTGGAAGAAACTCATAGATTACTGCAACAGTTCCCCCTGGACCT
GGAAAAGTTTCTTGCCTGGCTTACAGAAGCTGAAACAACTGCCAATGTCCTACAGGATGCTACCCGTAAG
GAAAGGCTCCTAGAAGACTCCAAGGGAGTAAAAGAGCTGATGAAACAATGGCAAGACCTCCAAGGTGAAA
TTGAAGCTCACACAGATGTTTATCACAACCTGGATGAAAACAGCCAAAAAATCCTGAGATCCCTGGAAGG
TTCCGATGATGCAGTCCTGTTACAAAGACGTTTGGATAACATGAACTTCAAGTGGAGTGAACTTCGGAAA
AAGTCTCTCAACATTAGGTCCCATTTGGAAGCCAGTTCTGACCAGTGGAAGCGTCTGCACCTTTCTCTGC
AGGAACTTCTGGTGTGGCTACAGCTGAAAGATGATGAATTAAGCCGGCAGGCACCTATTGGAGGCGACTT
TCCAGCAGTTCAGAAGCAGAACGATGTACATAGGGCCTTCAAGAGGGAATTGAAAACTAAAGAACCTGTA
ATCATGAGTACTCTTGAGACTGTACGAATATTTCTGACAGAGCAGCCTTTGGAAGGACTAGAGAAACTCT
ACCAGGAGCCCAGAGAGCTGCCTCCTGAGGAGAGAGCCCAGAATGTCACTCGGCTTCTACGAAAGCAGGC
TGAGGAGGTCAATACTGAGTGGGAAAAATTGAACCTGCACTCCGCTGACTGGCAGAGAAAAATAGATGAG
ACCCTTGAAAGACTCCGGGAACTTCAAGAGGCCACGGATGAGCTGGACCTCAAGCTGCGCCAAGCTGAGG
TGATCAAGGGATCCTGGCAGCCCGTGGGCGATCTCCTCATTGACTCTCTCCAAGATCACCTCGAGAAAGT
CAAGGCACTTCGAGGAGAAATTGCGCCTCTGAAAGAGAACGTGAGCCACGTCAATGACCTTGCTCGCCAG
CTTACCACTTTGGGCATTCAGCTCTCACCGTATAACCTCAGCACTCTGGAAGACCTGAACACCAGATGGA
AGCTTCTGCAGGTGGCCGTCGAGGACCGAGTCAGGCAGCTGCATGAAGCCCACAGGGACTTTGGTCCAGC
ATCTCAGCACTTTCTTTCCACGTCTGTCCAGGGTCCCTGGGAGAGAGCCATCTCGCCAAACAAAGTGCCC
TACTATATCAACCACGAGACTCAAACAACTTGCTGGGACCATCCCAAAATGACAGAGCTCTACCAGTCTT
TAGCTGACCTGAATAATGTCAGATTCTCAGCTTATAGGACTGCCATGAAACTCCGAAGACTGCAGAAGGC
CCTTTGCTTGGATCTCTTGAGCCTGTCAGCTGCATGTGATGCCTTGGACCAGCACAACCTCAAGCAAAAT
GACCAGCCCATGGATATCCTGCAGATTATTAATTGTTTGACCACTATTTATGACCGCCTGGAGCAAGAGC
ACAACAATTTGGTCAACGTCCCTCTCTGCGTGGATATGTGTCTGAACTGGCTGCTGAATGTTTATGATAC
GGGACGAACAGGGAGGATCCGTGTCCTGTCTTTTAAAACTGGCATCATTTCCCTGTGTAAAGCACATTTG
GAAGACAAGTACAGATACCTTTTCAAGCAAGTGGCAAGTTCAACAGGATTTTGTGACCAGCGCAGGCTGG
GCCTCCTTCTGCATGATTCTATCCAAATTCCAAGACAGTTGGGTGAAGTTGCATCCTTTGGGGGCAGTAA
CATTGAGCCAAGTGTCCGGAGCTGCTTCCAATTTGCTAATAATAAGCCAGAGATCGAAGCGGCCCTCTTC
CTAGACTGGATGAGACTGGAACCCCAGTCCATGGTGTGGCTGCCCGTCCTGCACAGAGTGGCTGCTGCAG
AAACTGCCAAGCATCAGGCCAAATGTAACATCTGCAAAGAGTGTCCAATCATTGGATTCAGGTACAGGAG
TCTAAAGCACTTTAATTATGACATCTGCCAAAGCTGCTTTTTTTCTGGTCGAGTTGCAAAAGGCCATAAA
ATGCACTATCCCATGGTGGAATATTGCACTCCGACTACATCAGGAGAAGATGTTCGAGACTTTGCCAAGG
TACTAAAAAACAAATTTCGAACCAAAAGGTATTTTGCGAAGCATCCCCGAATGGGCTACCTGCCAGTGCA
GACTGTCTTAGAGGGGGACAACATGGAAACTCCCGTTACTCTGATCAACTTCTGGCCAGTAGATTCTGCG
CCTGCCTCGTCCCCTCAGCTTTCACACGATGATACTCATTCACGCATTGAACATTATGCTAGCAGGCTAG
CAGAAATGGAAAACAGCAATGGATCTTATCTAAATGATAGCATCTCTCCTAATGAGAGCATAGATGATGA
ACATTTGTTAATCCAGCATTACTGCCAAAGTTTGAACCAGGACTCCCCCCTGAGCCAGCCTCGTAGTCCT
GCCCAGATCTTGATTTCCTTAGAGAGTGAGGAAAGAGGGGAGCTAGAGAGAATCCTAGCAGATCTTGAGG
AAGAAAACAGGAATCTGCAAGCAGAATATGACCGTCTAAAGCAGCAGCACGAACATAAAGGCCTGTCCCC
ACTGCCGTCCCCTCCTGAAATGATGCCCACCTCTCCCCAGAGTCCCCGGGATGCTGAGCTCATTGCTGAG
GCCAAGCTACTGCGTCAACACAAAGGCCGCCTGGAAGCCAGGATGCAAATCCTGGAAGACCACAATAAAC
AGCTGGAGTCACAGTTACACAGGCTAAGGCAGCTGCTGGAGCAACCCCAGGCAGAGGCCAAAGTGAATGG
CACAACGGTGTCCTCTCCTTCTACCTCTCTACAGAGGTCCGACAGCAGTCAGCCTATGCTGCTCCGAGTG
GTTGGCAGTCAAACTTCGGACTCCATGGGTGAGGAAGATCTTCTCAGTCCTCCCCAGGACACAAGCACAG
GGTTAGAGGAGGTGATGGAGCAACTCAACAACTCCTTCCCTAGTTCAAGAGGAAGAAATACCCCTGGAAA
GCCAATGAGAGAGGACACAATGTAGGAAGTCTTTTCCACATGGCAGATGATTTGGGCAGAGCGATGGAGT
CCTTAGTATCAGTCATGACAGATGAAGAAGGAGCAGAATAAATGTTTTACAACTCCTGATTCCCGCATGG
TTTTTATAATATTCATACAACAAAGAGGATTAGACAGTAAGAGTTTACAAGAAATAAATCTATATTTTTG
TGAAGGGTAGTGGTATTATACTGTAGATTTCAGTAGTTTCTAAGTCTGTTATTGTTTTGTTAACAATGGC
AGGTTTTACACGTCTATGCAATTGTACAAAAAAGTTATAAGAAAACTACATGTAAAATCTTGATAGCTAA
ATAACTTGCCATTTCTTTATATGGAACGCATTTTGGGTTGTTTAAAAATTTATAACAGTTATAAAGAAAG
ATTGTAAACTAAAGTGTGCTTTATAAAAAAAAGTTGTTTATAAAAACCCCTAAAAACAAAACAAACACAC
ACACACACACATACACACACACACACAAAACTTTGAGGCAGCGCATTGTTTTGCATCCTTTTGGCGTGAT
ATCCATATGAAATTCATGGCTTTTTCTTTTTTTGCATATTAAAGATAAGACTTCCTCTACCACCACACCA
AATGACTACTACACACTGCTCATTTGAGAACTGTCAGCTGAGTGGGGCAGGCTTGAGTTTTCATTTCATA
TATCTATATGTCTATAAGTATATAAATACTATAGTTATATAGATAAAGAGATACGAATTTCTATAGACTG
ACTTTTTCCATTTTTTAAATGTTCATGTCACATCCTAATAGAAAGAAATTACTTCTAGTCAGTCATCCAG
GCTTACCTGCTTGGTCTAGAATGGATTTTTCCCGGAGCCGGAAGCCAGGAGGAAACTACACCACACTAAA
ACATTGTCTACAGCTCCAGATGTTTCTCATTTTAAACAACTTTCCACTGACAACGAAAGTAAAGTAAAGT
ATTGGATTTTTTTAAAGGGAACATGTGAATGAATACACAGGACTTATTATATCAGAGTGAGTAATCGGTT
GGTTGGTTGATTGATTGATTGATTGATACATTCAGCTTCCTGCTGCTAGCAATGCCACGATTTAGATTTA
ATGATGCTTCAGTGGAAATCAATCAGAAGGTATTCTGACCTTGTGAACATCAGAAGGTATTTTTTAACTC
CCAAGCAGTAGCAGGACGATGATAGGGCTGGAGGGCTATGGATTCCCAGCCCATCCCTGTGAAGGAGTAG
GCCACTCTTTAAGTGAAGGATTGGATGATTGTTCATAATACATAAAGTTCTCTGTAATTACAACTAAATT
ATTATGCCCTCTTCTCACAGTCAAAAGGAACTGGGTGGTTTGGTTTTTGTTGCTTTTTTAGATTTATTGT
CCCATGTGGGATGAGTTTTTAAATGCCACAAGACATAATTTAAAATAAATAAACTTTGGGAAAAGGTGTA
AAACAGTAGCCCCATCACATTTGTGATACTGACAGGTATCAACCCAGAAGCCCATGAACTGTGTTTCCAT
CCTTTGCATTTCTCTGCGAGTAGTTCCACACAGGTTTGTAAGTAAGTAAGAAAGAAGGCAAATTGATTCA
AATGTTACAAAAAAACCCTTCTTGGTGGATTAGACAGGTTAAATATATAAACAAACAAACAAAAATTGCT
CAAAAAAGAGGAGAAAAGCTCAAGAGGAAAAGCTAAGGACTGGTAGGAAAAAGCTTTACTCTTTCATGCC
ATTTTATTTCTTTTTGATTTTTAAATCATTCATTCAATAGATACCACCGTGTGACCTATAATTTTGCAAA
TCTGTTACCTCTGACATCAAGTGTAATTAGCTTTTGGAGAGTGGGCTGACATCAAGTGTAATTAGCTTTT
GGAGAGTGGGTTTTGTCCATTATTAATAATTAATTAATTAACATCAAACACGGCTTCTCATGCTATTTCT
ACCTCACTTTGGTTTTGGGGTGTTCCTGATAATTGTGCACACCTGAGTTCACAGCTTCACCACTTGTCCA
TTGCGTTATTTTCTTTTTCCTTTATAATTCTTTCTTTTTCCTTCATAATTTTCAAAAGAAAACCCAAAGC
TCTAAGGTAACAAATTACCAAATTACATGAAGATTTGGTTTTTGTCTTGCATTTTTTTCCTTTATGTGAC
GCTGGACCTTTTCTTTACCCAAGGATTTTTAAAACTCAGATTTAAAACAAGGGGTTACTTTACATCCTAC
TAAGAAGTTTAAGTAAGTAAGTTTCATTCTAAAATCAGAGGTAAATAGAGTGCATAAATAATTTTGTTTT
AATCTTTTTGTTTTTCTTTTAGACACATTAGCTCTGGAGTGAGTCTGTCATAATATTTGAACAAAAATTG
AGAGCTTTATTGCTGCATTTTAAGCATAATTAATTTGGACATTATTTCGTGTTGTGTTCTTTATAACCAC
CAAGTATTAAACTGTAAATCATAATGTAACTGAAGCATAAACATCACATGGCATGTTTTGTCATTGTTTT
CAGGTACTGAGTTCTTACTTGAGTATCATAATATATTGTGTTTTAACACCAACACTGTAACATTTACGAA
TTATTTTTTTAAACTTCAGTTTTACTGCATTTTCACAACATATCAGACTTCACCAAATATATGCCTTACT
ATTGTATTATAGTACTGCTTTACTGTGTATCTCAATAAAGCACGCAGTTATGTTACAAAAAA
SEQ ID NO: 33-[Homo sapiens] dystrophin isoform Dp427m amino acid sequence
>NP_003997.2 dystrophin isoform Dp427m sapiens
MLWWEEVEDCYEREDVQKKTFTKWVNAQFSKFGKQHIENLFSDLQDGRRLLDLLEGLTGQKLPKEKGSTR
VHALNNVNKALRVLQNNNVDLVNIGSTDIVDGNHKLTLGLIWNIILHWQVKNVMKNIMAGLQQTNSEKIL
LSWVRQSTRNYPQVNVINFTTSWSDGLALNALIHSHRPDLFDWNSVVCQQSATQRLEHAFNIARYQLGIE
KLLDPEDVDTTYPDKKSILMYITSLFQVLPQQVSIEAIQEVEMLPRPPKVTKEEHFQLHHQMHYSQQITV
SLAQGYERTSSPKPRFKSYAYTQAAYVTTSDPTRSPFPSQHLEAPEDKSFGSSLMESEVNLDRYQTALEE
VLSWLLSAEDTLQAQGEISNDVEVVKDQFHTHEGYMMDLTAHQGRVGNILQLGSKLIGTGKLSEDEETEV
QEQMNLLNSRWECLRVASMEKQSNLHRVLMDLQNQKLKELNDWLTKTEERTRKMEEEPLGPDLEDLKRQV
QQHKVLQEDLEQEQVRVNSLTHMVVVVDESSGDHATAALEEQLKVLGDRWANICRWTEDRWVLLQDILLK
WQRLTEEQCLFSAWLSEKEDAVNKIHTTGEKDQNEMLSSLQKLAVLKADLEKKKQSMGKLYSLKQDLLST
LKNKSVTQKTEAWLDNFARCWDNLVQKLEKSTAQISQAVTTTQPSLTQTTVMETVTTVTTREQILVKHAQ
EELPPPPPQKKRQITVDSEIRKRLDVDITELHSWITRSEAVLQSPEFAIFRKEGNFSDLKEKVNAIEREK
AEKFRKLQDASRSAQALVEQMVNEGVNADSIKQASEQLNSRWIEFCQLLSERLNWLEYQNNIIAFYNQLQ
QLEQMTTTAENWLKIQPTTPSEPTAIKSQLKICKDEVNRLSDLQPQIERLKIQSIALKEKGQGPMFLDAD
EVAFTNHFKQVFSDVQAREKELQTIFDTLPPMRYQETMSAIRTWVQQSETKLSIPQLSVTDYEIMEQRLG
ELQALQSSLQEQQSGLYYLSTTVKEMSKKAPSEISRKYQSEFEEIEGRWKKLSSQLVEHCQKLEEQMNKL
RKIQNHIQTLKKWMAEVDVFLKEEWPALGDSEILKKQLKQCRLLVSDIQTIQPSLNSVNEGGQKIKNEAE
PEFASRLETELKELNTQWDHMCQQVYARKEALKGGLEKTVSLQKDLSEMHEWMTQAEEEYLERDFEYKTP
DELQKAVEEMKRAKEEAQQKEAKVKLLTESVNSVIAQAPPVAQEALKKELETLTTNYQWLCTRLNGKCKT
LEEVWACWHELLSYLEKANKWLNEVEFKLKTTENIPGGAEEISEVLDSLENLMRHSEDNPNQIRILAQTL
TDGGVMDELINEELETENSRWRELHEEAVRRQKLLEQSIQSAQETEKSLHLIQESLTFIDKQLAAYIADK
VDAAQMPQEAQKIQSDLTSHEISLEEMKKHNQGKEAAQRVLSQIDVAQKKLQDVSMKFRLFQKPANFEQR
LQESKMILDEVKMHLPALETKSVEQEVVQSQLNHCVNLYKSLSEVKSEVEMVIKTGRQIVQKKQTENPKE
LDERVTALKLHYNELGAKVTERKQQLEKCLKLSRKMRKEMNVLTEWLAATDMELTKRSAVEGMPSNLDSE
VAWGKATQKEIEKQKVHLKSITEVGEALKTVLGKKETLVEDKLSLLNSNWIAVTSRAEEWLNLLLEYQKH
METFDQNVDHITKWIIQADTLLDESEKKKPQQKEDVLKRLKAELNDIRPKVDSTRDQAANLMANRGDHCR
KLVEPQISELNHRFAAISHRIKTGKASIPLKELEQENSDIQKLLEPLEAEIQQGVNLKEEDFNKDMNEDN
EGTVKELLQRGDNLQQRITDERKREEIKIKQQLLQTKHNALKDLRSQRRKKALEISHQWYQYKRQADDLL
KCLDDIEKKLASLPEPRDERKIKEIDRELQKKKEELNAVRRQAEGLSEDGAAMAVEPTQIQLSKRWREIE
SKFAQFRRLNFAQIHTVREETMMVMTEDMPLEISYVPSTYLTEITHVSQALLEVEQLLNAPDLCAKDFED
LFKQEESLKNIKDSLQQSSGRIDIIHSKKTAALQSATPVERVKLQEALSQLDFQWEKVNKMYKDRQGRED
RSVEKWRRFHYDIKIFNQWLTEAEQFLRKTQIPENWEHAKYKWYLKELQDGIGQRQTVVRTLNATGEEII
QQSSKTDASILQEKLGSLNLRWQEVCKQLSDRKKRLEEQKNILSEFQRDLNEFVLWLEEADNIASIPLEP
GKEQQLKEKLEQVKLLVEELPLRQGILKQLNETGGPVLVSAPISPEEQDKLENKLKQTNLQWIKVSRALP
EKQGEIEAQIKDLGQLEKKLEDLEEQLNHLLLWLSPIRNQLEIYNQPNQEGPFDVKETEIAVQAKQPDVE
EILSKGQHLYKEKPATQPVKRKLEDLSSEWKAVNRLLQELRAKQPDLAPGLTTIGASPTQTVTLVTQPVV
TKETAISKLEMPSSLMLEVPALADFNRAWTELTDWLSLLDQVIKSQRVMVGDLEDINEMIIKQKATMQDL
EQRRPQLEELITAAQNLKNKTSNQEARTIITDRIERIQNQWDEVQEHLQNRRQQLNEMLKDSTQWLEAKE
EAEQVLGQARAKLESWKEGPYTVDAIQKKITETKQLAKDLRQWQTNVDVANDLALKLLRDYSADDTRKVH
MITENINASWRSIHKRVSEREAALEETHRLLQQFPLDLEKFLAWLTEAETTANVLQDATRKERLLEDSKG
VKELMKQWQDLQGEIEAHTDVYHNLDENSQKILRSLEGSDDAVLLQRRLDNMNFKWSELRKKSLNIRSHL
EASSDQWKRLHLSLQELLVWLQLKDDELSRQAPIGGDFPAVQKQNDVHRAFKRELKTKEPVIMSTLETVR
IFLTEQPLEGLEKLYQEPRELPPEERAQNVTRLLRKQAEEVNTEWEKLNLHSADWQRKIDETLERLRELQ
EATDELDLKLRQAEVIKGSWQPVGDLLIDSLQDHLEKVKALRGEIAPLKENVSHVNDLARQLTTLGIQLS
PYNLSTLEDLNTRWKLLQVAVEDRVRQLHEAHRDFGPASQHFLSTSVQGPWERAISPNKVPYYINHETQT
TCWDHPKMTELYQSLADLNNVRFSAYRTAMKLRRLQKALCLDLLSLSAACDALDQHNLKQNDQPMDILQI
INCLTTIYDRLEQEHNNLVNVPLCVDMCLNWLLNVYDTGRTGRIRVLSFKTGIISLCKAHLEDKYRYLFK
QVASSTGFCDQRRLGLLLHDSIQIPRQLGEVASFGGSNIEPSVRSCFQFANNKPEIEAALFLDWMRLEPQ
SMVWLPVLHRVAAAETAKHQAKCNICKECPIIGFRYRSLKHFNYDICQSCFFSGRVAKGHKMHYPMVEYC
TPTTSGEDVRDFAKVLKNKFRTKRYFAKHPRMGYLPVQTVLEGDNMETPVTLINFWPVDSAPASSPQLSH
DDTHSRIEHYASRLAEMENSNGSYLNDSISPNESIDDEHLLIQHYCQSLNQDSPLSQPRSPAQILISLES
EERGELERILADLEEENRNLQAEYDRLKQQHEHKGLSPLPSPPEMMPTSPQSPRDAELIAEAKLLRQHKG
RLEARMQILEDHNKQLESQLHRLRQLLEQPQAEAKVNGTTVSSPSTSLQRSDSSQPMLLRVVGSQTSDSM
GEEDLLSPPQDTSTGLEEVMEQLNNSFPSSRGRNTPGKPMREDTM
SEQ ID NO: 34-micro-dystrophin (uDys5, SEQ ID NO: 4 in U.S. Pat. No. 10,479,821 B2)
Met Leu Trp Trp Glu Glu Val Glu Asp Cys Tyr Glu Arg Glu Asp Val
1 5 10 15
Gln Lys Lys Thr Phe Thr Lys Trp Val Asn Ala Gln Phe Ser Lys Phe
                        20 25 30
Gly Lys Gln His Ile Glu Asn Leu Phe Ser Asp Leu Gln Asp Gly Arg
                35 40 45
Arg Leu Leu Asp Leu Leu Glu Gly Leu Thr Gly Gln Lys Leu Pro Lys
        50 55 60
Glu Lys Gly Ser Thr Arg Val His Ala Leu Asn Asn Val Asn Lys Ala
65 70 75 80
Leu Arg Val Leu Gln Asn Asn Asn Val Asp Leu Val Asn Ile Gly Ser
                                85 90 95
Thr Asp Ile Val Asp Gly Asn His Lys Leu Thr Leu Gly Leu Ile Trp
                        100 105 110
Asn Ile Ile Leu His Trp Gln Val Lys Asn Val Met Lys Asn Ile Met
                115 120 125
Ala Gly Leu Gln Gln Thr Asn Ser Glu Lys Ile Leu Leu Ser Trp Val
        130 135 140
Arg Gln Ser Thr Arg Asn Tyr Pro Gln Val Asn Val Ile Asn Phe Thr
145 150 155 160
Thr Ser Trp Ser Asp Gly Leu Ala Leu Asn Ala Leu Ile His Ser His
                                165 170 175
Arg Pro Asp Leu Phe Asp Trp Asn Ser Val Val Cys Gln Gln Ser Ala
                        180 165 190
Thr Gln Arg Leu Glu His Ala Phe Asn Ile Ala Arg Tyr Gln Leu Gly
                195 200 205
Ile Glu Lys Leu Leu Asp Pro Glu Asp Val Asp Thr Thr Tyr Pro Asp
        210 215 220
Lys Lys Ser Ile Leu Met Tyr Ile Thr Ser Leu Phe Gln Val Leu Pro
225 230 235 240
Gln Gln Val Ser Ile Glu Ala Ile Gln Glu Val Glu Met Leu Pro Arg
                                245 250 255
Pro Pro Lys Val Thr Lys Glu Glu His Phe Gln Leu His His Gln Met
                        260 265 270
His Tyr Ser Gln Gln Ile Thr Val Ser Leu Ala Gln Gly Tyr Glu Arg
                275 280 285
Thr Ser Ser Pro Lys Pro Arg Phe Lys Ser Tyr Ala Tyr Thr Gln Ala
        290 295 300
Ala Tyr Val Thr Thr Ser Asp Pro Thr Arg Ser Pro Phe Pro Ser Gln
305 310 315 320
His Leu Glu Ala Pro Glu Asp Lys Ser Phe Gly Ser Ser Leu Met Glu
                                325 330 335
Ser Glu Val Asn Leu Asp Arg Tyr Gln Thr Ala Leu Glu Glu Val Leu
                        340 345 350
Ser Trp Leu Leu Ser Ala Glu Asp Thr Leu Gln Ala Gln Gly Glu Ile
                355 360 365
Ser Asn Asp Val Glu Val Val Lys Asp Gln Phe His Thr His Glu Gly
        370 375 380
Tyr Met Met Asp Leu Thr Ala His Gln Gly Arg Val Gly Asn Ile Leu
365 390 395 400
Gln Leu Gly Ser Lys Leu Ile Gly Thr Gly Lys Leu Ser Glu Asp Glu
                                405 410 415
Glu Thr Glu Val Gln Glu Gln Met Asn Leu Leu Asn Ser Arg Trp Glu
                        420 425 430
Cys Leu Arg Val Ala Ser Met Glu Lys Gln Ser Asn Leu His Ser Tyr
                435 440 445
Val Pro Ser Thr Tyr Leu Thr Glu Ile Thr His Val Ser Gln Ala Leu
        450 455 460
Leu Glu Val Glu Gln Leu Leu Asn Ala Pro Asp Leu Cys Ala Lys Asp
465 470 475 480
Phe Glu Asp Leu Phe Lys Gln Glu Glu Ser Leu Lys Asn Ile Lys Asp
                                465 490 495
Ser Leu Gln Gln Ser Ser Gly Arg Ile Asp Ile Ile His Ser Lys Lys
                        500 505 510
Thr Ala Ala Leu Gln Ser Ala Thr Pro Val Glu Arg Val Lys Leu Gln
                515 520 525
Glu Ala Leu Ser Gln Leu Asp Phe Gln Trp Glu Lys Val Asn Lys Met
        530 535 540
Tyr Lys Asp Arg Gln Gly Arg Phe Asp Arg Ser Val Glu Lys Trp Arg
545 550 555 560
Arg Phe His Tyr Asp Ile Lys Ile Phe Asn Gln Trp Leu Thr Glu Ala
                                565 570 575
Glu Gln Phe Leu Arg Lys Thr Gln Ile Pro Glu Asn Trp Glu His Ala
                        560 565 590
Lys Tyr Lys Trp Tyr Leu Lys Glu Leu Gln Asp Gly Ile Gly Gln Arg
                595 600 605
Gln Thr Val Val Arg Thr Leu Asn Ala Thr Gly Glu Glu Ile Ile Gln
        610 615 620
Gln Ser Ser Lys Thr Asp Ala Ser Ile Leu Gln Glu Lys Leu Gly Ser
625 630 635 640
Leu Asn Leu Arg Trp Gln Glu Val Cys Lys Gln Leu Ser Asp Arg Lys
                                645 650 655
Lys Arg Leu Glu Glu Gln Ser Asp Gln Trp Lys Arg Leu His Leu Ser
                        660 665 670
Leu Gln Glu Leu Leu Val Trp Leu Gln Leu Lys Asp Asp Glu Leu Ser
                  675 680 685
Arg Gln Ala Pro Ile Gly Gly Asp Phe Pro Ala Val Gln Lys Gln Asn
        690 695 700
Asp Val His Arg Ala Phe Lys Arg Glu Leu Lys Thr Lys Glu Pro Val
705 710 715 720
Ile Met Ser Thr Leu Glu Thr Val Arg Ile Phe Leu Thr Glu Gln Pro
                                725 730 735
Leu Glu Gly Leu Glu Lys Leu Tyr Gln Glu Pro Arg Glu Leu Pro Pro
                        740 745 750
Glu Glu Arg Ala Gln Asn Val Thr Arg Leu Leu Arg Lys Gln Ala Glu
                755 760 765
Glu Val Asn Thr Glu Trp Glu Lys Leu Asn Leu His Ser Ala Asp Trp
        770 775 780
Gln Arg Lys Ile Asp Glu Thr Leu Glu Arg Leu Gln Glu Leu Gln Glu
785 790 795 800
Ala Thr Asp Glu Leu Asp Leu Lys Leu Arg Gln Ala Glu Val Ile Lys
                                805 810 815
Gly Ser Trp Gln Pro Val Gly Asp Leu Leu Ile Asp Ser Leu Gln Asp
                        820 825 830
His Leu Glu Lys Val Lys Ala Leu Arg Gly Glu Ile Ala Pro Leu Lys
                835 840 845
Glu Asn Val Ser His Val Asn Asp Leu Ala Arg Gln Leu Thr Thr Leu
        850 855 860
Gly Ile Gln Leu Ser Pro Tyr Asn Leu Ser Thr Leu Glu Asp Leu Asn
865 870 875 880
Thr Arg Trp Lys Leu Leu Gln Val Ala Val Glu Asp Arg Val Arg Gln
                                885 890 895
Leu His Glu Ala His Arg Asp Phe Gly Pro Ala Ser Gln His Phe Leu
                        900 905 910
Ser Thr Ser Val Gln Gly Pro Trp Glu Arg Ala Ile Ser Pro Asn Lys
                915 920 925
Val Pro Tyr Tyr Ile Asn His Glu Thr Gln Thr Thr Cys Trp Asp His
        930 935 940
Pro Lys Met Thr Glu Leu Tyr Gln Ser Leu Ala Asp Leu Asn Asn Val
945 950 955 960
Arg Phe Ser Ala Tyr Arg Thr Ala Met Lys Leu Arg Arg Leu Gln Lys
                                965 970 975
Ala Leu Cys Leu Asp Leu Leu Ser Leu Ser Ala Ala Cys Asp Ala Leu
                        980 985 990
Asp Gln His Asn Leu Lys Gln Asn Asp Gln Pro Met Asp Ile Leu Gln
                995 1000 1005
Ile Ile Asn Cys Leu Thr Thr Ile Tyr Asp Arg Leu Glu Gln Glu
        1010 1015 1020
His Asn Asn Leu Val Asn Val Pro Leu Cys Val Asp Met Cys Leu
        1025 1030 1035
Asn Trp Leu Leu Asn Val Tyr Asp Thr Gly Arg Thr Gly Arg Ile
        1040 1045 1050
Arg Val Leu Ser Phe Lys Thr Gly Ile Ile Ser Leu Cys Lys Ala
        1055 1060 1065
His Leu Glu Asp Lys Tyr Arg Tyr Leu Phe Lys Gln Val Ala Ser
        1070 1075 1080
Ser Thr Gly Phe Cys Asp Gln Arg Arg Leu Gly Leu Leu Leu His
        1085 1090 1095
Asp Ser Ile Gln Ile Pro Arg Gln Leu Gly Glu Val Ala Ser Phe
        1100 1105 1110
Gly Gly Ser Asn Ile Glu Pro Ser Val Arg Ser Cys Phe Gln Phe
        1115 1120 1125
Ala Asn Asn Lys Pro Glu Ile Glu Ala Ala Leu Phe Leu Asp Trp
        1130 1135 1140
Met Arg Leu Glu Pro Gln Ser Met Val Trp Leu Pro Val Leu His
        1145 1150 1155
Arg Val Ala Ala Ala Glu Thr Ala Lys His Gln Ala Lys Cys Asn
        1160 1165 1170
Ile Cys Lys Glu Cys Pro Ile Ile Gly Phe Arg Tyr Arg Ser Leu
        1175 1180 1185
Lys His Phe Asn Tyr Asp Ile Cys Gln Ser Cys Phe Phe Ser Gly
        1190 1195 1200
Arg Val Ala Lys Gly His Lys Met His Tyr Pro Met Val Glu Tyr
        1205 1210 1215
Cys Thr Pro Thr Thr Ser Gly Glu Asp Val Arg Asp Phe Ala Lys
        1220 1225 1230
Val Leu Lys Asn Lys Phe Arg Thr Lys Arg Tyr Phe Ala Lys His
        1235 1240 1245
Pro Arg Met Gly Tyr Leu Pro Val Gln Thr Val Leu Glu Gly Asp
        1250 1255 1260
Asn Met Glu Thr Asp Thr Met
        1265 1270
```

1. A method for treating a subject having muscular dystrophy, comprising:
administering to the subject a therapeutically effective amount of a first pharmaceutical composition comprising an RRM1 gene and an RRM2 gene operably coupled to a first regulatory cassette.

2. The method of claim 1, wherein the first pharmaceutical composition further comprises a first delivery vehicle.

3. The method of claim 1, further comprising:
administering to the subject a therapeutically effective amount of a second pharmaceutical composition comprising a micro-dystrophin gene operably coupled to a second regulatory cassette.

4. The method of claim 3, wherein the second pharmaceutical composition further comprises a second delivery vehicle.

5. The method of claim 3, wherein the first regulatory cassette comprises a cardiac muscle-specific regulatory cassette, and the second regulatory cassette comprises a striated muscle-specific regulatory cassette.

6. A method for treating a subject having muscular dystrophy, comprising:
administering to the subject a therapeutically effective amount of a first pharmaceutical composition comprising an RRM1 gene operably coupled to a first regulatory cassette in a first delivery vehicle.

7. The method of claim 6, further comprising:
administering to the subject a therapeutically effective amount of a second pharmaceutical composition comprising an RRM2 gene operably coupled to a second regulatory cassette in a second delivery vehicle.

8. The method of claim 7, further comprising:
administering to the subject a therapeutically effective amount of a third pharmaceutical composition comprising a micro-dystrophin gene operably coupled to a third regulatory cassette in a third delivery vehicle.

9. A method for prophylactically treating a subject at risk of developing muscular dystrophy, comprising:
administering to the subject a therapeutically effective amount of a first pharmaceutical composition comprising an RRM1 gene operably coupled to a first regulatory cassette in a first delivery vehicle.

10. The method of claim 9, further comprising:
administering to the subject a therapeutically effective amount of a second pharmaceutical composition comprising an RRM2 gene operably coupled to a second regulatory cassette in a second delivery vehicle.

11. The method of claim 10, further comprising:
administering to the subject a therapeutically effective amount of a third pharmaceutical composition comprising a micro-dystrophin gene operably coupled to a third regulatory cassette in a third delivery vehicle.

12. The method of claim 10, wherein the first delivery vehicle and the second delivery vehicle are separate delivery vehicles.

13. The method of claim 11, wherein the first delivery vehicle, the second delivery vehicle and the third delivery vehicles are separate delivery vehicles.

14. The method of claim 1, wherein the regulatory cassette is selected from the group consisting of a cardiac troponin T (cTnT) regulatory cassette and a miniaturized creatine kinase-based (CK8) regulatory cassette.

15. The method of claim 1, wherein the delivery vehicle is selected from the group consisting of an adeno-associated virus (AAV) vector or a recombinant adeno-associated virus (rAAV) vector.

16. The method of claim 1, wherein the muscular dystrophy is selected from at least one of myotonic muscular dystrophy, Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, and Emery-Dreifuss muscular dystrophy.

17. The method of claim 1, wherein the muscular dystrophy is selected from at least one of Duchenne muscular dystrophy and Becker muscular dystrophy.

18. The method of claim 1, wherein the delivery vehicle is a recombinant adeno-associated virus type 6 (rAAV6) vector.

19. The method of claim 1, wherein the subject is a mammal.

20. The method of claim 1, wherein the subject is a human.
